(19) United States
(12) Patent Application Publication
(10) Pub. No.: US 2005/0203119 A1
(43) Pub. Date: Sep. 15, 2005

(54) Dihydropyridine compounds for treating or preventing metabolic disorders

(76) Inventors: Mitsunori Ono, Lexington, MA (US); Yumiko Wada, Billerica, MA (US); Lijun Sun, Harvard, MA (US); Shoujun Chen, Bedford, MA (US); Teresa Przewloka, Tewksbury, MA (US); Shijie Zhang, Nashua, NH (US); Christopher Borella, Cambridge, MA (US); Keizo Koya, Chestnut Hill, MA (US); Kevin Foley, Cambridge, MA (US); Zhi-Qiang Xia, Acton, MA (US); Hao Li, Brookline, MA (US); Dan Zhou, Lexington, MA (US)

Correspondence Address: JONES DAY, 222 EAST 41ST ST, NEW YORK, NY, 10017, US

(21) Appl. No.: 10/939,252

(22) Filed: Sep. 10, 2004

Related U.S. Application Data
(60) Provisional application No. 60/561,264, filed on Apr. 9, 2004.
(60) Provisional application No. 60/502,353, filed on Sep. 10, 2003.

(51) Int. Cl. C07D 471/02; C07D 491/02; C07D 498/02; A61K 31/4745; A61K 31/4743; A61K 31/4741; A61K 31/47

(52) U.S. Cl. 514/300; 546/178; 546/122; 546/115; 546/114; 514/311; 514/302; 514/301

(57) ABSTRACT

This invention relates to dihydropyridine compounds of formula (I): 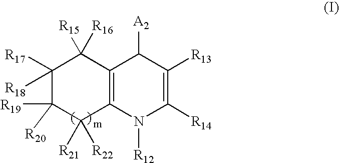
or a pharmaceutically acceptable salt, solvate, clathrate, or prodrug thereof wherein A₂, R₁₂, R₁₃, R₁₄, R₁₅, R₁₆, R₁₇, R₁₈, R₁₉, R₂₀, R₂₁, R₂₂, and m are defined herein, and compositions comprising such compounds. The invention also relates to methods of preventing or treating metabolic disorders, such as diabetes mellitus, and conditions and complications associated with diabetes mellitus, comprising administering to a subject in need thereof a compound of formula (1) or a composition comprising such a compound. The invention further relates to kits comprising a compound of formula (I).

### RELATED APPLICATIONS

This application claims the benefit of U.S. Provisional Application No. 60/502,353, filed on Sep. 10, 2003 and U.S. Provisional Application No. 60/561,264, filed on Apr. 9, 2004. The entire teaching of the above referenced provisional applications are incorporated herein by reference.

### FIELD OF THE INVENTION

The present invention relates to substituted dihydropyridine compounds and compositions comprising substituted dihydropyridine compounds. The invention further relates to methods for preventing or treating metabolic disorders, such as diabetes mellitus, and conditions and complications associated with diabetes mellitus, comprising administering to a subject in need thereof a substituted dihydropyridine compound, or a composition comprising such a compound. The invention still further relates to kits comprising a substituted dihydropyridine compound.

### BACKGROUND OF THE INVENTION

Metabolic disorders are conditions characterized by defective metabolism of sources used to store or use energy to produce the proteins, fats, and sugars needed by the body. For example, diabetes mellitus is a chronic, systemic disease characterized by abnormalities in the metabolism of carbohydrates, proteins, fats and insulin. The disease is generally characterized by hyperglycemia resulting from the body's inability to properly metabolize blood glucose. In a non-diabetic subject, the pancreas appropriately increases production of insulin to reduce glucose levels. Insulin is a hormone that induces the liver to metabolize glucose to glycogen. Diabetics, however, produce too little insulin, entirely cease producing insulin or progressively become resistant to the action of insulin. As a result, circulating levels of glucose remain dangerously high and in some cases, blood levels of insulin and lipids are also abnormal.

Approximately 17 million people in the United States, or 6% of the population, have diabetes. An estimated 11 million have been diagnosed, with another 6 million people unaware that they have the disease. Type I diabetes comprises 7 to 10 percent of all cases. Type I diabetes is typically an early onset disease characterized by the inability of the body to produce insulin. This is believed to be a result of an autoimmune response against the insulin-producing β-cells (or islet cells) of the pancreas. Because the β-cells are destroyed, sufferers of Type I diabetes do not produce insulin and must be treated with exogenous insulin for life.

Type II diabetes is a gradual onset disease that usually presents in middle age. The majority of Type II diabetics are obese, with most suffering from visceral obesity. These individuals tend to have high levels of circulating lipids (including cholesterol), which contributes to development of vascular complications. Type II diabetics usually present with a combination of insulin resistance (i.e., an impairment in the body's ability to respond to insulin) and reduced insulin production by the pancreas (β-cell exhaustion). Secondary complications of diabetes have serious clinical implications. Approximately 25 percent of all new cases of end-stage renal failure occur in patients with diabetes. About 20,000 amputations are carried out in patients with diabetes each year, representing approximately half of the non-traumatic amputations performed in the United States. Furthermore, diabetes is the leading cause of new cases of blindness, with approximately 5000 new cases occurring annually.

In Type II diabetics, dietary and other lifestyle modifications are the starting point for disease management. Oral hypoglycemic agents may also be used with the goal of trying to control blood glucose at normal or close to normal limits. The most common agents fall into five general categories: biguanides (such as metformin (Glucophage, Bristol Myers Squibb)), Perioxisomes Proliferator Activated Receptor γ (PPARγ) agonists (including thiazolidinediones such as pioglitazone (Actos, Lilly) and rosiglitazone (A vandia, GlaxoSmithKline)), insulinotropic agents (including secretagogues such as repaglinide (Prandin, Novo Nordisk)), sulphonylureas (such as glimepiride (Amaryl, A ventis) and glipizide (Glucotrol XL, Pfizer)) and α-glucosidase inhibitors (such as acarbose (Glucobay, Bayer)). Patients who do not respond fully to monotherapy may experience improved responses when combination therapy selected from two or more different categories is employed. Combination drugs include A vandaryl (PPAR gamma agonist (A vandia) and sulphonylurea (Amaryl), GSK/A ventis), Avandamet (PPAR gamma agonist (A vandia) and metformin, GSK), Glucovance (sulphonylurea and metformin, BMS), and Metaglip (glipizide and metformin, BMS). New drugs in development fall into additional categories, including PPAR α/γ agonists such as tesaglitazar (Galida, AstraZeneca), PPAR α/γ/δ agonists such as 677954 (GlaxoSmithKline), GLP-1 (such as exenatide, Lilly/Amylin) and dipeptidyl peptidase IV inhibitors (such as LAF 237, Novartis and MK-0431, Merck), glycogen phosphorylase inhibitors, tyrosine phophatase inhibitors, GLUT 4 mediated glucose transport modulators, imnunoregulatory vaccines and β3 adrenergic agonists.

Several dihydropyridine compounds are known to have cardiovascular activity (against, for example, hypertension, ischemic disorders, and congestive heart failure) and in some cases, CNS activity (against stroke, for example). Amlodipine is a member of this class of compounds. In addition, certain dihydropyridine compounds have been noted as having anti-diabetic activity (such as Cerebrocrast (Latvian Institute of Organic Synthesis); BAY-U-6751, BAY-R-3401, BAY-W-1807 and compounds set forth in U.S. Pat. No. 5,026,714 (Bayer); and U6751 (Merck)).

Despite these and other recent advances, there remains a need for additional agents that can reduce glucose levels, improve other abnormal parameters characterizing metabolic diseases (such as, for example, elevated lipid and insulin levels or reduced insulin sensitivity). prevent or delay the onset or progression of metabolic diseases. and/or reduce side effects associated with conventional metabolic disease therapy. In particular, there is a need for agents that can reduce glucose levels, improve other abnormal parameters characterizing metabolic diseases, prevent or delay the onset or progression of metabolic diseases, and/or reduce side effects associated with conventional metabolic disease therapy which have little or no cardiovascular effect. A need also remains for agents with new mechanisms of action that can be used alone or in combination with conventional active agents.

Citation of any reference in this section of this application is not to be construed as an admission that such reference is prior art to the present application.

### SUMMARY OF THE INVENTION

The present invention provides novel compounds and uses of those compounds in the prevention, treatment or management of a metabolic disorder, a symptom or complication thereof. The present invention also provides new uses for previously disclosed compounds. In particular, the invention provides methods for preventing, managing or treating metabolic disorders, such as diabetes mellitus, and conditions and complications associated with diabetes mellitus, refractory or non-responsive to previously disclosed therapies for such metabolic disorders.

The present invention provides compounds having the formula (I): 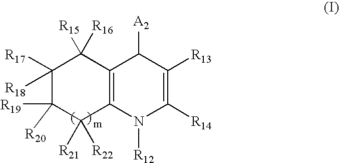
or a pharmaceutically acceptable salt, solvate, clathrate, or prodrug thereof wherein A₂, R₁₂, R₁₃, R₁₄, R₁₅, R₁₆, R₁₇, R₁₈, R₁₉, R₂₀, R₂₁, R₂₂, and m are defined below.

The present invention also provides compounds having the formula (II): 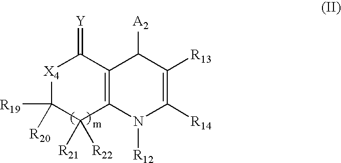
or a pharmaceutically acceptable salt, solvate, clathrate, or prodrug thereof wherein A₂, Y, X₄, R₁₂, R₁₃, R₁₄, R₁₉, R₂₀, R₂₁, R₂₂, and m are defined below.

The present invention also provides compounds having the formula (III) 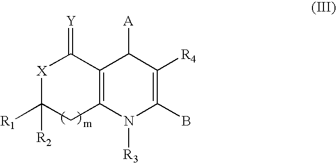
or a pharmaceutically acceptable salt, solvate, clathrate, or prodrug thereof wherein A, B, X, Y, R₁, R₂, R₃, R₄ and m are as defined below.

The invention also provides compounds having the formula (IV): 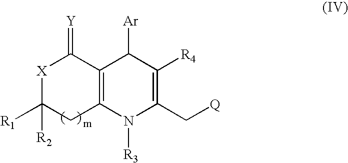
or a pharmaceutically acceptable salt, solvate, clathrate, hydrate, polymorph or prodrugs thereof wherein Ar, Q, X, Y, R₁, R₂, R₃, R₄ and m are as defined below.

The invention also provides compounds having the formula (V): 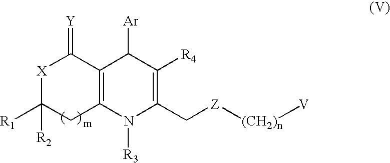
or a pharmaceutically acceptable salt, solvate, clathrate, hydrate, polymorph or prodrug thereof, wherein Ar, X, Y, Z, V, R₁, R₂, R₃, R₄, m and n are as defined below.

The invention also provides compounds having the formula (VI): 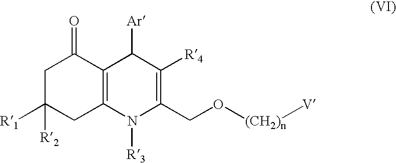
or a pharmaceutically acceptable salt, solvate, clathrate, hydrate, polymorph or prodrug thereof, wherein Ar′, V′, R₁′, R₂′, R₃′, R₄′ and n are as defined below.

The invention also provides compounds having the formula (VII): 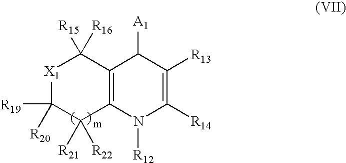
or a pharmaceutically acceptable salt, solvate, clathrate, or prodrug thereof wherein A₁, X₁, R₁₂, R₁₃, R₁₄, R₁₅, R₁₆, R₁₉, R₂₀, R₂₁, R₂₂, and m are defined below.

The invention also provides compounds having the formula (VIII): 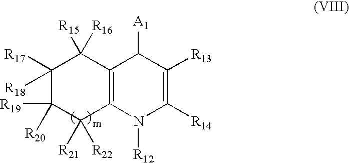
or a pharmaceutically acceptable salt, solvate, clathrate, or prodrug thereof wherein A₁, R₁₂, R₁₃, R₁₄, R₁₅, R₁₆, R₁₇, R₁₈, R₁₉, R₂₀, R₂₁, R₂₂, and m are defined below.

The invention also provides compounds having the formula (IX): 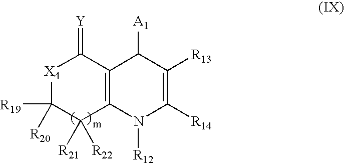
or a pharmaceutically acceptable salt, solvate, clathrate, or prodrug thereof wherein A₁, X₄, Y, R₁₂, R₁₃, R₁₄, R₁₉, R₂₀, R₂₁, R₂₂, and m are defined below.

The invention also provides compounds having the formula (X): 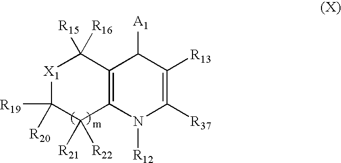
or a pharmaceutically acceptable salt, solvate, clathrate, or prodrug thereof wherein A₁, X₁, R₁₂, R₁₃, R₁₄, R₁₅, R₁₆, R₁₉, R₂₀, R₂₁, R₂₂, and m are defined below.

In one embodiment, the compounds of the invention are 3-substituted-dihydropyridine compounds or 4-substituted-1,4,5,6,7,8-hexahydroquinoline compounds characterized by an ability to reduce elevated blood glucose levels without a significant cardiovascular effect.

In another embodiment, the compounds of the invention are 3-substituted-dihydropyridine compounds or 4-substituted-1,4,5,6,7,8-hexahydroquinoline compounds characterized by an ability to reduce elevated blood glucose levels without significant acute toxicity.

The compounds of formula (I), (II), (III), (IV), (V), (VI), (VII), (VIII), (IX), (X), or Table 1, or pharmaceutically acceptable salts, solvates, clathrates, hydrates, polymorphs or prodrugs thereof, are particularly useful for preventing, treating, managing or ameliorating metabolic disorders (including, but not limited to diabetes mellitus, conditions associated with diabetes mellitus and certain complications thereof) or a symptom thereof. In a specific embodiment, the compounds of formula (I), (II), (III), (IV), (V), (VI), (VII), (VIII), (IX), (X), or Table 1, or pharmaceutically acceptable salts, solvates, clathrates, hydrates, polymorphs or prodrugs thereof, are used for preventing, treating, managing or ameliorating diabetes mellitus type I and/or type II, and conditions and complications associated therewith.

The present invention provides pharmaceutical compositions comprising an effective amount of a compound formula (I), (II), (III), (IV), (V), (VI), (VII), (VIII), (IX), (X), or Table 1, or pharmaceutically acceptable salts, solvates, clathrates, hydrates, polymorphs or prodrugs thereof, and a pharmaceutically acceptable carrier or vehicle. These compositions may further comprise additional agents. These compositions are useful for treating or preventing metabolic disorders, such as diabetes mellitus, conditions associated with diabetes mellitus and certain complications thereof.

The present invention also provides also methods for treating, preventing or managing a metabolic disorder, said methods comprising administering to a subject in need thereof a compound of formula (I), (II), (III), (IV), (V), (VI), (VII), (VIII), (IX), (X), or Table 1, or a pharmaceutically acceptable salt, solvate, clathrate, or prodrug thereof, or administering a pharmaceutical composition comprising a compound of formula (I), (II), (III), (IV), (V), (VI), (VII), (VIII), (IX), (X), or Table 1, or a pharmaceutically acceptable salt, solvate, clathrate, or prodrug thereof. These methods may also comprise administering to the subject an additional agent separately or in a combination composition with the compound of formula (I), (II), (III), (IV), (V), (VI), (VII), (VIII), (IX), (X), or Table 1, or a pharmaceutically acceptable salt, solvate, clathrate, or prodrug thereof.

In a specific embodiment, the invention provides a method for reducing blood glucose levels, said method comprising administering to a subject in need thereof an effective amount of a compound of formula (I), (II), (III), (IV), (V), (VI), (VII), (VIII), (IX), (X), or Table 1, or a pharmaceutically acceptable salt, solvate, clathrate, or prodrug thereof, or administering a pharmaceutical composition comprising an effective amount of a compound of formula (I), (II), (III), (IV), (V), (VI), (VII), (VIII), (IX), (X), or Table 1, or a pharmaceutically acceptable salt, solvate, clathrate, or prodrug thereof.

In another embodiment, the invention provides a method of improving blood lipid levels in a subject in need thereof, said method comprising administering to a subject in need thereof an effective amount of a compound of formula (I), (II), (III), (IV), (V), (VI), (VII), (VIII), (IX), (X), or Table 1, or a pharmaceutically acceptable salt, solvate, clathrate, or prodrug thereof, or administering a pharmaceutical composition comprising an effective amount of a compound of formula (I), (II), (III), (IV), (V), (VI), (VII), (VIII), (IX), (X), or Table 1, or a pharmaceutically acceptable salt, solvate, clathrate, or prodrug thereof. In accordance with these embodiments, a compound of formula (I), (II), (III), (IV), (V), (VI), (VII), (VIII), (IX), (X), or Table 1, may be administered in combination with other therapies (e.g., prophylactic or therapeutic agents). Examples of such therapies include, but are not limited to, dietary therapy, anti-diabetic agents, anti-obesity agents and lipid lowering agents.

In another embodiment, the invention provides a method of improving blood insulin levels, said method comprising administering to a subject in need thereof an effective amount of a compound of formula (I), (II), (III), (IV), (V), (VI), (VII), (VIII), (IX), (X), or Table 1, or a pharmaceutically acceptable salt, solvate, clathrate, or prodrug thereof, or administering a pharmaceutical composition comprising an effective amount of a compound of formula (I), (II), (III), (IV), (V), (VI), (VII), (VIII), (IX), (X), or Table 1, or a pharmaceutically acceptable salt, solvate, clathrate, or prodrug thereof.

In another embodiment, the invention provides a method of improving insulin sensitivity, said method comprising administering to a subject in need thereof an effective amount of a compound of formula (I), (II), (III), (IV), (V), (VI), (VII), (VIII), (IX), (X), or Table 1, or a pharmaceutically acceptable salt, solvate, clathrate, or prodrug thereof, or administering a pharmaceutical composition comprising an effective amount of a compound of formula (I), (II), (III), (IV), (V), (VI), (VII), (VIII), (IX), (X), or Table 1, or a pharmaceutically acceptable salt, solvate, clathrate, or prodrug thereof. In accordance with these embodiments, a compound of formula (I), (II), (III), (IV), (V), (VI), (VII), (VIII), (IX), (X), or Table 1 can be administered in combination with other therapies (e.g., prophylactic or therapeutic agents).

In another embodiment, the invention provides a method of achieving two or more of the following: (i) reducing blood glucose levels, (ii) improving blood lipid levels, (iii) improving blood insulin levels, and (iv) improving insulin sensitivity, said method comprising administering to a subject in need thereof an effective amount of a compound of formula (I), (II), (III), (IV), (V), (VI), (VII), (VIII), (IX), (X), or Table 1, or a pharmaceutically acceptable salt, solvate, clathrate, or prodrug thereof or a pharmaceutical composition comprising an effective amount of a compound of formula (I), (II), (III), (IV), (V), (VI), (VII), (VIII), (IX), (X), or Table 1, or a pharmaceutically acceptable salt, solvate, clathrate, or prodrug thereof. In accordance with these embodiments, a compound of formula (I), (II), (III), (IV), (V), (VI), (VII), (VIII), (IX), (X), or Table 1 may be administered in combination with other therapies (e.g., prophylactic or therapeutic agents).

In certain embodiments, the present invention encompasses prophylactic and/or therapeutic protocols that provide better prophylactic or therapeutic profiles than current single agent therapies or combination therapies for metabolic disorders, such as diabetes mellitus, and conditions associated and complications associated with diabetes mellitus.

The present invention provides kits comprising, in one or more containers, one or more compound of formula (I), (II), (III), (IV), (V), (VI), (VII), (VIII), (IX), (X), or Table 1. In certain embodiments, a kit of the invention comprises one or more compound of formula (I), (II), (III), (IV), (V), (VI), (VII), (VIII), (IX), (X), or Table 1 and a device for administering the one or more compounds.

### BRIEF DESCRIPTION OF THE FIGURES

FIG. 1 displays the results of an oral glucose tolerance test in the ob/ob mouse model of Type II diabetes using Compound 36 (25 mg/kg), rosiglitazone (1 mg/kg) and metformin (100 mg.kg).

FIG. 2 is a bar chart showing the effects of administration of Compound 1 (50 mg/kg) alone and in combination with rosiglitazone (1 mg/kg) on blood glucose reduction in the db/db mouse model of Type II diabetes.

FIG. 3 displays the results of an intraperitoneal glucose tolerance test in db/db mice dosed with vehicle, Compound 12, 199, 1, or 39.

FIG. 4 displays the results of an oral glucose tolerance test in db/db mice dosed with vehicle, Compound 39 or metformin.

FIG. 5 displays the results of an intraperitoneal glucose tolerance test in db/db mice dosed with vehicle, Compound 39, metformin, or a combination of Compound 39 and metformin.

FIG. 6 displays the results of a seven day baseline glucose study in db/db mice in which mice were orally dosed once daily with vehicle, metformin, Compound 16, Compound 39, Compound 45, Compound 65, Compound 66, Compound 68, Compound 69, Compound 197, Compound 215, or Compound 229.

FIG. 7 displays the results of a seven day baseling glucose study in KK-A^{y} mice in which mice were orally dosed once daily with vehicle, Compound 39, rosiglitazone, Compound 39 and rosiglitazone, metformin, or Compound 39 and metformin.

FIG. 8 displays the results of a seven day baseline glucose study in db/db mice in which mice were orally dosed once daily with vehicle, rosiglitazone, Compound 39 and rosiglitazone, metformin, or Compound 39 and metformin.

FIG. 9 displays the results of a seven day baseline glucose study in ZDF rats in which rats were orally dosed once daily with vehicle, Compound 39, metformin, or a combination of Compound 39 and metformin.

FIG. 10 displays the results of an oral glucose tolerance test in ZDF rats in which the rats were orally dosed with vehicle, Compoud 39, metformin, or Compound 39 and metformin.

### DETAILED DESCRIPTION OF THE INVENTION

The present invention provides compounds and uses of said compounds. The present invention encompasses the use of the compounds of the invention for the prevention, treatment, management and/or amelioration of a metabolic disorder or a symptom thereof. In particular, the present invention encompasses the use of compounds of the invention to reduce blood glucose levels (preferably, normalize blood glucose levels), improve abnormal blood insulin levels (preferably, normalize blood insulin levels), improve lipid metabolism, reduce cholesterol, and/or improve insulin sensitivity (preferably, normalize insulin sensitivity).

In certain embodiments, the present invention encompasses treatment protocols that provide better prophylactic or therapeutic profiles than current single agent therapies or combination therapies for a metabolic disorder or one or more symptoms thereof. In particular, the invention provides prophylactic and therapeutic protocols for the prevention, treatment, management, and/or amelioration of a metabolic disorder or a symptom thereof, comprising administering to a subject in need thereof an effective amount of one or more compounds of the invention alone or in combination with an effective amount of at least one other therapy other than a compound of the invention.

The present invention provides for pharmaceutical compositions and kits comprising one or more compounds of the invention for use in the prevention, treatment, management or amelioration of a metabolic disorder or a symptom thereof. The present invention also provides for pharmaceutical compositions and kits comprising one or more compounds of the invention and one or more additional agents for use in the prevention, treatment, management, or amelioration of a metabolic disorder or a symptom thereof.

A. Terminology

Unless otherwise specified, the below terms used herein are defined as follows:

As used herein, the term “alkyl” or “(C₁-C₁₀)alkyl” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. The term “(C₁-C₆)alkyl” means a saturated straight chain or branched non-cyclic hydrocarbon having from 1 to 6 carbon atoms. Representative (C₁-C₆)alkyl groups are those shown above having from 1 to 6 carbon atoms. Alkyl groups included in compounds of this invention may be optionally substituted with one or more conventionally used alkyl substituents, such as —NH₂, —NH—(C₁-C₆)alkyl, —N[(C₁-C₆)alkyl]₂, —O—(C₁-C₆)alkyl, —S—(C₁-C₆)alkyl, oxo, halo, acyl (e.g., —C(O)R₃₀, —C(O)OR₃₀, —OC(O)R₃₀, —C(O)NR₂₈R₂₉, and NR₃₀C(O)R₂₈, wherein R₂₈, R₂₉, and R₃₀ are defined below), nitro, hydroxyl, cyano, aryl, —(C₁-C₆)-aryl, —O-aryl, —S-aryl, —NH-aryl, —N(aryl)₂-(C₃-C₁₀)cycloalkyl, —O—(C₃-C₁₀)cycloalkyl, —S—(C₃-C₁₀)cycloalkyl, —NH—(C₃-C₁₀ )cycloalkyl, —N—[(C₃-C₁₀)cycloalkyl]₂, 3-7 membered monocyclic heterocycle, —O—(3-7 membered monocyclic heterocycle), —NH—(3-7 membered monocyclic heterocycle), —N-[(3-7 membered monocyclic heterocycle)]2, —S—(3-7 membered monocyclic heterocycle), and the like. In addition, any carbon in the alkyl segment may be substituted with carbonyl (C═O) or thiocarbonyl (C═S).

As used herein, the term “alkenyl” or “(C₂-C₁₀)alkenyl” means a saturated straight chain or branched non-cyclic hydrocarbon having from 2 to 10 carbon atoms and having at least one carbon-carbon double bond. Representative straight chain and branched (C₂-C₁₀)alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl and the like.

As used herein, the term “alkynyl” or “(C₂-C₁₀)alkynyl” means a saturated straight chain or branched non-cyclic hydrocarbon having from 2 to 10 carbon atoms and having at lease one carbon-carbon triple bond. Representative straight chain and branched (C₂-C₁₀)alkynyls include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl and the like.

As used herein, the term “cycloalkyl” or “(C₃-C₁₀)cycloalkyl” means a saturated cyclic alkyl radical having from 3 to 10 carbon atoms. Representative (C₃-C₁₀)cycloalkyls include cyclopropyl, 1-methylcyclopropyl, cyclobutyl, cyclopentyl, cyclohexyl, cycloheptyl, cyclooctyl, cyclononyl, and cyclodecyl.

As used herein, the term “bicycloalkyl” or “(C₈-C₁₄)bicycloalkyl” means a bi-cyclic alkyl system having from 8 to 14 carbon atoms and at least one saturated cyclic alkyl ring. Representative (C₈-C₁₄)bicyclocycloalkyls include indanyl, 1,2,3,4-tetrahydronaphthyl, 5,6,7,8-tetrahydronaphthyl, perhydronaphthyl and the like.

As used herein, the term “cycloalkenyl” or “(C₅-C₁₀)cycloalkenyl” means a cyclic non-aromatic alkyl radical having at least one carbon-carbon double bond in the cyclic system and from 5 to 10 carbon atoms. Representative (C₅-C₁₀)cycloalkenyls include cyclopentenyl, cyclopentadienyl, cyclohexenyl, cyclohexadienyl, cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl and the like.

As used herein, Uhe term “haloalkyl” means and alkyl group in which one or more (including all) the hydrogen radicals are replaced by a halo group, wherein each halo group is independently selected from —F, —Cl, —Br, and —I. The term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

As used herein, the term “heteroalkyl” is an alkyl group in which one or more carbon atoms have been substituted with a heteroatom, wherein each heteroatom substitution is, independently, selected from the group consisting of oxygen (—O—), sulfur (—S—), or nitrogen (NR₂₇—), wherein R₂₇ is defined below.

As used herein, the term an “aromatic ring” or “aryl” means a monocyclic or polycyclic-aromatic radical comprising carbon and hydrogen atoms. Examples of suitable aryl groups include, but are not limited to, phenyl, tolyl, anthracenyl, quinolinyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more conventional aryl substituents (including without limitation alkyl (preferably, lower alkyl), hydroxy, alkoxy (preferably, lower alkoxy), alkylthio, cyano, halo, amino, and nitro). In one embodiment, the aryl group is substituted with deuterium (e.g., one or more hydrogen radicals are replaced with a deuterium atom). Preferably, the aryl group is a monocyclic ring, wherein the ring comprises 6 carbon atoms, referred to herein as “(C₆)aryl.”

As used herein, the term “aralkyl” means an aryl group that is attached to another group by a (C₁-C₆)alkylene group. Representative aralkyl groups include benzyl, 2-phenyl-ethyl, naphth-3-yl-methyl and the like.

As used herein, the term “alkylene” refers to an alkyl group that has two points of attachment. The term “(C₁-C₆)alkylene” refers to an alkylene group that has from one to six carbon atoms. Non-limiting examples of alkylene groups include methylene (—CH₂—), ethylene (—CH₂CH₂—), n-propylene (—CH₂CH₂CH₂—), isopropylene (—CH₂CH(CH₃)—), and the like.

As used herein, the term “3 to 7 membered monocycle” means a monocyclic group having at least one heteroatom selected from O, N or S, and which has 2-6 carbon atoms, which may be saturated, unsaturated or aromatic, including (but not limited to): piperidinyl, piperazinyl, 2-oxopiperazinyl, 2-oxopiperidinyl, 2-oxopyrrolidinyl, 2-oxazepinyl, azepinyl, 4-piperidonyl, pyridyl, N-oxo-pyridyl, pyridazinyl, pyrimidinyl, pyrazinyl, triazinyl, thiazoly, imidazolyl, oxazolyl, isoxazolyl, pyazolyl, pyrrolyl, [1,2,4]oxadiazolyl, triazolyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolane, furanyl, dihydrofuranyl-2-one, thienyl, and tetrahydro-1,1-dioxothienyl. Preferred 3 to 7 membered monocyclic heterocycles are 5 membered monocyclic heterocycles. A heteroatom may be substituted with a protecting group known to those of ordinary skill in the art, for example, the hydrogen on a nitrogen may be substituted with a tert-butoxycarbonyl group. Furthermore, the monocyclic heterocyclic ring may be optionally substituted with one or more conventional heterocyclic ring substituents (including without limitation a halogen atom, an alkyl radical, or aryl radical). In addition, the point of attachment of the monocyclic heterocyclic ring to another group may be at either a carbon atom or a heteroatom of the monocyclic heterocyclic ring. Only stable isomers of such substituted heterocyclic groups are contemplated in this definition.

As used herein, the term “8 to 12 membered bicyclic heterocycle” means a bicyclic group having at least one atom selected from O, N or S, and which has 7-11 carbon atoms, which may be saturated, unsaturated or aromatic, including (but not limited to) quinolinyl, benzo[1,3]dioxolyl, benzo[1,4]dioxinyl, chromenyl, indolyl, indolizinyl, imidazo[1,5-a]pyridyl, imidazo[1,2-a]pyridyl, isoindolyl (e.g., isoindole-1,3-dione), and thiochromenyl. A heteroatom may be substituted with a protecting group known to those of ordinary skill in the art, for example, the hydrogen on a nitrogen may be substituted with a tert-butoxycarbonyl group. Furthermore, the bicyclic heterocyclic rings may be optionally substituted with one or more conventional heterocyclic ring substituents (including without limitation a halogen atom, an alkyl radical, or aryl radical). In addition, the point of attachment of the bicyclic heterocyclic ring to another group may be at either a carbon atom or a heteroatom of the bicyclic heterocyclic ring. Only stable isomers of such substituted heterocyclic groups are contemplated in this definition.

As used herein, the term “heterocycle” refers collectively to moncyclic heterocycles and bicyclic heterocycles.

As used herein, the term “heteroaromatic”, “heteroaryl” or like terms means a monocyclic or polycyclic heteroaromatic ring comprising carbon atom ring members and one or more heteroatom ring members (such as, for example, oxygen, sulfur or nitrogen). Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, a isoxazolyl, quinolinyl, pyrazolyl, isothiazolyl, pyridazinyl, pyrimidinyl, pyrazinyl, a triazinyl, triazolyl, thiadiazolyl, isoquinolinyl, indazolyl, a substituted or unsubstituted benzoxazolyl, a substituted or unsubstituted benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzo(b)thienyl. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.

As used herein, the term “(C₅)heteroaryl” means an aromatic heterocyclic ring of 5 members, wherein at least one carbon atom of the ring is replaced with a heteroatom such as, for example, oxygen, sulfur or nitrogen. Representative (C₅)heteroaryls include furanyl, thienyl, pyrrolyl, oxazolyl, imidazolyl, thiazolyl, isoxazolyl, pyrazolyl, isothiazolyl, pyrazinyl, triazolyl, thiadiazolyl, and the like.

As used herein, the term “(C₆)heteroaryl” means an aromatic heterocyclic ring of 6 members, wherein at least one carbon atom of the ring is replaced with a heteroatom such as, for example, oxygen, nitrogen or sulfur. Representative (C₆)heteroaryls include pyridyl, pyridazinyl, pyrazinyl, triazinyl, tetrazinyl and the like.

As used herein, the term “heteroaralkyl” means a heteroaryl group that is attached to another group by a (C₁-C₆)alkylene. Representative heteroaralkyls include 2-(pyridin-4-yl)-propyl, 2-(thien-3-yl)-ethyl, imidazol-4-yl-methyl and the like.

As used herein, the term “heteroaralkoxy” refers to a heteroaryl group which is linked to another group by a —(C₁-C₆)alkyl-O— linker, wherein the heteroaryl group is attached to the alkyl portion of the linker and other group is attached to the oxygen atom. Representative heteroaralkoxy groups include pyridine-3-yl-methoxy, 2-(furan-2-yl)-ethoxy and the like.

As used herein, the term “heterocycloalkyl” means a cycloalkyl group in which at one to four carbon atoms have been replaced with a heteroatom, wherein each heteroatom is independently selected from —O—, —S—, and —NR₂₇—, wherein R₂₇ is defined below. Representative heterocycloalkyl groups include piperidinyl, piperazinyl, 2-oxopiperazinyl, 2-oxopiperidinyl, 2-oxopyrrolidinyl, 4-piperidonyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolane, tetrahydrofuranyl, and tetrahydrothienyl. A heteroatom may be substituted with a protecting group known to those of ordinary skill in the art, for example, the hydrogen on a nitrogen may be substituted with a tert-butoxycarbonyl group. Furthermore, the heterocycloalkyl ring may be optionally substituted with one or more conventional heterocycloalkyl ring substituents (including without limitation a halogen atom, an alkyl radical, or aryl radical). In addition, the point of attachment of the heterocycloalkyl ring to another group may be at either a carbon atom or a heteroatom of the heterocycloalkyl ring. Only stable isomers of such substituted heterocycloalkyl groups are contemplated in this definition.

As used herein, the term “halogen” or “halo” means —F, —Cl, —Br or —I.

As used herein the term “substituent” or “substituted” means that a hydrogen radical on a compound or group is replaced with any desired group that do not substantially adversely affect the desired activity of the compound. Examples of preferred substituents are those found in the exemplary compounds and embodiments disclosed herein, as well as halogen (chloro, iodo, bromo, or fluoro); C₁₋₆ alkyl; C₂₋₆ alkenyl; C₂₋₆ alkynyl; hydroxyl; C₁₋₆ alkoxyl; C₁₋₆ alkyl-O—C₁₋₆ alkyl (substituted or unsubstituted); amino; nitro; thiol; thioether; imine; cyano; amido; phosphonato; phosphine; carboxyl; thiocarbonyl; sulfonyl; sulfonamide; ketone; aldehyde; ester; oxygen (═O); haloalkyl (e.g., trifluoromethyl); carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl); carbocyclic or heterocyclic, monocyclic or fused or non-fused polycyclic aryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); o-lower alkyl; o-aryl, aryl; aryl-lower alkyl; CO₂CH₃; CONH₂; OCH₂CONH₂; NH₂; SO₂NH₂; OCHF₂; CF₃; OCF₃; and such moieties may also be optionally substituted by a fused-ring structure or bridge, for example —OCH₂O—. These substituents may optionally be further substituted with a substituent selected from such groups. In certain embodiments, the term “substituent” or the adjective “substituted” refers to a substituent selected from the group consisting of an alkyl, an alkenyl, an alkynyl, a cycloalkyl, a cycloalkenyl, a heterocycloalkyl, an aryl, a heteroaryl, an aralkyl, a heteraralkyl, a haloalkyl, —C(O)NR₂₈R₂₉, —NR₃₀C(O)R₃₁, a halo, —OR₃₀, cyano, nitro, a haloalkoxy, —C(O)R₃₀, —NR₂₈R₂₉, —SR₃₀, —C(O)OR₃₀, —OC(O)R₃₀, —NR₃₀C(O)NR₂₈R₂₉, —OC(O)NR₂₈R₂₉, —NR₃₀C(O)OR₃₁, —S(O)ᵣR₃₀, —S(O)ᵣNR₂₈R₂₉, ═O, ═S, and ═N—R₃₀, wherein R₂₈ and R₂₉, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₈ and R₂₉ taken together with the nitrogen to which they are attached is optionally substituted heterocycloalkyl or optionally substituted heteroaryl; and R₃₀ and R₃₁ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl.

A substituent has a substantially adverse affect on the desired activity of a compound if the compound is about 20% less active with the substituent than without it.

The terms “bioisostere” and “bioisosteric replacement” have the same meanings as those generally recognized in the art. Bioisosteres are atoms, ions, or molecules in which the peripheral layers of electrons can be considered identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Preferred bioisosteres of esters are compounds containing two sites for hydrogen bond acceptance. In one embodiment, the ester bioisostere is a 5 membered monocyclic heterocyclic ring.

As used herein, the terms “subject”, “patient” and “animal” are used interchangeably. The terms “subject” and “patient” refer to an animal (e.g., a bird such as a chicken, quail or turkey, or a mammal), preferably a mammal including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject is a human. In another embodiment the subject is refractory or non-responsive to current therapies for a metabolic disorder (e.g., diabetes mellitus type I and/or diabetes mellitus type II).

As used herein, the term “lower” refers to a group having up to four atoms. For example, a “lower alkyl” refers to an alkyl radical having from 1 to 4 carbon atoms, “lower alkoxy” refers to “—O—(C₁-C₄)alkyl and a “lower alkenyl” or “lower alkynyl” refers to an alkenyl or alkynyl radical having from 2 to 4 carbon atoms, respectively.

Unless indicated otherwise, the compounds of the invention containing reactive functional groups (such as (without limitation) carboxy, hydroxy, thiol, and amino moieties) also include protected derivatives thereof. “Protected derivatives” are those compounds in which a reactive site or sites are blocked with one ore more protecting groups. Examples of suitable protecting groups for hydroxyl groups include benzyl, methoxymethyl, allyl, trimethylsilyl, tert-butyldimethylsilyl, acetate, and the like. Examples of suitable amine protecting groups include benzyloxycarbonyl, tert-butoxycarbonyl, tert-butyl, benzyl and fluorenylmethyloxy-carbonyl (Fmoc). Examples of suitable thiol protecting groups include benzyl, tert-butyl, acetyl, methoxymethyl and the like. Other suitable protecting groups are well known to those of ordinary skill in the art and include those found in T. W. Greene, Protecting Groups in Organic Synthesis, John Wiley & Sons, Inc. 1981, incorporated by reference herein in its entirety.

As used herein, the term “compound(s) of this invention” and similar terms refers to a compound of formula (I), (II), (III), (IV), (V), (VI), (VII), (VIII), (IX), (X), or Table 1, or a pharmaceutically acceptable salt, solvate, clathrate, hydrate, polymorph or prodrug thereof, and also include protected derivatives thereof. In another embodiment, a “compound of the invention” is a 3-substituted-dihydropyridine compound or a 4-substituted-1,4,5,6,7,8-hexahydroquinoline compound characterized by an ability to reduce elevated blood glucose levels without a significant cardiovascular effect, wherein the core scaffold of the compounds is a dihydropyridine or a 1,4,5,6,7,8-hexahydroquinoline, respectively. In another embodiment, a “compound of the invention” is a 3-substituted-dihydropyridine or a 4-substituted-1,4,5,6,7,8-hexahydroquinoline compound characterized by an ability to reduce elevated blood glucose levels without significant acute toxicity, wherein the core scaffold of the compounds is a dihydropyridine or a 1,4,5,6,7,8-hexahydroquinoline, respectively.

The compounds of the invention may contain one or more chiral centers and/or double bonds and, therefore, exist as stereoisomers, such as double-bond isomers (i.e., geometric isomers), enantiomers, or diastereomers. According to this invention, the chemical structures depicted herein, including the compounds of this invention, encompass all of the corresponding compounds' enantiomers, diastereomers and geometric isomers, that is, both the stereomerically pure form (e.g., geometrically pure, enantiomerically pure, or diastereomerically pure) and isomeric mixtures (e.g., enantiomeric, diastereomeric and geometric isomeric mixtures). In some cases, one enantiomer, diastereomer or geometric isomer will possess superior activity or an improved toxicity or kinetic profile compared to other isomers. In those cases, such enantiomers, diastereomers and geometric isomers of compounds of this invention are preferred.

As used herein, the term “polymorph” means solid crystalline forms of a compound of the present invention or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “hydrate” means a compound of the present invention or a salt thereof, that further includes a stoichiometric or non-stoichiometric amount of water bound by non-covalent intermolecular forces.

As used herein, he term “clathrate” means a compound of the present invention or a salt thereof in the form of a crystal lattice that contains spaces (e.g., channels) that have a guest molecule (e.g., a solvent or water) trapped within.

As used herein and unless otherwise indicated, the term “prodrug” means a derivative of a compound that can hydrolyze, oxidize, or otherwise react under biological conditions (in vitro or in vivo) to provide a compound of this invention Prodrugs may only become active upon such reaction under biological conditions, or they may have activity in their unreacted forms. Examples of prodrugs contemplated in this invention include, but are not limited to, analogs or derivatives of compounds of formula formula (I), (II), (III), (IV), (V), (VI), (VII), (VIII), (IX), (X), or Table 1 that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of formula (I), (II), (III), (IV), (V), (VI), (VII), (VIII), (IX), (X), or Table 1 that comprise —NO, —NO₂, —ONO, or —ONO₂ moieties. Prodrugs can typically be prepared using well-known methods, such as those described by 1 BURGER'S MEDICINAL CHEMISTRY AND DRUG DISCOVERY (1995) 172-178, 949-982 (Manfred E. Wolff ed., 5^{th} ed).

As used herein and unless otherwise indicated, the terms “biohydrolyzable amide”, “biohydrolyzable ester”, “biohydrolyzable carbamate”, “biohydrolyzable carbonate”, “biohydrolyzable ureide” and “biohydrolyzable phosphate analogue” mean an amide, ester, carbamate, carbonate, ureide, or phosphate analogue, respectively, that either: 1) does not destroy the biological activity of the compound and confers upon that compound advantageous properties in vivo, such as uptake, duration of action, or onset of action; or 2) is itself biologically inactive but is converted in vivo to a biologically active compound. Examples of biohydrolyzable amides include, but are not limited to, lower alkyl amides, α-amino acid amides, alkoxyacyl amides, and alkylaminoalkylcarbonyl amides. Examples of biohydrolyzable esters include, but are not limited to, lower alkyl esters, alkoxyacyloxy esters, alkyl acylamino alkyl esters, and choline esters. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein, the terms “metabolic disease” and “metabolic disorder” are used interchangeably to refer to diseases and disorders associated with abnormal anabolism or assimilation and/or catabolism, including, without limitation diseases and disorders associated with abnormal carbohydrate metabolism, fat metabolism, and protein metabolism. Non-limiting examples of metabolic disorders include metabolic syndrome X diseases (including diabetes mellitus, obesity, hypertension, dyslipidemias and heart disease), Tangier disease, Wilson's disease (hepatolenticular degeneration), acromegaly, Addison's disease. Cushing's syndrome, Creutzfeldt-Jakob disease, hyperparathyroidism, multiple endocrine neoplasia Type 1, prolactinoma, galactosemia, glycogen storage diseases (e.g., Type O Liver, von Gierke's disease (Type IA), Type IB, Pompe's disease (Type II), Forbes' disease (Type III), Andersen's disease (Type IV), McArdle's disease (Type V), Hers' disease (Type VI), and Tarui's disease (Type VII)), hypoglycemia, Gaucher's disease, Fabry's disease, Mucopolysaccharidoses, Sandhoff Disease, Niemann-Pick Disease, aspartylglusomarinuria, biotinidase deficiency, carbohydrate deficient glycoprotein syndrome (CDGS), Crigler-Najjar syndrome, cystinosis, diabetes insipidus, glutaric aciduria, Hurler, lactic acidosis, long chain 3 hydroxyacyl CoA dehydrogenase deficiency (LCHAD) and also includes without limitation diseases and conditions associated with diabetes mellitus (diabetes mellitus type I and/or type II).

As used herein, the term “diabetes mellitus” refer to diabetes mellitus type I and/or type II. In certain embodiment, the term “diabetes mellitus” refers to diabetes mellitus type I. In other embodiments, the term “diabetes mellitus” refers to diabetes mellitus type II. In yet other embodiments, the term “diabetes mellitus” refers to diabetes mellitus type I and type II.

As used herein, the term “diseases and conditions associated with diabetes mellitus” and similar terms, refer to conditions associated with diabetes mellitus type I and/or type II, including, without limitation, hyperglycemia, hyperinsulinaemia, dyslipidemia (e.g., hyperlipidaemia), insulin resistance, impaired glucose metabolism, obesity, diabetic retinopathy, chronic microvascular complications, macular degeneration, cataracts, diabetic nephropathy, glomerulosclerosis, diabetic neuropathy, erectile dysfunction, premenstrual syndrome, vascular restenosis, and ulcerative colitis. Furthermore, “complications of diabetes mellitus” comprise, but are not restricted to: coronary heart disease, hypertension, angina pectoris, pain, numbness, muscle weakness, incontinence, myocardial infarction, arteriosclerosis, stroke, skin and connective tissue disorders, foot ulcerations, polyneuropathy, kidney disease, renal failure, metabolic acidosis, arthritis, osteoporosis and conditions of impaired glucose tolerance.

As used herein, the term “pharmaceutically acceptable salt,” is a salt formed from, for example, an acid and a basic group of one of the compounds of formula (I), (II), (III), (IV), (V), (VI), (VII), (VIII), (IX), (X), or Table 1. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The term “pharmaceutically acceptable salt” also refers to a salt prepared from a compound of formula (I), (II), (III), (IV), (V), (VI), (VII), (VIII), (IX), (X), or Table 1 having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N, N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like.

As used herein, the term “pharmaceutically acceptable solvate, ” is a solvate formed from the association of one or more pharmaceutically acceptable solvent molecules to one of the compounds of formula (I), (II), (III), (IV), (V), (VI), (VII), (VIII), (IX), (X), or Table 1. The term solvate includes hydrates (e.g., hemihydrate, monohydrate, dihydrate, trihydrate, tetrahydrate, and the like).

As used herein, the term “effective amount” refers to an amount of a compound of this invention which is sufficient to reduce or ameliorate the severity, duration, progression, or onset of a metabolic disorder, prevent the advancement of a metabolic disorder, cause the regression of a metabolic disorder, prevent the recurrence, development, onset or progression of a symptom associated with a metabolic disorder, or enhance or improve the prophylactic or therapeutic effect(s) of another therapy. In a specific embodiment, an “effective amount” refers to an amount of a compound which is sufficient to reduce blood glucose levels (preferably, normalize glucose levels), improve abnormal blood levels of insulin (preferably, normalize blood insulin levels), improve lipid metabolism, improve cholesterol levels and/or improve insulin sensitivity in a subject in need thereof or in an animal model of a particular metabolic disorder characterized by abnormal glucose levels, insulin levels, lipid metabolism or insulin sensitivity.

As used herein, the terms “improve” or “improving” mean to increase or decrease the level so that it is closer to or at a normal level (e.g., to increase or decrease glucose, insulin or lipid levels in the blood so that they are closer to a normal level). In one embodiment, “improve” or “improving” mean to lower the level. In one embodiment, “improve” or “improving” mean to increase the level.

Non-limiting examples of an effective amount of a compound of the invention are provided herein below. An effective amount of the compound when administered orally will typically range from about 0.1 mg/day to about 5000 mg/day (and preferably, about 1 mg/day to about 1000 mg/day and more preferably, about 10 to about 500 mg/day). These amounts may be administered in a single dosage form or may be administered in several (e.g., two to six, preferably two to four and more preferably, two or three) doses per day. Effective amounts will also vary, as recognized by those skilled in the art, depending on the diseases treated, route of administration, excipient usage, and the possibility of co-usage with other therapeutic treatments such as use of other agents.

As used herein, the terms “treat”, “treatment” and “treating” refer to the reduction or amelioration of the progression, severity and/or duration of a metabolic disorder, or the amelioration of one or more symptoms (preferably, one or more discernible symptoms) of a metabolic disorder resulting from the administration of one or more therapies (e.g., one or more therapeutic agents such as a compound of the invention). In specific embodiments, the terms “treat”, “treatment” and “treating” refer to the amelioration of at least one measurable physical parameter of a metabolic disorder, not necessarily discernible by the patient. In other embodiments the terms “treat”, “treatment” and “treating” refer to the inhibition of the progression of a metabolic disorder, either physically by, e.g., stabilization of a discernible symptom, physiologically by, e.g., stabilization of a physical parameter, or both. In other embodiments the terms “treat”, “treatment” and “treating” refer to the reduction in blood glucose levels (preferably, the normalization of blood glucose levels), the improvement in blood insulin levels (preferably, the normalization of blood insulin levels), the improvement in lipid metabolism, the reduction in cholesterol levels, the improvement in insulin sensitivity (preferably, the normalization of insulin sensitivity) and/or the inhibition or reduction in the onset, development or progression of one or more symptoms associated with a metabolic disorder. In yet other embodiments, the terms “treat”, “treatment” and “treating” refer to an improvement in the score in a diabetes assessment test, such as the Audit of Diabetes-Dependent Quality of Life, Appraisal of Diabetes Scale, Diabetes Care Profile, Diabetes Impact Measurement Scales, Diabetes Quality of Life Measure, Diabetes-Specific Quality-of-Life Scale, and Well-being Enquiry for Diabetics.

As used herein, the terms “prevent”, “prevention” and “preventing” refer to the reduction in the risk of acquiring or developing a given metabolic disorder, or the reduction or inhibition of the recurrence, onset or development of one or more symptoms of a given metabolic disorder. In a preferred embodiment, a compound of the invention is administered as a preventative measure to a patient, preferably a human, having a genetic predisposition to any of the disorders described herein.

As used herein, the terms “prophylactic agent” and “prophylactic agents” refer to any agent(s) which can be used in the prevention of a metabolic disorder or one or more symptoms thereof. In certain embodiments, the term “prophylactic agent” refers to a compound of the invention. In certain other embodiments, the term “prophylactic agent” does not refer a compound of the invention. Preferably, a prophylactic agent is an agent which is known to be useful for, or has been or is currently being used to prevent or impede the onset, development, progression and/or severity of a metabolic disorder.

As used herein, the terms “therapeutic agent” and “therapeutic agents” refer to any agent(s) which can be used in the treatment, management, or amelioration of a metabolic disorder or one or more symptoms thereof. In certain embodiments, the term “therapeutic agent” refers to a compound of the invention. In certain other embodiments, the term “therapeutic agent” refers does not refer to a compound of the invention. Preferably, a therapeutic agent is an agent which is known to be useful for, or has been or is currently being used for the treatment, management, prevention, or amelioration a metabolic disorder or one or more symptoms thereof.

As used herein, the term “synergistic” refers to a combination of a compound of the invention and another therapy (e.g., a prophylactic or therapeutic agent), which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with a metabolic disorder. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention, management or treatment of a metabolic disorder. In addition, a synergistic effect can result in improved efficacy of agents in the prevention, management or treatment of a metabolic disorder. Finally, a synergistic effect of a combination of therapies (e.g., a combination of prophylactic or therapeutic agents) may avoid or reduce adverse or unwanted side effects associated with the use of either therapy alone.

As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

As used herein, the term “in combination” refers to the use of more than one therapies (e.g., one or more prophylactic and/or therapeutic agents). The use of the term “in combination” does not restrict the order in which therapies (e.g., prophylactic and/or therapeutic agents) are administered to a subject with a metabolic disorder. A first therapy (e.g., a prophylactic or therapeutic agent such as a compound of the invention) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-diabetic agent) to a subject with a metabolic disorder.

As used herein, the terms “therapies” and “therapy” can refer to any protocol(s), method(s), and/or agent(s) that can be used in the prevention, treatment, management, or amelioration of a metabolic disorder or one or more symptoms thereof. In certain embodiments, the terms “therapy” and “therapies” refer to hormonal therapy, biological therapy, and/or other therapies useful in the prevention, management, treatment or amelioration of a metabolic disorder or one or more symptoms thereof known to one of skill in the area (e.g., skilled medical personnel).

A used herein, a “protocol” includes dosing schedules and dosing regimens. The protocols herein are methods of use and include prophylactic and therapeutic protocols.

As used herein, the terms “manage, ” “managing, ” and “management” refer to the beneficial effects that a subject derives from a therapy (e.g., a prophylactic or therapeutic agent), which does not result in a cure of the disease. In certain embodiments, a subject is administered one or more therapies (e.g., one or more prophylactic or therapeutic agents) to “manage” a disease so as to prevent the progression or worsening of the disease.

As used herein, the terms “non-responsive” and “refractory” describe patients treated with a currently available therapy (e.g., a prophylactic or therapeutic agent) for a metabolic disorder, which is not clinically adequate to relieve one or more symptoms associated with such disorder. Typically, such patients suffer from severe, persistently active disease and require additional therapy to ameliorate the symptoms associated with their metabolic disorder.

As used herein, a composition that “substantially” comprises a compound means that the composition contains more than about 80% by weight, more preferably more than about 90% by weight, even more preferably more than about 95% by weight, and most preferably more than about 97% by weight of the compound.

As used herein, a reaction that is “substantially complete” means that the reaction contains more than about 80% by percent yield of the desired product, more preferably more than about 90% by percent yield of the desired product, even more preferably more than about 95% by percent yield of the desired product, and most preferably more than about 97% by percent yield of the desired product.

As used herein, a racemic mixture means about 50% of one enantiomer and about 50% of is corresponding enantiomer relative to a chiral center in the molecule. The invention encompasses all enantiomerically-pure, enantiomerically-enriched, diastereomerically pure, diastereomerically enriched, and racemic mixtures of the compounds of the invention.

Enantiomeric and diastereomeric mixtures can be resolved into their component enantiomers or diastereomers by well known methods, such as chiral-phase gas chromatography, chiral-phase high performance liquid chromatography, crystallizing the compound as a chiral salt complex, or crystallizing the compound in a chiral solvent. In particular, complete separation of the enantiomers of Compound 39 is accomplished using a Chiralpak AS (4.5 mm×25 cm) column eluting with ethanol/2% triethylamine/CO₂ (15/85) (flow rate: 2 mL/min, 135/100 bar, 35° C.). Enantiomers and diastereomers can also be obtained from diastereomerically- or enantiomerically-pure intermediates, reagents, and catalysts by well known asymmetric synthetic methods.

The compounds of the invention are defined herein by their chemical structures and/or chemical names. Where a compound is referred to by both a chemical structure and a chemical name, and the chemical structure and chemical name conflict, the chemical structure is determinative of the compound's identity.

When administered to a patient, e.g., to a non-human animal for veterinary use or for improvement of livestock, or to a human for clinical use, the compounds of the invention are administered in isolated form or as the isolated form in a pharmaceutical composition. As used herein, “isolated” means that the compounds of the invention are separated from other components of either (a) a natural source, such as a plant or cell, preferably bacterial culture, or (b) a synthetic organic chemical reaction mixture. Preferably, the compounds of the invention are purified via conventional techniques. As used herein, “purified” means that when isolated, the isolate contains at least 95%, preferably at least 98%, of a compound of the invention by weight of the isolate either as a mixture of stereoisomers or as a diastereomeric or enantiomeric pure isolate.

As used herein, a composition that is “substantially free” of a compound means that the composition contains less than about 20% by weight, more preferably less than about 10% by weight, even more preferably less than about 5% by weight, and most preferably less than about 3% by weight of the compound.

Only those choices and combinations of substituents that result in a stable structure are contemplated. Such choices and combinations will be apparent to those of ordinary skill in the art and may be determined without undue experimentation.

The invention can be understood more fully by reference to the following detailed description and illustrative examples, which are intended to exemplify non-limiting embodiments of the invention.

B. The Compounds of the Invention

The present invention encompasses compounds having formulas (I), (II), (III), (IV), (V), (VI), (VII), (VIII), (IX), (X), and those set forth in Table 1 and pharmaceutically acceptable salts, solvates, clathrates, hydrates, polymorphs and prodrugs thereof. In one aspect, the invention provides compounds formula (I) as set forth below:

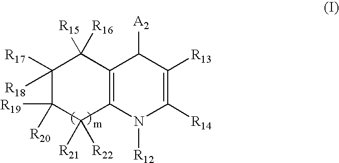

and pharmaceutically acceptable salts, solvates, clathrates, hydrates, polymorphs or prodrugs thereof, wherein:

mis 0, 1, or2;

A₂ is an optionally substituted aryl or an optionally substituted heteroaryl;

R₁₂ is —H or an alkyl;

R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;

R₁₄ is H or a substituent;

R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;

R₁₇ and R₁₈ are each, independently, —H or a substituent;

R₁₉, and R₂₀ are each, independently, —H or a substituent; or R₁₉ and R₂₀, together with the carbon to which they are attached, form an optionally substituted cycloalkyl;

R₂₁, and R₂₂, for each occurrence, are, independently, —H or a substituent;

R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;

R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;

R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and

R₂₇ is H, alkyl, aryl or acetyl.

Compounds of formula (I) and a pharmaceutically acceptable salts, solvates, clathrates, hydrates, polymorphs and prodrugs thereof are particularly useful for treating or preventing metabolic disorders, including diabetes mellitus, conditions associated with diabetes mellitus and certain complications thereof.

In one embodiment, in compounds represented by formula (I), there applies one or more (including all) of the following provisos:

1) R₁₄ is not lower alkyl, cyclopentyl, phenyl, bromomethyl, trifluoromethyl, —NH₂, nitro, —NHC(O)NH-phenyl, —SH, —SS-heterocycle, —S—(lower alkenyl), or —S—(cycloalkenyl);

2) when A₂ is o-chlorophenyl, R₁₄ is not a methyl substituted with a heteroaralkoxy;

3) when A₂ is o-(trifluoromethyl)-phenyl, R₁₄ is not —CH₂—S(O)ᵣ-phenyl, —CH₂—S(O)ᵣ-pyridyl, or —CH₂(CH₃)—S(O)ᵣ-phenyl, wherein r is 0, 1, or 2;

4) when A₂ is a chlorophenyl, R₁₄ is not —SH, —SCH₃, —SCH₂CH₃, —SCH(CH₂)CH₃, —SCH₂C(O)NH₂, or —SCH₂C(O)NH-(bromophenyl); and

5) when R₁₄ is —H, A₂ is not thiazolyl.

In another embodiment, in the compounds of formula (I), there applies one or more (including all) of the following provisos:

1) when R₁₄ is methyl, A₂ is not chlorophenyl, dichlorophenyl, p-nitrophenyl, or 5-chloro-benzo[1,3]dioxolyl;

2) when R₁₄ is isopropyl or cyclopentyl, either R₁₃ is not p-(trifluoromethyl)benzoyl or A₂ is not p-fluorophenyl; and

3) R₁₄ is not —NH₂.

In another embodiment, in the compounds of formula (I), there applies one or more (including all) of the following provisos:

1) when R₁₄ is methyl, A₂ is not chlorophenyl or 5-chloro-benzo[1,3]dioxolyl; and

2) when R₁₄ is cyclopentyl, R₁₃ is not p-(trifluoromethyl)-benzoyl.

In another embodiment, R₁₅ and R₁₆ together are ═O in compounds represented by formula (I).

In another embodiment, m is 1 in compounds represented by formula (I).

In another embodiment, in compounds represented by formula (I), A₂, m, R₁₂, R₁₃, R₁₄, R₁₅, R₁₆, R₁₇, R₁₈, R₁₉, R₂₀, R₂₁, and R₂₂ are selected from those included in specific exemplified compounds described herein.

In another aspect, the invention provides compounds of formula (II) as set forth below:

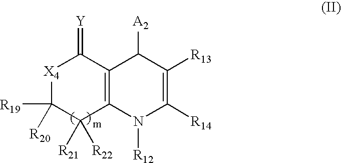

or a pharmaceutically acceptable salt, solvate, clathrate, or prodrug thereof wherein m, A₂, R₁₂, R₁₃, R₁₄, R₁₉, R₂₀, R₂₁, and R₂₂ are defined above; X₄ is O, S, or or —NR₂₃—; and Y is O or S.

Compounds of formula (II) and a pharmaceutically acceptable salts, solvates, clathrates, hydrates, polymorphs and prodrugs thereof are particularly useful for treating or preventing metabolic disorders, including diabetes mellitus, conditions associated with diabetes mellitus and certain complications thereof.

In one embodiment, in compounds represented by formula (II), there applies one or more (including all) of the following provisos:

1) R₁₄ is not a lower alkyl, a halomethyl, phenyl, cyano, or hydroxymethyl;

2) when R₁₄ is H or —NH₂, A₂ is not a substituted quinolinyl; and

3) when R₁₄ is 2-(N,N-dimethylamino)-ethyl or methoxymethyl, A₂ is not o-chlorophenyl or o-(trifluoromethyl)-phenyl.

In another embodiment, in compounds represented by formula (II), there applies one or two of the following provisos:

1) when R₁₄ is methyl, A₂ is not chlorothienyl, methylthienyl, 1-oxo-pyridin-3-yl, 1-oxo-2-chloropyridin-3-yl, 1-oxo-2-methylpyridin-3-yl, 2-phenyl-4-oxo-thiochromenyl, a substituted 4-oxo-benzopyranyl, or a substituted phenyl;

2) when R₁₄ is methyl, A₂ is not chlorothienyl, methylthienyl, 2-phenyl-4-oxo-thiochromenyl, a substituted 4-oxo-benzopyranyl, or a substituted phenyl;

3) when R₁₄ is methyl, A₂ is not 1-oxo-pyridin-3-yl, 1-oxo-2-chloropyridin-3-yl, or 1-oxo-2-methylpyridin-3-yl; and

4) when R₁₄ is methoxymethyl or (CH₃)₂NCH₂CH₂—, A₂ is not o-chlorophenyl.

In another embodiment, Y is ═O in compounds represented by formula (II).

In another embodiment, X₄ is —O— in compounds represented by formula (II).

In another embodiment, m is 1 in compounds represented by formula (II).

In another embodiment, in compounds represented by formula (II), A₂, Y, m, R₁₂, R₁₃, R₁₄, R₁₉, R₂₀, R₂₁, and R₂₂ are selected from those included in specific exemplified compounds described herein.

In another embodiment, in compounds represented by formula (I) or (II), A₂ is selected from the group consisting of a substituted or unsubstituted phenyl, a substituted or unsubstituted pyridyl, a substituted or unsubstituted 1-oxo-pyridyl, a substituted or unsubstituted furanyl, a substituted or unsubstituted anthracenyl, a substituted or unsubstituted fluorenyl, a substituted or unsubstituted indenyl, a substituted or unsubstituted azulenyl, a substituted or unsubstituted naphthyl, a substituted or unsubstituted 5,6,7,8-tetrahydronaphthyl, a substituted or unsubstituted benzo[1,3]dioxolyl, a substituted or unsubstituted thienyl, a substituted or unsubstituted pyrrolyl, a substituted or unsubstituted oxazolyl, a substituted or unsubstituted imidazolyl, a substituted or unsubstituted thiazolyl, a substituted or unsubstituted isoxazolyl, a substituted or unsubstituted quinoliny, a substituted or unsubstituted pyrazolyl, a substituted or unsubstituted isothiazolyl, a substituted or unsubstituted pyridazinyl, a substituted or unsubstituted pyrimidinyl, a substituted or unsubstituted pyrazinyl, a substituted or unsubstituted triazinyl, a substituted or unsubstituted triazolyl, a substituted or unsubstituted thiadiazolyl, a substituted or unsubstituted quinolyl, a substituted or unsubstituted isoquniolyl, a substituted or unsubstituted indazolyl, a substituted or unsubstituted benzoxazolyl, a substituted or unsubstituted benzofuryl, a substituted or unsubstituted benzothiazolyl, a substituted or unsubstituted indolizinyl, a substituted or unsubstituted imidazopyridyl, a substituted or unsubstituted isothiazolyl, a substituted or unsubstituted tetrazolyl, a substituted or unsubstituted benzimidazolyl, a substituted or unsubstituted benzoxazolyl, a substituted or unsubstituted benzothiazolyl, a substituted or unsubstituted benzothiadiazolyl, a substituted or unsubstituted benzoxadiazolyl, a substituted or unsubstituted indolyl, a substituted or unsubstituted tetrahydroindolyl, a substituted or unsubstituted azaindolyl, a substituted or unsubstituted imidazopyridyl, a substituted or unsubstituted qunizaolinyl, a substituted or unsubstituted purinyl, a substituted or unsubstituted pyrrolo[2,3]pyrimidyl, a substituted or unsubstituted pyrazolo[3,4]pyrimidyl, a substituted or unsubstituted imidazo[1,2-a]pyridyl, or a substituted or unsubstituted benzo(b)thienyl.

In another embodiment, in compounds represented by formula (I) or (II), A₂ is substituted with one or more substituents selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR₂₈R₂₉, —NR₃₀C(O)R₃₁, a halo, —OR₃₀, cyano, nitro, a haloalkoxy, —C(O)R₃₀, —NR₂₈R₂₉, —SR₃₀, —C(O)OR₃₀, —OC(O)R₃₀, —NR₃₀C(O)NR₂₈R₂₉, —OC(O)NR₂₈R₂₉, —NR₃₀C(O)OR₃₁, —S(O)ᵣR₃₀, and —S(O)ₚNR₂₈R₂₉, wherein:

R₂₈ and R₂₉, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₈ and R₂₉ taken together with the nitrogen to which they are attached is optionally substituted heterocycloalkyl or optionally substituted heteroaryl; and

R₃₀ and R₃₁ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl.

In another embodiment, in compounds represented by formula (I), A₂ is selected from the group consisting of a substituted or unsubstituted phenyl, a substituted or unsubstituted benzo[1,3]dioxolyl, a substituted or unsubstituted pyridyl, a substituted or unsubstituted indolyl, a substituted or unsubstituted quinolinyl, a substituted or unsubstituted 1-oxo-pyridyl, a substituted or unsubstituted pyridazinyl, a substituted or unsubstituted pyrimidinyl, a substituted or unsubstituted pyrazinyl, a substituted or unsubstituted furanyl, a substituted or unsubstituted thienyl, a substituted or unsubstituted [1,3,5]triazinyl, a substituted or unsubstituted thiazolyl, a substituted or unsubstituted imidazolyl, a substituted or unsubstituted oxazolyl, a substituted or unsubstituted indolizinyl, a substituted or unsubstituted imidazo[1,2-a]pyridyl, a substituted or unsubstituted 2,3-dihydro-benzo[1,4]dioxinyl, and a substituted or unsubstituted naphthyl.

In another embodiment, in compounds represented by formula (I) or (II), A₂ is substituted with one, two or three substituents selected from the group consisting of halo, nitro, —NR₃₂R₃₂, lower alkyl, lower alkoxy, lower alkyl sulfanyl, lower haloalkyl, phenyl, hydroxyl, cyano, and lower alkyl sulfonyl, wherein R₃₂, for each occurrence, is —H or a lower alkyl.

In another embodiment, in compounds represented by formula (I) or (II), A₂ is a phenyl or pyridyl ring substituted with one or more halo groups.

In another embodiment, in compounds represented by formula (I) or (II), A₂ is an unsubstituted pyridyl or 1-oxo-pyridyl;

In another embodiment, in compounds represented by formula (I) or (II), A₂ is a phenyl or pyridyl ring substituted with one or more —O-lower alkyl-NH—CH₂—CH(OH)—CH₂—O-phenyl groups.

In another embodiment, in compounds represented by formula (I) or (II), A₂ is a phenyl or pyridyl ring substituted with one or more methyl, phenyl, —NH₂, —CN, —CF₃, —OH, —OCH₃, methanesulfonyl, methylsufanyl, or —N₃ groups.

In another aspect, the invention provides compounds of formula (III) as set forth below:

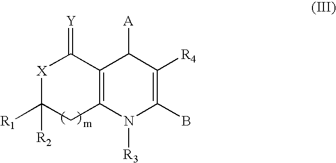

and pharmaceutically acceptable salts, solvates, clathrates, hydrates, polymorphs or prodrugs thereof wherein:

A and B are independently selected from —H, -halo, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —(C₁-C₆)alkyl-Z-(C₁-C₁₀)alkyl-R₁₁, —(C₁-C₁₀)alkyl-R₁₁, —(C₁-C₁₀)alkyl-N(R₅)(R₅), —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, —S(O)₂R₅ and a substituted or unsubstituted aromatic or heteroaromatic ring, wherein if the ring is substituted, the substituents are independently selected from the group consisting of substituted or unsubstituted lower alkyl, -halo, —CN, —N(R₅)(R₅), —OR₆, —C(O)R₅, —C(O)₂R₅, —OC(O)R₅, —NO₂, and —C(O)N(R₅)(R₅), or two adjacent carbon atoms on the ring are linked by the group —O—(CH₂)_{q}—O— to form a bicyclic ring system, wherein q is an integer selected from 1, 2, 3 or 4;

X is selected from the group consisting of O, S, —NR₅, and —C(R₅)(R₅);

Y is O or S;

Z is at each occurrence independently-O—, —S—, —N(R₅)—, —C(O)—, —OC(O)—, —C(O)N(R₅)C(O)—, substituted or unsubstituted —(C₁-C₁₀)alkyl-, substituted or unsubstituted —(C₂-C₁₀)alkenyl-, substituted or unsubstituted —(C₂-C₁₀)alkynyl-, substituted or unsubstituted —(C₃-C₁₀)cycloalkyl-, substituted or unsubstituted —(C₈-C₁₄)bicycloalkyl-, substituted or unsubstituted —(C₅-C₁₀)cycloalkenyl-, substituted or unsubstituted —(C₃-C₁₀)heterocycle-, substituted or unsubstituted phenyl, substituted or unsubstituted naphthyl, substituted or unsubstituted benzyl, —C(O)O—, —C(O)OC(R₅)(R₅)—, —N(R₅)C(O)—, —N(R₅)C(O)NR₅—, —C(O)NR₅—, —OC(O)O—, —S(O)N(R₅)—, —S(O)— or —S(O)₂—;

R₁ and R₂ are at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubsttuted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;

R₃ is at each occurrence independently —H, —C(O)R₅ or substituted or unsubstituted —(C₁-C₁₀)alkyl;

R₄ is at each occurrence independently —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)OR₅, —S(O)N(R₅)(R₅), —SR₅, —S(O)R₅, —S(O)₂R₅ or a substituted or unsubstituted bioisosteric replacement of an ester;

each R₅ is at each occurrence independently H or substituted or unsubstituted —(C₁-C₁₀)alkyl;

each R₆ is at each occurrence independently H, substituted or unsubstituted —(C₁-C₁₀)alkyl or —(CH₂)ₚ—N(R₅)—(C₁-C₆)alkyl optionally substituted with one or more —OR₅ —O-aryl groups;

R₁₁ is at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;

m is an integer selected from 0-2; and

p is an integer selected from 1-6.

Compounds of formula (III) and a pharmaceutically acceptable salts, solvates, clathrates, hydrates, polymorphs and prodrugs thereof are particularly useful for treating or preventing metabolic disorders, including diabetes mellitus, conditions associated with diabetes mellitus and certain complications thereof.

In one embodiment, in compounds represented by formula (III), R₁ and R₂ are both alkyl, preferably lower alkyl more preferably methyl.

In another embodiment, in compounds represented by formula (III), one of R₁ and R₂ is H and the other is alkyl, preferably lower alkyl more preferably isopropyl or methyl.

In another embodiment, in compounds represented by formula (III), R₃ is H.

In another embodiment, in compounds represented by formula (III), R₃ is other than H, such as methyl or ethyl.

In another embodiment, in compounds represented by formula (III), Y is O.

In another embodiment, in compounds represented by formula (III), A is a phenyl or pyridyl ring substituted with one or more halo groups.

In another embodiment, in compounds represented by formula (III), A is a phenyl or pyridyl ring substituted with one or more —O-lower alkyl-NH—CH₂—CH(OH)—CH₂—O-phenyl groups.

In another embodiment, in compounds represented by formula (III), A is a phenyl or pyridyl ring substituted with one or more methyl, phenyl, —NH₂, —CN, —CF₃, —OH or —N₃ groups.

In another embodiment, in compounds represented by formula (III), A is quinoline, indole, pyridine oxide, pyradizine, pyrimidine, pyrazine, furan, thiophene, triazine, thiazole, imidazole, oxazole, indolizine, imidazo pyridine, naphthalene, dihydrobenzodioxine or benzo(1,3)dioxole.

In another embodiment, in compounds represented by formula (III), B is —(C₁-C₆)alkyl-O—(C₁-C₁₀)alkyl-3-7 membered monocyclic heterocycle.

In another embodiment, in compounds represented by formula (III), B is —(CH₂)—O—(CH₂)₂-3-7 membered monocyclic heterocycle.

In another embodiment, in compounds represented by formula (III), B is —(CH₂)—O—(CH₂)-3-7 membered monocyclic heterocycle.

In another embodiment, in compounds represented by formula (III), B is —(C₁-C₆)alkyl-O—(C₁-C₁₀)alkyl-NH₂.

In another embodiment, in compounds represented by formula (III), B is —(C₁-C₆)alkyl-O—(C₁-C₁₀)alkyl-N₃.

In another embodiment, in compounds represented by formula (III), B is —(CH₂)—O—(C₁-C₁₀)—NR₅R₅.

In another embodiment, in compounds represented by formula (III), B is —(CH₂)—O—(C₁-C₁₀)—NH—(C₁-C₆)alkyl.

In another embodiment, in compounds represented by formula (III), B is —(CH₂)—O—(C₁-C₁₀)—N((C₁-C₆)alkyl)₂.

In another embodiment, in compounds represented by formula (III), B is —(CH₂)—O—(C₁-C₁₀)—NH₂.

In another embodiment, in compounds represented by formula (III), B is —(CH₂)—O—(C₁-C₁₀)—N₃.

In another embodiment, in compounds represented by formula (III), B is —(CH₂)—O—(CH₂)₂—NR₅R₅.

In another embodiment, in compounds represented by formula (III), B is —(CH₂)—O—(CH₂)₂—NH—(C₁-C₆)alkyl.

In another embodiment, in compounds represented by formula (III), B is —(CH₂)—O—(CH₂)₂—N((C₁-C₆)alkyl)₂.

In another embodiment, in compounds represented by formula (III), B is —(CH₂)—O—(CH₂)₂—NH₂.

In another embodiment, in compounds represented by formula (III), B is —(CH₂)—O—(CH₂)₂—N₃.

In another embodiment, in compounds represented by formula (III), X is —CH₂—.

In another embodiment, in compounds represented by formula (III), Z is O or S.

In another embodiment, in compounds represented by formula (III), Z is —N(CH₃)—.

In another embodiment, in compounds represented by formula (III), Z is —CH₂—.

In another embodiment, in compounds represented by formula (III), Z is O or S and R₁ is a substituted or unsubstituted 3-7 membered monocyclic heterocycle or a substituted or unsubstituted 8-12 membered bicyclic heterocycle.

In another embodiment, in compounds represented by formLula (III), R₁₁ is amino or azido.

In another embodiment, in compounds represented by formula (III), R₁₁ is —NH—CH₂—CH(OH)—CH₂—O-phenyl.

In another embodiment, in compounds represented by formula (III), R₁₁ is —NH—C(O)-lower alkyl.

In another embodiment, in compounds represented by formula (III), R₁₁ is —C(O)—O-lower alkyl.

In another embodiment, in compounds represented by formula (III), R₁₁ is —NH-lower alkyl or —N-(lower alkyl)₂, wherein lower alkyl is preferably methyl or ethyl.

In another embodiment, in compounds represented by formula (III), R₁₁ is an isoindole-1,3-dione.

In another embodiment, in compounds represented by formula (III), R₁₁ is piperazine or morpholine.

In another embodiment, in compounds represented by formula (III), R₁₁ is a 5 membered monocyclic heterocycle.

In another embodiment, in compounds represented by formula (III), R₁₁ is a nitrogen containing 5 membered monocyclic heterocycle, such as pyrrole, imidazole or triazole.

In another embodiment, in compounds represented by formula (III), R₄ is —OC(O)R₅, wherein R₅ is substituted or unsubstituted —(C₁-C₁₀)alkyl. Preferably, R₅ is methyl, ethyl or propyl.

In another embodiment, in compounds represented by formula (III), R₄ is —C(O)OR₅, wherein R₅ is substituted or unsubstituted —(C₁-C₁₀)alkyl. Preferably, R₅ is methyl, ethyl or propyl.

In another embodiment, in compounds represented by formula (III), R₄ is —C(O)OCH₂CH₃.

In another embodiment, in compounds represented by formula (III), R₄ is —C(O)OH.

In another embodiment, in compounds represented by formula (III), R₄ is —C(O)NHR₅, where R₅ is H, methyl, ethyl or propyl.

In another embodiment, in compounds represented by formula (III), R₄ is a substituted or unsubstituted 5 membered monocyclic heterocycle, wherein the dihydropyridine core structure can be bound to either a carbon atom or a heteroatom of the 5 membered monocyclic heterocycle.

In another embodiment, in compounds represented by formula (III), R₄ is a bioisosteric replacement of an ester including, but not limited to, oxazole and oxadiazole.

In another embodiment, in compounds represented by formula (III), R₄ is —CN.

In another embodiment, in compounds represented by formula (III), R₅ is —(C₁-C₁₀)alkyl substituted with a 3-7 membered monocyclic heterocycle, a 8-12 membered bicyclic heterocycle or —CN.

In another embodiment, in compounds represented by formula (III), q is an integer selected from 1 or 2.

In another embodiment, in compounds represented by formula (III), there applies a proviso that B is not substituted or unsubstituted —(C₁-C₁₀)alkyl, —(C₃-C₁₀)cycloalkyl, —S(O)R₅, —S(O)₂R₅ or —C(O)NHR₅.

In another embodiment, in compounds represented by formula (III), there applies a proviso that R₁₁ is not a 3-7 membered monocyclic heterocycle or 8-12 membered bicyclic heterocycle.

In another embodiment, in compounds represented by formula (III), there applies a proviso that R₁ and R₂ are not both H.

In another embodiment, in compounds represented by formula (III), there applies a proviso that if X is CH₂, R₁ and R₂ are not both H.

In another embodiment, in compounds represented by formula (III), there applies a proviso that if X is O, m is not 0.

In another embodiment, in compounds represented by formula (III), there applies a proviso that if A is a bicyclic ring, R₁ and R₂ are not both H.

In another embodiment, in compounds represented by formula (III), there applies a proviso that if X is —(CH₂)— or A is a bicyclic ring, R₁ and R₂ are not both H.

In another embodiment, in compounds represented by formula (III), there applies a proviso that if R₃ is other than H, R₁ and R₂ are not both H.

In another embodiment, in compounds represented by formula (III), there applies a proviso that if X is O, m is not 0 and if R₃ is other than H, R₁ and R₂ are not both H.

In another aspect, the invention provides compounds of formula (IV) as set forth below:

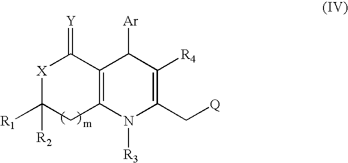

and pharmaceutically acceptable salts, solvates, clathrates, hydrates, polymorphs or prodrugs thereof wherein:

Ar is a substituted or unsubstituted aromatic or heteroaromatic ring, wherein if the ring is substituted, the substituents are independently selected from the group consisting of substituted or unsubstituted lower alkyl, -halo, —CN, —N(R₅)(R₅), —OR₆, —C(O)R₅, —C(O)₂R₅, —OC(O)R₅, —NO₂, and —C(O)N(R₅)(R₅), or two adjacent carbon atoms on the ring are linked by the group —O—(CH2)_{q}—O— to form a bicyclic ring system, wherein q is an integer selected from 1, 2, 3 or 4;

Q is H, -halo, —NO₂, —CN, —OH, —N(R₅)(R₅), —OR₅, —C(O)R₅, —OC(O)R₅, —(O)NHC(O)R₅, substituted or unsubstituted —(C₂-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —(C₀-C₆)alkyl-Z-(C₁-C₁₀)alkyl-R₁₁, —(C₁-C₁₀)alkyl-R₁₁, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, —S(O)₂R₅ or a substituted or unsubstituted aromatic or heteroaromatic ring, wherein if the ring is substituted, the substituents are independently selected from the group consisting of substituted or unsubstituted lower alkyl, -halo, —CN, —N(R₅)(R₅), —OR₆, —C(O)R₅, —C(O)₂R₅, —OC(O)R₅, —NO₂, and C(O)N(R₅)(R₅);

X is selected from the group consisting of O, S, —NR₅, and —C(R₅)(R₅);

Y is O or S;

Z is at each occurrence independently —O—, —S—, —N(R₅)—, —C(O)—, —OC(O)—, —C(O)N(R₅)C(O)—, substituted or unsubstituted —(C₁-C₁₀)alkyl-, substituted or unsubstituted —(C₂-C₁₀)alkenyl-, substituted or unsubstituted —(C₂-C₁₀)alkynyl-, substituted or unsubstituted —(C₃-C₁₀)cycloalkyl-, substituted or unsubstituted —(C₈-C₁₄)bicycloalkyl-, substituted or unsubstituted —(C₅-C₁₀)cycloalkenyl-, substituted or unsubstituted —(C₃-C₁₀)heterocycle-, substituted or unsubstituted phenyl, substituted or unsubstituted naphthyl, substituted or unsubstituted benzyl, —C(O)O—, —C(O)OC(R₅)(R₅)—, —N(R₅)C(O)—, —N(R₅)C(O)NR₅—, —C(O)NR₅—, —OC(O)O—, —S(O)N(R₅)—, —S(O)—, or —S(O)₂—;

R₁ and R₂ are at each occurrence independently selected from —H, -halo, —CN, —N₃, —NO₂, —CN, —OH, —N(R₅)(R₅), OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;

R₃ is at each occurrence independently —H, —C(O)R₅ or substituted or unsubstituted —(C₁-C₁₀)alkyl;

R₄ is at each occurrence independently —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)OR₅, —S(O)N(R₅)(R₅), —SR₅, —S(O)R₅, —S(O)₂R₅ or a substituted or unsubstituted bioisosteric replacement of an ester;

each R₅ is at each occurrence independently H or substituted or unsubstituted —(C₁-C₁₀)alkyl;

each R₆ is at each occurrence H, substituted or unsubstituted —(C₁-C₁₀)alkyl or —(CH₂)ₚ—N(R₅)—(C₁-C₆)alkyl optionally substituted with one or more —OR₅ or —O-aryl groups;

R₁₁ is at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;

m is an integer selected from 0-2; and

p is an integer selected from 1-6.

Compounds of formula (IV) and a pharmaceutically acceptable salts, solvates, clathrates, hydrates, polymorphs and prodrugs thereof are particularly useful for treating or preventing metabolic disorders, including diabetes mellitus, conditions associated with diabetes mellitus and certain complications thereof.

In one embodiment, in compounds represented by formula (IV), Ar is a phenyl or pyridyl ring which can be substituted with one or more halo (e.g., chloro or fluoro), methyl, phenyl, —NH₂, —CN, —NO₂, —CF₃, OH, O-lower alkyl, or O—R₆.

In another embodiment, in compounds represented by formula (IV), Ar is a phenyl or pyridyl ring substituted with one or more —O-lower alkyl-NH—CH₂—CH(OH)—CH₂—O-phenyl groups.

In another embodiment, in compounds represented by formula (IV), Ar is quinoline, indole, pyridine oxide, pyradizine, pyrimidine, pyrazine, furan, thiophene, triazine, thiazole, imidazole, oxazole, indolizine, imidazo pyridine, naphthalene, dihydrobenzodioxine or benzo(1,3)dioxole.

In another embodiment, in compounds represented by formula (IV), R₁ and R₂ are independently selected from H and lower alkyl and R₄ is CO₂-lower alkyl (e.g., CO₂CH₂CH₃ or CO₂CH₃).

In another embodiment, in compounds represented by formula (IV), R₁ and R₂ are alkyl, preferably lower alkyl, more preferably methyl.

In one embodiment, in compounds represented by formula (IV), one of R₁ and R₂ is H and the other is alkyl, preferably lower alkyl more preferably isopropyl or methyl.

In another embodiment, in compounds represented by formula (IV), R₃ is H or lower alkyl (e.g., methyl or ethyl).

In another embodiment, in compounds represented by formula (IV), R₅ is at each occurrence independently H or lower alkyl.

In another embodiment, in compounds represented by formula (IV), R₅ is —(C₁-C₁₀)alkyl substituted with a 3-7 membered monocyclic heterocycle, a 8-12 membered bicyclic heterocycle or —CN.

In another embodiment, in compounds represented by formula (IV), R₆, when substituted, is substituted with —OC₆H₅.

In another embodiment, m is 1.

In another embodiment, in compounds represented by formula (IV), p is an integer selected from 3 or 4.

In another embodiment, in compounds represented by formula (IV), R₄ is a substituted or unsubstituted 5 membered monocyclic heterocycle, wherein the dihydropyridine core structure can be bound to either a carbon atom or a heteroatom of the 5 membered monocyclic heterocycle.

In another embodiment, in compounds represented by formula (IV), R₄ is a bioisosteric replacement of an ester including, but not limited to, oxazole and oxadiazole.

In another embodiment, in compounds represented by formula (IV), R₄ is —CN.

In another embodiment, in compounds represented by formula (IV), R₄ is —OC(O)R₅, wherein R₅ is substituted or unsubstituted —(C₁-C₁₀)alkyl. Preferably, R₅ is methyl, ethyl or propyl.

In another embodiment, in compounds represented by formula (IV), R₄ is —C(O)OR₅, wherein R₅ is substituted or unsubstituted —(C₁-C₁₀)alkyl. Preferably, R₅ is methyl, ethyl or propyl.

In another embodiment, in compounds represented by formula (IV), R₄ is —C(O)OH.

In another embodiment, in compounds represented by formula (IV), R₄ is —C(O)NHR₅, where R₅ is H, methyl, ethyl or propyl.

In another embodiment, in compounds represented by formula (IV), X is —CH₂—.

In another embodiment, in compounds represented by formula (IV), Z is O or S.

In another embodiment, in compounds represented by formula (IV), Z is —N(CH₃)—.

In another embodiment, in compounds represented by formula (IV), Z is —CH₂—.

In another embodiment, in compounds represented by formula (IV), Z is O or S and R₁ is a substituted or unsubstituted 3-7 membered monocyclic heterocycle or a substituted or unsubstituted 8-12 membered bicyclic heterocycle.

In another embodiment, in compounds represented by formula (IV), Q is —(C₁-C₁₀)alkyl-3-7 membered monocyclic heterocycle.

In another embodiment, in compounds represented by formula (IV), Q is —O—(CH₂)₂— 3-7 membered monocyclic heterocycle.

In another embodiment, in compounds represented by formula (IV), Q is —O—(CH₂)—3-7 membered monocyclic heterocycle.

In another embodiment, in compounds represented by formula (IV), Q is —O—(C₁-C₁₀)alkyl-NH₂.

In another embodiment, in compounds represented by formula (IV), Q is —O—(C₁-C₁₀)alkyl-N₃.

In another embodiment, in compounds represented by formula (IV), Q is —O—(C₁-C₁₀)—NR₅R₅.

In another embodiment, in compounds represented by formula (IV), Q is —O—(C₁-C₁₀)—NH—(C_{1 -C}₆)alkyl.

In another embodiment, in compounds represented by formula (IV), Q is —O—(C₁-C₁₀)—N((C₁-C₆)alkyl)₂.

In another embodiment, in compounds represented by formula (IV), Q is —O—(C₁-C₁₀)—NH₂.

In another embodiment, in compounds represented by formula (IV), Q is —O—(C₁-C₁₀)—N₃.

In another embodiment, in compounds represented by formula (IV), Q is —O—(CH₂)₂—NR₅R₅.

In another embodiment, in compounds represented by formula (IV), Q is —O—(CH₂)₂—NH—(C₁-C₆)alkyl.

In another embodiment, in compounds represented by formula (IV), Q is —O—(CH₂)₂—N((C₁-C₆)alkyl)₂.

In another embodiment, in compounds represented by formula (IV), Q is —O—(CH₂)₂—NH₂.

In another embodiment, in compounds represented by formula (IV), Q is —O—(CH₂)₂—N₃.

In another embodiment, in compounds represented by formula (IV), Q is piperazine.

In another embodiment, in compounds represented by formula (IV), R₁₁ is —NH₂ or —N₃.

In another embodiment, in compounds represented by formula (IV), R₁₁ is —NH—CH₂—CH(OH)—CH₂—O-phenyl.

In another embodiment, in compounds represented by formula (IV), R₁₁ is —NH—C(O)-lower alkyl.

In another embodiment, in compounds represented by formula (IV), R₁₁ is —NH—lower alkyl or —N-(lower alkyl)₂, wherein lower alkyl is preferably methyl or ethyl.

In another embodiment, in compounds represented by formula (IV), R₁₁ is —C(O)—O-lower alkyl.

In another embodiment, in compounds represented by formula (IV), R₁₁ is an isoindole-1,3-dione.

In another embodiment, in compounds represented by formula (IV), R₁₁ is piperazine or morpholine.

In another embodiment, in compounds represented by formula (IV), R₁₁ is a 5 membered monocyclic heterocycle.

In another embodiment, in compounds represented by formula (IV), R₁₁ is a nitrogen containing 5 membered monocyclic heterocycle, such as pyrrole, imidazole or triazole.

In another embodiment, in compounds represented by formula (IV), q is an integer selected from 1 or 2.

In another embodiment, in compounds represented by formula (IV), there applies a proviso that R₁ and R₂ are not both H.

In another embodiment, in compounds represented by formula (IV), there applies a proviso that if X is CH₂, R₁ and R₂ are not both H.

In another embodiment, in compounds represented by formula (IV), there applies a proviso that Q is not H.

In another embodiment, in compounds represented by formula (IV), there applies a proviso that if X is O, m is not 0.

In another embodiment, in compounds represented by formula (IV), there applies a proviso that if Ar is a bicyclic ring, R₁ and R₂ are not both H.

In another embodiment, in compounds represented by formula (IV), there applies a proviso that if X is —(CH₂)— or A is a bicyclic ring, R₁ and R₂ are not both H.

In another embodiment, in compounds represented by formula (IV), there applies a proviso that if R₃ is other than H, R₁ and R₂ are not both H.

In another embodiment, in compounds represented by formula (IV), Q is -halo, —NO₂, —CN, —OH, —N(R₅)(R₅), —OR₅, —C(O)R₅, —OC(O)R₅, —C(O)NHC(O)R₅, substituted or unsubstituted —(C₂-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —(C₀-C₆)alkyl-Z-(C₁-C₁₀)alkyl-R₁, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, —S(O)₂R₅ or a substituted or unsubstituted aromatic or heteroaromatic ring, wherein if the ring is substituted, the substituents are independently selected from the group consisting of substituted or unsubstituted lower alkyl, -halo, —CN, —N(R₅)(R₅), —OR₆, —C(O)R₅, —C(O)₂R₅, —OC(O)R₅, —NO₂, and —C(O)N(R₅)(R₅).

In another embodiment, in compounds represented by formula (IV), one or more of the substituents R₁, R₂, R₃, R₄, R₅, R₆, Ar, X, Y and m are selected from those included in the specific exemplified compounds described herein.

In another aspect, the invention provides compounds of formula (V) as set forth below:

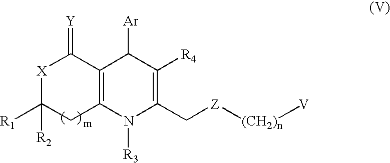

and pharmaceutically acceptable salts, solvates, clathrates, hydrates, polymorphs or prodrugs thereof wherein:

Ar is a mono- or poly-substituted or unsubstituted aromatic or heteroaromatic ring, wherein if the ring is substituted, the substituents are independently selected from the group consisting of substituted or unsubstituted lower alkyl, -halo, —CN, —N(R₅)(R₅), —OR₆, —C(O)R₅, —C(O)₂R₅, —OC(O)R₅, —NO₂, and —C(O)N(R₅)(R₅), or two adjacent carbon atoms on the ring are linked by the group —O—(CH₂)_{q}—O— to form a bicyclic ring system, wherein q is an integer selected from 1, 2, 3 or 4;

V is H, -halo, N₃, —NO₂, —CN, —OH, —N(R₅)(R₇), —OR₅, —C(O)R₅, —OC(O)R₅, —C(O)NHC(O)R₅, substituted or unsubstituted 3-7 membered monocyclic heterocycle or substituted or unsubstituted 8-12 membered bicyclic heterocycle;

X is selected from the group consisting of O, S, —NR₅, and —C(R₅)(R₅);

Y is O or S;

Z is —O—, —S—, —N(R₅)—, —C(O)—, —OC(O)—, —C(O)N(R₅)C(O)—, substituted or unsubstituted —(C₁-C₁₀)alkyl-, substituted or unsubstituted —(C₂-C₁₀)alkenyl-, substituted or unsubstituted —(C₂-C₁₀)alkynyl-, substituted or unsubstituted —(C₃-C₁₀)cycloalkyl-, substituted or unsubstituted —(C₈-C₁₄)bicycloalkyl-, substituted or unsubstituted —(C₅-C₁₀)cycloalkenyl-, substituted or unsubstituted —(C₃-C₁₀)heterocycle-, substituted or unsubstituted phenyl, substituted or unsubstituted naphthyl, substituted or unsubstituted benzyl, —C(O)O—, —C(O)OC(R₅)(R₅)—, —N(R₅)C(O)—, —N(R₅)C(O)NR₅—, —C(O)NR₅—, —OC(O)O—, —S(O)N(R₅)—, —S(O)—, or —S(O)₂—;

R₁ and R₂ are at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;

R₃ is at each occurrence independently —H, —C(O)R₅ or substituted or unsubstituted —(C₁-C₁₀)alkyl;

R₄ is at each occurrence independently —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)OR₅, —S(O)N(R₅)(R₅), —SR₅, —S(O)R₅, —S(O)₂R₅ or a substituted or unsubstituted bioisosteric replacement of an ester;

each R₅ is at each occurrence independently H or substituted or unsubstituted —(C₁-C₁₀)alkyl;

each R₆ is at each occurrence independently H, substituted or unsubstituted —(C₁-C₁₀)alkyl or —(CH₂)ₚ—N(R₅)—(C₁-C₆)alkyl optionally substituted with one or more —OR₅ or —O— aryl groups;

R₇ is selected from the group consisting of H and substituted or unsubstituted —(C₁-C₁₀)alkyl optionally substituted with one or more —OR₅ or —O-aryl groups;

n is an integer selected from 1-10;

m is an integer selected from 0-2; and

p is an integer selected from 1-6.

Compounds of formula (V) and a pharmaceutically acceptable salts, solvates, clathrates, hydrates, polymorphs and prodrugs thereof are particularly useful for treating or preventing metabolic disorders, including diabetes mellitus, conditions associated with diabetes mellitus and certain complications thereof.

In one embodiment, in compounds represented by formula (V), Ar is a phenyl or pyridyl ring which can be substituted with one or more halo (e.g., chloro or fluoro), methyl, phenyl, —NH₂, —CN, —NO₂, OH, —CF₃, O-lower alkyl, or O—R₆.

In another embodiment, in compounds represented by formula (V), Ar is a phenyl or pyridyl ring substituted with one or more —O-lower alkyl-NH—CH₂—CH(OH)—CH₂—O-phenyl groups.

In another embodiment, in compounds represented by formula (V), Ar is quinoline, indole, pyridine oxide, pyradizine, pyrimidine, pyrazine, furan, thiophene, triazine, thiazole, imidazole, oxazole, indolizine, imidazo pyridine, naphthalene, dihydrobenzodioxine or benzo(1,3)dioxole.

In another embodiment, in compounds represented by formula (V), V is -halo, N₃, —NO₂, —CN, —OH, —N(R₅)(R₇), —OR₅, —C(O)R₅, —OC(O)R₅ or —C(O)NHC(O)R₅.

In another embodiment, in compounds represented by formula (V), V is NH₂ or N₃.

In another embodiment, in compounds represented by formula (V), V is —NH—CH₂—CH(OH)—CH₂—O-phenyl.

In another embodiment, in compounds represented by formula (V), V is —NH—C(O)-lower alkyl.

In another embodiment, in compounds represented by formula (V), V is —NH-lower alkyl or —N-(lower alkyl)₂, wherein lower alkyl is preferably methyl or ethyl.

In another embodiment, in compounds represented by formula (V), V is —C(O)—O-lower alkyl

In another embodiment, in compounds represented by formula (V), V is an isoindole-1,3-dione.

In another embodiment, in compounds represented by formula (V), V is piperazine or morpholine.

In another embodiment, in compounds represented by formula (V), V is a 5 membered monocyclic heterocycle.

In another embodiment, in compounds represented by formula (V), V is a nitrogen containing 5 membered monocyclic heterocycle, such as pyrrole, imidazole or triazole.

In embodiments where V is a heterocycle in compounds represented by formula (V), the —(CH₂)— group can be bound to a carbon atom or a heteroatom of V.

In another embodiment, in compounds represented by formula (V), X is O or CH₂.

In another embodiment, in compounds represented by formula (V), R₁and R₂ are independently selected from H and lower alkyl and R₄ is CO₂-lower alkyl (e.g., CO₂CH₂CH₃ or CO₂CH₃).

In another embodiment, in compounds represented by formula (V), R₁ and R₂ are alkyl, preferably lower alkyl, more preferably methyl.

In one embodiment, in compounds represented by formula (V), one of R₁ and R₂ is H and the other is alkyl, preferably lower alkyl more preferably isopropyl or methyl.

In another embodiment, in compounds represented by formula (V), R₅ is —(C₁-C₁₀)alkyl substituted with a 3-7 membered monocyclic heterocycle, a 8-12 membered bicyclic heterocycle or —CN.

In another embodiment, in compounds represented by formula (V), R₃ is H or lower alkyl (e.g., methyl or ethyl).

In another embodiment, in compounds represented by formula (V), R₄ is a substituted or unsubstituted 5 membered monocyclic heterocycle, wherein the dihydropyridine core structure can be bound to either a carbon atom or a heteroatom of the 5 membered monocyclic heterocycle.

In another embodiment, in compounds represented by formula (V), R₄ is a bioisosteric replacement of an ester including, but not limited to, oxazole and oxadiazole.

In another embodiment, in compounds represented by formula (V), R₄ is —CN.

In another embodiment, in compounds represented by formula (V), R₄ is —OC(O)R₅, wherein R₅ is substituted or unsubstituted —(C₁-C₁₀)alkyl. Preferably, R₅ is methyl, ethyl or propyl.

In another embodiment, in compounds represented by formula (V), R₄ is —C(O)OR₅, wherein R₅ is substituted or unsubstituted —(C₁-C₁₀)alkyl. Preferably, R₅ is methyl, ethyl or propyl.

In another embodiment, in compounds represented by formula (V), R₄ is —C(O)OH.

In another embodiment, in compounds represented by formula (V), R₄ is —C(O)NHR₅, where R₅ is H, methyl, ethyl or propyl.

In another embodiment, in compounds represented by formula (V), X is —CH₂—.

In another embodiment, in compounds represented by formula (V), Z is O or S.

In another embodiment, in compounds represented by formula (V), Z is —N(CH₃)—.

In another embodiment, in compounds represented by formula (V), Z is —CH₂—.

In another embodiment, in compounds represented by formula (V), Z is O or S and V is a substituted or unsubstituted 3-7 membered monocyclic heterocycle or a substituted or unsubstituted 8-12 membered bicyclic heterocycle, wherein the —(CH₂)n— group can be bound to a carbon atom or a heteroatom of V.

In another embodiment, in compounds represented by formula (V), R₅ is at each occurrence independently H or lower alkyl.

In another embodiment, in compounds represented by formula (V), R₆, when substituted, is substituted with —OC₆H₅.

In another embodiment, in compounds represented by formula (V), n is an integer selected from 1, 2, 3, 4 or 5.

In another embodiment, in compounds represented by formula (V), m is 1.

In another embodiment, in compounds represented by formula (V), p is an integer selected from 3 or 4.

In another embodiment, in compounds represented by formula (V), q is an integer selected from 1 or 2.

In another embodiment, in compounds represented by formula (V), there applies a proviso that R₁ and R₂ are not both H.

In another embodiment, in compounds represented by formula (V), there applies a proviso that if Ar is a bicyclic ring, R₁ and R₂ are not both H.

In another embodiment, in compounds represented by formula (V), there applies a proviso that if X is —(CH₂)—, R₁ and R₂ are not both H.

In another embodiment, in compounds represented by formula (V), there applies a proviso that if X is —(CH₂)— or A is a bicyclic ring, R₁ and R₂ are not both H.

In another embodiment, in compounds represented by formula (V), there applies a proviso that when Z is substituted or unsubstituted —(C₁-C₁₀)alkyl or fluoroalkyl, then V is not H or halo.

In another embodiment, in compounds represented by formula (V), there applies a proviso that if R₃ is other than H, R₁ and R₂ are not both H.

In another embodiment, in compounds represented by formula (V), one or more of the substituents R₁, R₂, R₃, R₄, R₅, R₆, R₇, V, X, Y, n, m, and p are selected from those included in the specific exemplified compounds described herein.

In another aspect, the invention provides compounds of formula (VI) as set forth below:

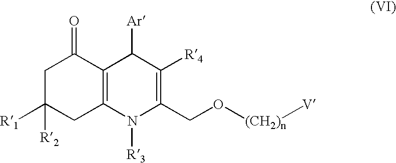

and pharmaceutically acceptable salts, solvates, clathrates, hydrates, polymorphs or prodrugs thereof wherein:

Ar′ is phenyl or pyridyl, which may be unsubstituted or independently substituted with one or more substituted or unsubstituted lower alkyl, -halo, —CN, —N(R′₅)(R′₅), —OR′₅, —C(O)R′₅, —C(O)₂R′₅, —OC(O)R′₅, —NO₂, or —C(O)N(R′₅)(R′₅) groups, or two adjacent carbon atoms on the phenyl or pyridyl are linked by the group —O—(CH₂)_{q}—O— to form a bicyclic ring system, wherein q is an integer selected from 1, 2, 3 or 4;

V′ is H, N(R′₁₁)(R′₁₁), N₃, substituted or unsubstituted 3-7 membered monocyclic heterocycle or substituted or unsubstituted 8-12 membered bicyclic heterocycle;

each R′, and R′₂ may be independently selected from H and substituted or unsubstituted lower alkyl;

R′₃ is —C(O)R₅, —H, or substituted or unsubstituted lower alkyl;

R′₄ is —CN, —CO₂-lower alkyl, —C(O)NHR₅ or a bioisosteric replacement of an ester;

each R′₅ is at each occurrence independently H or substituted or unsubstituted —(C₁-C₁₀) alkyl;

R₁₁′ is at each occurrence independently selected from —H, —OH, —N(R₅)(R₅), —OR₅, —C(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —S(O)N(R₅)(R₅), —SR₅, —S(O)R₅, and —S(O)₂R₅; and

n is an integer selected from the group consisting of 1, 2, 3 and 4.

Compounds of formula (VI) and a pharmaceutically acceptable salts, solvates, clathrates, hydrates, polymorphs and prodrugs thereof are particularly useful for treating or preventing metabolic disorders, including diabetes mellitus, conditions associated with diabetes mellitus and certain complications thereof.

More specific compounds of formula (VI) include those wherein:

Ar′ is an ortho-substituted or unsubstituted phenyl or pyridyl, wherein if the phenyl or pyridyl is substituted, the substituents are independently selected from the group consisting of lower alkyl, -halo, —CN, —N(R′₅)(R′₅), —OR′₅, —C(O)R′₅, —C(O)₂R′₅, —OC(O)R′₅, —NO₂, and —C(O)N(R′₅)(R′₅);

V′ is —NH₂ or —N₃;

R′₁ and R′₂ may be independently selected from —H and substituted or unsubstituted-lower alkyl;

R′₃ is —C(O)R₅, —H, or substituted or unsubstituted-lower alkyl;

R′₄ is —CN or —CO₂-lower alkyl;

each R′₅ is independently —H or substituted or unsubstituted —(C₁-C₁₀)alkyl; and

n is 2 or 3.

In another embodiment, in compounds represented by formula (VI), Ar′ is phenyl substituted with one or more -halo, methyl, phenyl, —NO₂, —CF₃, OH, lower alkoxy, —CN or —NH₂ groups.

In another embodiment, in compounds represented by formula (VI), Ar′ is quinoline, indole, pyridine oxide, pyradizine, pyrimidine, pyrazine, furan, thiophene, triazine, thiazole, imidazole, oxazole, indolizine, imidazo pyridine, naphthalene, dihydrobenzodioxine or benzo(1,3)dioxole.

In another embodiment, in compounds represented by formula (VI), n is 2 and V′ is —NH₂.

In another embodiment, in compounds represented by formula (VI), q is an integer selected from 1 or 2.

In another embodiment, in compounds represented by formula (VI), R′₃ is H, methyl or ethyl.

In another embodiment, in compounds represented by formula (VI), R′₄ is —CN.

In another embodiment, in compounds represented by formula (VI), R′₄ is —C(O)OR₅, wherein R₅ is substituted or unsubstituted —(C₁-C₁₀)alkyl. Preferably, R₅ is methyl, ethyl or propyl.

In another embodiment, in compounds represented by formula (VI), R′₄ is —OC(O)OH.

In another embodiment, in compounds represented by formula (VI), R′₄ is a bioisosteric replacement of an ester including, but not limited to, oxazole and oxadiazole.

In another embodiment, in compounds represented by formula (VI), R′₄ is —C(O)NHR₅, where R₅ is H, methyl, ethyl or propyl.

In another embodiment, in compounds represented by formula (VI), V′ is N(R′₁)(R′₁) or N₃.

In another embodiment, in compounds represented by formula (VI), V′ is NH₂.

In another embodiment, in compounds represented by formula (VI), V′ is —NH—CH₂—CH(OH)—CH₂—O-phenyl.

In another embodiment, in compounds represented by formula (VI), V′ is —NH—C(O)-lower alkyl.

In another embodiment, in compounds represented by formula (VI), V′ is —C(O)—O-lower alkyl.

In another embodiment, in compounds represented by formula (VI), V′ is —NH-lower alkyl or —N-(lower alkyl)₂, wherein lower alkyl is preferably methyl or ethyl.

In another embodiment, in compounds represented by formula (VI), V′ is an isoindole-1,3-dione.

In another embodiment, in compounds represented by formula (VI), V′ is piperazine or morpholine.

In another embodiment, in compounds represented by formula (VI), V′ is a 5 membered monocyclic heterocycle.

In another embodiment, in compounds represented by formula (VI), V′ is a nitrogen containing 5 membered monocyclic heterocycle, such as pyrrole, imidazole or triazole.

In embodiments where V′ is a heterocycle in compounds represented by formula (VI), the —(CH₂)— group can be bound to a carbon atom or a heteroatom of V′.

In another embodiment, in compounds represented by formula (VI), R′₁ and R′₂ are alkyl, preferably lower alkyl more preferably methyl.

In another embodiment, in compounds represented by formula (VI), one of R′₁ and R′₂ is H and the other is alkyl, preferably lower alkyl more preferably isopropyl or methyl.

In another embodiment, in compounds represented by formula (VI), there applies a proviso that R′₁ and R′₂ are not both H.

In another embodiment, in compounds represented by formula (VI), there applies a proviso that if Ar′ is a bicyclic ring, R′₁ and R′₂ are not both H.

In another embodiment, in compounds represented by formula (VI), there applies a proviso that if R′₃ is other than H, R′₁ and R′₂ are not both H.

In another aspect, the invention provides compounds represented by formula (VII) as set forth below:

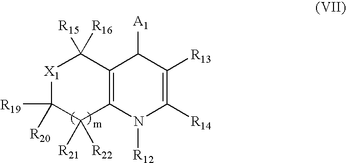

or a pharmaceutically acceptable salt, solvate, clathrate, or prodrug thereof wherein m, R₁₂, R₁₃, R₁₄, R₁₅, R₁₆, R₁₉, R₂₀, R₂₁, and R₂₂ are defined above; A₁ is an optionally substituted alkyl, an optionally substituted alkenyl, an optionally substituted alkynyl, an optionally substituted cycloalkyl, an optionally substituted cycloalkenyl, or an optionally substituted heterocycloalkyl; X₁ is O, S, —NR₂₃—, or >CR₁₇R₁₈ ; and R₁₇ and R₁₈ are each, independently, —H or a substituent.

Compounds of formula (VII) and a pharmaceutically acceptable salts, solvates, clathrates, hydrates, polymorphs and prodrugs thereof are particularly useful for treating or preventing metabolic disorders, including diabetes mellitus, conditions associated with diabetes mellitus and certain complications thereof.

In one embodiment, in compounds represented by formula (VII), there applies one or more (including all) of the following provisos:

1) when R₁₄ is methyl, A₁ is not a lower alkyl or a lower alkenyl group, wherein the lower alkyl or the lower alkenyl group is substituted with phenyl, nitrophenyl, pyridyl, cyclohexyl, phenylmethoxy, or —S(O)ᵣCH₃, wherein r is 0, 1, or 2;

2) when R₁₄ is isopropyl or cyclopentyl, R₁₃ is not p-(trifluoromethyl)benzoyl;

3) when R₁₃ is cyano, R₁₄ is not —SR₂₃;

4) when R₁₃ is cyano, —C(O)OCH₂CH₃, benzoyl, or acetyl, all of A₁, R₁₄, R₁₉, and R₂₀ are not methyl; and

5) provided that the compound is not 2,7,7-trimethyl-4-(hex-1-yl)-5-oxo-1,4,5,6,7,8-hexahydroquinoline-3-carboxylic acid ethyl ester, 2-amino-4-ethyl-5-oxo-7,7-dimethyl-1,4,5,6,7,8-hexahydroquinoline-3-carbonitrile, 2-amino-4-methyl-5-oxo-1,4,5,6,7,8-hexahydroquinoline-3-carboxylic acid ethyl ester, 2-phenyl-4-(2-phenylethyn-1-yl)-5-oxo-1,4,5,6,7,8-hexahydroquinoline-3-carboxylic acid ethyl ester, 2-methyl-4-tetrahydrothienyl-5-oxo-1,4,5,6,7,8-hexahydro-quinoline-3-carboxylic acid ethyl ester, 2,7,7-trimethyl-4-cyclohexyl-5-oxo-1,4,5,6,7,8-hexahydroquinoline-3-carbonitrile, 2,7,7-trimethyl-4-isopropyl-5-oxo-1,4,5,6,7,8-hexahydroquinoline-3-carboxylic acid ethyl ester, 2,7,7-trimethyl-4-cyclopentyl-5-oxo-1,4,5,6,7,8-hexahydroquinoline-3-carboxylic acid ethyl ester, 2,4-dimethyl-5-oxo-1,4,5,6,7,8-hexahydroquinoline-3-carboxylic acid ethyl ester, or 2,4-dimethyl-5-oxo-4,7-dihydro-1H-furo[3,4-b]pyridine-3-carboxylic acid methyl ester.

In another embodiment, in compounds represented by formula (VII), R₁₅ and R₁₆ taken together are ═O.

In another embodiment, in compounds represented by formula (VII), m is 1.

In another embodiment, in compounds represented by formula (VII), X₁ is —O—.

In another embodiment, in compounds represented by formula (VII), X₁ is >CR₁₇R₁₈.

In another embodiment, in compounds represented by formula (VII), A₁, m, X₁, R₁₂, R₁₃, R₁₄, R₁₅, R₁₆, R₁₉, R₂₀, R₂₁, and R₂₂ are selected from those included in specific exemplified compounds described herein.

In another aspect, the invention provides compounds represented by formula (VIII) as set forth below:

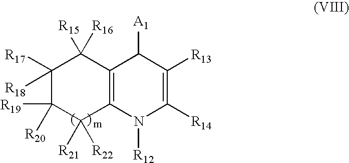

or a pharmaceutically acceptable salt, solvate, clathrate, or prodrug thereof wherein m, A₁, R₁₂, R₁₃, R₁₄, R₁₅, R₁₆, R₁₇, R₁₈, R₁₉, R₂₀, R₂₁ and R₂₂ are defined above.

Compounds of formula (VIII) and a pharmaceutically acceptable salts, solvates, clathrates, hydrates, polymorphs and prodrugs thereof are particularly useful for treating or preventing metabolic disorders, including diabetes mellitus, conditions associated with diabetes mellitus and certain complications thereof.

In one embodiment, in compounds represented by formula (VIII), there applies one or more (including all) of the following provisos:

1) when R₁₄ is methyl, A₁ is not a lower alkyl or a lower alkenyl group, wherein the lower alkyl or the lower alkenyl group is substituted with phenyl, nitrophenyl, pyridyl, cyclohexyl, phenylmethoxy, or —S(O)ᵣCH₃, wherein r is 0, 1, or 2;

2) provided that when R₁₄ is isopropyl or cyclopentyl, R₁₃ is not p-(trifluoromethyl)benzoyl;

3) provided that when R₁₃ is cyano, R₁₄ is not —SR₂₃;

4) provided that when R₁₃ is cyano, —C(O)OCH₂CH₃, benzoyl, or acetyl, all of A₁, R₁₄, R₁₉, and R₂₀ are not methyl; and

5) provided that the compound is not 2,7,7-trimethyl-4-(hex-1-yl)-5-oxo-1,4,5,6,7,8-hexahydroquinoline-3-carboxylic acid ethyl ester, 2-amino-4-ethyl-5-oxo-7,7-dimethyl-1,4,5,6,7,8-hexahydroquinoline-3-carbonitrile, 2-amino-4-methyl-5-oxo-1,4,5,6,7,8-hexahydroquinoline-3-carboxylic acid ethyl ester, 2-phenyl-4-(2-phenylethyn-1-yl)-5-oxo-1,4,5,6,7,8-hexahydroquinoline-3-carboxylic acid ethyl ester, 2-methyl-4-tetrahydrothienyl-5-oxo-1,4,5,6,7,8-hexahydro-quinoline-3-carboxylic acid ethyl ester, 2,7,7-trimethyl-4-cyclohexyl-5-oxo-1,4,5,6,7,8-hexahydroquinoline-3-carbonitrile, 2,7,7-trimethyl-4-isopropyl-5-oxo-1,4,5,6,7,8-hexahydroquinoline-3-carboxylic acid ethyl ester, 2,7,7-trimethyl-4-cyclopentyl-5-oxo-1,4,5,6,7,8-hexahydroquinoline-3-carboxylic acid ethyl ester, or 2,4-dimethyl-5-oxo-1,4,5,6,7,8-hexahydroquinoline-3-carboxylic acid ethyl ester.

In another embodiment, in compounds represented by formula (VIII), R₁₅ and R₁₆ together are ═O.

In another embodiment, in compounds represented by formula (VIII), m is 1.

In another embodiment, in compounds represented by formula (VIII), A₁, m, R₁₂, R₁₃, R₁₄, R₁₅, R₁₆, R₁₇, R₁₈, R₁₉, R₂₀, R₂₁, and R₂₂ are selected from those included in specific exemplified compounds described herein.

In another aspect, the invention provides compounds represented by formula (IX) as set forth below:

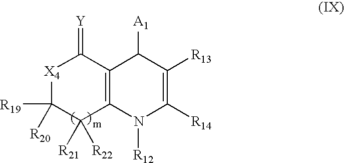

or a pharmaceutically acceptable salt, solvate, clathrate, or prodrug thereof wherein m, A₁, Y, X₄, R₁₂, R₁₃, R₁₄, R₁₉, R₂₀, R₂₁, and R₂₂ are defined above.

Compounds of formula (IX) and a pharmaceutically acceptable salts, solvates, clathrates, hydrates, polymorphs and prodrugs thereof are particularly useful for treating or preventing metabolic disorders, including diabetes mellitus, conditions associated with diabetes mellitus and certain complications thereof.

In one embodiment, in compounds represented by formula (IX), there applies the provisos that the compound is not 2,4-dimethyl-5-oxo-4,7-dihydro-1H-furo[3,4-b]pyridine-3-carboxylic acid methyl ester.

In another embodiment, in compounds represented by formula (IX), Y is ═O.

In another embodiment, in compounds represented by formula (IX), X₄ is —O—.

In another embodiment, in compounds represented by formula (IX), m is 1.

In another embodiment, in compounds represented by formula (IX), A₁, m, Y, X₄, R₁₂, R₁₃, R₁₄, R₁₉, R₂₀, R₂₁, and R₂₂ are selected from those included in specific exemplified compounds described herein.

In another aspect, the invention provides compounds represented by formula (X) as set forth below:

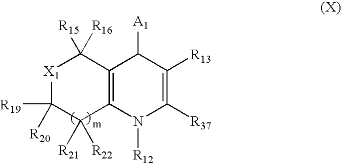

or a pharmaceutically acceptable salt, solvate, clathrate, or prodrug thereof wherein m, A₁, X₁, R₁₂, R₁₃, R₁₅, R₁₆, R₁₉, R₂₀, R₂₁, and R₂₂ are defined above.

R₃₇ is -halo, —NO₂, —CN, —OH, —N(R₃₃)(R₃₃), —OR₃₃, —C(O)R₃₄, —OC(O)R₃₄, —C(O)NHC(O)R₃₃, substituted or unsubstituted —(C₂-C₁₀)alkenyl, substituted or unsubstituted —(C₂-C₁₀)alkynyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted naphthyl, substituted or unsubstituted benzyl, a substituted or unsubstituted heteroalkyl, —(C₁-C₆)alkyl-Z₁-(C₁-C₁₀)alkyl-R₃₅, —(C₁-C₁₀)alkyl-R₃₆, —(C₁-C₁₀)alkyl-N(R₃₄)(R₃₄), —CO₂R₃₄, —NHC(O)R₃₄, —NHC(O)NHR₃₄, —C(O)NHR₃₄, —OC(O)R₃₄, —OC(O)OR₃₄, or —S(O)N(R₃₄)(R₃₄).

Z₁, for each occurrence, is independently, —O—, —S—, —N(R₃₄)—, —C(O)—, —OC(O)—, —C(O)N(R₃₄)C(O)—, substituted or unsubstituted —(C₃-C₁₀)cycloalkyl-, substituted or unsubstituted —(C₈-C₁₄)bicycloalkyl-, substituted or unsubstituted —(C₅-C₁₀)cycloalkenyl-, substituted or unsubstituted —(C₃-C₁₀)heterocycle-, substituted or unsubstituted phenyl, substituted or unsubstituted naphthyl, substituted or unsubstituted benzyl, —C(O)O—, —C(O)OC(R₃₄)(R₃₄)—, —N(R₃₄)C(O)—, —N(R₃₄)C(O)NR₃₄—, —C(O)NR₃₄—, —OC(O)O—, —S(O)N(R₃₄)—, —S(O)—, or —S(O)₂—.

R₃₃, for each occurrence, is, independently, a substituted or unsubstituted alkyl.

R₃₄, for each occurrence, is, independently, —H or a substituted or unsubstituted alkyl.

R₃₅, for each occurrence, is, independently, selected from —H, halo, —CN, —N₃, —NO₂, —CN, —OH, —N(R₃₄)(R₃₄), —OR₃₄, —C(O)R₃₄, —OC(O)R₃₄, —C(O)NHC(O)R₃₄, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₃₄, —C(O)OCH(R₃₄)(R₃₄), —NHC(O)R₃₄, —NHC(O)NHR₃₄, —C(O)NHR₃₄, —OC(O)R₃₄, —OC(O)OR₃₄, —NR₃₄S(O)₂R₃₃, —S(O)N(R₃₄)(R₃₄), —SR₃₄, —S(O)R₃₄, and —S(O)₂R₃₄.

R₃₆, for each occurrence, is, independently, selected from halo, —CN, —N₃, —NO₂, —CN, —OH, —N(R₃₄)(R₃₄), —OR₃₄, —C(O)R₃₄, —OC(O)R₃₄, —C(O)NHC(O)R₃₄, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₃₄, —C(O)OCH(R₃₄)(R₃₄), —NHC(O)R₃₄, —NHC(O)NHR₃₄, —C(O)NHR₃₄, —OC(O)R₃₄, —OC(O)OR₃₄, —S(O)N(R₃₄)(R₃₄), —SR₃₄, —S(O)R₃₄, and —S(O)₂R₃₄.

Compounds of formula (X) and a pharmaceutically acceptable salts, solvates, clathrates, hydrates, polymorphs and prodrugs thereof are particularly useful for treating or preventing metabolic disorders, including diabetes mellitus, conditions associated with diabetes mellitus and certain complications thereof.

In one embodiment, in compounds represented by formula (X), R₁₅ and R₁₆ together are ═O.

In another embodiment, in compounds represented by formula (X), X₁ is >CR₁₇R₁₈.

In another embodiment, in compounds represented by formula (X), m is 1.

In another embodiment, in compounds represented by formula (X), A₁, m, R₁₂, R₁₃, R₁₅, R₁₆, R₁₉, R₂₀, R₂₁, R₂₂ and R₃₇ are selected from those included in specific exemplified compounds described herein.

In another embodiment, in compounds represented by formula (VII), (VIII), (IX) or (X), A₁ is a substituted or unsubstituted alkyl or a substituted or unsubstituted cycloalkyl.

In another embodiment, in compounds represented by formula (VII), (VIII), (IX) or (X), A₁ is substituted with one or more substituents selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR₂₈R₂₉, —NR₃₀C(O)R₃₁, a halo, —OR₃₀, cyano, nitro, a haloalkoxy, —C(O)R₃₀, —NR₂₈R₂₉, —SR₃₀, —C(O)OR₃₀, —OC(O)R₃₀, —NR₃₀C(O)NR₂₈R₂₉, —OC(O)NR₂₈R₂₉, —NR₃₀C(O)OR₃₁, —S(O)ᵣR₃₀, —S(O)ᵣNR₂₈R₂₉, ═O, ═S, and ═N—R₃₀, wherein R₂₈, R₂₉, R₃₀ and R₃₁ are defined above.

In another embodiment, in compounds represented by formula (VII), (VIII), (IX) or (X), A₁ is selected from the group consisting of methyl, ethyl, n-propyl, n-butyl, n-pentyl, n-hexyl, n-heptyl, n-octyl, n-nonyl, n-decyl, isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylpentyl, 2,2-dimethylpentyl, 2,2-dimethylhexyl, 3,3-dimethylpentyl, 3,3-dimethylpentyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, cyclopropyl, 1-methylcyclopropyl, cyclopropylmethyl, cyclobutyl, cyclopentyl, cyclohexyl, cycloheptyl, cyclooctyl, cyclononyl, and cyclodecyl.

In another embodiment, in compounds represented by formula (VII), (VIII), (IX) or (X), A₁ is methyl, isopropyl, cyclopropyl, cyclopentyl, cyclohexyl, 1-methylcyclopropyl, or cyclopropylmethyl.

In another embodiment, R₁₇ and R₁₈ are —H in compounds represented by formula (I), (VII), (VIII), or (X).

In another embodiment, R₂₁ and R₂₂, for each occurrence, are —H iin compounds represented by formula (I), (II), (VII), (VIII), (IX) or (X).

In another embodiment, in compounds represented by formula (I), (II), (VII), (VIII), (IX) or (X), R₁₉ and R₂₀ are both alkyl, preferably lower alkyl more preferably methyl.

In another embodiment, in compounds represented by formula (I), (II), (VII), (VIII), (IX) or (X), one of R₁₉ and R₂₀ is H and the other is alkyl, preferably lower alkyl more preferably isopropyl or methyl.

In another embodiment, in compounds represented by formula (I), (II), (VII), (VIII), (IX) or (X), R₁₉ and R₂₀ together with the carbon to which they are attached form a (C₃-C₇)cycloalkyl, preferably a cyclopropyl, cyclobutyl, cyclopentyl or cyclohexyl.

In another embodiment, in compounds represented by formula (I), (II), (VII), (VIII), (IX) or (X), R₁₂ is H.

In another embodiment, in compounds represented by formula (I), (II), (VII), (VIII), (IX) or (X), R₁₂ is a lower alkyl, such as methyl or ethyl.

In another embodiment, in compounds represented by formula (I), (II), (VII), (VIII), (IX) or (X), R₁₃ is —C(O)O-(lower alkyl), —C(O)OH, cyano, —C(O)NR₃₂R₃₂, —C(O)-(lower alkyl), wherein R₃₂, for each occurrence, is —H or a lower alkyl.

In another embodiment, in compounds represented by formula (I), (II), (VII), (VIII), (IX) or (X), R₁₃ is —OC(O)R₃₂, wherein R₃₂ is H or a lower alkyl. Preferably, R₃₂ is methyl, ethyl or propyl.

In another embodiment, in compounds represented by formula (I), (II), (VII), (VIII), (IX) or (X), R₁₃ is —C(O)OR₃₂, wherein R₃₂ is H or a lower alkyl. Preferably, R₃₂ is methyl, ethyl or propyl.

In another embodiment, in compounds represented by formula (I), (II), (VII), (VIII), (IX) or (X), R₁₃ is —C(O)OCH₂CH₃.

In another embodiment, in compounds represented by formula (I), (II), (VII), (VIII), (IX) or (X), R₁₃ is —C(O)OH.

In another embodiment, in compounds represented by formula (I), (II), (VII), (VIII), (IX) or (X), R₁₃ is —C(O)NHR₃₂, wherein R₃₂ is H or a lower alkyl. Preferably, R₃₂ is methyl, ethyl or propyl.

In another embodiment, in compounds represented by formula (I), (II), (VII), (VIII), (IX) or (X), R₁₃ is a substituted or unsubstituted 5 membered monocyclic heterocycle, wherein the dihydropyridine core structure can be bound to either a carbon atom or a heteroatom of the 5 membered monocyclic heterocycle.

In another embodiment, in compounds represented by formula (I), (II), (VII), (VIII), (IX) or (X), R₁₃ is a bioisosteric replacement of an ester including, but not limited to, oxazole and oxadiazole.

In another embodiment, in compounds represented by formula (I), (II), (VII), (VIII), (IX) or (X), R₁₃ is —CN.

In another embodiment, in compounds represented by formula (I), (II), (VII), (VIII), (IX) or (X), R₁₄ or R₃₇ is —(C₁-C₆)alkyl-O—(C₁-C₁₀)alkyl-3-7 membered monocyclic heterocycle.

In another embodiment, in compounds represented by formula (I), (II), (VII), (VIII), (IX) or (X), R₁₄ or R₃₇ is —(CH₂)—O—(CH₂)₂-3-7 membered monocyclic heterocycle.

In another embodiment, in compounds represented by formula (I), (II), (VII), (VIII), (IX) or (X), R₁₄ or R₃₇ is —(CH₂)—O—(CH₂)-3-7 membered monocyclic heterocycle.

In another embodiment, in compounds represented by formula (I), (II), (VII), (VIII), (IX) or (X), R₁₄ or R₃₇ is —(C₁-C₆)alkyl-O—(C₁-C₁₀)alkyl-NH₂.

In another embodiment, in compounds represented by formula (I), (II), (VII), (VIII), (IX) or (X), R₁₄ or R₃₇ is —(C₁-C₆)alkyl-O—(C₁-C₁₀)alkyl-N₃.

In another embodiment, in compounds represented by formula (I), (II), (VII), (VIII), (IX) or (X), R₁₄ or R₃₇ is —(CH₂)—O—(C₁-C₁₀)—NR₂₈R₂₉.

In another embodiment, in compounds represented by formula (I), (II), (VII), (VIII), (IX) or (X), R₁₄ or R₃₇ is —(CH₂)—O—(C₁-C₁₀)—NH—(C₁-C₆)alkyl.

In another embodiment, in compounds represented by formula (I), (II), (VII), (VIII), (IX) or (X), R₁₄ or R₃₇ is —(CH₂)—O—(C₁-C₁₀)—N((C₁-C₆)alkyl)₂.

In another embodiment, in compounds represented by formula (I), (II), (VII), (VIII), (IX) or (X), R₁₄ or R₃₇ is —(CH₂)—O—(C₁-C₁₀)—NH₂.

In another embodiment, in compounds represented by formula (I), (II), (VII), (VIII), (IX) or (X), R₁₄ or R₃₇ is —(CH₂)—O—(C₁-C₁₀)—N₃.

In another embodiment, in compounds represented by formula (I), (II), (VII), (VIII), (IX) or (X), R₁₄ or R₃₇ is —(CH₂)—O—(CH₂)₂—NR₂₈R₂₉.

In another embodiment, in compounds represented by formula (I), (II), (VII), (VIII), (IX) or (X), R₁₄ or R₃₇ is —(CH₂)—O—(CH₂)₂—NH—(C₁-C₆)alkyl.

In another embodiment, in compounds represented by formula (I), (II), (VII), (VIII), (IX) or (X), R₁₄ or R₃₇ is —(CH₂)—O—(CH₂)₂—N((C₁-C₆)alkyl)₂.

In another embodiment, in compounds represented by formula (I), (II), (VII), (VIII), (IX) or (X), R₁₄ or R₃₇ is —(CH₂)—O—(CH₂)₂—NH₂.

In another embodiment, in compounds represented by formula (I), (II), (VII), (VIII), (IX) or (X), R₁₄ or R₃₇ is —(CH₂)—O—(CH₂)₂—N₃.

In another embodiment, in compounds represented by formula (I), (II), (VII), (VIII), (IX) or (X), R₁₄ or R₃₇ is cyclopropyl, ethoxymethyl, 2-amino-ethoxymethyl, 2-azido-ethoxymethyl, 2-(2-hydroxy-3-phenoxy-propylamino)-ethoxymethyl, propoxymethyl, isopropoxymethyl, N-mesyl-2-aminoethoxymethyl, N-acetyl-2-aminoethoxymethyl, N-ethyl-2-aminoethoxymethyl, N-methyl-2-aminoethoxymethyl, 2-(1,3-dioxo-1,3-dihydro-isoindol-2-yl)-ethoxymethyl, morpholin-4-yl-methyl, 2-morpholin-4-yl-ethoxymethyl, N,N-dimethylaminomethyl, carbethoxycarbonylmethoxymethyl, N-(2-hydroxyethyl)-N-methylaminomethyl, piperazin-1-yl-methyl, 2-hydroxyethoxymethyl, N,N-dimethylamino-ethoxymethyl, 4-aminobutyl, imidazol-5-yl-methoxymethyl, imidazol-4-yl-methoxymethyl, 2-imidazol-1-yl-ethoxymethyl, 3-imidazol-1-yl-propyl, 3-pyrazol-1-yl-propyl, propoxymethyl, isopropoxymethyl, methoxyethoxymethyl, pyrrol-3-yl-methoxymethyl, pyrrol-2-yl-methoxymethyl, [1,2,4]triazol-3-yl-methoxymethyl, 2H-pyrazol-3-yl-methoxymethyl, 3H-[1,2,3]triazol-4-yl-methoxymethyl, or 2-pyrrol-1-yl-ethoxymethyl. The compound of Claim 1, wherein R₁₄ is —NR₃₉R₄₀ or —OR₄₁, wherein:

In another embodiment, in compounds represented by formula (I), (II), (VII), (VIII), (IX) or (X), R₁₄ or R₃₇ are —NR₃₉R₄₀ or —OR₄₁, wherein R₃₉ and R₄₀ are each, independently, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂; or R₃₉ and R₄₀, taken together with the nitrogen to which they are attached are an optionally substituted heterocycloalkyl or optionally substituted heteroaryl; R₄₁ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)N₄₃R₄₄, —S(O)₂R₄₂, or —S(O)R₄₂; R₄₂ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;and R₄₃ and R₄₄ are each, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl, or R₄₃ and R₄₄ taken together with the nitrogen to which they are attached are an optionally substituted heterocycloalkyl or optionally substituted heteroaryl.

In a preferred embodiment, in compounds represented by formula (I), (II), (VII), (VIII), (IX) or (X), R₁₄ or R₃₇ is cyclopropyl, ethoxymethyl, propoxymethyl, isopropoxymethyl, N-mesyl-2-aminoethoxymethyl, N-acetyl-2-aminoethoxymethyl, 2-(1,3-dioxo-1,3-dihydro-isoindol-2-yl)-ethoxymethyl, carbethoxycarbonylmethoxymethyl, 2-hydroxyethoxymethyl, imidazol-5-yl-methoxymethyl, imidazol-4-yl-methoxymethyl, 2-imidazol-1-yl-ethoxymethyl, 3-imidazol-1-yl-propyl, 3-pyrazol-1-yl-propyl, isopropoxymethyl, methoxyethoxymethyl, pyrrol-3-yl-methoxymethyl, pyrrol-2-yl-methoxymethyl, [1,2,4]triazol-3-yl-methoxymethyl, 2H-pyrazol-3-yl-methoxymethyl, 3H-[1,2,3]triazol-4-yl-methoxymethyl, or 2-pyrrol-1-yl-ethoxymethyl.

In another preferred embodiment, R₁₄ or R₃₇ is -halo, —NO₂, —CN, —OH, —OR₃₃, —C(O)R₃₄, —OC(O)R₃₄, —C(O)NHC(O)R₃₃, —(C₂-C₁₀)alkenyl, —(C₂-C₁₀)alkynyl, —(C₈-C₁₄)bicycloalkyl, —(C₅-C₁₀)cycloalkenyl, naphthyl, benzyl, —(C₁-C₆)alkyl-Z₁-(C₁-C₁₀)alkyl-R₃₉, —(C₁-C₁₀)alkyl-R₃₉, —(C₁-C₁₀)alkyl-NHR₃₈, —CO₂R₃₄, —NHC(O)R₃₄, —NHC(O)NHR₃₄, —C(O)NHR₃₄, —OC(O)R₃₄, —OC(O)OR₃₄, or —S(O)N(R₃₄)(R₃₄), wherein R₃₉, for each occurrence, is, independently, selected from —H, halo, —CN, —NO₂, —CN, —OH, —OR₃₄, —C(O)R₃₄, —OC(O)R₃₄, —C(O)NHC(O)R₃₄, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, phenyl, naphthyl, benzyl, —CO₂R₃₄, —C(O)OCH(R₃₄)(R₃₄), —NHC(O)R₃₄, —NHC(O)NHR₃₄, —C(O)NHR₃₄, —OC(O)R₃₄, —OC(O)OR₃₄, —NR₃₄S(O)₂R₃₃, —S(O)N(R₃₄)(R₃₄), —SR₃₄, —S(O)R₃₄, and —S(O)₂R₃₄.

In another preferred embodiment, in compounds represented by formula (I), (II), (VII), (VIII), (IX) or (X), R₁₄ or R₃₇ is a lower alkyl, a lower haloalkyl, a cycloalkyl, a —(C₁-C₆)alkyl-NHR₃₈, a —(C₁-C₆)alkyl-O—(C₁-C₆)alkyl-NHR₃₈, wherein R₃₈, for each occurrence, is —S(O)—(C₁-C₆)alkyl, —S(O)₂—(C₁-C₆)alkyl, and —C(O)—(C₁-C₆)alkyl.

Without wishing to be bound by any theory, it is believed that compounds having preferred R₁₄ and R₃₇ groups have no significant modulatory effect on L-type calcium channels. In this regard, compounds of the invention do not significantly inhibit L-type calcium channels if they inhibit activity of the channel by less than 50% at a concentration of 50 mM, preferably less than 50% at a concentration of 10 mM, more preferably less than 20% at a concentration of 10 mM, and still more preferably, less than 10% at a concentration of 10 mM.

Two or more of the embodiments listed for each of formulas (I), (II), (III), (IV), (V), (VI), (VII), (VIII), (IX), and (X) may be combined in any order to form additional embodiments of the invention provided that each of the combined embodiments are compatible with each other.

In one embodiment, compounds of the invention are dihydropyridine compounds characterized by an ability to reduce elevated blood glucose levels without a significant cardiovascular effect, wherein the core scaffold of the compounds is 1,4-dihydropyridine. In a preferred embodiment, compounds of the invention are 3-substituted-1,4-dihydropyridine compounds characterized by an ability to reduce elevated blood glucose levels without a significant cardiovascular effect. In another embodiment, the compounds of the invention are 4-substituted-1,4,5,6,7,8-hexahydroquinoline compounds characterized by the ability to reduce elevated blood glucose levels without a significant cardiovascular effect. In a more preferred embodiment, the 4-substituted-1,4,5,6,7,8-hexahydroquinoline compounds have a 5-oxo group.

Preferred 3-substituted-1,4-dihydropyridine compounds or the 4-substituted-1,4,5,6,7,8-hexahydroquinoline are those with a molecular weight of about 300 g/mol to about 500 g/mol, from about 350 g/mol to about 450 g/mol, or from about 400 g/mol to about 450 g/mol.

In one embodiment, when a variable of a compound of formula (I), (II), (III), (IV), (V), (VI), (VII), (VIII), (IX), (X) is defined as being a “substituent”, the variable can be halogen (i.e., chloro, iodo, bromo, or fluoro); C₁₋₆ alkyl; C₂₋₆ alkenyl; C₂₋₆ alkynyl; hydroxyl; C₁₋₆ alkoxyl; C₁₋₆ alkyl-O—C₁₋₆ alkyl (substituted or unsubstituted); amino; nitro; thiol; thioether; imine; cyano; amido; phosphonato; phosphine; carboxyl; thiocarbonyl; sulfonyl; sulfonamide; ketone; aldehyde; ester; oxygen (═O); haloalkyl (e.g., trifluoromethyl); carbocyclic cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl); carbocyclic or heterocyclic, monocyclic or fused or non-fused polycyclic aryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); o-lower alkyl; o-aryl, aryl; aryl-lower alkyl; CO₂CH₃; CONH₂; OCH₂CONH₂; NH₂; SO₂NH₂; OCHF₂; CF₃; OCF₃; and such moieties may also be optionally substituted by a fused-ring structure or bridge, for example —OCH₂O—. These substituents may optionally be further substituted with a substituent selected from such groups. In certain embodiments, the term “substituent” or the adjective “substituted” refers to a substituent selected from the group consisting of an alkyl, an alkenyl, an alkynyl, a cycloalkyl, a cycloalkenyl, a heterocycloalkyl, an aryl, a heteroaryl, an aralkyl, a heteraralkyl, a haloalkyl, —C(O)NR₂₈R₂₉, —NR₃₀C(O)R₃₁, a halo, —OR₃₀, cyano, nitro, a haloalkoxy, —C(O)R₃₀, —NR₂₈R₂₉, —SR₃₀, —C(O)OR₃₀, —OC(O)R₃₀, —NR₃₀C(O)NR₂₈R₂₉, —OC(O)NR₂₈R₂₉, —NR₃₀C(O)OR₃₁, —S(O)ᵣR₃₀, —S(O)ᵣNR₂₈R₂₉, ═O, ═S, and ═N—R₃₀, wherein R₂₈ and R₂₉, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₈ and R₂₉ taken together with the nitrogen to which they are attached is optionally substituted heterocycloalkyl or optionally substituted heteroaryl; and R₃₀ and R₃₁ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl.

The substituents used for compounds of formula (I), (II), (III), (IV), (V), (VI), (VII), (VIII), (IX), (X) or any of the specific compounds shown below can be used in any combination that results in the formation of a stable compound. All such combinations are expressly encompassed in this invention.

Compounds of the invention advantageously can possess one or more desired biological activities. Such activities include, but are not limited to, one or more of the following: the ability to reduce blood glucose levels, reduce cholesterol levels, normalize blood levels of lipids and insulin and/or improve insulin sensitivity in a patient in need thereof.

In one embodiment, compounds of the invention are useful for reducing blood glucose levels, reducing cholesterol levels, normalizing blood levels of lipids and insulin and/or improving insulin sensitivity in a patient in need thereof.

Without wishing to be bound by theory, it should be noted that compounds of the invention are 1,4-dihydropyridines, which is a class of compounds that includes particular antihypertensive agents used in the treatment of angina and/or other vascular diseases. Exemplary 1,4-dihydropyridines compounds being used for those cardiovascular indications include amlodipine, nifedipine, felodipine, nicardipine, and nisoldipine. In one embodiment, preferred compounds of this invention have activity against metabolic disorders without having a significant cardiovascular effect. In this context, a “significant cardiovascular effect” is no more than 75% (preferably, no more than 60% and more preferably, no more than 50%) of the cardiovascular activity of amlodipine in a standard procedure measuring mean arterial blood pressure (MAP) in a suitable cardiovascular animal model. In another embodiment, preferred compounds of this invention have activity against metabolic disorders without having significant acute toxicity. In this context, “without having significant acute toxicity” means having an LD₅₀ above about 250 mg/kg, above about 500 mg/kg, above about 750 mg/kg or above about 1000 mg/kg.

i) Exemplary Compounds of the Invention

Exemplary compounds of the invention are depicted in Table 1 below, including pharmaceutically acceptable salts, solvates, clathrates, hydrates, polymorphs or prodrugs thereof .

TABLE 1

| No. | Structure | Name |
|---|---|---|
| 1 | 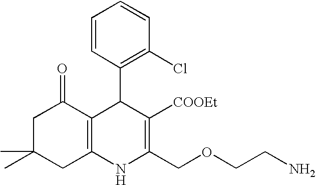 | 2-(2-amino-ethoxymethyl)-4-(2- chloro-phenyl)-7,7-dimethyl-5-oxo- 1,4,5,6,7,8-hexahydro-quinoline-3- carboxylic acid ethyl ester |
| 2 | 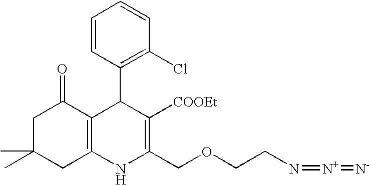 | 2-(2-azido-ethoxymethyl)-4-(2- chloro-phenyl)-7,7-dimethyl-5-oxo- 1,4,5,6,7,8-hexahydro-quinoline-3- carboxylic acid ethyl ester |
| 3 | 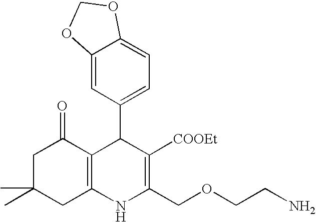 | 2-(2-amino-ethoxymethyl)-4- benzo[1,3]dioxol-5-yl-7,7-dimethyl- 5-oxo-1,4,5,6,7,8-hexahydro- quinoline-3-carboxylic acid ethyl ester |
| 4 | 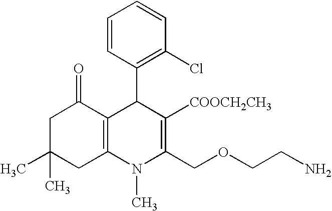 | 2-(2-amino-ethoxymethyl)-4-(2- chloro-phenyl)-1,7,7-trimethyl-5-oxo- 1,4,5,6,7,8-hexahydro-quinoline-3- carboxylic acid ethyl ester |
| 5 | 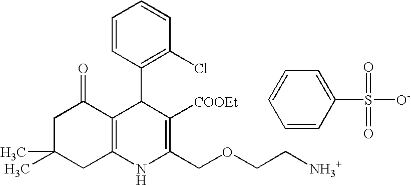 | 2-(2-amino-ethoxymethyl)-4-(2- chloro-phenyl)-7,7-dimethyl-5-oxo- 1,4,5,6,7,8-hexahydro-quinoline-3- carboxylic acid ethyl ester; complex with benzenesulfonic acid |
| 6 | 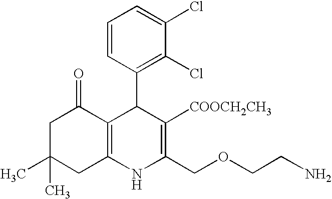 | 2-(2-amino-ethoxymethyl)-4-(2,3- dichloro-phenyl)-7,7-dimethyl-5-oxo- 1,4,5,6,7,8-hexahydro-quinoline-3- carboxylic acid ethyl ester |
| 7 | 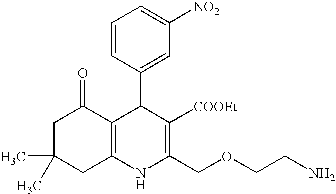 | 2-(2-amino-ethoxymethyl)-7,7- dimethyl-4-(3-nitro-phenyl)-5-oxo- 1,4,5,6,7,8-hexahydro-quinoline-3- carboxylic acid ethyl ester |
| 8 | 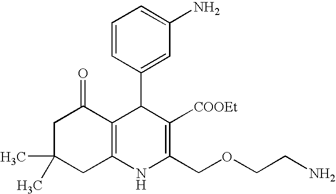 | 2-(2-amino-ethoxymethyl)-4-(3- amino-phenyl)-7,7-dimethyl-5-oxo- 1,4,5,6,7,8-hexahydro-quinoline-3- carboxylic acid ethyl ester |
| 9 | 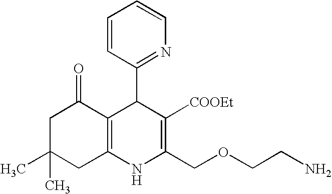 | 2-(2-amino-ethoxymethyl)-7,7- dimethyl-5-oxo-4-pyridin-2-yl- 1,4,5,6,7,8-hexahydro-quinoline-3- carboxylic acid ethyl ester |
| 10 | 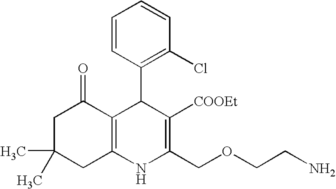 | 2-(2-amino-ethoxymethyl)-4-(2- chloro-phenyl)-5-oxo-1,4,5,6,7,8- hexahydro-quinoline-3-carboxylic acid ethyl ester |
| 11 | 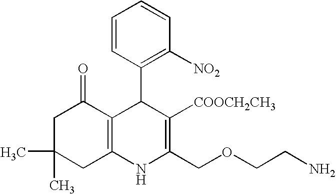 | 2-(2-amino-ethoxymethyl)-7,7- dimethyl-4-(2-nitro-phenyl)-5-oxo- 1,4,5,6,7,8-hexahydro-quinoline-3- carboxylic acid ethyl ester |
| 12 | 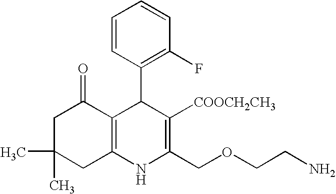 | 2-(2-amino-ethoxymethyl)-4-(2- fluoro-phenyl)-7,7-dimethyl-5-oxo- 1,4,5,6,7,8-hexahydro-quinoline-3- carboxylic acid ethyl ester |
| 13 | 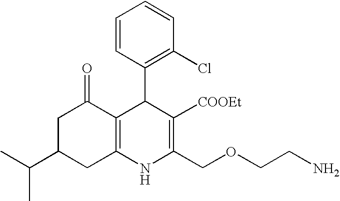 | 2-(2-amino-ethoxymethyl)-4-(2- chloro-phenyl)-7-isopropyl-5-oxo- 1,4,5,6,7,8-hexa-hydroquinoline-3- carboxylic acid ethyl ester |
| 14 | 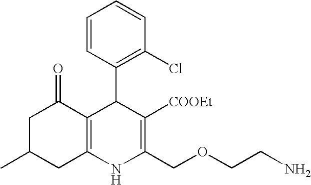 | 2-(2-amino-ethoxymethyl)-4-(2- chloro-phenyl)-7-methyl-5-oxo- 1,4,5,6,7,8-hexa-hydroquinoline-3- carboxylic acid ethyl ester |
| 15 | 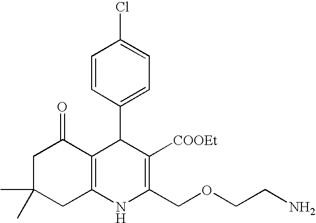 | 2-(2-Amino-ethoxymethyl)-4-(4- chloro-phenyl)-7,7-dimethyl-5-oxo- 1,4,5,6,7,8-hexahydro-quinoline-3- carboxylic acid ethyl ester |
| 16 | 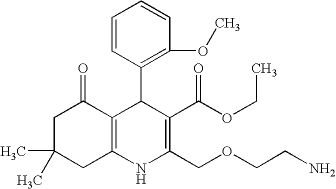 | 2-(2-Amino-ethoxymethyl)-4-(2- methoxy-phenyl)-7,7-dimethyl-5- oxo-1,4,5,6,7,8-hexahydro-quinoline- 3-carboxylic acid ethyl ester |
| 17 | 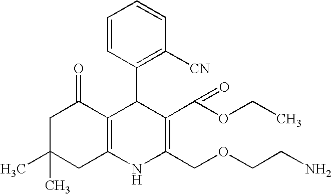 | 2-(2-Amino-ethoxymethyl)-4-(2- cyano-phenyl)-7,7-dimethyl-5-oxo- 1,4,5,6,7,8-hexahydro-quinoline-3- carboxylic acid ethyl ester |
| 18 | 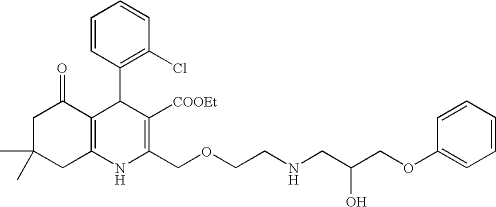 | 4-(2-Chloro-phenyl)-2-[2-(2-hydroxy- 3-phenoxy-propylamino)- ethoxymethyl]-7,7-dimethyl-5-oxo- 1,4,5,6,7,8-hexahydro-quinoline-3- carboxylic acid ethyl ester |
| 19 | 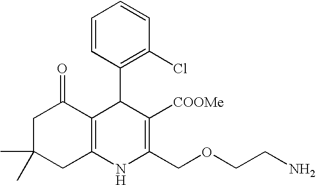 | 2-(2-amino-ethoxymethyl)-4-(2- chloro-phenyl)-7,7-dimethyl-5-oxo- 1,4,5,6,7,8-hexahydro-quinoline-3- carboxylic acid methyl ester |
| 20 | 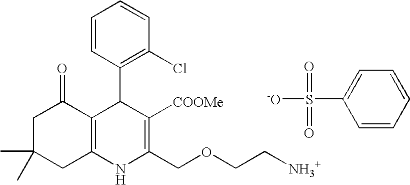 | 2-(2-amino-ethoxymethyl)-4-(2- chloro-phenyl)-7,7-dimethyl-5-oxo- 1,4,5,6,7,8-hexahydroquinoline-3- carboxylic acid methyl ester; complex with benzenesulfonic acid |
| 21 | 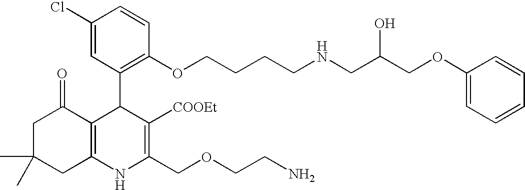 | 2-(2-Amino-ethoxymethyl)-4-{5- chloro-2-[4-(2-hydroxy-3-phenoxy- propylamino)-butoxy]-phenyl}-7,7- dimethyl-5-oxo-1,4,5,6,7,8- hexahydroquinoline-3-carboxylic acid ethyl ester |
| 22 | 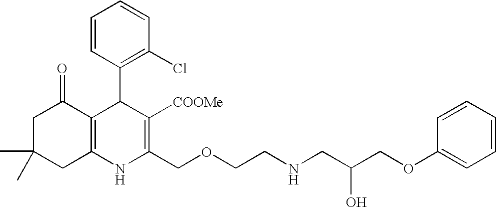 | 4-(2-Chloro-phenyl)-2-[2-(2-hydroxy- 3-phenoxy-propylamino)- ethoxymethyl]-7,7-dimethyl-5-oxo- 1,4,5,6,7,8-hexahydro-quinoline-3- carboxylic acid methyl ester |
| 23 | 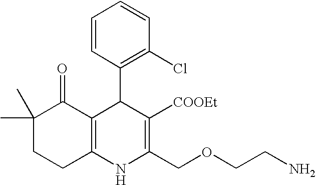 | 2-(2-Amino-ethoxymethyi)-4-(2- chloro-phenyl)-6,6-dimethyl-5-oxo- 1,4,5,6,7,8-hexahydro-quinoline-3- carboxylic acid ethyl ester |
| 24 | 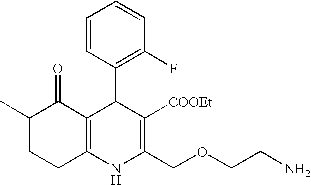 | 2-(2-Amino-ethoxymethyl)-4-(2- fluoro-phenyl)-7-methyl-5-oxo- 1,4,5,6,7,8-hexahydroquinoline-3- carboxylic acid ethyl ester |
| 25 | 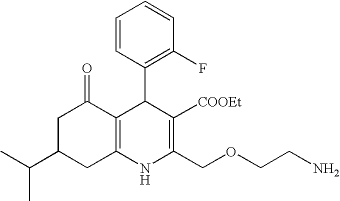 | 2-(2-Amino-ethoxymethyl)-4-(2- fluoro-phenyl)-7-isopropyl-5-oxo- 1,4,5,6,7,8-hexahydro-quinoline-3- carboxylic acid ethyl ester |
| 26 | 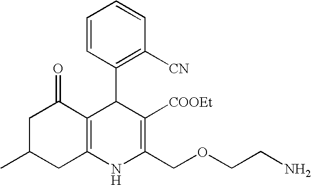 | 2-(2-Amino-ethoxymethyl)-4-(2- cyano-phenyl)-7-methyl-5-oxo- 1,4,5,6,7,8-hexahydro-quinoline-3- carboxylic acid ethyl ester |
| 27 | 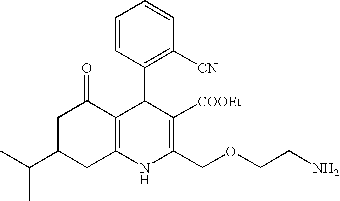 | 2-(2-Amino-ethoxymethyl)-4-(2- cyano-phenyl)-7-isopropyl-5-oxo- 1,4,5,6,7,8-hexahydro-quinolone-3- carboxylic acid ethyl ester |
| 28 | 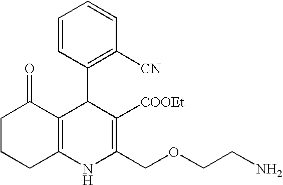 | 2-(2-Amino-ethoxymethyl)-4-(2- cyano-phenyl)-5-oxo-1,4,5,6,7,8- hexahydro-quinoline-3-carboxylic acid ethyl ester |
| 29 | 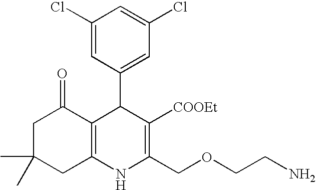 | 2-(2-Amino-ethoxymethyl)-4-(3,5- dichloro-phenyl)-7,7-dimethyl-5-oxo- 1,4,5,6,7,8-hexahydro-quinoline-3- carboxylic acid ethyl ester |
| 30 | 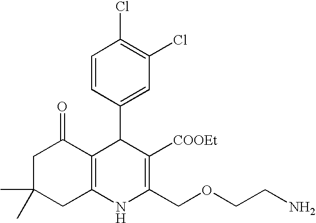 | 2-(2-Amino-ethoxymethyl)-4-(3,4- dichloro-phenyl)-7,7-dimethyl-5-oxo- 1,4,5,6,7,8-hexahydro-quinoline-3- carboxylic acid ethyl ester |
| 31 | 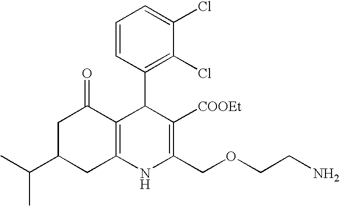 | 2-(2-Amino-ethoxymethyl)-4-(2,3- dichloro-phenyl)-7-isopropyl-5-oxo- 1,4,5,6,7,8-hexahydro-quinoline-3- carboxylic acid ethyl ester |
| 32 | 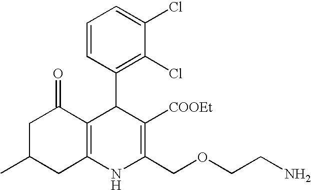 | 2-(2-Amino-ethoxymethyl)-4-(2,3- dichloro-phenyl)-7-methyl-5-oxo- 1,4,5,6,7,8-hexahydro-quinoline-3- carboxylic acid ethyl ester |
| 33 | 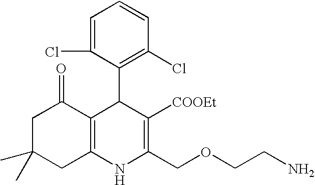 | 2-(2-Amino-ethoxymethyl)-4-(2,6- dichloro-phenyl)-7,7-dimethyl-5-oxo- 1,4,5,6,7,8-hexahydroquinoline-3- carboxylic acid ethyl ester |
| 34 | 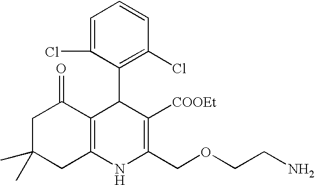 | 2-(2-Amino-ethoxymethyl)-4-(2,5- dichloro-phenyl)-7-methyl-5-oxo- 1,4,5,6,7,8-hexahydro-quinoline-3- carboxylic acid ethyl ester |
| 35 | 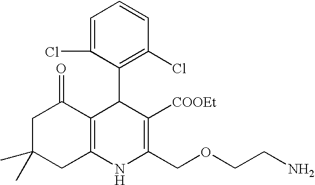 | 2-(2-Amino-ethoxymethyl)-4-(2,4- dichloro-phenyl)-7-methyl-5-oxo- 1,4,5,6,7,8-hexahydro-quinoline-3- carboxylic acid ethyl ester |
| 36 | 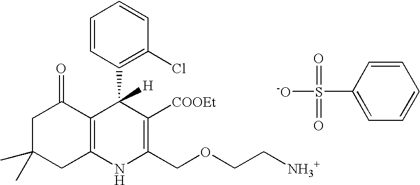 | S-(−)-2-(2-amino-ethoxymethyl)-4-(2- chloro-phenyl)-7,7-dimethyl-5-oxo- 1,4,5,6,7,8-hexahydro-quinoline-3- carboxylic acid ethyl ester; complex with benzenesulfonic acid |
| 37 | 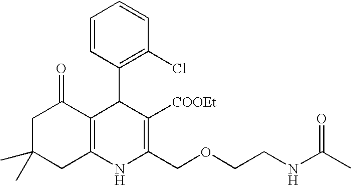 | 2-(2-Acetylamino-ethoxymethyl)-4- (2-chloro-phenyl)-7,7-dimethyl-5- oxo-1,4,5,6,7,8-hexahydro-quinoline- 3-carboxylic acid ethyl ester |
| 38 | 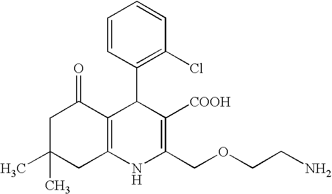 | 2-(2-Amino-ethoxymethyl)-4-(2- chloro-phenyl)-7,7-dimethyl-5-oxo- 1,4,5,6,7,8-hexahydro-quinoline-3- carboxylic acid |
| 39 | 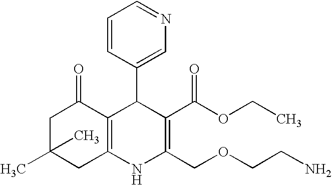 | 2-(2-Amino-ethoxymethyl)-7,7- dimethyl-5-oxo-4-pyridin-3-yl- 1,4,5,6,7,8-hexahydro-quinoline-3- carboxylic acid ethyl ester |
| 40 | 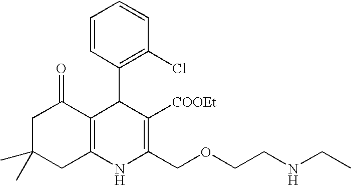 | 4-(2-Chloro-phenyl)-2-(2-ethylamino- ethoxymethyl)-7,7-dimethyl-5-oxo- 1,4,5,6,7,8-hexahydro-quinoline-3- carboxylic acid ethyl ester |
| 41 | 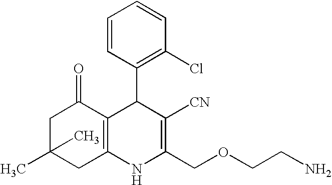 | 2-(2-Amino-ethoxymethyl)-4-(2- chloro-phenyl)-7,7-dimethyl-5-oxo- 1,4,5,6,7,8-hexahydro-quinoline-3- carbonitrile |
| 42 | 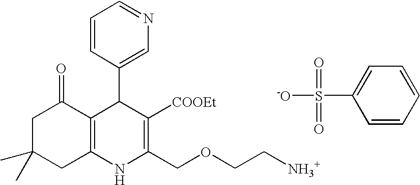 | Benzenesulfonate2-(3- ethoxycarbonyl-7,7-dimethyl-5-oxo- 4-pyridin-3-yl-1,4,5,6,7,8-hexahydro- quinolin-2-ylmethoxy)-ethyl- ammonium |
| 43 | 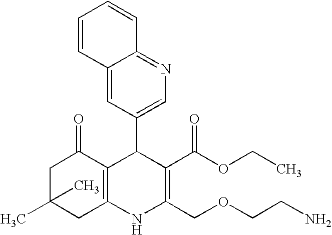 | 2-(2-Amino-ethoxymethyl)-7,7- dimethyl-5-oxo-4-quinolin-3-yl- 1,4,5,6,7,8-hexahydro-quinoline-3- carboxylic acid ethyl ester |
| 44 | 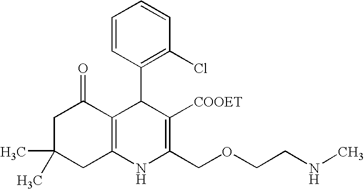 | 4-(2-Chloro-phenyl)-7,7-dimethyl-2- (2-methylamino-ethoxymethyl)-5- oxo-1,4,5,6,7,8-hexahydro-quinoline- acid ethyl ester |
| 45 | 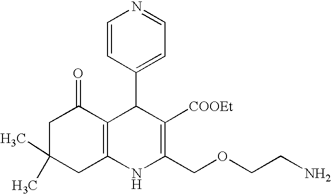 | 2-(2-Amino-ethoxymethyl)-7,7- dimethyl-5-oxo-4-pyridin-4-yl- 1,4,5,6,7,8-hexahydro-quinoline-3- carboxylic acid ethyl ester |
| 46 | 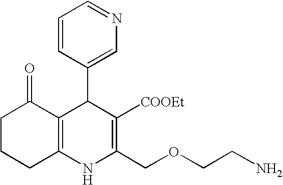 | 2-(2-Amino-ethoxymethyl)-5-oxo-4- pyridin-3-yl-1,4,5,6,7,8-hexahydro- quinoline-3-carboxylic acid ethyl ester |
| 47 | 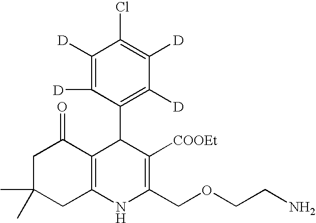 | 2-(2-Amino-ethoxymethyl)-4-(4- chloro-2,3,5,6-tetradeuterium- phenyl)-7,7-dimethyl-5-oxo- 1,4,5,6,7,8-hexahydro-quinoline-3- carboxylic acid ethyl ester |
| 48 | 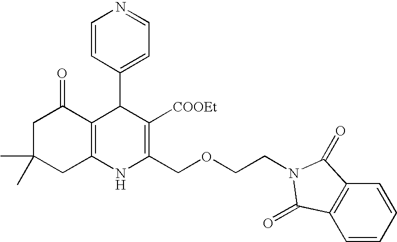 | 2-[2-(1,3-Dioxo-1,3-dihydro-isoindol- 2-yl)-ethoxymethyl]-7,7-dimethyl-5- oxo-4-pyridin-4-yl-1,4,5,6,7,8- hexahydro-quinoline-3-carboxylic acid ethyl ester |
| 49 | 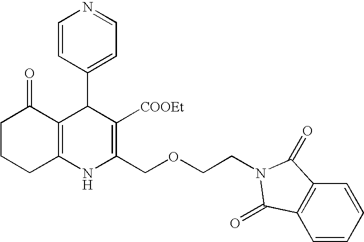 | 2-[2-(1,3-Dioxo-1,3-dihydro-isoindol- 2-yl)-ethoxymethyl]-5-oxo-4-pyridin- 3-yl-1,4,5,6,7,8-hexahydro-quinoline- 3-carboxylic acid ethyl ester |
| 50 | 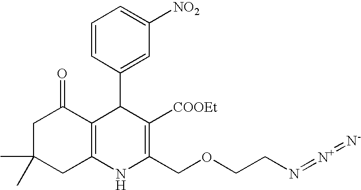 | 2-(2-Azido-ethoxymethyl)-7,7- dimethyl-4-(3-nitro-phenyl)-5-oxo- 1,4,5,6,7,8-hexahydro-quinoline-3- carboxylic acid ethyl ester |
| 51 | 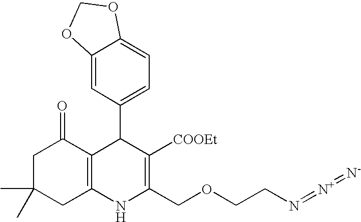 | 2-(2-azido-ethoxymethyl)-4- benzo[1,3]dioxol-5-yl-7,7-dimethyl- 5-oxo-1,4,5,6,7,8-hexahydro- quinoline-3-carboxylic acid ethyl ester |
| 52 | 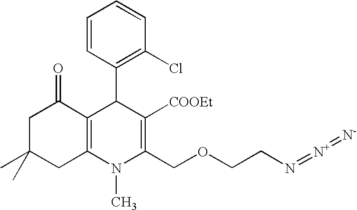 | 2-(2-azido-ethoxymethyl)-4-(2- chloro-phenyl)-1,7,7-trimethyl-5-oxo- 1,4,5,6,7,8-hexahydro-quinoline-3- carboxylic acid ethyl ester |
| 53 | 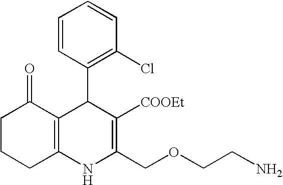 | 2-(2-Amino-ethoxymethyl)-4-(2- chloro-phenyl)-5-oxo-1,4,5,6,7,8- hexahydro-quinoline-3-carboxylic acid ethyl ester |
| 54 | 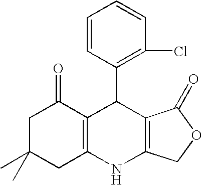 | 9-(2-chloro-phenyl)-6,6-dimethyl- 5,6,7,9-tetrahydro-3H,4H-furo[3,4- b]quinoline-1,8-dione |
| 55 | 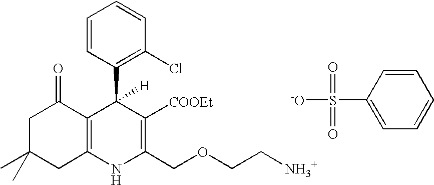 | (+)-(R)-2-(2-amino-ethoxymethyl)-4- (2-chloro-phenyl)-7,7-dimethyl-5- oxo-1,4,5,6,7,8-hexahydro-quinoline- 3-carboxylic acid ethyl ester; compound with benzenesulfonic acid |
| 56 | 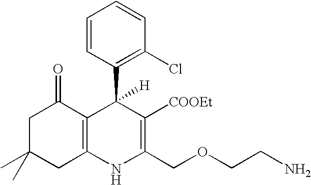 | (+)-(R)-2-(2-amino-ethoxymethyl)-4- (2-chloro-phenyl)-7,7-dimethyl-5- oxo-1,4,5,6,7,8-hexahydro-quinoline- 3-carboxylic acid ethyl ester |
| 57 | 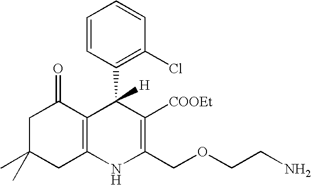 | (−)-(S)-2-(2-amino-ethoxymethyl)-4- (2-chloro-phenyl)-7,7-dimethyl-5- oxo-1,4,5,6,7,8-hexahydro-quinoline- 3-carboxylic acid ethyl ester |
| 58 | 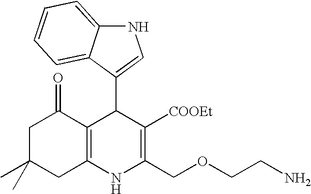 | 2-(2-Amino-ethoxymethyl)-4-(1H- indol-3-yl)-7,7-dimethyl-5-oxo- 1,4,5,6,7,8-hexahydro-quinoline-3- carboxylic acid ethyl ester |
| 59 | 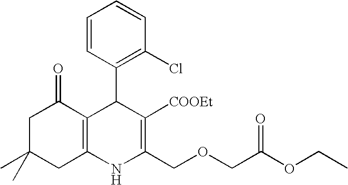 | 4-(2-Chloro-phenyl)-2- ethoxycarbonylmethoxymethyl-7,7- dimethyl-5-oxo-1,4,5,6,7,8- hexahydro-quinoline-3-carboxylic acid ethyl ester |
| 60 | 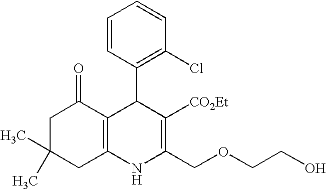 | 4-(2-Chloro-phenyl)-2-(hydroxyl- ethoxymethyl)-7,7-dimethyl-5-oxo- 1,4,5,6,7,8-hexahydro-quinoline-3- carboxylic acid ethyl ester |
| 61 | 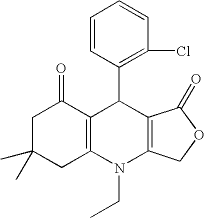 | 9-(2-Chloro-phenyl)-4-ethyl-6,6- dimethyl-5,6,7,9-tetrahydro-3H,4H- furo[3,4b]quinoline-1,8-dione |
| 62 | 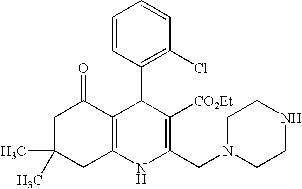 | 4-(2-Chloro-phenyl)-7,7-dimethyl-5- oxo-2-piperazin-1-ylmethyl- 1,4,5,6,7,8-hexahydro-quinoline-3- carboxylic acid ethyl ester |
| 63 | 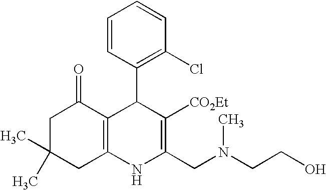 | 4-(2-Chloro-phenyl)-2-{[(2-hydroxy- ethyl)-methyl-amino]-methyl}-7,7- dimethyl-5-oxo-1,4,5,6,7,8- hexahydro-quinoline-3-carboxylic acid ethyl ester |
| 64 | 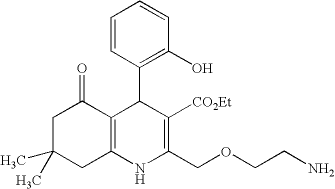 | 2-(2-Amino-ethoxymethyl)-4-(2- hydroxy-phenyl)-7,7-dimethyl-5-oxo- 1,4,5,6,7,8-hexahydro-quinoline-3- carboxylic acid ethyl ester |
| 65 | 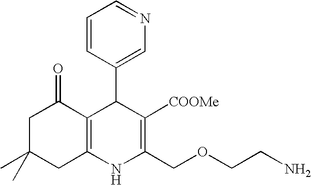 | 2-(2-Amino-ethoxymethyl)-7,7- dimethyl-5-oxo-4-pyridin-3-yl- 1,4,5,6,7,8-hexahydro-quinoline-3- carboxylic acid methyl ester |
| 66 | 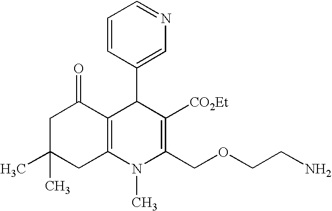 | 2-(2-Amino-ethoxymethyl)-1,7,7- trimethyl-5-oxo-4-pyridin-3-yl- 1,4,5,6,7,8-hexahydro-quinoline-3- carboxylic acid ethyl ester |
| 67 | 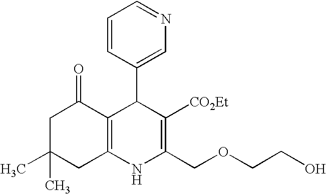 | 2-(2-Hydroxy-ethoxymethyl)-7,7- dimethyl-5-oxo-4-pyridin-3-yl- 1,4,5,6,7,8-hexahydro-quinoline-3- carboxylic acid ethyl ester |
| 68 | 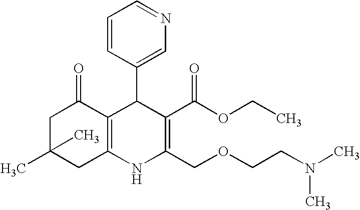 | 2-(2-Dimethylamino-ethoxymethyl)- 7,7-dimethyl-5-oxo-4-pyridin-3yl- 1,4,5,6,7,8,-hexahydro-quinoline-3- carboxylic acid ethyl ester |
| 69 | 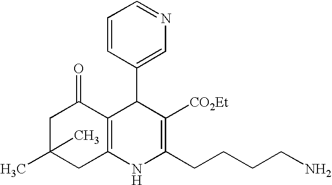 | 2-(4-Amino-butyl)-7,7-dimethyl-5- oxo-4-pyridin-3yl-1,4,5,6,7,8,- hexahydro-quinoline-3-carboxylic acid ethyl ester |
| 70 | 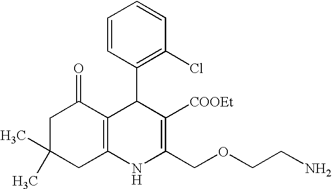 | 2-(2-Amino-ethoxymethyl)-4-(2- chloro-phenyl)-7,7-dimethyl-4,6,7,8- tetrahydro-1H-quinolin-5-one |
| 71 | 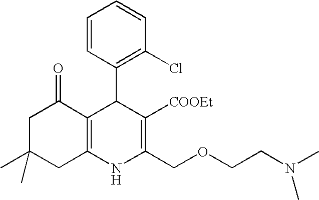 | 4-(2-Chloro-phenyl)-2-(2- dimethylamino-ethoxymethyl)-7,7- dimethyl-5-oxo-1,4,5,6,7,8- hexahydro-quinoline-3-carboxylic acid ethyl ester |
| 72 | 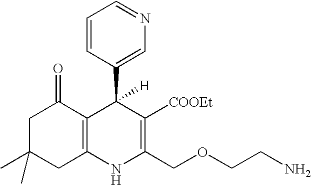 | (+)-(R)-2-(2-Amino-ethoxymethyl)- 7,7-dimethyl-5-oxo-4-pyridin-3-yl- 1,4,5,6,7,8-hexahydro-quinoline-3- carboxylic acid ethyl ester |
| 73 | 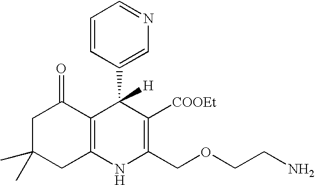 | (−)-(S)-2-(2-Amino-ethoxymethyl)- 7,7-dimethyl-5-oxo-4-pyridin-3-yl- 1,4,5,6,7,8-hexahydro-quinoline-3- carboxylic acid ethyl ester |
| 74 | 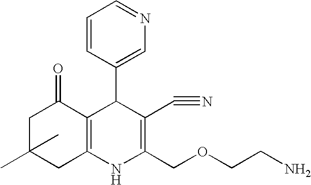 | 2-(2-Amino-ethoxymethyl)-7,7- dimethyl-5-oxo-4-pyridin-3-yl- 1,4,5,6,7,8-hexahydro-quinoline-3- carbonitrile |
| 75 | 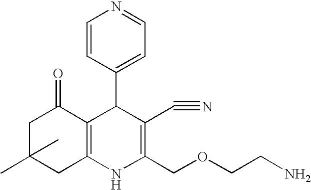 | 2-(2-Amino-ethoxymethyl)-7,7- dimethyl-5-oxo-4-pyridin-4-yl- 1,4,5,6,7,8-hexahydro-quinoline-3- carbonitrile |
| 76 | 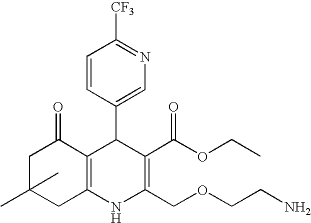 | 2-(2-Amino-ethoxymethyl)-7,7- dimethyl-5-oxo-4-(6-trifluoromethyl- pyridin-3-yl)-1,4,5,6,7,8-hexahydro- quinoline-3-carboxylic acid ethyl ester |
| 77 | 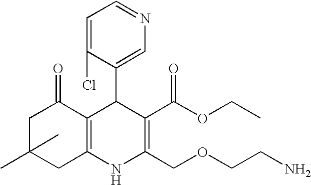 | 2-(2-Amino-ethoxymethyl)-4-(4- chloro-pyridin-3-yl)-7,7-dimethyl-5- oxo-1,4,5,6,7,8-hexahydro-quinoline- 3-carboxylic acid ethyl ester |
| 78 | 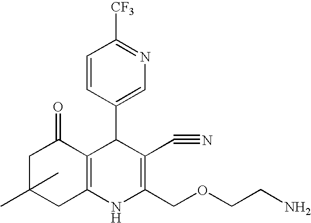 | 2-(2-Amino-ethoxymethyl)-7,7- dimethyl-5-oxo-4-(6-trifluoromethyl- pyridin-3-yl)-1,4,5,6,7,8-hexahydro- quinoline-3-carbonitrile |
| 79 | 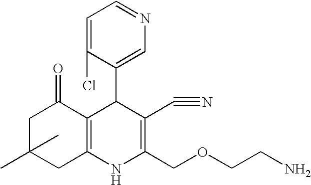 | 2-(2-Amino-ethoxymethyl)-4-(4- chloro-pyridin-3-yl)-7,7-dimethyl-5- oxo-1,4,5,6,7,8-hexahydro-quinoline- 3-carbonitrile |
| 80 | 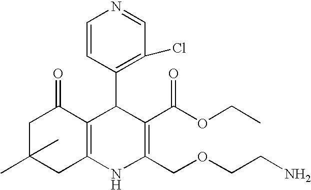 | 2-(2-Amino-ethoxymethyl)-4-(3- chloro-pyridin-4-yl)-7,7-dimethyl-5- oxo-1,4,5,6,7,8-hexahydro-quinoline- 3-carboxylic acid ethyl ester |
| 81 | 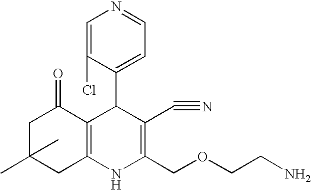 | 2-(2-Amino-ethoxymethyl)-4-(3- chloro-pyridin-4-yl)-7,7-dimethyl-5- oxo-1,4,5,6,7,8-hexahydro-quinoline- 3-carbonitrile |
| 82 | 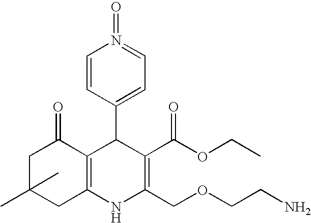 | 2-(2-Amino-ethoxymethyl)-7,7- dimethyl-5-oxo-4-(1-oxy-pyridin-4- yl)-1,4,5,6,7,8-hexahydro-quinoline- 3-carboxylic acid ethyl ester |
| 83 | 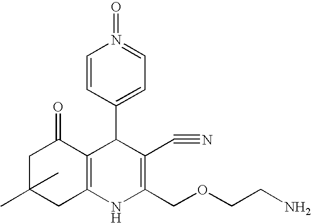 | 2-(2-Amino-ethoxymethyl)-7,7- dimethyl-5-oxo-4-(1-oxy-pyridin-4- yl)-1,4,5,6,7,8-hexahydro-quinoline- 3-carbonitrile |
| 84 | 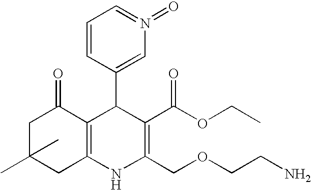 | 2-(2-Amino-ethoxymethyl)-7,7- dimethyl-5-oxo-4-(1-oxy-pyridin-3- yl)-1,4,5,6,7,8-hexahydro-quinoline- 3-carboxylic acid ethyl ester |
| 85 | 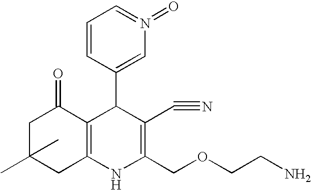 | 2-(2-Amino-ethoxymethyl)-7,7- dimethyl-5-oxo-4-(1-oxy-pyridin-3- yl)-1,4,5,6,7,8-hexahydro-quinoline- 3-carbonitrile |
| 86 | 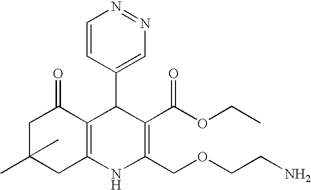 | 2-(2-Amino-ethoxymethyl)-7,7- dimethyl-5-oxo-4-pyridazin-4-yl- 1,4,5,6,7,8-hexahydro-quinoline-3- carboxylic acid ethyl ester |
| 87 | 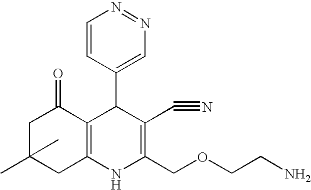 | 2-(2-Amino-ethoxymethyl)-7,7- dimethyl-5-oxo-4-pyridazin-4-yl- 1,4,5,6,7,8-hexahydro-quinoline-3- carbonitrile |
| 88 | 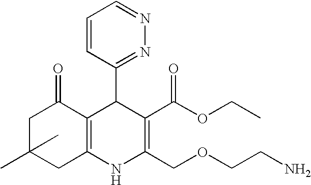 | 2-(2-Amino-ethoxymethyl)-7,7- dimethyl-5-oxo-4-pyridazin-3-yl- 1,4,5,6,7,8-hexahydro-quinoline-3- carboxylic acid ethyl ester |
| 89 | 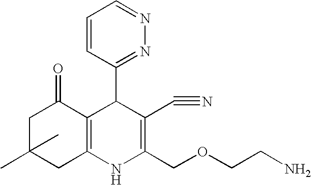 | 2-(2-Amino-ethoxymethyl)-7,7- dimethyl-5-oxo-4-pyridazin-3-yl- 1,4,5,6,7,8-hexahydro-quinoline-3- carbonitrile |
| 90 | 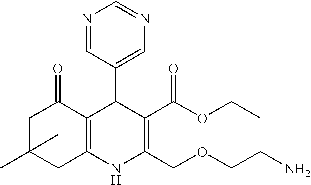 | 2-(2-Amino-ethoxymethyl)-7,7- dimethyl-5-oxo-4-pyrimidin-5-yl- 1,4,5,6,7,8-hexahydro-quinoline-3- carboxylic acid ethyl ester |
| 91 | 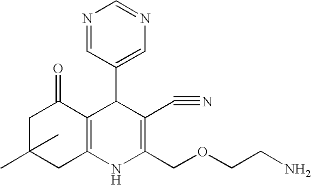 | 2-(2-Amino-ethoxymethyl)-7,7- dimethyl-5-oxo-4-pyrimidin-5-yl- 1,4,5,6,7,8-hexahydro-quinoline-3- carbonitrile |
| 92 | 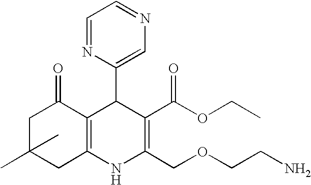 | 2-(2-Amino-ethoxymethyl)-7,7- dimethyl-5-oxo-4-pyrazin-2-yl- 1,4,5,6,7,8-hexahydro-quinoline-3- carboxylic acid ethyl ester |
| 93 | 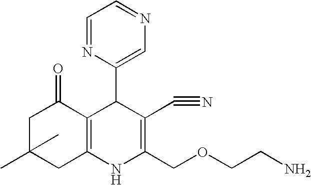 | 2-(2-Amino-ethoxymethyl)-7,7- dimethyl-5-oxo-4-pyrazin-2-yl- 1,4,5,6,7,8-hexahydro-quinoline-3- carbonitrile |
| 94 | 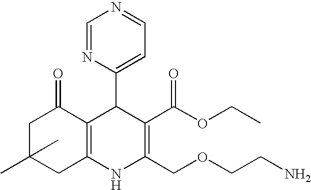 | 2-(2-Amino-ethoxymethyl)-7,7- dimethyl-5-oxo-4-pyrimidin-4-yl- 1,4,5,6,7,8-hexahydro-quinoline-3- carboxylic acid ethyl ester |
| 95 | 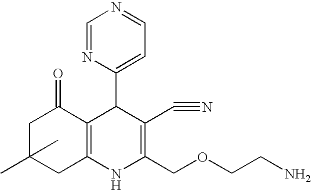 | 2-(2-Amino-ethoxymethyl)-7,7- dimethyl-5-oxo-4-pyrimidin-4-yl- 1,4,5,6,7,8-hexahydro-quinoline-3- carbonitrile |
| 96 | 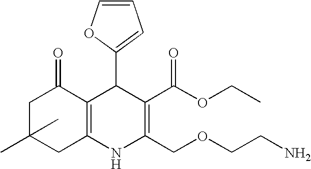 | 2-(2-Amino-ethoxymethyl)-4-furan- 2-yl-7,7-dimethyl-5-oxo-1,4,5,6,7,8- hexahydro-quinoline-3-carboxylic acid ethyl ester |
| 97 | 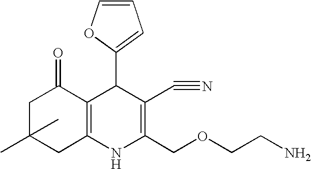 | 2-(2-Amino-ethoxymethyl)-4-furan- 2-yl-7,7-dimethyl-5-oxo-1,4,5,6,7,8- hexahydro-quinoline-3-carbonitrile |
| 98 | 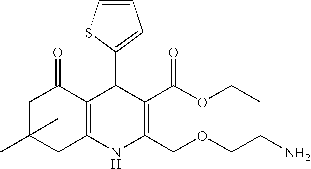 | 2-(2-Amino-ethoxymethyl)-7,7- dimethyl-5-oxo-4-thiophen-2-yl- 1,4,5,6,7,8-hexahydro-quinoline-3- carboxylic acid ethyl ester |
| 99 | 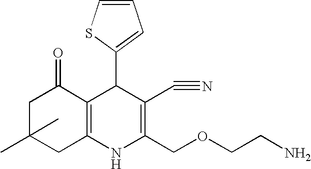 | 2-(2-Amino-ethoxymethyl)-7,7- dimethyl-5-oxo-4-thiophen-2-yl- 1,4,5,6,7,8-hexahydro-quinoline-3- carbonitrile |
| 100 | 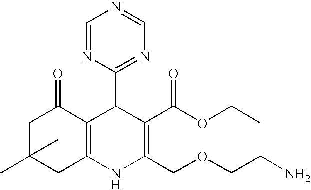 | 2-(2-Amino-ethoxymethyl)-7,7- dimethyl-5-oxo-4-[1,3,5]triazin-2-yl- 1,4,5,6,7,8-hexahydro-quinoline-3- carboxylic acid ethyl ester |
| 101 | 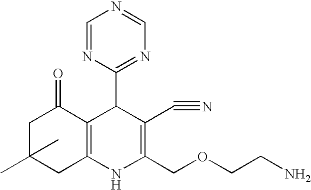 | 2-(2-Amino-ethoxymethyl)-7,7- dimethyl-5-oxo-4-[1,3,5]triazin-2-yl- 1,4,5,6,7,8-hexahydro-quinoline-3- carbonitrile |
| 102 | 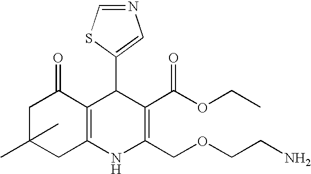 | 2-(2-Amino-ethoxymethyl)-7,7- dimethyl-5-oxo-4-thiazol-5-yl- 1,4,5,6,7,8-hexahydro-quinoline-3- carboxylic acid ethyl ester |
| 103 | 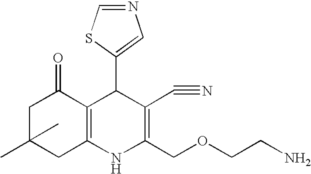 | 2-(2-Amino-ethoxymethyl)-7,7- dimethyl-5-oxo-4-thiazol-5-yl- 1,4,5,6,7,8-hexahydro-quinoline-3- carbonitrile |
| 104 | 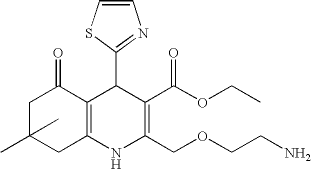 | 2-(2-Amino-ethoxymethyl)-7,7- dimethyl-5-oxo-4-thiazol-2-yl- 1,4,5,6,7,8-hexahydro-quinoline-3- carboxylic acid ethyl ester |
| 105 | 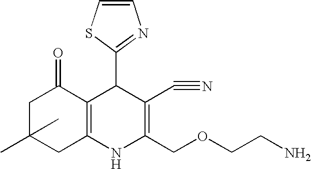 | 2-(2-Amino-ethoxymethyl)-7,7- dimethyl-5-oxo-4-thiazol-2-yl- 1,4,5,6,7,8-hexahydro-quinoline-3- carbonitrile |
| 106 | 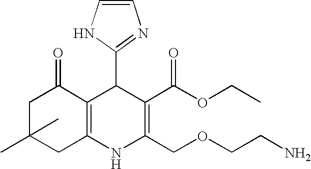 | 2-(2-Amino-ethoxymethyl)-4-(1H- imidazol-2-yl)-7,7-dimethyl-5-oxo- 1,4,5,6,7,8-hexahydro-quinoline-3- carboxylic acid ethyl ester |
| 107 | 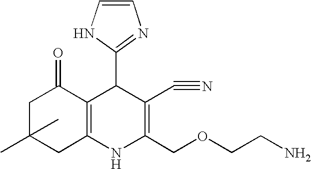 | 2-(2-Amino-ethoxymethyl)-4-(1H- imidazol-2-yl)-7,7-dimethyl-5-oxo- 1,4,5,6,7,8-hexahydro-quinoline-3- carbonitrile |
| 108 | 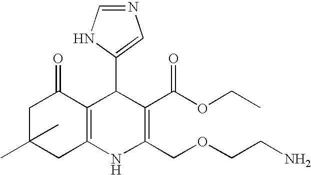 | 2-(2-Amino-ethoxymethyl)-4-(3H- imidazol-4-yl)-7,7-dimethyl-5-oxo- 1,4,5,6,7,8-hexahydro-quinoline-3- carboxylic acid ethyl ester |
| 109 | 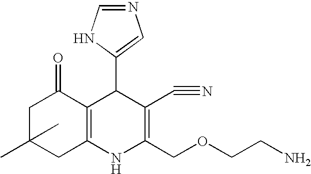 | 2-(2-Amino-ethoxymethyl)-4-(3H- imidazol-4-yl)-7,7-dimethyl-5-oxo- 1,4,5,6,7,8-hexahydro-quinoline-3- carbonitrile |
| 110 | 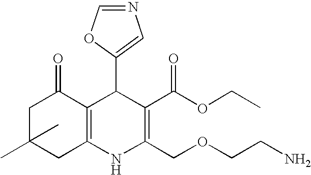 | 2-(2-Amino-ethoxymethyl)-7,7- dimethyl-4-oxazol-5-yl-5-oxo- 1,4,5,6,7,8-hexahydro-quinoline-3- carboxylic acid ethyl ester |
| 111 | 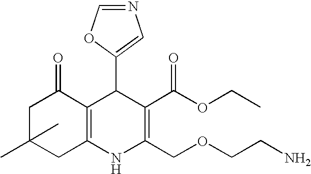 | 2-(2-Amino-ethoxymethyl)-7,7- dimethyl-4-oxazol-5-yl-5-oxo- 1,4,5,6,7,8-hexahydro-quinoline-3- carbonitrile |
| 112 | 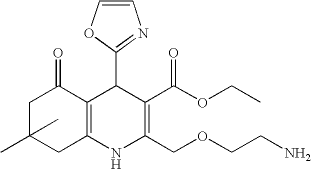 | 2-(2-Amino-ethoxymethyl)-7,7- dimethyl-4-oxazol-2-yl-5-oxo- 1,4,5,6,7,8-hexahydro-quinoline-3- carboxylic acid ethyl ester |
| 113 | 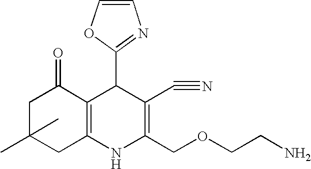 | 2-(2-Amino-ethoxymethyl)-7,7- dimethyl-4-oxazol-2-yl-5-oxo- 1,4,5,6,7,8-hexahydro-quinoline-3- carbonitrile |
| 114 | 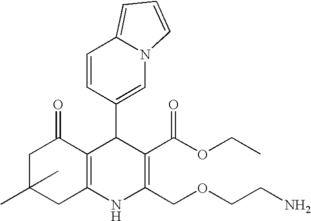 | 2-(2-Amino-ethoxymethyl)-4- indolizin-6-yl-7,7-dimethyl-5-oxo- 1,4,5,6,7,8-hexahydro-quinoline-3- carboxylic acid ethyl ester |
| 115 | 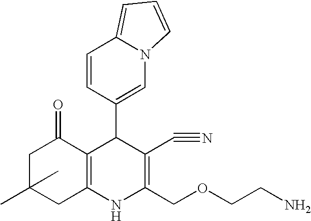 | 2-(2-Amino-ethoxymethyl)-4- indolizin-6-yl-7,7-dimethyl-5-oxo- 1,4,5,6,7,8-hexahydro-quinoline-3- carbonitrile |
| 116 | 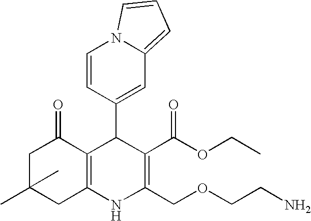 | 2-(2-Amino-ethoxymethyl)-4- indolizin-7-yl-7,7-dimethyl-5-oxo- 1,4,5,6,7,8-hexahydro-quinoline-3- carboxylic acid ethyl ester |
| 117 | 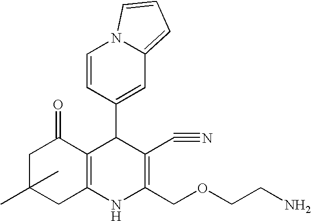 | 2-(2-Amino-ethoxymethyl)-4- indolizin-7-yl-7,7-dimethyl-5-oxo- 1,4,5,6,7,8-hexahydro-quinoline-3- carbonitrile |
| 118 | 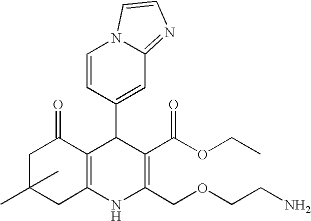 | 2-(2-Amino-ethoxymethyl)-4- imidazo[1,2-a]pyridin-7-yl-7,7- dimethyl-5-oxo-1,4,5,6,7,8- hexahydro-quinoline-3-carboxylic acid ethyl ester |
| 119 | 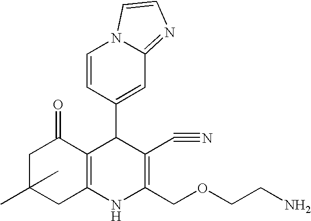 | 2-(2-Amino-ethoxymethyl)-4- imidazo[1,2-a]pyridin-7-yl-7,7- dimethyl-5-oxo-1,4,5,6,7,8- hexahydro-quinoline-3-carbonitrile |
| 120 | 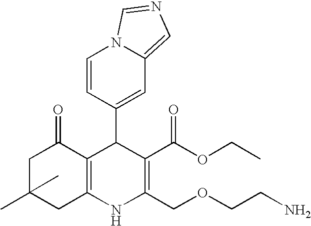 | 2-(2-Amino-ethoxymethyl)-4- imidazo[1,5-a]pyridin-7-yl-7,7- dimethyl-5-oxo-1,4,5,6,7,8- hexahydro-quinoline-3-carboxylic acid ethyl ester |
| 121 | 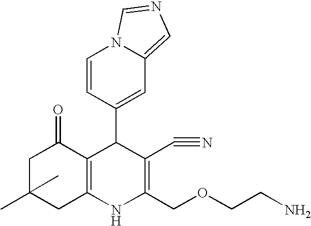 | 2-(2-Amino-ethoxymethyl)-4- imidazo[1,5-a]pyridin-7-yl-7,7- dimethyl-5-oxo-1,4,5,6,7,8- hexahydro-quinoline-3-carbonitrile |
| 122 | 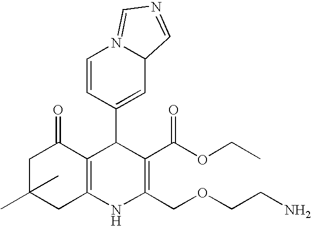 | 2-(2-Amino-ethoxymethyl)-4- imidazo[1,5-a]pyridin-6-yl-7,7- dimethyl-5-oxo-1,4,5,6,7,8- hexahydro-quinoline-3-carboxylic acid ethyl ester |
| 123 | 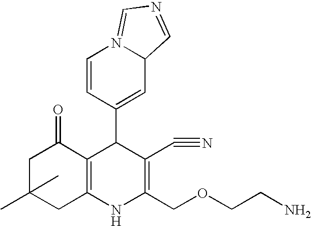 | 2-(2-Amino-ethoxymethyl)-4- imidazo[1,5-a]pyridin-6-yl-7,7- dimethyl-5-oxo-1,4,5,6,7,8- hexahydro-quinoline-3-carbonitrile |
| 124 | 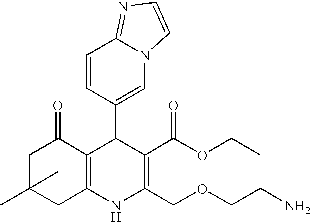 | 2-(2-Amino-ethoxymethyl)-4- imidazo[1,2-a]pyridin-6-yl-7,7- dimethyl-5-oxo-1,4,5,6,7,8- hexahydro-quinoline-3-carboxylic acid ethyl ester |
| 125 | 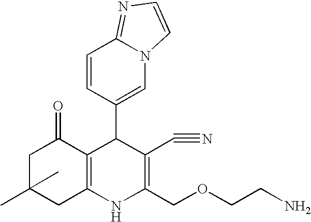 | 2-(2-Amino-ethoxymethyl)-4- imidazo[1,2-a]pyridin-6-yl-7,7- dimethyl-5-oxo-1,4,5,6,7,8- hexahydro-quinoline-3-carbonitrile |
| 126 | 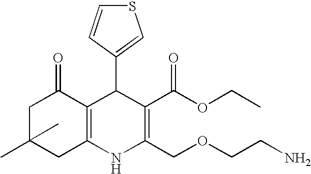 | 2-(2-Amino-ethoxymethyl)-7,7- dimethyl-5-oxo-4-thiophen-3-yl- 1,4,5,6,7,8-hexahydro-quinoline-3- carboxylic acid ethyl ester |
| 127 | 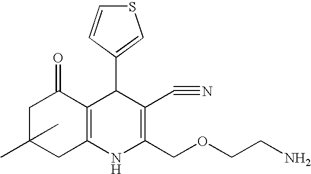 | 2-(2-Amino-ethoxymethyl)-7,7- dimethyl-5-oxo-4-thiophen-3-yl- 1,4,5,6,7,8-hexahydro-quinoline-3- carbonitrile |
| 128 | 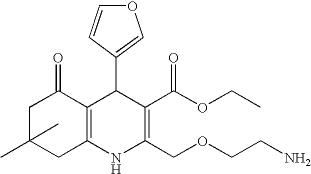 | 2-(2-Amino-ethoxymethyl)-4-furan- 3-yl-7,7-dimethyl-5-oxo-1,4,5,6,7,8- hexahydro-quinoline-3-carboxylic acid ethyl ester |
| 129 | 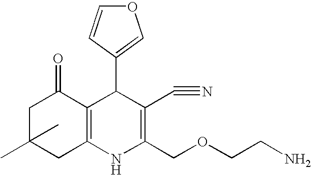 | 2-(2-Amino-ethoxymethyl)-4-furan- 3-yl-7,7-dimethyl-5-oxo-1,4,5,6,7,8- hexahydro-quinoline-3-carbonitrile |
| 130 | 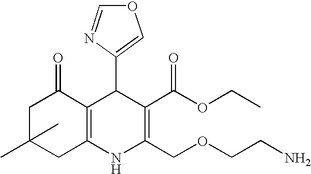 | 2-(2-Amino-ethoxymethyl)-7,7- dimethyl-4-oxazol-4-yl-5-oxo- 1,4,5,6,7,8-hexahydro-quinoline-3- carboxylic acid ethyl ester |
| 131 | 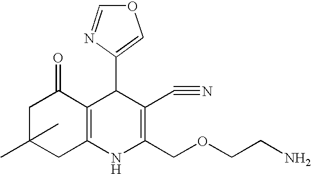 | 2-(2-Amino-ethoxymethyl)-7,7- dimethyl-4-oxazol-4-yl-5-oxo- 1,4,5,6,7,8-hexahydro-quinoline-3- carbonitrile |
| 132 | 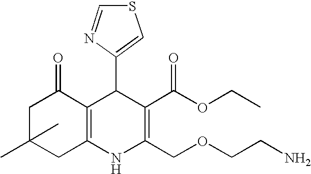 | 2-(2-Amino-ethoxymethyl)-7,7- dimethyl-5-oxo-4-thiazol-4-yl- 1,4,5,6,7,8-hexahydro-quinoline-3- carboxylic acid ethyl ester |
| 133 | 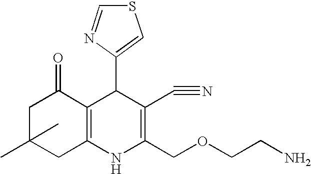 | 2-(2-Amino-ethoxymethyl)-7,7- dimethyl-5-oxo-4-thiazol-4-yl- 1,4,5,6,7,8-hexahydro-quinoline-3- carbonitrile |
| 134 | 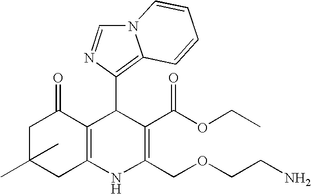 | 2-(2-Amino-ethoxymethyl)-4- imidazo[1,5-a]pyridin-1-yl-7,7- dimethyl-5-oxo-1,4,5,6,7,8- hexahydro-quinoline-3-carboxylic acid ethyl ester |
| 135 | 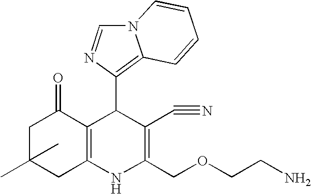 | 2-(2-Amino-ethoxymethyl)-4- imidazo[1,5-a]pyridin-1-yl-7,7- dimethyl-5-oxo-1,4,5,6,7,8- hexahydro-quinoline-3-carbonitrile |
| 136 | 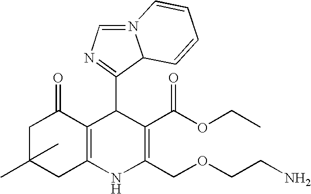 | 2-(2-Amino-ethoxymethyl)-4- imidazo[1,5-a]pyridin-3-yl-7,7- dimethyl-5-oxo-1,4,5,6,7,8- hexahydro-quinoline-3-carboxylic acid ethyl ester |
| 137 | 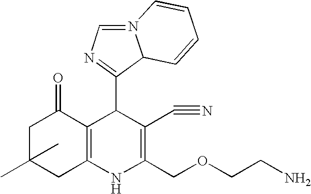 | 2-(2-Amino-ethoxymethyl)-4- imidazo[1,5-a]pyridin-3-yl-7,7- dimethyl-5-oxo-1,4,5,6,7,8- hexahydro-quinoline-3-carbonitrile |
| 138 | 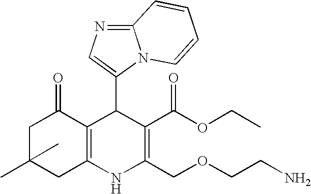 | 2-(2-Amino-ethoxymethyl)-4- imidazo[1,2-a]pyridin-3-yl-7,7- dimethyl-5-oxo-1,4,5,6,7,8- hexahydro-quinoline-3-carboxylic acid ethyl ester |
| 139 | 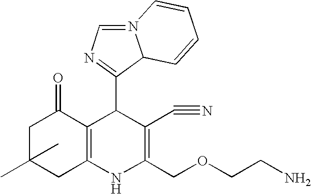 | 2-(2-Amino-ethoxymethyl)-4- imidazo[1,2-a]pyridin-3-yl-7,7- dimethyl-5-oxo-1,4,5,6,7,8- hexahydro-quinoline-3-carbonitrile |
| 140 | 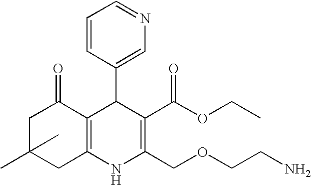 | 2-(2-Amino-ethoxymethyl)-7,7- dimethyl-5-oxo-4-pyridin-3-yl- 1,4,5,6,7,8-hexahydro-quinoline-3- carboxylic acid propyl ester |
| 141 | 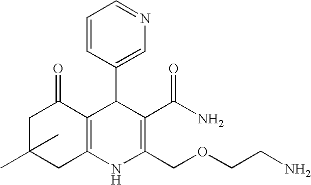 | 2-(2-Amino-ethoxymethyl)-7,7- dimethyl-5-oxo-4-pyridin-3-yl- 1,4,5,6,7,8-hexahydro-quinoline-3- carboxylic acid amide |
| 142 | 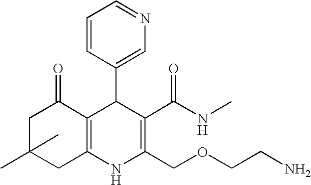 | 2-(2-Amino-ethoxymethyl)-7,7- dimethyl-5-oxo-4-pyridin-3-yl- 1,4,5,6,7,8-hexahydro-quinoline-3- carboxylic acid methylamide |
| 143 | 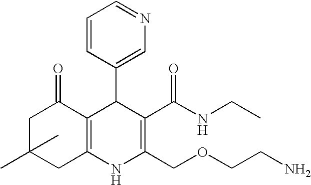 | 2-(2-Amino-ethoxymethyl)-7,7- dimethyl-5-oxo-4-pyridin-3-yl- 1,4,5,6,7,8-hexahydro-quinoline-3- carboxylic acid ethylamide |
| 144 | 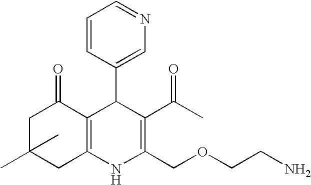 | 3-Acetyl-2-(2-amino-ethoxymethyl)- 7,7-dimethyl-4-pyridin-3-yl-4,6,7,8- tetrahydro-1H-quinolin-5-one |
| 145 | 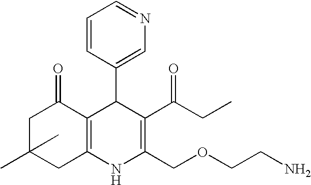 | 2-(2-Amino-ethoxymethyl)-7,7- dimethyl-3-propionyl-4-pyridin-3-yl- 4,6,7,8-tetrahydro-1H-quinolin-5-one |
| 146 | 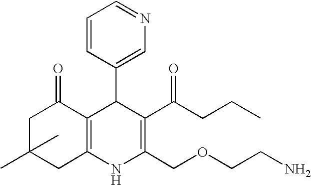 | 2-(2-Amino-ethoxymethyl)-3-butyryl- 7,7-dimethyl-4-pyridin-3-yl-4,6,7,8- tetrahydro-1H-quinolin-5-one |
| 147 | 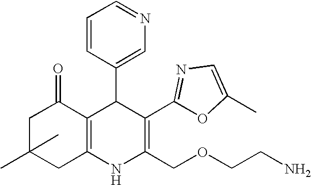 | 2-(2-Amino-ethoxymethyl)-7,7- dimethyl-3-(5-methyl-oxazol-2-yl)-4- pyridin-3-yl-4,6,7,8-tetrahydro-1H- quinolin-5-one |
| 148 | 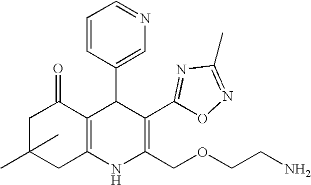 | 2-(2-Amino-ethoxymethyl)-7,7- dimethyl-3-(3-methyl- [1,2,4]oxadiazol-5-yl)-4-pyridin-3-yl- 4,6,7,8-tetrahydro-1H-quinolin-5-one |
| 149 | 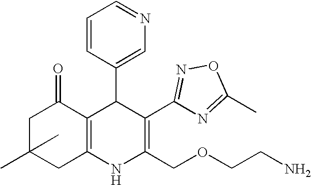 | 2-(2-Amino-ethoxymethyl)-7,7- dimethyl-3-(5-methyl- [1,2,4]oxadiazol-3-yl)-4-pyridin-3-yl- 4,6,7,8-tetrahydro-1H-quinolin-5-one |
| 150 | 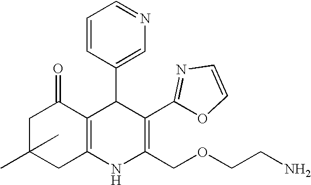 | 2-(2-Amino-ethoxymethyl)-7,7- dimethyl-3-oxazol-2-yl-4-pyridin-3- yl-4,6,7,8-tetrahydro-1H-quinolin-5- one |
| 151 | 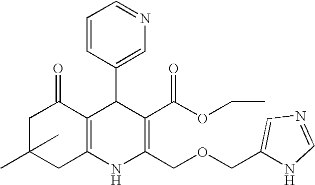 | 2-(3H-Imidazol-4-ylmethoxymethyl)- 7,7-dimethyl-5-oxo-4-pyridin-3-yl- 1,4,5,6,7,8-hexahydro-quinoline-3- carboxylic acid ethyl ester |
| 152 | 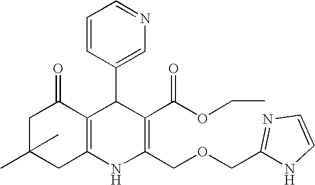 | 2-(1H-Imidazol-2-ylmethoxymethyl)- 7,7-dimethyl-5-oxo-4-pyridin-3-yl- 1,4,5,6,7,8-hexahydro-quinoline-3- carboxylic acid ethyl ester |
| 153 | 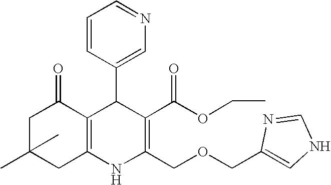 | 2-(1H-Imidazol-4-ylmethoxymethyl)- 7,7-dimethyl-5-oxo-4-pyridin-3-yl- 1,4,5,6,7,8-hexahydro-quinoline-3- carboxylic acid ethyl ester |
| 154 | 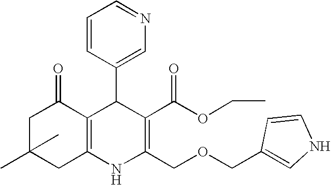 | 7,7-Dimethyl-5-oxo-4-pyridin-3-yl-2- (1H-pyrrol-3-ylmethoxymethyl)- 1,4,5,6,7,8-hexahydro-quinoline-3- carboxylic acid ethyl ester |
| 155 | 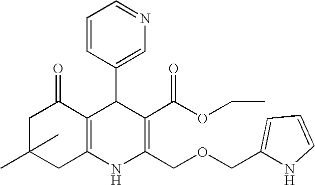 | 7,7-Dimethyl-5-oxo-4-pyridin-3-yl-2- (1H-pyrrol-2-ylmethoxymethyl)- 1,4,5,6,7,8-hexahydro-quinoline-3- carboxylic acid ethyl ester |
| 156 | 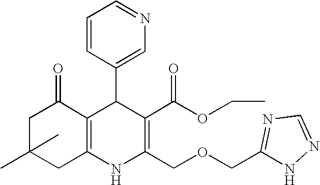 | 7,7-Dimethyl-5-oxo-4-pyridin-3-yl-2- (2H-[1,2,4]triazol-3- ylmethoxymethyl)-1,4,5,6,7,8- hexahydro-quinoline-3-carboxylic acid ethyl ester |
| 157 | 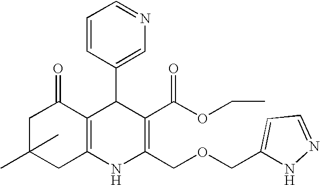 | 7,7-Dimethyl-5-oxo-2-(2H-pyrazol-3- ylmethoxymethyl)-4-pyridin-3-yl- 1,4,5,6,7,8-hexahydro-quinoline-3- carboxylic acid ethyl ester |
| 158 | 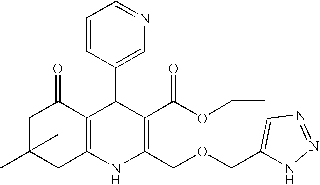 | 7,7-Dimethyl-5-oxo-4-pyridin-3-yl-2- (3H-[1,2,3]triazol-4- ylmethoxymethyl)-1,4,5,6,7,8- hexahydro-quinoline-3-carboxylic acid ethyl ester |
| 159 | 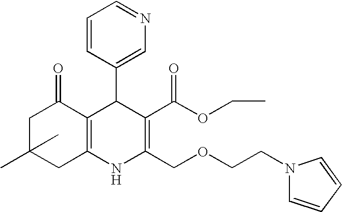 | 7,7-Dimethyl-5-oxo-4-pyridin-3-yl-2- (2-pyrrol-1-yl-ethoxymethyl)- 1,4,5,6,7,8-hexahydro-quinoline-3- carboxylic acid ethyl ester |
| 160 | 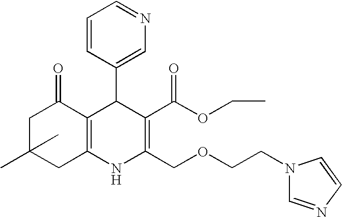 | 2-(2-Imidazol-1-yl-ethoxymethyl)- 7,7-dimethyl-5-oxo-4-pyridin-3-yl- 1,4,5,6,7,8-hexahydro-quinoline-3- carboxylic acid ethyl ester |
| 161 | 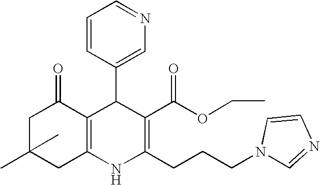 | 2-(3-Imidazol-1-yl-propyl)-7,7- dimethyl-5-oxo-4-pyridin-3-yl- 1,4,5,6,7,8-hexahydro-quinoline-3- carboxylic acid ethyl ester |
| 162 | 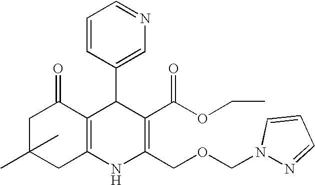 | 7,7-Dimethyl-5-oxo-2-(3-pyrazol-1- yl-propyl)-4-pyridin-3-yl-1,4,5,6,7,8- hexahydro-quinoline-3-carboxylic acid ethyl ester |
| 163 | 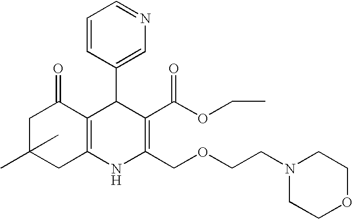 | 7,7-Dimethyl-2-(2-morpholin-4-yl- ethoxymethyl)-5-oxo-4-pyridin-3-yl- 1,4,5,6,7,8-hexahydro-quinoline-3- carboxylic acid ethyl ester |
| 164 | 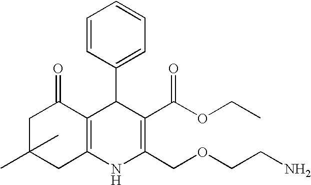 | 2-(2-Amino-ethoxymethyl)-7,7- dimethyl-5-oxo-4-phenyl-1,4,5,6,7,8- hexahydro-quinoline-3-carboxylic acid ethyl ester |
| 165 | 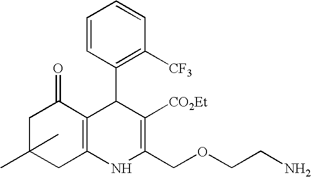 | 2-(2-Amino-ethoxymethyl)-7,7- dimethyl-5-oxo-4-(2-trifluoromethyl- phenyl)-1,4,5,6,7,8-hexahydro- quinoline-3-carboxylic acid ethyl ester |
| 166 | 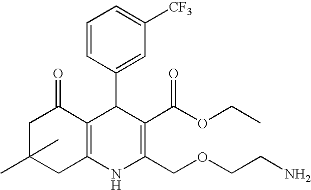 | 2-(2-Amino-ethoxymethyl)-7,7- dimethyl-5-oxo-4-(3-trifluoromethyl- phenyl)-1,4,5,6,7,8-hexahydro- quinoline-3-carboxylic acid ethyl ester |
| 167 | 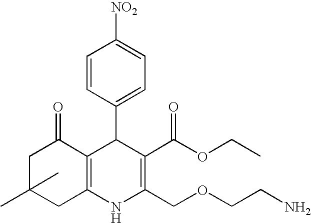 | 2-(2-Amino-ethoxymethyl)-7,7- dimethyl-4-(4-nitro-phenyl)-5-oxo- 1,4,5,6,7,8-hexahydro-quinoline-3- carboxylic acid ethyl ester |
| 168 | 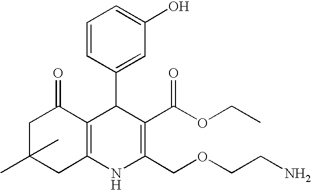 | 2-(2-Amino-ethoxymethyl)-4-(3- hydroxy-phenyl)-7,7-dimethyl-5-oxo- 1,4,5,6,7,8-hexahydro-quinoline-3- carboxylic acid ethyl ester |
| 169 | 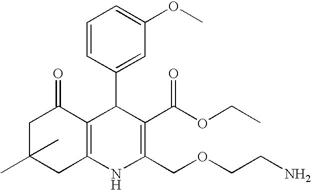 | 2-(2-Amino-ethoxymethyl)-4-(3- methoxy-phenyl)-7,7-dimethyl-5- oxo-1,4,5,6,7,8-hexahydro-quinoline- 3-carboxylic acid ethyl ester |
| 170 | 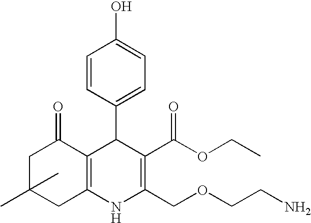 | 2-(2-Amino-ethoxymethyl)-4-(4- hydroxy-phenyl)-7,7-dimethyl-5-oxo- 1,4,5,6,7,8-hexahydro-quinoline-3- carboxylic acid ethyl ester |
| 171 | 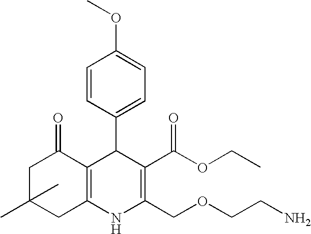 | 2-(2-Amino-ethoxymethyl)-4-(4- methoxy-phenyl)-7,7-dimethyl-5- oxo-1,4,5,6,7,8-hexahydro-quinoline- 3-carboxylic acid ethyl ester |
| 172 | 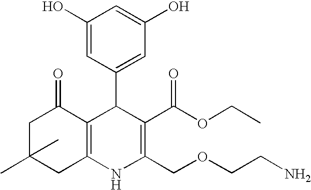 | 2-(2-Amino-ethoxymethyl)-4-(3,5- dihydroxy-phenyl)-7,7-dimethyl-5- oxo-1,4,5,6,7,8-hexahydro-quinoline- 3-carboxylic acid ethyl ester |
| 173 | 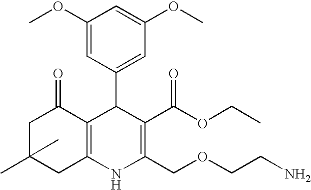 | 2-(2-Amino-ethoxymethyl)-4-(3,5- dimethoxy-phenyl)-7,7-dimethyl-5- oxo-1,4,5,6,7,8-hexahydro-quinoline- 3-carboxylic acid ethyl ester |
| 174 | 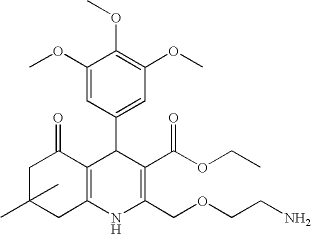 | 2-(2-Amino-ethoxymethyl)-7,7- dimethyl-5-oxo-4-(3,4,5-trimethoxy- phenyl)-1,4,5,6,7,8-hexahydro- quinoline-3-carboxylic acid ethyl ester |
| 175 | 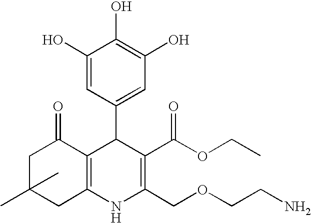 | 2-(2-Amino-ethoxymethyl)-7,7- dimethyl-5-oxo-4-(3,4,5-trihydroxy- phenyl)-1,4,5,6,7,8-hexahydro- quinoline-3-carboxylic acid ethyl ester |
| 176 | 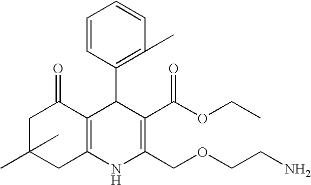 | 2-(2-Amino-ethoxymethyl)-7,7- dimethyl-5-oxo-4-o-tolyl-1,4,5,6,7,8- hexahydro-quinoline-3-carboxylic acid ethyl ester |
| 177 | 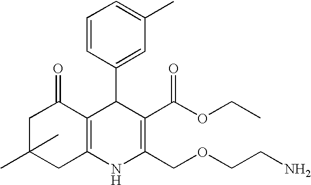 | 2-(2-Amino-ethoxymethyl)-7,7- dimethyl-5-oxo-4-m-tolyl-1,4,5,6,7,8- hexahydro-quinoline-3-carboxylic acid ethyl ester |
| 178 | 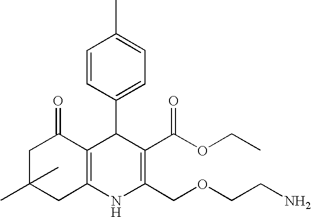 | 2-(2-Amino-ethoxymethyl)-7,7- dimethyl-5-oxo-4-p-tolyl-1,4,5,6,7,8- hexahydro-quinoline-3-carboxylic acid ethyl ester |
| 179 | 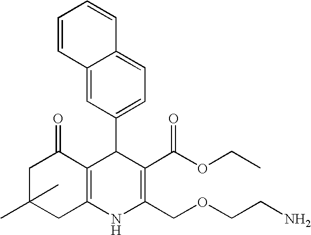 | 2-(2-Amino-ethoxymethyl)-7,7- dimethyl-4-naphthalen-2-yl-5-oxo- 1,4,5,6,7,8-hexahydro-quinoline-3- carboxylic acid ethyl ester |
| 180 | 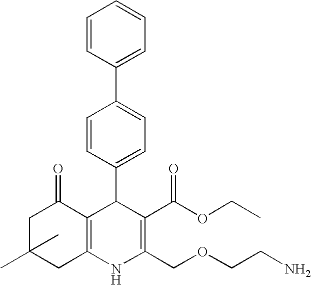 | 2-(2-Amino-ethoxymethyl)-4- biphenyl-4-yl-7,7-dimethyl-5-oxo- 1,4,5,6,7,8-hexahydro-quinoline-3- carboxylic acid ethyl ester |
| 181 | 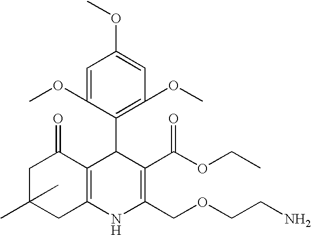 | 2-(2-Amino-ethoxymethyl)-7,7- dimethyl-5-oxo-4-(2,4,6-trimethoxy- phenyl)-1,4,5,6,7,8-hexahydro- quinoline-3-carboxylic acid ethyl ester |
| 182 | 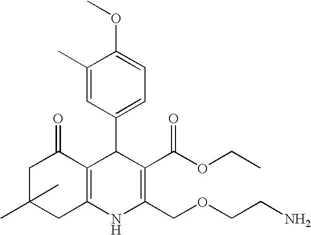 | 2-(2-Amino-ethoxymethyl)-4-(4- methoxy-3-methyl-phenyl)-7,7- dimethyl-5-oxo-1,4,5,6,7,8- hexahydro-quinoline-3-carboxylic acid ethyl ester |
| 183 | 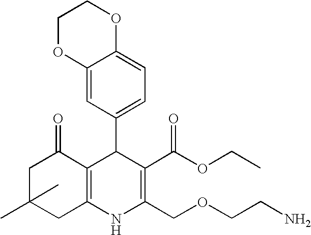 | 2-(2-Amino-ethoxymethyl)-4-(2,3- dihydro-benzo[1,4]dioxin-6-yl)-7,7- dimethyl-5-oxo-1,4,5,6,7,8- hexahydro-quinoline-3-carboxylic acid ethyl ester |
| 184 | 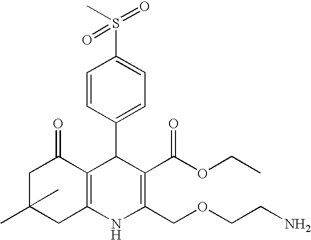 | 2-(2-Amino-ethoxymethyl)-4-(4- methanesulfonyl-phenyl)-7,7- dimethyl-5-oxo-1,4,5,6,7,8- hexahydro-quinoline-3-carboxylic acid ethyl ester |
| 185 | 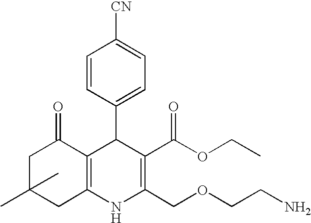 | 2-(2-Amino-ethoxymethyl)-4-(4- cyano-phenyl)-7,7-dimethyl-5-oxo- 1,4,5,6,7,8-hexahydro-quinoline-3- carboxylic acid ethyl ester |
| 186 | 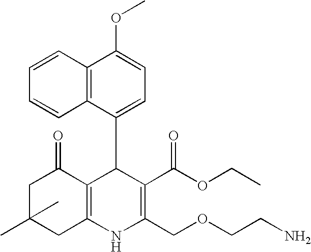 | 2-(2-Amino-ethoxymethyl)-4-(4- methoxy-naphthalen-1-yl)-7,7- dimethyl-5-oxo-1,4,5,6,7,8- hexahydro-quinoline-3-carboxylic acid ethyl ester |
| 187 | 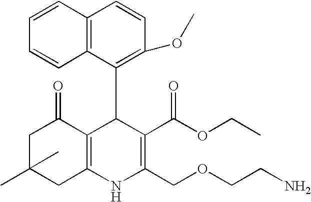 | 2-(2-Amino-ethoxymethyl)-4-(2- methoxy-naphthalen-1-yl)-7,7- dimethyl-5-oxo-1,4,5,6,7,8- hexahydro-quinoline-3-carboxylic acid ethyl ester |
| 188 | 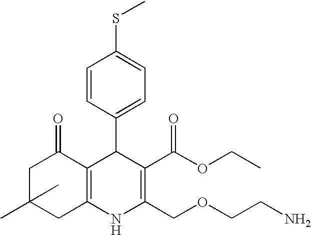 | 2-(2-Amino-ethoxymethyl)-7,7- dimethyl-4-(4-methylsulfanyl- phenyl)-5-oxo-1,4,5,6,7,8-hexahydro- quinoline-3-carboxylic acid ethyl ester |
| 189 | 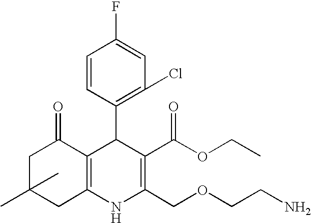 | 2-(2-Amino-ethoxymethyl)-4-(2- chloro-4-fluoro-phenyl)-7,7- dimethyl-5-oxo-1,4,5,6,7,8- hexahydro-quinoline-3-carboxylic acid ethyl ester |
| 190 | 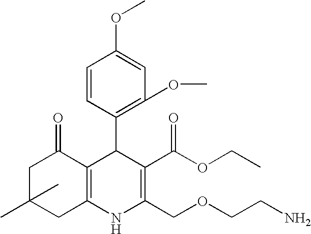 | 2-(2-Amino-ethoxymethyl)-4-(2,4- dimethoxy-phenyl)-7,7-dimethyl-5- oxo-1,4,5,6,7,8-hexahydro-quinoline- 3-carboxylic acid ethyl ester |
| 191 | 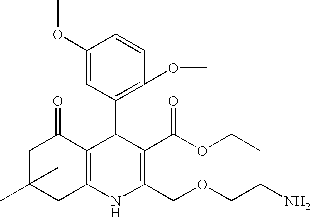 | 2-(2-Amino-ethoxymethyl)-4-(2,5- dimethoxy-phenyl)-7,7-dimethyl-5- oxo-1,4,5,6,7,8-hexahydro-quinoline- 3-carboxylic acid ethyl ester |
| 192 | 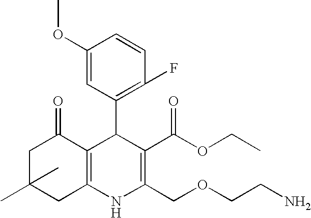 | 2-(2-Amino-ethoxymethyl)-4-(2- fluoro-5-methoxy-phenyl)-7,7- hexahydro-quinoline-3-carboxylic acid ethyl ester |
| 193 | 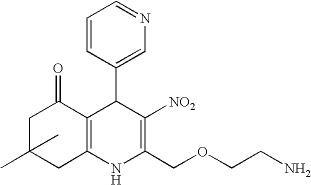 | 2-(2-Amino-ethoxymethyl)-7,7- dimethyl-3-nitro-4-pyridin-3-yl- 1,4,5,6,7,8-hexahydro-quinolin-5-one |
| 194 | 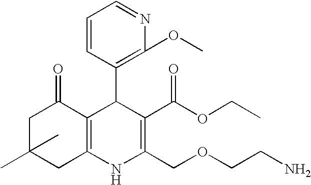 | 2-(2-Amino-ethoxymethyl)-7,7- dimethyl-4-(2-methoxypyrid-3-yl)-5- oxo-1,4,5,6,7,8-hexahydro-quinoline- 3-carboxylic acid ethyl ester |
| 195 | 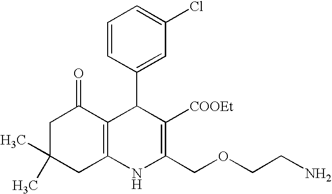 | 2-(2-Amino-ethoxymethyl)-7,7- dimethyl-4-(3-chlorophenyl)-5-oxo- 1,4,5,6,7,8-hexahydro-quinoline-3- carboxylic acid ethyl ester |
| 196 | 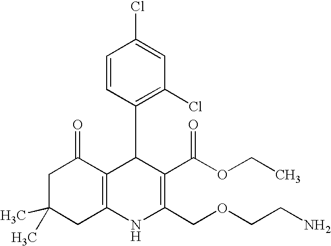 | 2-(2-Amino-ethoxymethyl)-7,7- dimethyl-4-(2,4-dichlorophenyl)-5- oxo-1,4,5,6,7,8-hexahydro-quinoline- 3-carboxylic acid ethyl ester |
| 197 | 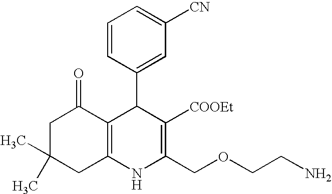 | 2-(2-Amino-ethoxymethyl)-7,7- dimethyl-4-(3-cyanophenyl)-5-oxo- 1,4,5,6,7,8-hexahydro-quinoline-3- carboxylic acid ethyl ester |
| 198 | 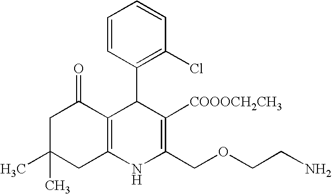 | 2-(2-Amino-ethoxymethyl)-7,7- dimethyl-4-(2-chlorophenyl)-5-oxo- 1,4,5,6,7,8-hexahydro-quinoline-3- carboxylic acid ethyl ester |
| 199 | 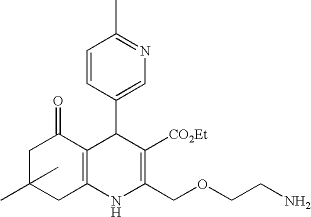 | 2-(2-Amino-ethoxymethyl)-7,7- dimethyl-4-(4-methylpyrid-3-yl)-5- oxo-1,4,5,6,7,8-hexahydro-quinoline- 3-carboxylic acid ethyl ester |
| 200 | 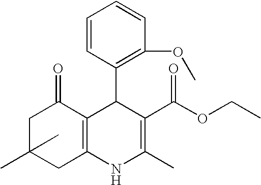 | 2,7,7-Trimethyl-4-(2- methoxyphenyl)-5-oxo-1,4,5,6,7,8- hexahydro-quinoline-3-carboxylic acid ethyl ester |
| 201 | 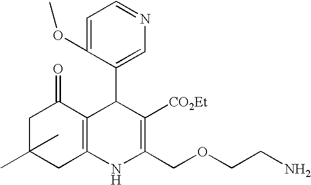 | 2-(2-Amino-ethoxymethyl)-7,7- dimethyl-4-(4-methoxypyrid-3-yl)-5- oxo-1,4,5,6,7,8-hexahydro-quinoline- 3-carboxylic acid ethyl ester |
| 202 | 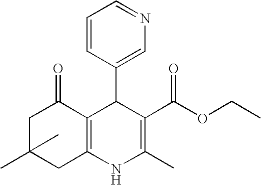 | 2,7,7-Trimethyl-4-(pyrid-3-yl)-5-oxo- 1,4,5,6,7,8-hexahydro-quinoline-3- carboxylic acid ethyl ester |
| 203 | 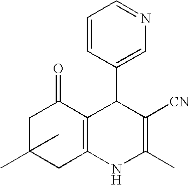 | 2,7,7-Trimethyl-4-(pyrid-3-yl)-5-oxo- 1,4,5,6,7,8-hexahydro-quinoline-3- carbonitrile |
| 204 | 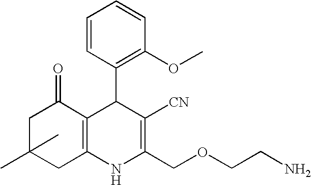 | 2-(2-Amino-ethyoxymethyl)-7,7- dimethyl-4-(2-methoxyphenyl)-5- oxo-1,4,5,6,7,8-hexahydro-quinoline- 3-carbonitrile |
| 205 | 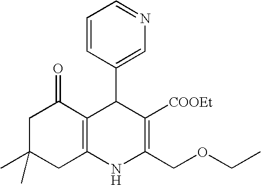 | 2-(2-Amino-ethoxymethyl)-7,7- dimethyl-4-(pyrid-3-yl)-5-oxo- 1,4,5,6,7,8-hexahydro-quinoline-3- carboxylic acid ethyl ester |
| 206 | 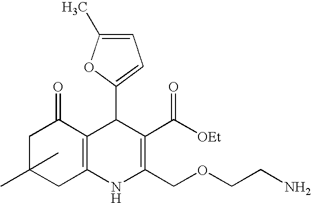 | 2-(2-Amino-ethoxymethyl)-7,7- dimethyl-4-(5-methyl-furan-2-yl)-5- oxo-1,4,5,6,7,8-hexahydro-quinoline- 3-carboxylic acid ethyl ester |
| 207 | 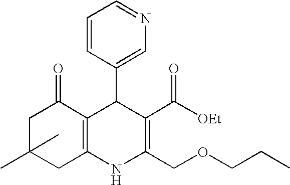 | 2-Propoxymethyl-7,7-dimethyl-4- (pyrid-3-yl)-5-oxo-1,4,5,6,7,8- hexahydro-quinoline-3-carboxylic acid ethyl ester |
| 208 | 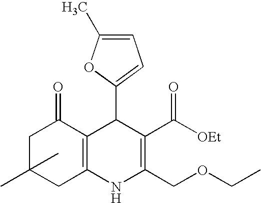 | 2-Ethoxymethyl-7,7-dimethyl-4-(5- methyl-furan-2-yl)-5-oxo-1,4,5,6,7,8- hexahydro-quinoline-3-carboxylic acid ethyl ester |
| 209 | 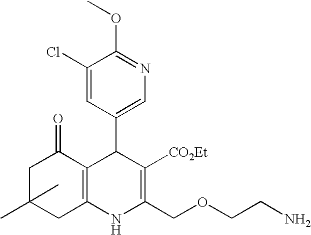 | 2-(2-Amino-ethoxymethyl)-7,7- dimethyl-4-(5-chloro-6-methoxy- pyrid-3-yl)-5-oxo-1,4,5,6,7,8- hexahydro-quinoline-3-carboxylic acid ethyl ester |
| 210 | 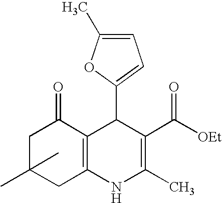 | 2,7,7-Trimethyl-4-(5-methyl-furan-2- yl)-5-oxo-1,4,5,6,7,8-hexahydro- quinoline-3-carboxylic acid ethyl ester |
| 211 | 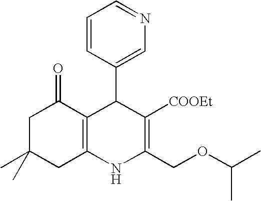 | 2-Isopropoxymethyl-7,7-dimethyl-4- (pyrid-3-yl)-5-oxo-1,4,5,6,7,8- hexahydro-quinoline-3-carboxylic acid ethyl ester |
| 212 | 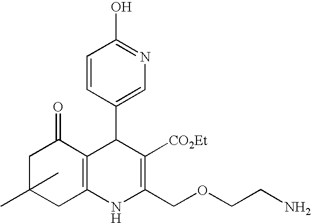 | 2-(2-Amino-ethoxymethyl)-7,7- dimethyl-4-(6-hydroxypyrid-3-yl)-5- oxo-1,4,5,6,7,8-hexahydro-quinoline- 3-carboxylic acid ethyl ester |
| 213 | 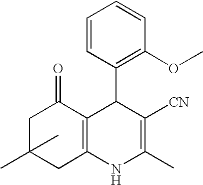 | 2,7,7-Trimethyl-4-(2- methoxyphenyl)-5-oxo-1,4,5,6,7,8- hexahydro-quinoline-3-carbonitrile |
| 214 | 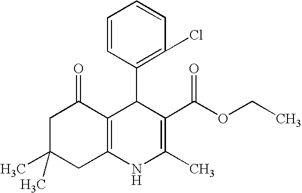 | 2,7,7-Trimethyl-4-(2-chlorophenyl)- 5-oxo-1,4,5,6,7,8-hexahydro- quinoline-3-carboxylic acid ethyl ester |
| 215 | 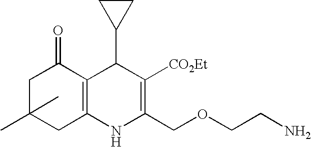 | 2-Methoxymethyl-4-cyclopropyl-5- oxo-7,7-dimethyl-1,4,5,6,7,8- hexahydro-quinoline-3-carboxylic acid ethyl ester |
| 216 | 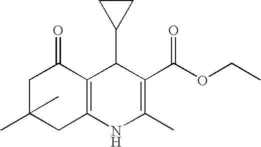 | 2,7,7-Trimethyl-4-cyclopropyl-5-oxo- 1,4,5,6,7,8-hexahydro-quinoline-3- carboxylic acid methyl ester |
| 217 | 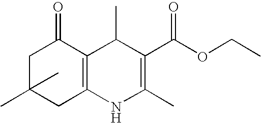 | 2,4,7,7-Tetramethyl-5-oxo- 1,4,5,6,7,8-hexahydro-quinoline-3- carboxylic acid ethyl ester |
| 218 | 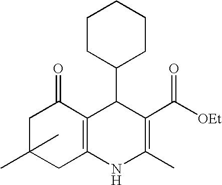 | 2,7,7-Trimethyl-4-cyclohexyl-5-oxo- 1,4,5,6,7,8-hexahydro-quinoline-3- carboxylic acid ethyl ester |
| 219 | 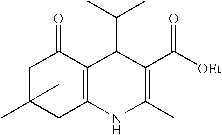 | 2,7,7-Trimethyl-4-propyl-5-oxo- 1,4,5,6,7,8-hexahydro-quinoline-3- carboxylic acid ethyl ester |
| 220 | 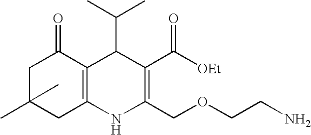 | 2-(2-Amino-ethoxymethyl)-4-propyl- 5-oxo-7,7-dimethyl-1,4,5,6,7,8- hexahydro-quinoline-3-carboxylic acid ethyl ester |
| 221 | 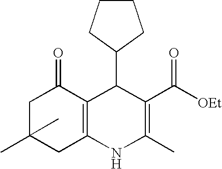 | 2,7,7-Trimethyl-4-cyclopentyl-5-oxo- 1,4,5,6,7,8-hexahydro-quinoline-3- carboxylic acid ethyl ester |
| 222 | 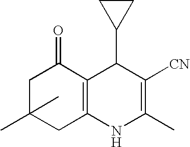 | 2,7,7-Trimethyl-4-cyclopropyl-5-oxo- 1,4,5,6,7,8-hexahydro-quinoline-3- carbonitrile |
| 223 | 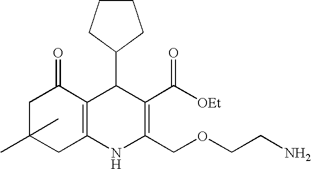 | 2-(Amino-ethoxymethyl)-4- 1,4,5,6,7,8-hexahydro-quinoline-3- carboxylic acid ethyl ester |
| 224 | 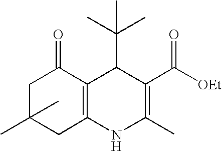 | 2,7,7-Trimethyl-4-(t-butyl)-5-oxo- 1,4,5,6,7,8-hexahydro-quinoline-3- carboxylic acid ethyl ester |
| 225 | 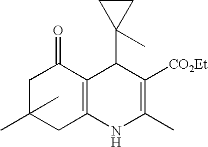 | 2,7,7-Trimethyl-4-(1-methyl- cyclopropyl)-5-oxo-1,4,5,6,7,8- hexahydro-quinoline-3-carboxylic acid ethyl ester |
| 226 | 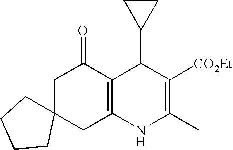 | 2-Methyl-4-cyclopropyl-5-oxo- spiro[1,4,5,6,7,8-hexahydro- quinoline-7,1′-cyclopentane]-3- carboxylic acid ethyl ester |
| 227 | 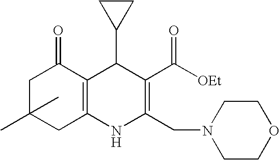 | 2-(Morpholin-4-yl-methyl)-4- cyclopropyl-5-oxo-7,7-dimethyl- 1,4,5,6,7,8-hexahydro-quinoline-3- carboxylic acid ethyl ester |
| 228 | 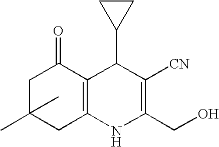 | 2-(Hydroxymethyl)-4-cyclopropyl-5- oxo-7,7-dimethyl-1,4,5,6,7,8- hexahydro-quinoline-3-carbonitrile |
| 229 | 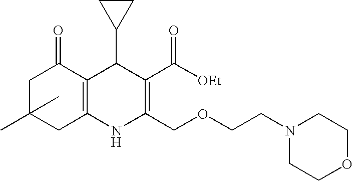 | 2-[2-(Morpholin-4-yl)- ethoxymethyl]-4-cyclopropyl-5-oxo- 7,7-dimethyl-1,4,5,6,7,8-hexahydro- quinoline-3-carboxylic acid ethyl ester |
| 230 | 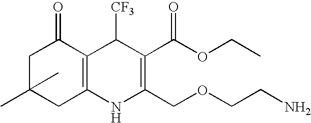 | 2-(2-Amino-ethoxymethyl)-4- trifluoromethyl-5-oxo-7,7-dimethyl- 1,4,5,6,7,8-hexahydro-quinoline-3- carboxylic acid ethyl ester |
| 231 | 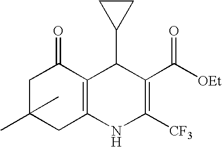 | 2-Trifluoromethyl-4-cyclopropyl-5- oxo-7,7-dimethyl-1,4,5,6,7,8- hexahydro-quinoline-3-carboxylic acid ethyl ester |
| 232 | 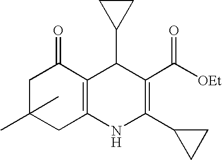 | 2,4-Dicyclopropyl-5-oxo-7,7- dimethyl-1,4,5,6,7,8-hexahydro- quinoline-3-carboxyiic acid ethyl ester |
| 233 | 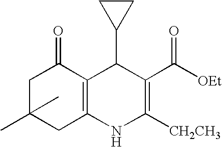 | 2-Ethyl-4-cyclopropyl-5-oxo-7,7- dimethyl-1,4,5,6,7,8-hexahydro- quinoline-3-carboxylic acid ethyl ester |
| 234 | 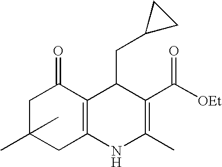 | 2,7,7-Trimethyl-4-(cyclopropyl- methyl)-5-oxo-1,4,5,6,7,8-hexahydro- quinoline-3-carboxylic acid ethyl ester |
| 235 | 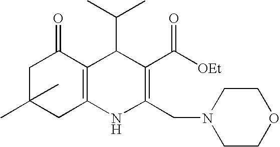 | 2-(Morpholin-4-yl-methyl)-4- (isopropyl)-5-oxo-7,7-dimethyl- 1,4,5,6,7,8-hexahydro-quinoline-3- carboxylic acid ethyl ester |
| 236 | 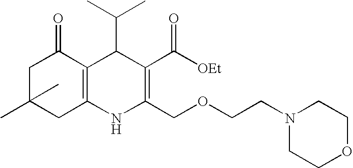 | 2-[2-(Morpholin-4-yl)- ethoxymethyl]-4-(isopropyl)-5-oxo- 7,7-dimethyl-1,4,5,6,7,8-hexahydro- quinoline-3-carboxylic acid ethyl ester |
| 237 | 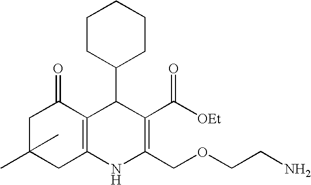 | 2-(2-Amino-ethoxymethyl)-4- cyclohexyl-5-oxo-7,7-dimethyl- 1,4,5,6,7,8-hexahydro-quinoline-3- carboxylic acid ethyl ester |

Preferred compounds of the invention are Compounds 16, 215, and 220. A more preferred compound is Compound 39.

Certain compounds of the invention may contain one or more chiral atoms. The present invention encompasses all stereoisomers (i.e., geometric isomers) including conformational and configurational (e.g., enantiomers, diastereoisomers, and mixtures thereof) of the compounds of the invention. In one embodiment, the invention includes the racemic of either the R- or S-enantiomers of all the compounds described herein. The enantiomers may each be provided in a form substantially free of the other enantiomer (e.g., at least 75% free (w/w), at least 90% free (w/w) or at least 99% free (w/w)) or as mixtures of enantiomers (e.g., racemic mixtures). Pure or substantially pure enantiomers or diastereomers of the compounds of the invention, or a pharmaceutically acceptable salt thereof, can be obtained by well known methods, such as chiral-phase gas chromatography, chiral-phase high performance liquid chromatography, crystallizing the compound as a chiral salt complex, or crystallizing the compound of the invention in a chiral solvent.

C. Methods for Making Compounds of the Invention Compounds of the invention can be obtained via standard, well-known synthetic methodology, see e.g., March, J. Advanced Organic Chemistry; Reactions Mechanisms, and Structure, 4th ed., 1992. In particular, compounds of the invention can be obtained by methods well-known in the art for preparing 1,4-dihydropyridine compounds (e.g., known Ca²⁺ ion-channel blockers). Certain compounds of the invention can be obtained by the processes set forth in U.S. provisional application No. 60/561,246, entitled “Methods for Synthesis of Dihydropyridine Compounds,” filed Apr. 9, 2004, which is incorporated by reference herein in its entirety. Starting materials useful for preparing compounds of the invention and intermediates therefore, are commercially available or can be prepared from commercially available materials using known synthetic methods and reagents.

In addition, compounds of the invention can also be prepared as shown below in Schemes I, II, III, IV and Examples 1-110. 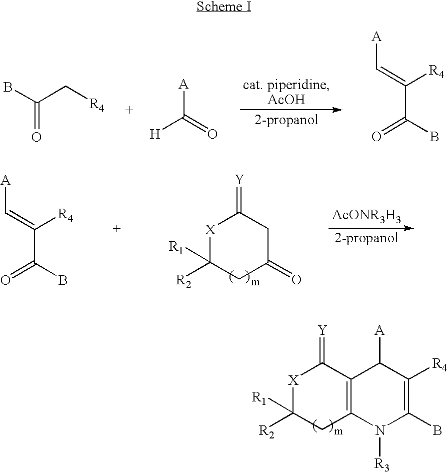 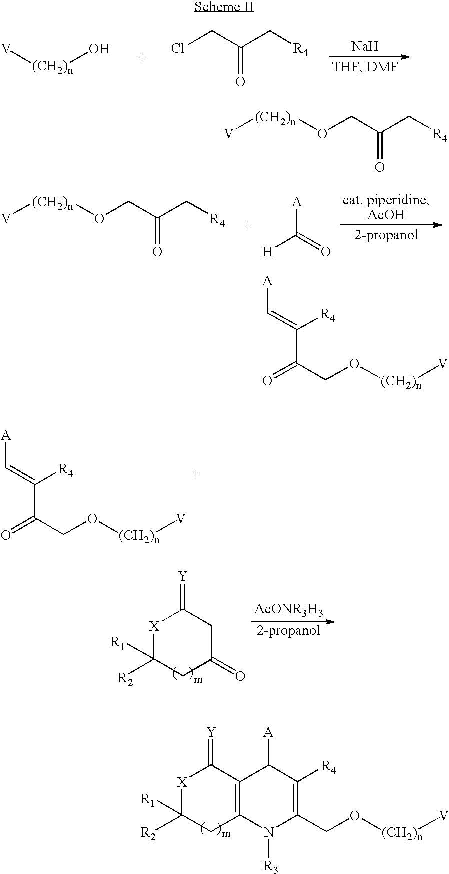 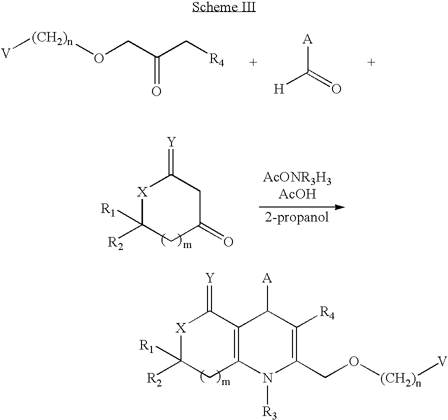 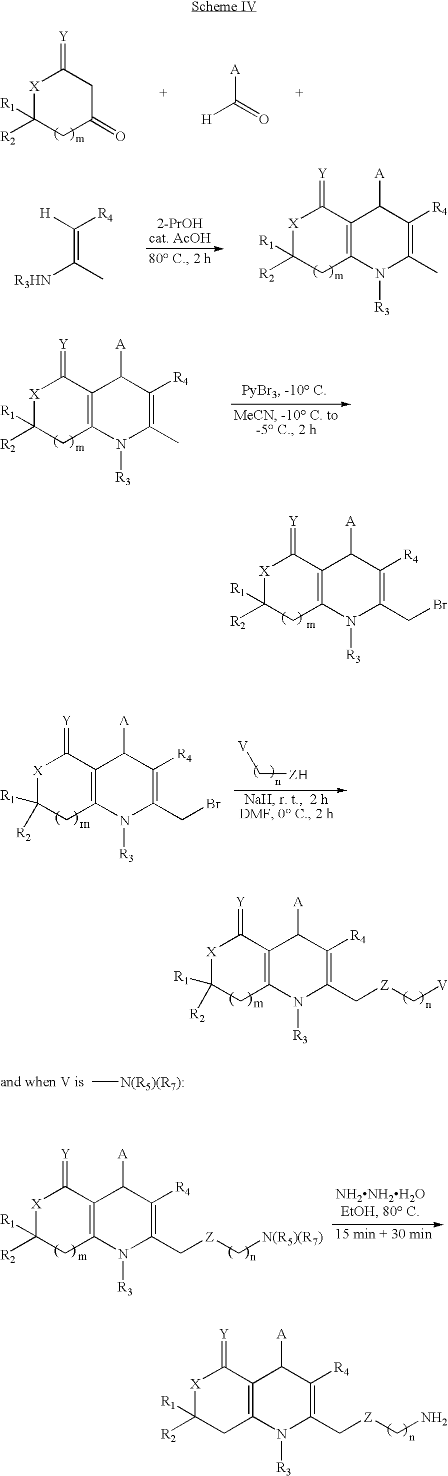
D. Uses of Compounds of the Invention

The present invention is directed to therapies which involve administering one of more compounds of the invention, and compositions comprising said compounds to a subject, preferably a human subject, for preventing, treating, managing, or ameliorating a metabolic disorder or one or more symptoms thereof.

In one embodiment, the invention provides a method of preventing, treating, managing, or ameliorating a metabolic disorder or one or more symptoms thereof, said method comprising administering to a subject in need thereof an effective amount of one or more compounds of the invention. In a specific embodiment, the invention provides a method of preventing, treating, managing, or ameliorating diabetes mellitus (type I and/or type II), and/or a symptom, condition and/or complication associated therewith, said method comprising administering to a subject in need thereof an effective amount of one or more compounds of the invention. In another specific embodiment, the invention provides a method of preventing, treating, managing, or ameliorating diabetes mellitus (type I and/or type II), and/or a symptom, condition and/or complication associated therewith without causing a subject to gain weight, said method comprising administering to a subject in need thereof an effective amount of one or more compounds of the invention. In another embodiment, the invention provides a method of achieving one, two, three or more of the following: (i) reducing blood glucose levels, (ii) improving blood lipid levels, (iii) improving blood insulin levels, and (iv) improving insulin sensitivity, said method comprising administering to a subject in need thereof an effective amount of a compound of formula (I), (II), (III), (IV), (V), (VI), (VII), (VIII), (IX), (X), or Table 1, or a pharmaceutically acceptable salt, solvate, clathrate, or prodrug thereof.

In one embodiment, the invention encompasses a method for preventing, treating, managing, or ameliorating a metabolic disorder (e.g., type I and/or type II diabetes mellitus) or one or more symptoms thereof, comprising administering an effective amount of one or more dihydropyridine compounds or derivatives thereof, including pharmaceutically acceptable salts, solvates, clathrates, or prodrugs thereof, to a patient in need thereof.

In another embodiment, the invention encompasses a method for preventing, treating, managing, or ameliorating a metabolic disorder (e.g., type I and/or type II diabetes mellitus) or one or more symptoms thereof, comprising administering an effective amount of a dihydropyridine compound characterized by an ability to reduce elevated blood glucose levels without a significant cardiovascular effect.

In another embodiment, the invention encompasses a method for preventing, treating, managing, or ameliorating a metabolic disorder (e.g., type I and/or type II diabetes mellitus) or one or more symptoms thereof, comprising administering an effective amount of a 3-substituted-1,4-dihydropyridine compound or a 1,4,5,6,7,8-hexahydroquinoline compound characterized by an ability to reduce elevated blood glucose levels without a significant cardiovascular effect.

In another embodiment, the invention encompasses a method for preventing, treating, managing, or ameliorating a metabolic disorder (e.g., type I and/or type II diabetes mellitus) or one or more symptoms thereof, comprising administering an effective amount of a 3-substituted-1,4-dihydropyridine compound or a 1,4,5,6,7,8-hexahydroquinoline compound which does not have significant toxicity.

In another embodiment, the invention encompasses a method for preventing, treating, managing, or ameliorating a metabolic disorder (e.g., type I and/or type II diabetes mellitus) or one or more symptoms thereof, comprising administering an effective amount of a compound of formula (I): 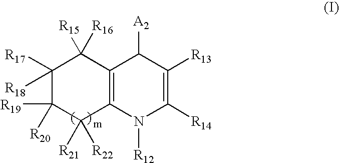
or a pharmaceutically acceptable salt, solvate, clathrate, or prodrug thereof wherein m, A₂, R₁₂, R₁₃, R₁₄, R₁₅, R₁₆, R₁₇, R₁₈, R₁₉, R₂₀, R₂₁, and R₂₂ are as defined herein, to a patient in need thereof.

In another embodiment, the invention encompasses a method for preventing, treating, managing, or ameliorating a metabolic disorder (e.g., type I and/or type II diabetes mellitus) or one or more symptoms thereof, comprising administering an effective amount of a compound of formula (II): 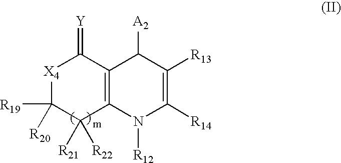
or a pharmaceutically acceptable salt, solvate, clathrate, or prodrug thereof wherein A₂, Y, X₄, R₁₂, R₁₃, R₁₄, R₁₉, R₂₀, R₂₁, R₂₂, and m are as defined herein, to a patient in need thereof.

In another embodiment, the invention encompasses a method for preventing, treating, managing, or ameliorating a metabolic disorder (e.g., type I and/or type II diabetes mellitus) or one or more symptoms thereof, comprising administering an effective amount of a compound of formula (III): 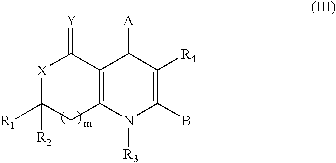
or a pharmaceutically acceptable salt, solvate, clathrate, or prodrug thereof wherein A, B, X, Y, R₁, R₂, R₃, R₄ and m are as defined herein, to a patient in need thereof.

In another embodiment, the invention encompasses a method for preventing, treating, managing, or ameliorating a metabolic disorder (e.g., type I and/or type II diabetes mellitus) or one or more symptoms thereof, comprising administering an effective amount of a compound of formula (IV): 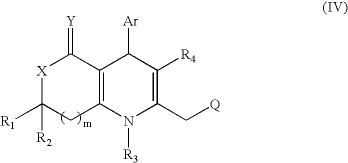
or a pharmaceutically acceptable salt, solvate, clathrate, hydrate, polymorph or prodrugs thereof wherein Ar, Q, X, Y, R₁, R₂, R₃, R₄ and m are as defined herein, to a patient in need thereof.

In another embodiment, the invention encompasses a method for preventing, treating, managing, or ameliorating a metabolic disorder (e.g., type I and/or type II diabetes mellitus) or one or more symptoms thereof, comprising administering an effective amount of a compound of formula (V): 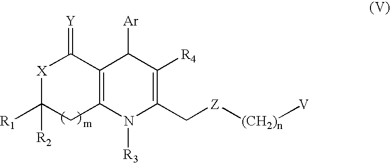
or a pharmaceutically acceptable salt, solvate, clathrate, hydrate, polymorph or prodrug thereof, wherein Ar, X, Y, Z, V, R₁, R₂, R₃, R₄, m and n are as defined herein, to a patient in need thereof.

In another embodiment, the invention encompasses a method for preventing, treating, managing, or ameliorating a metabolic disorder (e.g., type I and/or type II diabetes mellitus) or one or more symptoms thereof, comprising administering an effective amount of a compound of formula (VI): 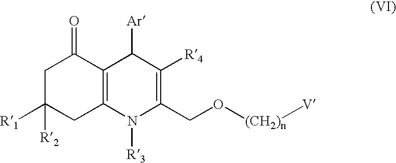
or a pharmaceutically acceptable salt, solvate, clathrate, hydrate, polymorph or prodrug thereof, wherein Ar′, V′, R₁′, R₂′, R₃′, R₄′ and n are as defined herein, to a patient in need thereof.

In another embodiment, the invention encompasses a method for preventing, treating, managing, or ameliorating a metabolic disorder (e.g., type I and/or type II diabetes mellitus) or one or more symptoms thereof, comprising administering an effective amount of a compound of formula (VII): 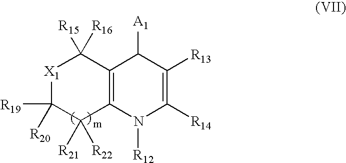
or a pharmaceutically acceptable salt, solvate, clathrate, or prodrug thereof wherein A₁, X₁, R₁₂, R₁₃, R₁₄, R₁₅, R₁₆, R₁₉, R₂₀, R₂₁, R₂₂, and m are as defined herein, to a patient in need thereof.

In another embodiment, the invention encompasses a method for preventing, treating, managing, or ameliorating a metabolic disorder (e.g., type I and/or type II diabetes mellitus) or one or more symptoms thereof, comprising administering an effective amount of a compound of formula (VIII): 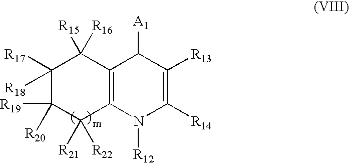
or a pharmaceutically acceptable salt, solvate, clathrate, or prodrug thereof wherein A₁, R₁₂, R₁₃, R₁₄, R₁₅, R₁₆, R₁₇, R₁₈, R₁₉, R₂₀, R₂₁, R₂₂, and m are as defined herein, to a patient in need thereof.

In another embodiment, the invention encompasses a method for preventing, treating, managing, or ameliorating a metabolic disorder (e.g., type I and/or type II diabetes mellitus) or one or more symptoms thereof, comprising administering an effective amount of a compound of formula (IX): 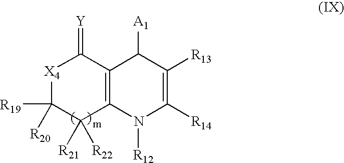
or a pharmaceutically acceptable salt, solvate, clathrate, or prodrug thereof wherein A₁, X₄, Y, R₁₂, R₁₃, R₁₄, R₁₉, R₂₀, R₂₁, R₂₂, and m are as defined herein, to a patient in need thereof.

In another embodiment, the invention encompasses a method for preventing, treating, managing, or ameliorating a metabolic disorder (e.g., type I and/or type II diabetes mellitus) or one or more symptoms thereof, comprising administering an effective amount of a compound of formula (X): 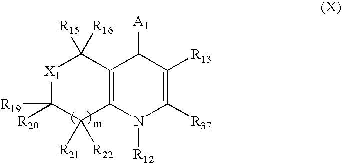
or a pharmaceutically acceptable salt, solvate, clathrate, or prodrug thereof wherein A₁, X₁, R₁₂, R₁₃, R₁₄, R₁₅, R₁₆, R₁₉, R₂₀, R₂₁, R₂₂, and m are as defined herein, to a patient in need thereof.

One or more of the compounds of the invention may be used as a first, second, third, fourth or fifth line for the treatment of a metabolic disorder. The invention provides methods for preventing, treating, managing, or ameliorating a metabolic disorder or one or more symptoms thereof in a subject refractory (either partially or completely) to conventional therapies for such a disorder, said methods comprising administering to said subject a dose of an effective amount of one or more compounds of the invention.

The invention also provides methods of preventing, treating, managing, or ameliorating a metabolic disorder or one or more symptoms thereof, said methods comprising administering to a subject in need thereof one or more compounds of the invention and one or more other therapies (e.g., one or more prophylactic or therapeutic agents that are currently being used, have been used, are known to be useful or in development for use in the prevention, treatment or amelioration of one or more symptoms associated with said metabolic disorder).

In one embodiment, the invention provides methods of preventing, treating, managing, or ameliorating a metabolic disorder or one or more symptoms thereof, said methods comprising administering to a subject in need thereof an effective amount of one or more compounds of the invention and an effective amount of one or more other therapies such as prophylactic or therapeutic agents. In a specific embodiment, the invention provides a method of preventing, treating, managing, or ameliorating diabetes mellitus (type I and/or type II), and/or a symptom, condition and/or complication associated therewith, said method comprising administering to a subject in need thereof a dose of an effective amount of one or more compounds of the invention and an effective amount of one or more other therapies such as prophylactic or therapeutic agents. In another embodiment, the invention provides a method of achieving one, two, three or more of the following: (i) reducing blood glucose levels, (ii) improving blood lipid levels, (iii) improving blood insulin levels, and (iv) improving insulin sensitivity, said method comprising administering to a subject in need thereof an effective amount of a compound of formula (I), (II), (III), (IV), (V), (VI), (VII), (VIII), (IX), (X), or Table 1, or a pharmaceutically acceptable salt, solvate, clathrate, or prodrug thereof, and an effective amount of one or more other therapies such as prophylactic or therapeutic agents. Non-limiting examples of such agents are included herein.

The prophylactic or therapeutic agents of the combination therapies of the invention can be administered sequentially or concurrently. In a specific embodiment, the combination therapies of the invention comprise one or more compounds and at least one other therapy (e.g., another prophylactic or therapeutic agent) which has the same mechanism of action as said compounds. In another specific embodiment, the combination therapies of the invention comprise one or more compounds of the invention and at least one other therapy (e.g., another prophylactic or therapeutic agent) which has a different mechanism of action than said compounds. In certain embodiments, the combination therapies of the present invention improve the prophylactic or therapeutic effect of one or more compounds of the invention by functioning together with the compounds to have an additive or synergistic effect. In certain embodiments, the combination therapies of the present invention reduce the side effects associated with the therapies (e.g., prophylactic or therapeutic agents).

The prophylactic or therapeutic agents of the combination therapies can be administered to a subject, preferably a human subject, in the same pharmaceutical composition. In alternative embodiments, the prophylactic or therapeutic agents of the combination therapies can be administered concurrently to a subject in separate pharmaceutical compositions. The prophylactic or therapeutic agents may be administered to a subject by the same or different routes of administration.

In a specific embodiment, a pharmaceutical composition comprising one or more compounds of the invention is administered to a subject, preferably a human, to prevent, treat, manage, or ameliorate one or more symptoms associated with a metabolic disorder. In accordance with the invention, pharmaceutical compositions of the invention may also comprise one or more other agents (e.g., prophylactic or therapeutic agents which are currently being used, have been used, or are known to be useful in the prevention, treatment or amelioration of said metabolic disorder or a symptom thereof).

The invention provides methods for preventing, managing, treating or ameliorating a metabolic disorder or one or more symptoms thereof in a subject refractory (either completely or partially) to existing single agent therapies for such a metabolic disorder, said methods comprising administering to said subject a dose of an effective amount of one or more compounds of the invention and a dose of an effective amount of one or more therapies (e.g., one or more prophylactic or therapeutic agents useful for the prevention, treatment, management, or amelioration of a metabolic disorder or a symptom thereof). The invention also provides methods for preventing, treating, managing, or ameliorating a metabolic disorder or a symptom thereof by administering one or more compounds of the invention in combination with any other therapy(ies) to patients who have proven refractory to other therapies but are no longer on these therapies.

The compounds of the invention and/or other therapies can be administered to a subject by any route known to one of skill in the art. Examples of routes of administration include, but are not limited to, parenteral, e.g., intravenous, intradernial, suubcutaneous, oral (e.g., inhalation), intranasal, transdermal (topical), transmucosal, and rectal administration.

1) Agents Useful in Combination with the Compounds of the Invention

Without wishing to be bound by theory, compounds of this invention may act by a new mechanism and may advantageously represent a new option for treating and preventing metabolic disorders. Compounds of the invention appear to reduce blood glucose levels, reduce insulin levels in hyperinsulinic patients, and alleviate insulin resistance in animal models of diabetes. These compounds have independent activity but surprisingly, can also act synergistically to enhance the activity of certain conventional diabetes drugs, such as metformin and rosiglitazone. As a result, compounds of this invention can be used as single agents or in combination therapy with other agents.

The present invention provides methods for preventing, managing, treating, or ameliorating metabolic disorders comprising administering to a subject in need thereof or one or more compounds of the invention and one or more therapies (e.g., one or more prophylactic or therapeutic agents) other than compounds of the invention. The present invention also provides compositions comprising one or more compounds of the invention and one or more prophylactic or therapeutic agents other than compounds of the invention and methods of preventing, managing, treating, or ameliorating a metabolic disorder utilizing said compositions. Therapeutic or prophylactic agents include, but are not limited to, small molecules, synthetic drugs, peptides, polypeptides, proteins, nucleic acids (e.g., DNA and RNA nucleotides including, but not limited to, antisense nucleotide sequences, RNAi, triple helices and nucleotide sequences encoding biologically active proteins, polypeptides or peptides), antibodies, synthetic or natural inorganic molecules, mimetic agents, and synthetic or natural organic molecules.

Any agent which is known to be useful, or which has been used, is currently being used for or is in development for the prevention, management, treatment, or amelioration of a metabolic disorder (such as diabetes mellitus, conditions associated with diabetes mellitus and certain complications thereof) or one or more symptoms thereof can be used in combination with a compound of the invention in accordance with the invention described herein. See, e.g., Gilman et al., Goodman and Gilman's: The Pharmacological Basis of Therapeutics, Tenth Ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; Cecil Textbook of Medicine, 20th Ed., Bennett and Plum (eds.), W. B. Saunders, Philadelphia, 1996 for information regarding prophylactic or therapeutic agents which have been or are currently being used for preventing, treating, managing, or ameliorating metabolic disorders or one or more symptoms thereof. Non-limiting examples of agents include anti-diabetic agents, anti-obesity agents, and lipid lowering agents.

Anti-diabetic agents include, without limitation, insulin and oral hypoglycemic agents.

Insulin can be in any form and delivered by any acceptable route. For example, insulin can be intravenously delivered as premixed insulin (such as Humalog Mix (Eli Lilly) and

NovoMix/Novolog Mix (Novo Nordisk)) or short-acting isophane. Human insulins include Humulin (Eli Lilly), Actrapid/Novolin (Novo Nordisk), Insuman (Aventis), and Wosulin (Wockhardt). Short-acting insulin analogues include Humalog (Eli Lilly), NovoRapid/Novolog (Novo Nordisk), and Insulin glulisine (Apidra, Aventis). There are also controlled-release insulins such as Basulin (BMS) and inhaled insulins such as Exubera (Pfizer/Aventis/Nektar).

Newer insulin-related agents in development include agents that switch on insulin receptors (e.g., PTP112 (American Home Products), fast acting insulin (1964 (Aventis)), insulin sensitizers (such as Dexlipotam (Aventis), FK614 (Fujisawa), balaglitazone (NN2344, Novo Nordisk), CRE 16336 and 16258 (Merck KGaA), MXC 3255 (Maxia), KP102 (Kinetek) and PNU 182716 (Pharmacia)), long acting insulin (such as Insulin detemir (Levemir, Novo Nordisk) and Lantus (Aventis) and Levemir (Novo Nordisk)), pulmonary delivered insulin, transdermal insulin (such as that under development by Dong shin) and oral insulin (such as Beodas (Elan) and the insulin and pro-insulin analogs, AI 401 and LY 197535 (Lilly)).

Oral hypoglycemic agents include, but are not limited to:

I. Biguanides. These compounds act by keeping the liver from releasing too much glucose. Non-limiting examples include metformin (Glucophage, Bristol-Myers Squibb) and glyburide/metformin (Glucovance, Bristol-Myers Squibb).

II. Perioxisomes Proliferator Activated Receptor γ (PPARγ) agonists of the thiazolidinedione class. These compounds enhance muscle cell sensitivity to insulin. Non-limiting examples include pioglitazone (Actos, Lilly), rosiglitazone (Avandia, GlaxoSmithKline), isaglitazone (such as MCC555 (Johnson & Johnson)) and troglitizone.

III. Insulinotropic agents. These compounds act by stimulating the pancreas to release more insulin. Non-limiting examples include the non-sulfonylurea secretagogues repaglinide (Prandin, Novo Nordisk), nateglinide (Starlix, Novartis) and glyburide (Micronase, Upjohn).

IV. Sulphonylureas. These compounds stimulate the pancreas to release more insulin. Non-limiting examples include glimepiride (Amaryl, Aventis) and glipizide (Glucotrol, Pfizer).

V. α-glucosidase inhibitors. These compounds slow carbohydrate metabolism. Non-limiting examples include miglitol (Glyset, Bayer) and acarbose (Glucobay and Precose, Bayer).

New diabetes drugs in development fall into a number of additional categories, including:

I. PPARγ agonists (non-thiazolidinediones) (one reported to be under development by Novo Nordisk).

II. PPAR α and PPAR α/γ agonists (such as NN622 (Novo Nordisk), AZ242 (Astra Zeneca), BMS 298585 (Bristol-Myers Squibb), PNU 182716 (Pharmacia), JEO297 (Merck) and DRF 4158 (Novartis)).

III. Glucagon-Like Peptides (GLPs such as the secretagogue GLP-1) and analogues (such as liraglutide (NN2211, Novo Nordisk), a GLP-1 analogue under development by Lilly, AC2993 (Amylin) and Ave-0010 (Aventis)).

IV. Dipeptidylpeptidase IV inhibitors (such as LAF237 (Novartis), P32/98 (ProBiodrug) and DPP 728 (Novartis)).

VI. Glycogen phosphorylase inhibitors (such as NN4201 (isofagamine, Novo Nordisk) and CP 368296 (Pfizer)).

VII. Tyrosine phosphatase inhibitors.

VIII. GLUT 4-mediated glucose transport modulators.

IX. Immunoregulatory vaccines.

X. Amylin receptor antagonists (such as Symlin (pramlintide acetate, Amylin)).

XI. Selective β adrenergic agonists (such as the β3 adrenergic agonists BMS 194449, 196085 and 201620 (BMS) and GW427353 and SB418790 (GlaxoSmithKline).

XII. gluconeogenesis inhibitors (such as CS-917 (Sankyo/Metabasis)).

XIII. potassium channel openers (such as NN414 (Novo Nordisk)).

XIV. PPAR pan agonists (such as 677954 (GSK)).

XV. T cell inhibitors (such as NBI-6024 (Neurocrine)).

XVI. T cell modulators (such as AVE-0277 (Aventis)).

XVII 11 beta HSD1 enzyme inhibitors (such as BVT3498 (Amgen/Biovitrum)).

Combination diabetic therapies are also under development (such as an Avandia/Metfomin combination being developed by GlaxoSmithKline and glipizide/metformin, BMS). In these combination therapies, the agents are typically selected from two or more classes of agents having different mechanisms of action.

Anti-obesity drugs can also be used in combination therapies according to this invention. Such drugs include, without limitation, appetite suppressants and fat blockers. Appetite suppressants include noradrenergic and serotonergic agents. Noradrenergic drugs affect weight loss through action in the appetite center and include phenylpropanolamine (Dexatrim) and phentermine (Ionamin) Phentermine was previously used in combination with fenfluramine (Pondimin) to improve weight loss and counteract the adverse effects of use of phentermine but because of the withdrawal of fenfluramine from the U.S. market, phentermine is now used as a single weight-loss agent.

Serotonergic drugs partially inhibit the reuptake of serotonin and release serotonin into the synaptic cleft, thus acting on the hypothalamus to decrease satiety. Fenfluramine and dexfenfluramine (Redux), the first serotonergic agents labeled for the treatment of obesity, were withdrawn from the U.S. market in September 1997 because of case reports of valvular heart disease and primary pulmonary hypertension. Selective serotonin reuptake inhibitors (SSRIs) may also be used as serotonergic drugs. For example, fluoxetine (Prozac) is a highly selective serotonin reuptake inhibitor that has also been studied in the treatment of obesity. Additional SSRIs currently on the market include Paxil, Effexor, Zoloft, Celexa and Luvox. Others are well known to those of ordinary skill in the art.

Adrenergic/serotonergic agents may also be used in combination with the compounds of this invention. Sibutramine (Meridia) is an adrenergic/serotonergic agent recently labeled by the FDA for use in the management of obesity. Sibutramine and its metabolite inhibit monoamine uptake, suppressing appetite in a fashion similar to SSRIs.

Thermogenic agents form another category of anti-obesity drugs that are useful in combination with the compounds of this invention. For example, the combination of ephedrine and caffeine possesses anorectic and thermogenic properties with only mild, transient side effects. Ephedrine increases the release of norepinephrine, which modulates food intake and acts as a sympathomimetic agent to stimulate heart rate and blood pressure, and enhance thermogenesis. Caffeine, an adenosine antagonist, reduces the breakdown of norepinephrine within the synaptic junction.

Digestive inhibitors interfere with the breakdown, digestion and absorption of dietary fat in the gastrointestinal tract. Gastric and pancreatic lipases aid in the digestion of dietary triglycerides by forming them into free fatty acids that are then absorbed at the brush border of the small intestine. Inhibition of these enzymes leads to inhibition of the digestion of dietary triglycerides and decreased cholesterol absorption, and may decrease absorption of lipid-soluble vitamins (A, D, E and K). Orlistat (Xenical), the first lipase inhibitor labeled by the FDA for treatment of obesity, is a potent and irreversible inhibitor of gastric and pancreatic lipases, preventing the absorption of about 30 percent of dietary fat.

The goal of fat substitutes is to decrease caloric value from fat while maintaining the creaminess and richness derived from fat. The most recent fat-based substitute, olestra (Olean), contains zero kcal per g. Olestra is a sucrose polyester, labeled by the FDA for use as a food additive in prepackaged snacks (potato, corn and tortilla chips, and crackers) to replace 100 percent of the fat. As a sucrose polyester with six to eight fatty-acid side chains, it is too large to be hydrolyzed by digestive enzymes and, therefore, is not absorbed and has no caloric value.

The gastrointestinal tract and central nervous system also contain several peptides and hormones that regulate feeding behavior. For example, cholecystokinin and serotonin act to decrease appetite and food intake. Conversely, neuropeptide Y increases food intake and decreases energy expenditure. Leptin may limit food intake, decrease plasma insulin and increase energy expenditure. Therefore, agonists and antagonists of these hormones and peptides are currently under investigation for the treatment of obesity and may be useful in the combination therapies of this invention.

Lipid lowering agents include without limitation cholestyramine, gemfibrozil, fenofibrate, nicotinic acid and related compounds and statins (such as pravastatin and lovastatin).

2) Compositions and Methods for Administering Therapies

The present invention provides compositions for the treatment, prophylaxis, and amelioration of metabolic disorders. In a specific embodiment, a composition comprises one or more compounds of the invention, or a pharmaceutically acceptable salt, solvate, or hydrate thereof. In another embodiment, a composition of the invention comprises one or more prophylactic or therapeutic agents other than a compound of the invention, or a pharmaceutically acceptable salt, solvate or hydrate thereof. In another embodiment, a composition of the invention comprises one or more compounds of the invention, or a pharmaceutically acceptable salt, solvate, or hydrate thereof, and one or more other prophylactic or therapeutic agents. In another embodiment, the composition comprises a compound of the invention, or a pharmaceutically acceptable salt, solvate, or hydrate thereof, and a pharmaceutically acceptable carrier, diluent or excipient.

In a preferred embodiment, a composition of the invention is a pharmaceutical composition or a single unit dosage form. Pharmaceutical compositions and dosage forms of the invention comprise one or more active ingredients in relative amounts and formulated in such a way that a given pharmaceutical composition or dosage form can be used to treat or prevent metabolic disorders, such as diabetes mellitus, conditions associated with diabetes mellitus and certain complications thereof. Preferred pharmaceutical compositions and dosage forms comprise a compound of formula (I), (II), (III), (IV), (V), (VI), (VII), (VIII), (IX), (X), or Table 1, or a pharmaceutically acceptable prodrug, salt, solvate, or clathrate thereof, optionally in combination with one or more additional active agents.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings.

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form suitable for mucosal administration may contain a smaller amount of active ingredient(s) than an oral dosage form used to treat the same indication. This aspect of the invention will be readily apparent to those skilled in the art. See, e.g., Remington's Pharmaceutical Sciences (1990) 18th ed., Mack Publishing, Easton Pa.

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms.

The suitability of a particular excipient may also depend on the specific active ingredients in the dosage form. For example, the decomposition of some active ingredients can be accelerated by some excipients such as lactose, or when exposed to water. Active ingredients that comprise primary or secondary amines (e.g., N-desmethylvenlafaxine and N,N-didesmethylvenlafaxine) are particularly susceptible to such accelerated decomposition. Consequently, this invention encompasses pharmaceutical compositions and dosage forms that contain little, if any, lactose. As used herein, the term “lactose-free” means that the amount of lactose present, if any, is insufficient to substantially increase the degradation rate of an active ingredient. Lactose-free compositions of the invention can comprise excipients that are well known in the art and are listed, for example, in the U.S. Pharmocopia (USP) SP (XXI)/NF (XVI). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen (1995) Drug Stability: Principles & Practice, 2d. Ed., Marcel Dekker, NY, N.Y., 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizer” include, but are not limited to, antioxidants such as ascorbic acid, pH buffers, or salt buffers.

i) Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences (1990) 18th ed., Mack Publishing, Easton Pa.

Typical oral dosage forms of the invention are prepared by combining the active ingredient(s) in an admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In one embodiment, an oral dosage form of the invention consists of one or more compounds of the invention in a capsule or caplet (i.e., without an excipent).

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, cuboxylllmuhlyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. One specific binder is a mixture of microcrystalline cellulose and sodium carboxymethyl cellulose sold as AVICEL RC-581. Suitable anhydrous or low moisture excipients or additives include AVICEL-PH-103J and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

ii) Controlled Release Dosage Forms

Active ingredients of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

A particular extended release formulation of this invention comprises a therapeutically or prophylactically effective amount of a compound of formula (I), (II), (III), (IV), (V), (VI), (VII), (VIII), (IX), (X), or Table 1, or a pharmaceutically acceptable salt, solvate, hydrate, clathrate, or prodrug thereof, in spheroids which further comprise microcrystalline cellulose and, optionally, hydroxypropylmethyl-cellulose coated with a mixture of ethyl cellulose and hydroxypropylmethylcellulose. Such extended release formulations can be prepared according to U.S. Pat. No. 6,274,171, the entirely of which is incorporated herein by reference.

A specific controlled-release formulation of this invention comprises from about 6% to about 40% a compound of formula (I), (II), (III), (IV), (V), (VI), (VII), (VIII), (IX), (X), or Table 1, or a pharmaceutically acceptable salt, solvate, hydrate, clathrate, or prodrug thereof, by weight, about 50% to about 94% microcrystalline cellulose, NF, by weight, and optionally from about 0.25% to about 1% by weight of hydroxypropyl-methylcellulose, USP, wherein the spheroids are coated with a film coating composition comprised of ethyl cellulose and hydroxypropylmethylcellulose.

iii) Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

iv) Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences (1980 & 1990) 16th and 18th eds., Mack Publishing, Easton Pa. and Introduction to Pharmaceutical Dosage Forms (1985) 4th ed., Lea & Febiger, Philadelphia. Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences (1980 & 1990) 16th and 18th eds., Mack Publishing, Easton Pa.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

v) Dosage & Frecuency of Administration

The amount of the compound or composition of the invention which will be effective in the prevention, treatment, management, or amelioration of a metabolic disorder or one or more symptoms thereof will vary with the nature and severity of the disease or condition, and the route by which the active ingredient is administered. The frequency and dosage will also vary according to factors specific for each patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in the Physician's Desk Reference (57th ed., 2003).

Exemplary doses of a small molecule include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). For antibodies, proteins, polypeptides, peptides and fusion proteins encompassed by the invention, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

In general, the recommended daily dose range of a compound of the invention for the conditions described herein lie within the range of from about 0.01 mg to about 1000 mg per day, given as a single once-a-day dose preferably as divided doses throughout a day. In one embodiment, the daily dose is administered twice daily in equally divided doses. Specifically, a daily dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable folr different metabolic diseases, as will be readily known by those of ordinary skill in the art. Similarly, amounts sufficient to prevent, manage, treat or ameliorate such metabolic disorders, but insufficient to cause, or sufficient to reduce, adverse effects associated with the compounds of the invention are also encompassed by the above described dosage amounts and dose frequency schedules. Further, when a patient is administered multiple dosages of a compound of the invention, not all of the dosages need be the same. For example, the dosage administered to the patient may be increased to improve the prophylactic or therapeutic effect of the compound or it may be decreased to reduce one or more side effects that a particular patient is experiencing.

In a specific embodiment, the dosage of the composition of the invention or a compound of the invention administered to prevent, treat, manage, or ameliorate a metabolic disorder or one or more symptoms thereof in a patient is 150 μg/kg, preferably 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of the composition of the invention or a compound of the invention administered to prevent, treat, manage, or ameliorate a metabolic disorder or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The dosages of prophylactic or therapeutic agents other than compounds of the invention, which have been or are currently being used to prevent, treat, manage, or ameliorate a metabolic disorder or one or more symptoms thereof can be used in the combination therapies of the invention. Preferably, dosages lower than those which have been or are currently being used to prevent, treat, manage, or ameliorate a metabolic disorder or one or more symptoms thereof are used in the combination therapies of the invention. The recommended dosages of agents currently used for the prevention, treatment, management, or amelioration of a metabolic disorder or one or more symptoms thereof can obtained from any reference in the art including, but not limited to, Hardman et al., eds., 1996, Goodman & Gilman's The Pharmacological Basis Of Basis Of Therapeutics 9^{th} Ed, Mc-Graw-Hill, New York; Physician's Desk Reference (PDR) 57^{th} Ed., 2003, Medical Economics Co., Inc., Montvale, N.J., which are incorporated herein by reference in its entirety.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies (e.g., prophylactic or therapeutic agents) are administered within the same patent visit.

In certain embodiments, one or more compounds of the invention and one or more other the therapies (e.g., prophylactic or therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound of the invention may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, the invention provides a method of preventing, treating, managing, or ameliorating a metabolic disorder or one or more symptoms thereof, said methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds of the invention once every day, preferably, once every 2 days, once every 3 days, once every 4 days, once every 5 days, once every 6 days, once every 7 days, once every 8 days, once every 10 days, once every two weeks, once every three weeks, or once a month.

The present invention provides methods of preventing, treating, managing, or preventing a metabolic disorder (e.g., diabetes mellitus), or one or more symptoms thereof, said method comprising: (a) administering to a subject in need thereof one or more doses of an effective amount of one or more compounds of the invention; and (b) monitoring the mean blood glucose levels, blood insulin levels and/or insulin sensitivity in said subject after administration of a certain number of doses of the said compounds of the invention. Moreover, preferably, said certain number of doses is 1, 2, 3, 4, 5, 6, 7, 8, 9, 10 or 12 of an effective amount of the one or more compounds of the invention.

In a specific embodiment, the invention provides a method of preventing, treating, managing, or ameliorating a metabolic disorder (e.g., diabetes mellitus) or one or more symptoms thereof, said method comprising: (a) administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds of the invention; and (b) administering one or more subsequent doses to said subject when the mean blood glucose levels, blood insulin levels and/or insulin sensitivity in said subject is not within normal range (i.e., the range obtained from normal subjects without a metabolic disorder).

In another embodiment, the invention provides a method of preventing, treating, managing, or ameliorating a metabolic disorder (e.g., diabetes mellitus), or one or more symptoms thereof, said method comprising: (a) administering to a subject in need thereof one or more doses of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds of the invention; (b) monitoring the mean blood glucose levels, blood insulin levels and/or insulin sensitivity in said subject after the administration of a certain number of doses (e.g., 1, 2, 3, 4, 5, 6, 7, 8, 9 or more doses); and (c) administering a subsequent dose of the compound(s) of the invention when the mean blood glucose levels, blood insulin levels and/or insulin sensitivity in said subject is not within normal range.

In another embodiment, the invention provides a method of preventing, treating, managing, or ameliorating a metabolic disorder (e.g., diabetes mellitus), or one or more symptoms thereof, said method comprising: (a) administering to a subject in need thereof one or more doses of an effective amount of one or more compounds of the invention; and (b) administering a subsequent dose of the compound(s) of the invention to maintain a normal range of blood glucose levels, blood insulin levels and/or insulin sensitivity. The normal range for blood glucose levels, blood insulin levels and/or insulin sensitivity can be obtained or determined by one of skill in the art using well-known techniques.

In another embodiment, the invention provides a method of preventing, treating, managing, or ameliorating a metabolic disorder (e.g., diabetes mellitus), or one or more symptoms thereof, said method comprising: (a) administering to a subject in need thereof one or more doses of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds of the invention; and (b) administering a subsequent dose of the compound(s) of the invention a normal range of blood glucose levels, blood insulin levels and/or insulin sensitivity.

E. Biological Assays

Several aspects of the pharmaceutical compositions or compounds of the invention are preferably tested in vitro, in a cell culture system, and in an animal model organism, such as a rodent animal model system, for the desired therapeutic activity prior to use in humans. For example, assays which can be used to determine whether administration of a specific pharmaceutical composition or a specific combination of therapies is indicated, include cell culture assays in which a patient tissue sample is grown in culture, and exposed to or otherwise contacted with a pharmaceutical composition, and the effect of such composition upon the tissue sample is observed. The tissue sample can be obtained by biopsy from the patient. This test allows the identification of the therapeutically most effective therapy (e.g., prophylactic or therapeutic agent(s)) for each individual patient. In various specific embodiments, in vitro assays can be carried out with representative cells of cell tyres involved in a metabolic disorder (e.g., insulin-producing cells or beta cells in the pancreas, steriodogenic cells and adipocytes), to determine if a pharmaceutical composition of the invention has a desired effect upon such cell types. As an alternative to the use of tissue, tissue samples, cell lines can be used in in vitro assays.

The pharmaceutical compositions and compounds of the invention can be assayed for their ability to modulate insulin production of beta cells of the pancreas. Modulation of insulin production by beta cells can be determined by measuring, e.g., changes in the level of expression insulin. Techniques known to those of skill in the art, including, but not limited to, immunoprecipitation followed by Western blot analysis, ELISAs, flow cytometry, Northern blot analysis, and RT-PCR can be used to measure the expression of insulin. The pharmaceutical compositions and compounds of the invention can also be assayed for their ability to modulate insulin sensitivity using techniques well-known in the art.

The pharmaceutical compositions and compounds of the invention can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, the pharmaceutical compositions and compounds of the invention are tested in a mouse model system. Such model systems are widely used and well-known to the skilled artisan. Examples of such animal models include, but are not limited to, leptin resistant animals (e.g., db/db mice), melanocortin-4 receptor knockout mice (MR-4−^{/-}), leptin-deficient mice (ob/ob), tubby mice (tubby protein deficiency), the fa/fa (Zucker Diabetic Fatty or ZDF) rat, melanocortin-3 receptor knockout mice, POMC-deficient mice, fat/fat mice, the Dgat1^{tm1Far} mice, Ins2^{Mody} mice, and Pparg^{tm2Rev} mice (see, e.g., Barsh et al., 2000, Nature 404:644-651; Fisher et al., 1999, Int. J. Obes. Rel. Metab. Disord. 23 Suppl:54-58; Giridharan, 1998, Indian J. Med. Res. 108:225-242; Zhang et al., 1994, Nature 372:425-432; Noben-Trauth et al., 1996, Nature 380:534-538; Iida et al., 1996, BBRC 224:597-604; Phillips et al., 1996, Nature Genetics 13:18-19; Chen et al., 2000, Nature Genetics 26:97-102; Butler et al., 2000, Endocrinology 141:3518-3521; Yawen et al., 1999, Nature Medicine 5:1066-1070; Naggert et al., 1995, Nature Genetics 10: 135-142; and Smith et al., 2000, Nature Genetics 25:87-90). In the case of diabetes mellitus, ICR-CDI mice are typically used for measuring the effect of test compound on controlling blood glucose levels. HbA1c is a valuable measure for monitoring the treatment of diabetes in humans and is often used as a parameter of efficacy in clinical trials. A 1-2% reduction is generally seen across most classes of diabetes drugs when used as a monotherapy. An additional 0.5% can sometimes be obtained when combining drugs having different mechanisms.

Further, any assays known to those skilled in the art can be used to evaluate the prophylactic and/or therapeutic utility of the pharmaceutical compositions and compounds of the invention for the disorders disclosed herein.

The toxicity and/or efficacy of the pharmaceutical compositions and compounds of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Pharmaceutical compositions and compounds of the invention that exhibit large therapeutic indices are preferred. While pharmaceutical compositions and compounds of the invention that exhibit toxic side effects may be used, care should be taken to design a delivery system that targets such compositions and compounds to the site of affected tissue in order to minimize potential damage to uninfected cells and, thereby, reduce side effects.

The data obtained from the cell culture assays and animal studies can be used in formulating a range of dosage of the pharmaceutical compositions and compounds of the invention for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography (HPLC) and radioimmunasssay (RIA). The pharmacokinetics of a prophylactic or therapeutic can be determined, e.g., by measuring parameters such as peak plasma level (Cₘₐₓ), area under the curve (AUC, which is measured by plotting plasma concentration of the agent versus time, and reflects bioavailability), half-life of the compound (t_{1/2}), and time at maximum concentration.

Efficacy in preventing or treating a metabolic disorder such as diabetes may be demonstrated, e.g., by detecting the ability of the pharmaceutical compositions and compounds of the invention to reduce the blood glucose, increase hypoinsulinemic insulin levels, increase insulin sensitivity, reduce the dose requirements of other anti-diabetic agents, or reduce the severity of one or more symptoms associated with diabetes are identified in human subjects having diabetes. In accordance with this embodiment, a compound of the invention or a control compound is administered to a human subject having diabetes, and the effect of the compound of the invention on blood glucose levels, blood insulin levels, insulin sensitivity, dose requirements of other anti-diabetic agents, or one or more symptoms of diabetes is determined. A compound of the invention that reduces the blood glucose, increase blood hypoinsulinemic insulin levels, increases insulin sensitivity, reduces the dose requirements of other anti-diabetic agents, or reduces one or more symptoms can be identified by comparing the subjects treated with a control compound to the subjects treated.

F. Kits

The invention encompasses kits that can simplify the administration of a compound of the invention to a subject. A typical kit of the invention comprises a unit dosage form of a compound. In one embodiment, the unit dosage form is a container, preferably a sterile container, containing an effective amount of a compound of the invention and a pharmaceutically acceptable carrier or excipient. The kit can further comprise a label or printed instructions regarding the use of compounds or other informational material that advises the physician, technician or patient on how to appropriately prevent or treat the metabolic disorder in question. In other words, the kit includes instruction means indicating or suggesting a dosing regimen including, but not limited to, actual doses, monitoring procedures (e.g., monitoring blood glucose levels and blood insulin levels), and other monitoring information. The kit can also further comprise a unit dosage form of another prophylactic or therapeutic agent, for example, a container containing an effective amount of another prophylactic or therapeutic agent. In a specific embodiment, the kit comprises a container containing an effective amount of a compound of the invention and a pharmaceutically acceptable carrier or excipieni and a container containing an effective amount of another prophylactic or therapeutic agent and a pharmaceutically acceptable carrier or excipient. Examples of other prophylactic or therapeutic agents include, but are not limited to, those listed above. As with any pharmaceutical product, the packaging material and container included in the kit are designed to protect the stability of the product during storage and shipment.

Kits of the invention can further comprise devices that are useful for administering the unit dosage forms. Examples of such devices include, but are not limited to, syringes, drip bags, patches, and inhalers.

Kits of the invention can further comprise pharmaceutically acceptable vehicles that can be used to administer one or more active ingredients (e.g., a compound of the invention). For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

G. Other Embodiments

The compounds of the invention may be used as research tools (for example, to evaluate the mechanism of action of new drug agents, to isolate new drug discovery targets using affinity chromatography, as antigens in an ELISA or ELISA-like assay, or as standards in in vitro or in vivo assays). These and other uses and embodiments of the compounds and compositions of this invention will be apparent to those of ordinary skill in the art.

The invention is further defined by reference to the following examples describing in detail the preparation of compounds of the invention. It will be apparent to those skilled in the art that many modifications, both to materials and methods, may be practiced without departing from the purpose and interest of this invention. The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLES

Reagents and solvents used below can be obtained from commercial sources such as Aldrich Chemical Co. (Milwaukee, Wis., USA). ¹H-NMR and ¹³C-NMR spectra were recorded on a Varian 300 MHz NMR spectrometer. Significant peaks are tabulated in the order: δ (ppm): chemical shift, multiplicity (s, singlet; d, doublet; t, triplet; q, quartet; m, multiplet; br s, broad singlet), coupling constant(s) in Hertz (Hz) and number of protons.

### Example 1

### Synthesis of 2-(2-azido-ethoxymethyl)-4-(2-chloro-phenyl)-7,7-dimethyl-5-oxo-1,4,5,6,7,8-hexa-hydro-quinoline-3-carboxylic acid ethyl ester

A. 4-(2-azido-ethoxy)-3-oxo-butyric acid ethyl ester

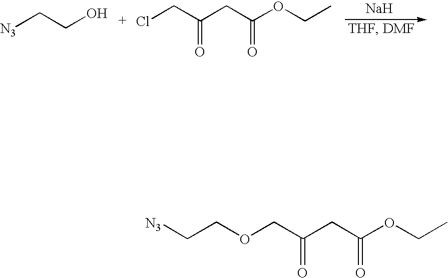

A solution of 2-azidoethanol (8.7 g, 100 mmol) in THF (20 ml) was added to a suspension of sodium hydride (8.8 g, 220 mmol, 60% dispersion in oil) in THF (150 ml). The mixture was stirred at room temperature for 1 h and then cooled to 0-5° C. Ethyl-4-chloroacetoacetate (16.5 g, 100 mmol) in THF (20 ml) was then added dropwise over a period of 0.5 h. The mixture was stirred at room temperature for additional 16 h and diluted with EtOAc (100 ml) and the pH was adjusted to 6-7 with 2 N HCL. Sufficient water was added to dissolve the solid, and the organic layer was then separated. The aqueous layer was further extracted with EtOAc. The combined organic extracts were washed with brine, dried over MgSO₄, filtered and evaporated. The product was purified by column chromatography on silica gel (Hexane-EtOAc, 95:5) to give 4-(2-azido-ethoxy)-3-oxo-butyric acid ethyl ester (16 g, 74.3%) as yellow oil. ¹H-NMR (CDCl₃) δ (ppm), 4.24-4.17(m, 4H), 3.70(t, J=4.8 Hz, 2H), 3.55(s, 2H), 3.43(t, J=5.1 Hz, 2H), 1.28(t, J=7.2 Hz, 3H).
B. 4-(2-azido-ethoxy)-2-(2-chloro-benzylidene)-3-oxo-butyric acid ethyl ester

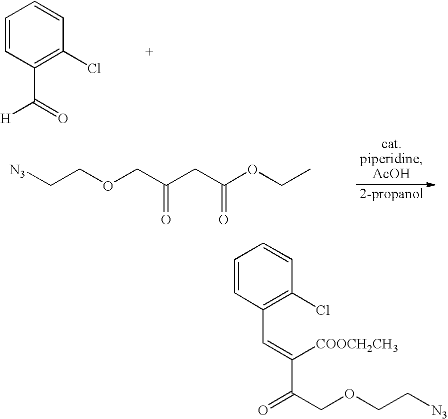

A solution of 2-chlorobenzaldehyde (1.41 g, 10 mmol), 4-(2-azido-ethoxy)-3-oxo-butyric acid ethyl ester (2.15 g, 10 mol), AcOH (0.05 ml), and piperidine (0.10 ml) in 2-propanol (8 ml) was heated under reflux for 20 min. After removal of the solvent, the residue was purified by flash chromatography on silica gel (Hexane-EtOAc, 95:5) to give 4-(2-azido-ethoxy)-2-(2-chloro-benzylidene)-3-oxo-butyric acid ethyl ester (2.8 g, 82.9%) as yellow oil. ¹H-NMR (CDCl₃) δ (ppm), (mix two isomer), 8.07 and 7.99(s, 1H), 7.46-7.22(m, 5H), 4.53 and 4.18(s, 2H), 4.33 and 4.21(q, J=7.2 Hz, J=15.0 Hz, 2H), 3.72 and 3.59(t, J=4.8 Hz, 2H), 3.45 and 3.38(t, J=5.1 Hz, 2H), 1.35 and 1.15(t, J=7.2 Hz, 3H); ESMS clcd for C₁₅H₁₆ClN₃O₄: 337.08. Found: 360.1(M+Na)⁺.
C. 2-(2-azido-ethoxymethyl)-4-(2-chloro-phenyl)-7,7-dimethyl-5-oxo-1,4,5,6,7,8-hexa-hydro-quinoline-3-carboxylic acid ethyl ester

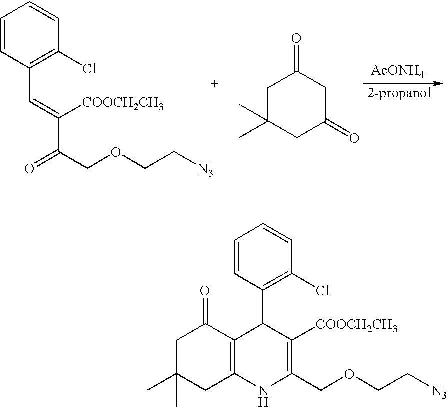

A mixture of 4-(2-azido-ethoxy)-2-(2-chloro-benzylidene)-3-oxo-butyric acid ethyl ester (2.70 g, 8.0 mmol), 5,5-dimethyl-1,3-cyclohexanedione (1.18 g, 8.0 mmol, 95% pure) and ammonium acetate (0.70 g, 9.1 mmol) in 2-propanol (8 ml) was heated under reflux for 16 h. After removal of the solvent, the residue was purified by column chromatography on silica gel (Hexane-EtOAc, 95:5, 9:1)) to give 2-(2-azido-ethoxymethyl)-4-(2-chloro-phenyl)-7,7-dimethyl-5-oxo-1,4,5,6,7,8-hexa-hydro-quinoline-3-carboxylic acid ethyl ester (2.28 g, 62%). ¹H-NMR (CDCl₃) δ (ppm), 7.40(d, J=7.8 Hz, 1H), 7.27-7.23(m, 2H), 7.16-7.02(m, 2H), 5.41(s, 1H), 4.83(d, J=15.0 Hz, 1H), 4.79 (d, J=15.0 Hz, 1H), 4.08-3.96(m, 2H), 3.82-3.75 (m, 2H), 3.54-3.48(m, 2H), 2.42-2.10(m, 4H), 1.17(t, J=7.2 Hz, 3H), 1.08(s, 3H), 0.96(s, 3H); ESMS clcd for C₂₃H₂₇ClN₄O₄: 458.17. Found: 459.2 (M+H)⁺.

### Example 2

### Synthesis of 2-(2-amino-ethoxymethyl)-4-(2-chloro-phenyl)-7,7-dimethyl-5-oxo-1,4,5,6,7,8-Hexahydro-quinoline-3-carboxylic acid ethyl ester

Compound 1 can be prepared by Route A or Route B, each set forth below.

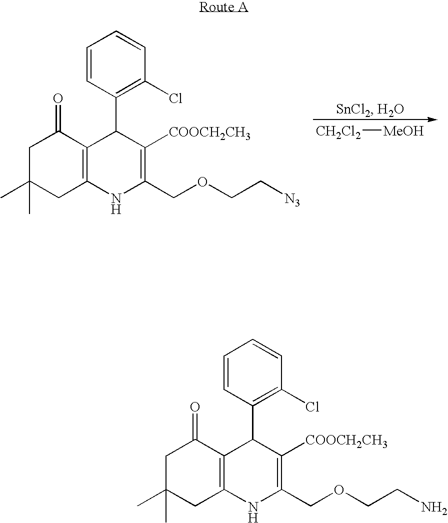

A mixture of 2-(2-azido-ethoxymethyl)-4-(2-chloro-phenyl)-7,7-dimethyl-5-oxo-1,4,5,6,7,8-hexahydroquinoline-3-carboxylic acid ethyl ester (2.12 g, 4.6 mmol) and tin (II) chloride (2.18 g, 11.5 mmol) in CH₂Cl₂/MeOH (2:1, 12 ml) containing 2 drops of water was stirred at room temperature for about 6 h. The reaction mixture was diluted with CH₂Cl₂ (100 ml) and a saturated solution of NaHCO₃ was added to adjust the pH to 9-10, and the layers were then separated. The aqueous layer was further extracted with CH₂Cl₂. The combined organic extracts were dried over MgSO₄, filtered and evaporated. The product was purified by column chromatography on silica gel (CH₂Cl₂:MeOH, 95:5) to give 2-(2-amino-ethoxymethyl)-(2-chloro-phenyl)-7,7-dimethyl-5-oxo-1,4,5,6,7,8-hexahydro-quinoline-3-carboxylic acid ethyl ester (1.65 g, 82.8%) as yellow sold. ¹H-NMR (CDCl₃) δ (ppm), 8.30(s, 1H), 7.43(d, J=6 Hz, 1H), 7.30-7.25(m, 1H), 7.18-7.02(m, 2H), 5.43(s, 1H), 4.85 and 4.78(2d, J=16.8 Hz, each 1H), 4.10-4.02(m, 2H), 3.65-3.60(m, 2H), 3.02(t, J=4.5 Hz, 2H), 2.46-2.30(m, 2H), 2.28-2.12(m, 2H), 1.35(bs, 2H), 1.19 (t, J=7.2 Hz, 3H), 1.10(s, 3H), 0.98(s, 3H); ESMS clcd for C₂₃H₂₉ClN₂O₄: 432.18. Found: 433.2 (M+H)⁺.

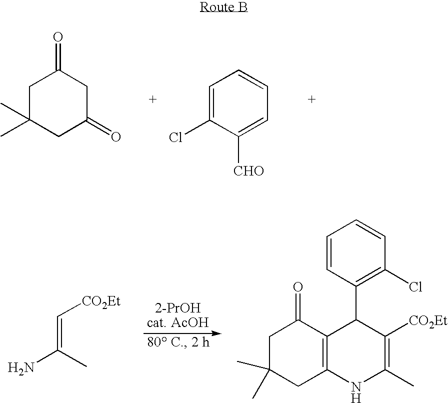

5,5-Dimethyl-cyclohexane-1,3-dione, 2-Chloro-benzaldehyde and 3-Amino-but-2-enoic acid ethyl ester in 2 L of 2-PrOH were added into a 5 L 4-necked round bottomed flask equipped with a mechanical stirrer, a thermometer and a heating mantle underneath. The mixture was stirred and the reaction temperature was brought to about 40° C. About 4 mL of acetic acid was added while stirring. The reaction was stirred for about 2 h at about 80° C. The reaction was then cooled overnight and the precipitate was collected via filtration using a Buchner funnel and washed successively with ethyl acetate (about 1.5L) to obtain a white crystalline material. The product was then vacuum dried in a round bottomed flask to give 4-(2-Chloro-phenyl)-2,7,7-trimethyl-5-oxo-1,4,5,6,7,8-hexahydro-quinoline-3-carboxylic acid ethyl ester (1.33 kg, 89% yield, 99% purity). ¹H-NMR (CDCl₃) δ (ppm), 7.39(d, J=7.5 Hz, 1H), 7.23(d, J=6.6 Hz, 1H), 7.15-7.08(m, 1H), 7.05-7.00(m, 1H), 6.06(s, 1H), 5.38(s, 1H), 4.10-4.00(m, 2H), 2.34-2.10(m, 4H), 2.30(s, 3H), 1.17(t, J=7.2 Hz, 3H), 1.07(s, 3H), 0.95(s, 3H). ESMS clcd for C₂₁H₂₄ClNO₃: 373.14. Found: 374.1 (M+H)⁺.

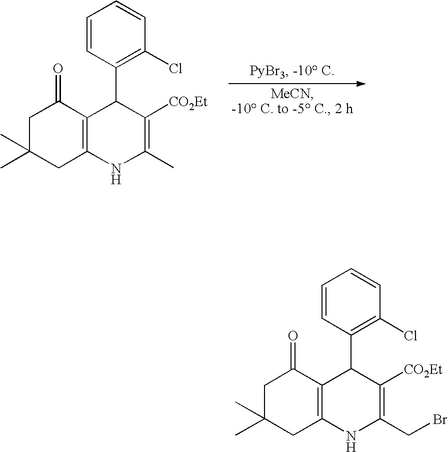

2-Bromomethyl-4-(2-chloro-phenyl)-7,7-dimethyl-5-oxo-1,4,5,6,7,8-hexahydro-quinoline-3-carboxylic acid ethyl ester (459.0 g, 1.23 mol) pyridine (110 ml, 1.35 mol) and MeCN were added to a 5 L 4-necked round bottomed flask equipped with a mechanical stirrer, a thermometer and a cooling bath underneath. The reaction mixture was cooled down to about −10° C. and Py.Br₃ (436.3 g, 1.23 mol) was added in portions over a period of about 10 min while maintaining the temperature of the reaction below about −5° C. The reaction was stirred for about an additional 1.5 h at about −5° C. and then poured into about 12 L of an ice-water mixture. The resulting slurry was stirred and left standing overnight. The precipitates were collected using filtration, washed with water (500 mL×4) and washed with with ether (200 mL×2). The resulting yellow solid was then dried under vacuum for about 10 h over a water bath of about 50° C. for 10 h to give 2-Bromomethyl-4-(2-chloro-phenyl)-7,7-dimethyl-5-oxo-1,4,5,6,7,8-hexahydro-quinoline-3-carboxylic acid ethyl ester (523.0 g, 94% yield, 78.6% purity). ¹H-NMR (CDCl₃) δ (ppm), 7.38(d, J=7.5 Hz, 1H), 7.25(d, J=6.6 Hz, 1H), 7.14-7.03(m, 2H), 6.42(s, 1H), 5.40(s, 1H), 4.73(q, J=9.3 Hz, J=15.3 Hz, 2H), 4.15-4.03(m, 2H), 2.40-2.10(m, 4H), 1.19(t, J=7.2 Hz, 3H), 1.09(s, 3H), 0.95(s, 3H). ESMS clcd for C₂₁H₂₃BrClNO₃: 451.05. Found: 452.0 (M+H)⁺.

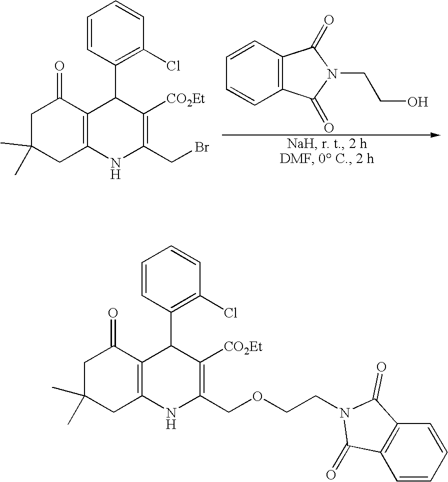

2-(2-Hydroxy-ethyl)-isoindole-1,3-dione (102.2 g, 0.534 mol) was added over a period of about 30 min to a stirred suspension of sodium hydride (12.82 g, 0.534 mol) in 2.25 L of anhydrous DMF at room temperature in a 5 L 4-necked round bottomed flask equipped with a mechanical stirrer and a thermometer. The reaction was stirred for about 1.5 h at room temperature until the solution solidified (i.e., was difficult to stir). 400 mL of anhydrous DMF and 240 mL of hexane were added resulting in a uniformed slurry. The reaction temperature was reduced to about 0° C. and 2-Bromomethyl-4-(2-chloro-phenyl)-7,7-dimethyl-5-oxo-1,4,5,6,7,8-hexahydro-quinoline-3-carboxylic acid ethyl ester (110.0 g, 0.243 mol) in solid form was added in portions over a period of about 10 min. The reaction mixture was then stirred for about 1.5 h at room temperature (until TLC analysis indicated the completion of the reaction) and the mixture was then poured into about 10 L of an ice-water mixture. The mixture was stirred left standing overnight. The precipitate was filtered, washed successively with water (500 mL×3), hexane (2L×1), and dried in vacuo for about 24 hours. The powdered product was then added in portions to about 1.6 L of warm MeOH (about 45° C.) with vigorous stirring, during which time the impurities were dissolved and 4-(2-Chloro-phenyl)-2-[2-(1,3-dioxo-1,3-dihydro-isoindol-2-yl)-ethoxymethyl]-7,7-dimethyl-5-oxo-1,4,5,6,7,8-hexahydro-quinoline-3-carboxylic acid ethyl ester appeared as a new precipitate. The solution was then cooled to about room temperature (about 1 h). The precipitate was collected using filtration, washed with 200 mL of MeOH and drained well. The solid crude product was taken up in about 1 L of MeOH in a 2 L round bottomed flask, stirred with heating (bath temperature of about 70° C.) for about 25 min to homogenize the product. The flask was then cooled to about room temperature (about 1 h) and the product was filtered, drained well and vacuum dried (405 g, 74% yield, 99.5% purity). ¹H-NMR (CDCl₃) δ (ppm), 7.89-7.86(m, 2H), 7.80-7.77(m, 2H), 7.60(s, 1H), 7.35(d, J=7.5 Hz, 1H), 7.20(d, J=7.8 Hz, 1H), 7.10-6.99(m, 2H), 5.36(s, 1H), 4.71-4.68(m, 2H), 4.03-3.96(m,4H),3.80-3.74(m, 2H), 2.46-2.44(m, 2H),2.17-2.15(m, 2H), 1.13(t, J=7.2 Hz, 3H), 1.11(s, 3H), 0.99(s, 3H). ESMS clcd for C₃₁H₃₁ClN₂O₆: 562.19. Found: 563.2 (M+H)⁺.

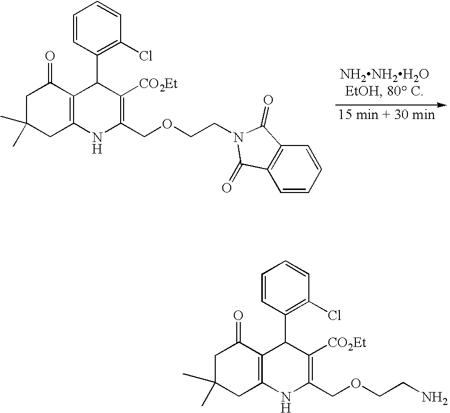

Hydrazine hydrate (26 ml, 0.520 mol) was added over a period of about 12 min to a stirred solution of 4-(2-Chloro-phenyl)-2-[2-(1,3-dioxo-1,3-dihydro-isoindol-2-yl)-ethoxymethyl]-7,7-dimethyl-5-oxo-1,4,5,6,7,8-hexahydro-quinoline-3-carboxylic acid ethyl ester in anhydorous ethanol (800 ml) in a 5 L 4-necked round bottomed flask equipped with a mechanical stirrer, a thermometer and a heating mantle underneath. The reaction mixture was then stirred at about 78° C. for about 30 min. and the heating mantle was removed. The mixture was allowed to cool overnight and and filtered. The filtrate was concentrated in vacuo (removing approximately 98% of solvent) while keeping the bath temperature less than about 50° C. The resulting residue was then taken up in a separatory funnel with about 1.0 L of CH₂Cl₂ and 700 mL of water and washed with about 50 mL of brine. The organic layer was washed again with about 600 mL of water and about 100 mL of brine, dried over MgSO₄ and concentrated. The crude product was taken into a 1:1 mixture of 400 mL of hexane/ethyl acetate and stirred at about 55° C. for about 30 min to homogenize the precipitate. After cooling, the precipitate was filtered, washed with about 100 mL of ether and vacuum dried to give 2-(2-Amino-ethoxymethyl)-4-(2-chloro-phenyl)-7,7-dimethyl-5-oxo-1,4,5,6,7,8-hexahydro-quinoline-3-carboxylic acid ethyl ester (236 g, 78.4% yield, 99.4% purity).

The benzenesulfonate salt of 2-(2-Amino-ethoxymethyl)-4-(2-chloro-phenyl)-7,7-dimethyl-5-oxo-1,4,5,6,7,8-hexahydro-quinoline-3-carboxylic acid ethyl ester was prepared by adding the compound (590.0 g, 1.37 mol) to ethyl alcohol (2360 mL:4 mL ethanol per gram of amine) in a 4 L flask followed by stirring with heat until a clear solution was obtained (about 40-45° C., solution temperature). After removal of the heating apparatus, benzenesulfonic acid (216.3 g, 1.37 mol) was added as a solid in one portion with stirring. After the addition of benzenesulfonic acid, the resulting mixture was stirred for about 30 seconds. The stirring was stopped and yellow precipitates started to form immediately. The reaction mixture was then allowed to stand for about 3 hours at room temperature. When the reaction mixture reached room temperature, the precipitate was collected by filtration, washed with ethanol 3 times (total 1 L). The collected solid was vacuum dried to give the benzenesulfonic acid salt product as a light yellow crystalline powder (738.2 g, 91.5% yield, 99.8% purity).

### Example 3

### Synthesis of 2-(2-amino-ethoxymethyl)-7,7-dimethyl-5-oxo-4-pyridin-2-yl-1,4,5,6,7,8-hexahydro-quinoline-3-carboxylic acid ethyl ester

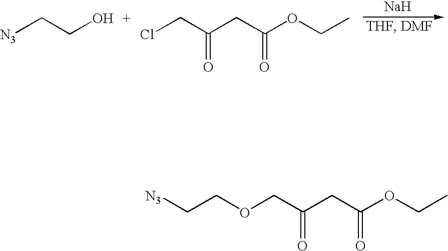

A solution of 2-azidoethanol (8.7 g, 100 mmol) in THF (20 ml) was added to suspension of sodium hydride (8.8 g, 220 mmol, 60% dispersion in oil) in THF (150 ml). The mixture was stirred at room temperature for about 1 h and then cooled to 0-5° C. Ethyl-4-chloroacetoacetate (16.5 g, 100 mmol) in THF (20 ml) was then added drop wise over a period of about 0.5 h. The mixture was stirred at room temperature for about 16 h and diluted with EtOAc (100 ml) and the pH was adjusted to 6-7 with 2 N HCl. Sufficient water was added to dissolve the solid, and the layers were then separated. The aqueous layer was further extracted with EtOAc. The combined organic extracts were washed with brine, dried over MgSO4, filtered and evaporated. The product was purified by column chromatography on silica gel (Hexane-EtOAc, 95:5) to give 4-(2-azido-ethoxy)-3-oxo-butyric acid ethyl ester (16 g, 74.3%) as yellow oil.

¹H-NMR (CDCl₃) δ (ppm), 4.24-4.17(m, 4H), 3.70(t, J=4.8 Hz, 2H), 3.55(s, 2H), 3.43(t, J=5.1 Hz, 2H), 1.28(t, J=7.2 Hz, 3H);

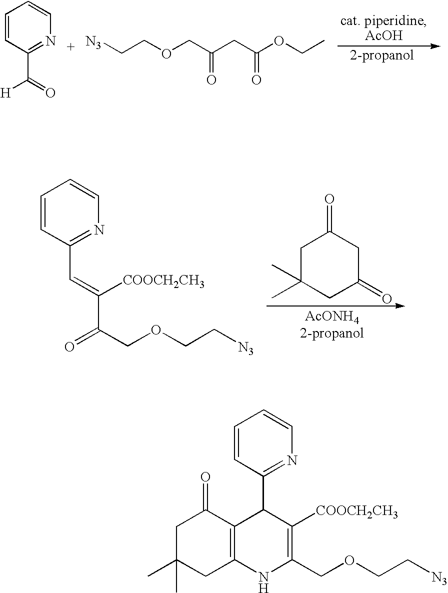

A solution of 2-pyridinecarboxaldehyde (0.540 mg, 5 mmol), 4-(2-azido-ethoxy)-3-oxo-butyric acid ethyl ester (1.075 g, 5 mmol), AcOH (0.03 ml), and piperidine (0.06 ml) in 2-propanol (8 ml) was heated under reflux for 20 min. After cooling, dimedone (5,5-dimethyl-1,3-cyclohexanedione) (0.740 g, 5.0 mmol, 95% pure) and ammonium acetate (0.470 g, 6.0 mmol) was added and the reaction was heated under reflux for 16 h. After removal of the solvent, the residue was purified by column chromatography on silica gel (Hexane-EtOAc 1:1 and EtOAc) to give 2-(2-azido-ethoxymethyl)-7,7-dimethyl-5-oxo-4-pyridin-2-yl-1,4,5,6,7,8-hexa-hydro-quinoline-3-carboxylic acid ethyl ester (0.870 g, 41%).

¹H-NMR (CDCl₃) δ (ppm), 8.52(s, 1H), 7.58-7.48(m, 2H), 7.30-7.28(m, 1H), 7.04-7.0(m, 1H), 5.26 (s, 1H), 4.95(d, J=15.0 Hz, 1H), 4.84 (d, J=15.0 Hz, 1H), 4.20-4.07(m, 2H), 3.84-3.78(m, 2H), 3.54-3.48(m, 2H), 2.42-2.10(m, 4H), 1.21(t, J=7.2 Hz, 3H), 1.14(s, 3H), 1.02 (s, 3H); ESMS clcd for C₂₂H₂₇N₅O₄: 425.21. Found: 426.2 (M+H)⁺.

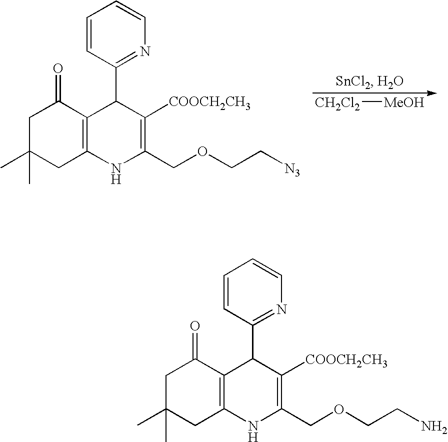

A mixture of 2-(2-azido-ethoxymethyl)-7,7-dimethyl-5-oxo-4-pyridin-2-yl-1,4,5,6,7,8-hexa-hydro-quinoline-3-carboxylic acid ethyl ester (0.430 g, 1.0 mmol) and tin (II) chloride (1.2 g, 6.0 mmol) in mixture CH₂Cl₂-MeOH (2:1, 8 ml) containing 2 drops of water was stirred at room temperature for 6 h. The reaction mixture was diluted with CH₂Cl₂ (20 ml) and saturated solution of NaHCO₃ was added to pH 9-10, and the layers were then separated. The aqueous layer was further extracted with CH₂Cl₂. The combined organic extracts were dried over MgSO₄, filtered and evaporated. The product was purified by column chromatography on silica gel (CH₂Cl₂:MeOH, 9:1) to give 2-(2-amino-ethoxymethyl)-7,7-dimethyl-5-oxo-4-pyridin-2-yl-1,4,5,6,7,8-hexahydro-quinoline-3-carboxylic acid ethyl ester (0.280 g, 69.5%) as yellow sold.

¹H-NMR (CDCl₃) δ (ppm), 8.42(s, 1H), 8.08(bs, 1H), 7.51-7.39(m, 2H), 7.00-6.96(m, 1H), 5.17(s, 1H), 4.79(s, 2H), 4.01(q, J=14.1 Hz, J=7.2 Hz, 2H), 3.67-3.57(m, 2H), 2.96(t, J=5.1 Hz, 2H), 239-2.08(m, 4H), 1.15(t, J=7.2 Hz, 3H), 1.06(s, 3H), 0.94(s, 3H), ESMS clcd for C₂₂H₂₉N₃O₄: 399.22. Found: 400.2 (M+H)⁺.

### Example 4

### Synthesis of S-(−)-2-(2-amino-ethoxymethyl)-4-(2-chloro-phenyl)-7,7-dimethyl-5-oxo-1,4,5,6,7,8-hexahydroquinoline-3-carboxylic acid ethyl ester

S-(−)-2-(2-amino-ethoxymethyl)-4-(2-chloro-phenyl)-7,7-dimethyl-5-oxo-1,4,5,6,7,8-hexahydro-quinoline-3-carboxylic acid ethyl ester was resolved from the racemic Compound 1 mixture on a supercritical fluid column (SFC) as follows:

| | |
|---|---|
| Column: | Chiralpak AS-H, 2.1 × 25 cm |
| Mobile Phase: | 20% MeOH in CO2 |
| Temperature: | 35° C. |
| Flow Rate: | 50 ml/min |
| Sample Solution: | 100 mg/mL in MeOH |
| Injection Volume: | 1.9 Ml |
| System: | Berger Multigram SFC + PDR-Chiral AutoPrep |
| | control software and Advanced Laser Polarimeter |

The following analytical data were obtained:

¹H-NMR (CDCl₃) δ (ppm): 8.23 (s, 1H), 7.32 (d, J=8.4 Hz, 1H), 7.26 (dd, J₁=8.4 Hz, J₂=2.1 Hz, 1H), 7.09 (dd, J₁=8.4 Hz, J₂=2.1 Hz, 1H), 5.33 (s, 1H), 4.69-4.84 (m, 2H), 4.00 (q, J=6.9 Hz, 2H), 3.67-3.69 (m, 2H), 3.06-3.09 (m, 2H), 2.00-2.35 (m, 4H), 1.16 (t, J=6.9 Hz, 3H), 1.05 (s, 3H), 0.93 (s, 3H); ESMS clcd for C₂₃H₂₈Cl₂N₂O₄: 466.14. Found: 467.2 (M+H)⁺.

The methods from Examples 1-3, shown above, were utilized with appropriate starting materials and reagents to produce the following compounds of the invention. Choice of the appropriate starting materials and reagents will be readily apparent to one of skill in the art for these and other compounds of this invention.

### Example 5

### Synthesis of 2-(2-amino-ethoxymethyl)-4-benzo[1,3]-dioxol-5-yl-7,7-dimethyl-5-oxo-1,4,5,6,7,8-hexahydroquinoline-3-carboxylic acid ethyl ester

¹H-NMR (CDCl₃) δ (ppm), 8.10(s, 1H), 6.77-6.74(m, 2H), 6.61(d, J=7.8 Hz, 1H), 5.84-5.82(m, 2H), 4.94(s, 1H), 4.79 and 4.72(2d, J=16.8 Hz each 1H), 4.02(q, J=14.1 Hz, J=7.2 Hz, 2H), 3.58-3.52(m,2H), 2.94(t, J=4.8 Hz, 2H),2.38-2.10(m, 4H), 1.48(bs,1H), 1.18(t, J=7.2 Hz, 3H), 1.04(s, 3H), 0.93(s, 3H); ESMS clcd for C₂₄H₃₀N₂O₆: 442.21. Found: 443.3 (M+H)⁺.

### Example 6

### Synthesis of 2-(2-amino-ethoxymethyl)-4-(2-chloro-phenyl)-1,7,7-trimethyl-5-oxo-1,4,5,6,7,8-hexahydro-quinoline-3-carboxylic acid ethyl ester

¹H-NMR (CDCl₃) δ (ppm), 7.23-7.19(m, 2H), 7.10-6.96(m, 2H), 5.48(s, 1H), 4.82(d, J=12.9 Hz, 1H), 4.6(d, J=12.9 Hz, 1H), 4.08-4.02(m, 2H), 3.50(t, J=5.4 Hz, 2H), 3.36(s, 3H), 2.84(t, J=5.1 Hz, 2H), 2.54 (d, J=16.8 Hz, 1H), 2.33(d, J=17.1 Hz, 1H), 2.15-2.13(m, 2H), 1.65(bs, 2H), 1.20(t, J=6.9 Hz, 3H), 1.05(s, 3H), 0.97(s, 3H); ESMS clcd for C₂₄H₃₁ClN₂O₄: 446.20. Found: 447.2 (M+H)⁺.

### Example 7

### Synthesis of 2-(2-amino-ethoxymethyl)-4-(2-chloro-phenyl)-7,7-dimethyl-5-oxo-1,4,5,6,7,8-hexahydro-quinoline-3-carboxylic acid ethyl ester; Complex with Benzene Sulfonic Acid

¹H-NMR (CDCl₃) δ (ppm), 7.88-7.80(m, 3H), 7-49-7.19(m, 4H), 7.08-7.00(m, 2H), 5.33(s, 1H), 4.74(d, J=15 Hz, 1 H), 4.64(d, J=15 Hz, 1 H), 4.08-3.95(m, 2H), 3.68-3.60(m, 2H), 3.10-3.05(m, 2H), 2.19-1.98(m, 4H), 1.14 (t, J=6.9 Hz, 3H), 0.81(s, 3H), 0.69(s, 3H).

### Example 8

### Synthesis of 2-(2-amino-ethoxymethyl)-4-(2,3-dichloro-phenyl)-7,7-dimethyl-5-oxo-1,4,5,6,7,8-hexahydro-quinoline-3-carboxylic acid ethyl ester

¹H-NMR (CDCl₃) δ (ppm), 8.37(s,1H), 7.34-7.21(m, 2H), 7.05(t, J=7.8 Hz, 1H), 5.44(s, 1H), 4.82(d, J=15 Hz, 1H), 4.73(d, J=15 Hz, 1H), 4.05-3.99(m, 2H), 3.59(t, J=5.7 Hz, 2H), 2.99(t, J=5.4 Hz, 2H), 2.40-2.12(m,4H), 1.14(t, J=6.9 Hz, 3H), 1.07(s, 3H), 0.95(s, 3H); ESMS clcd for C₂₃H₂₈Cl₂N₂O₄: 466.14. Found: 469.2.

### Example 9

### Synthesis of 2-(2-amino-ethoxymethyl)-7,7-dimethyl-4-(3-nitro-phenyl)-5-oxo-1,4,5,6,7,8-hexahydro-quinoline-3-carboxylic acid ethyl ester

¹H-NMR (CDCl₃) δ (ppm), 8.58(s, 1H), 8.13-8.05(m, 1H), 7.98-7.94(m, 1H), 7.73-7.70(m, 1H), 7.40-7.34(m, 1H), 5.15(s, 1H), 4.80(s, 2H), 4.043(q, J=14.1 Hz, J=6.9 Hz, 2H), 3.68-3.60(m, 2H), 2.99-2.94(m, 2H), 2.42-2.04 (m, 4H), 1.19(t, J=6.6 Hz, 3H),1.09(s, 3H), 0.93(s, 3H); ESMS clcd for C₂₃H₂₉N₃O₆: 443.21. Found: 444.3 (M+H)⁺.

### Example 10

### Synthesis of 2-(2-amino-ethoxymethyl)-4-(3-amino-phenyl)-7,7-dimethyl-5-oxo-1,4,5,6,7,8-hexahydroquinoline-3-carboxylic acid ethyl ester

¹H-NMR (CDCl₃) δ (ppm), 8.15(s, 1H), 6.99(t, J=7.8 Hz, 1H), 6.72-6.69(m, 2H), 6.47-6-44(m, 1H), 4.99(s, 1H), 4.81(s, 2H), 4.06(q, J=14.4 Hz, J=7.2 Hz, 2H), 3.63-3.58(m, 2H), 2.98(t, J=5.4 Hz, 2H), 2.40-2.18(m, 4H), 1.21(t, J=7.2 Hz, 3H), 1.08(s, 3H), 0.97(s, 3H); ESMS clcd for C₂₃H₃₁N₃O₄: 413.23. Found: 414.3 (M+H)⁺.

### Example 11

### Synthesis 2-(2-amino-ethoxymethyl)-4-(2-chloro-phenyl)-5-oxo-1,4,5,6,7,8-hexahydro-quinoline-3-carboxylic acid ethyl ester

¹H-NMR (CDCl₃) δ (ppm), 8.55(s, 1H), 7.51(d, J=7.8 Hz, 1H), 7.40-7.33(m, 1H), 7.26-7.12(m, 2H), 5.52(s, 1H), 4.94(d, J=18 Hz, 1H), 4.84(d, J=18 Hz, 1H), 4.16-4.08(m, 2H), 3.71(t, J=5.1 Hz, 2H), 3.10(t, J=4.8 Hz, 2H), 2.62-2.58(m, 2H), 2.44-2.39(m, 2H), 2.12-2.00(m, 2H), 1.27(t, J=7.2 Hz, 3H); ESMS clcd for C₂₁H₂₅ClN₂O₄: 404.15. Found: 405.2 (M+H)⁺.

### Example 12

### Synthesis of 2-(2-amino-ethoxymethyl)-7,7-dimethyl-4-(2-nitro-phenyl)-5-oxo-1,4,5,6,7,8-hexahydro-quinoline-3-carboxylic acid ethyl ester

¹H-NMR (CDCl₃) δ (ppm), 8.58(bs, 1H), 7.70(d, J=8.1 Hz, 1H), 7.49-7.40(m, 2H), 7.21(t, J=6.9 Hz, 1H), 5.86(s, 1H), 4.80(s, 2H), 4.10-3.92(m, 2H), 3.66-3.52(m, 2H), 2.99(t, J=5.1 Hz, 2H), 2.40-2.04(m, 4H), 1.06(t, J=6.9 Hz, 3H), 1.03(s, 3H), 0.87(s, 3H); ESMS clcd for C₂₃H₂₉N₃O₆: 443.21. Found: 444.2 (M+H)⁺.

### Example 13

### Synthesis of 2-(2-amino-ethoxymethyl)-4-(2-fluoro-phenyl)-7,7-dimethyl-5-oxo-1,4,5,6,7,8-hexahydroquinoline-3-carboxylic acid ethyl ester

¹H-NMR (CDCl₃) δ (ppm), 8.23(s, 1H), 7.38-7.7.23 (m, 1H), 7.18-6.82(m, 3H), 5.18(s, 1H), 4.80(d, J=17.7 Hz, 1H), 4.70(d, J=17.7 Hz, 1H), 3.97(q, J=14.4 Hz, J=7.2 Hz, 2H), 3.55 (t,J=5.1H,2H), 2.94(t,J=5.4 Hz,2H), 2.38-2.01(m,4H), 1.54(bs,2H), 1.14(t,J=7.2 Hz,3H), 1.04(s,3H), 0.90(s, 3H); ESMS clcd for C₂₃H₂₉FN₂O₄: 416.21. Found: 417.2 (M+H)⁺.

### Example 14

### Synthesis of 2-(2-amino-ethoxymethyl)-4-(2-chloro-phenyl)-7-isopropyl-5-oxo-1,4,5,6,7,8-hexahydro-quinoline-3-carboxylic acid ethyl ester

¹H-NMR (CDCl₃) δ (ppm), 8.34 (s, 0.6 H),8.24(s, 0.4 H),7.40-7.36(m, 1H), 7.26-7.20(m,1H), 7.12-7.00(m, 2H), 5.39(s, 0.6H), 5.35(s, 0.4H), 4.82-4.68(m, 2H), 4.02-3.96(m, 2H),3.59(t, J=4.8 Hz, 2H), 2.97(t, J=4.8 Hz, 2H), 2.5-2.25(m, 2H), 2.05-1.50(m, 4H), 1.14(t, J=7.2 Hz, 3H), 0.88(t, J=6.3 Hz, 6H); ESMS clcd for C₂₄H₃₁ClN₂O₄: 446.20. Found: 447.5 (M+H)⁺.

### Example 15

### Synthesis of 2-(2-amino-ethoxymethyl)-4-(2-chloro-phenyl)-7-methyl-5-oxo-1,4,5,6,7,8-hexahydro-quinoline-3-carboxylic acid ethyl ester

¹H-NMR (CDCl₃) δ (ppm), 8.40(s, 0.75H), 8.25(s, 0.25H), 7.37-7.33(m, 1H), 7.20-7.18(m, 1H), 7.10-6.95(m, 2H), 5.37-5.34(m, 1H), 4.80-4.68(m, 2H), 4.00-3.94(m, 2H), 3.58-3.56(m, 2H), 2.94(t, J=4.8 Hz, 2H), 2.50-1.92(m, 5H), 1.63(bs, 2H), 1.16-1.09(m, 3H), 1.02-0.96(m, 3H); ESMS clcd for C₂₂H₂₇ClN₂O₄: 418.17. Found: 419.1 (M+H)⁺.

### Example 16

### Synthesis of 2-(2-Amino-ethoxymethyl)-4-(4-chloro-phenyl)-7,7-dimethyl-5-oxo-1,4,5,6,7,8-hexahydro-quinoline-3-carboxylic acid ethyl ester

¹H-NMR (CDCl₃) δ (ppm), 8.17 (s, 1H), 7.24 (d, J=7.8 Hz, 2H), 7.16 (d, J=7.8 Hz, 2H), 5.02(s, 1H), 4.81 (s, 2H), 4.31 (bs, 1H), 4.03 (q, J=6.9 Hz), 3.67-3.63 (m, 2H), 3.05-3.02 (m, 2H), 2.44-2.31 (m, 2H), 2.20-2.11 (m, 2H), 2.04 & 2.00 (s, each 1H), 1.26 & 1.18 (t, J=6.9 Hz, each 3H), 1.07 (s, 3H), 0.92 (s, 3H); ESMS clcd for C₂₃H₂₉ClN₂O₄: 432.18. Found: 433.2 (M+H)⁺.

### Example 17

### Synthesis of 2-(2-Amino-ethoxymethyl)-4-(2-methoxy-phenyl)-7,7-dimethyl-5-oxo-1,4,5,6,7,8-hexahydro-quinoline-3-carboxylic acid ethyl ester

¹H-NMR (CDCl₃) δ (ppm), 8.01 (s, 1H), 7.34-7.27 (m, 1H), 7.11-7.06 (m, 1H), 6.86-6.77 (m, 1H), 5.31 & 5.30 (2s, each 1H), 4.83-4.68 (m, 2H), 4.01-3.98 (m, 2H), 3.80 (s, 3H), 3.65-3.56 (m, 2H), 2.41-2.04 (m, 8H), 1.19 (t, J=6.9 Hz), 1.08 (s, 3H), 0.94 (s, 3H); ESMS clcd for C₂₄H₃₂N₂O₅: 428.23. Found: 429.2 (M+H)⁺.

### Example 18

### Synthesis of 2-(2-Amino-ethoxymethyl)-4-(2-cyano-phenyl)-7,7-dimethyl-5-oxo-1,4,5,6,7,8-hexahydro-quinoline-3-carboxylic acid ethyl ester

¹H-NMR (CDCl₃) δ (ppm), 8.81 (bs, 1H), 7.59-7.53 (m, 4H), 7.44 (dd, J1=J27.5 Hz, 1H), 7.20 (dd, J1=J2=7.5 Hz, 1H), 5.39 (bs, 2H), 5.30 (s, 1H), 4.91-4.72 (m, 2H), 4.05-3.91 (m, 2H), 3.83-3.72 (m, 2H), 3.12 (bs, 2H), 2.56-2.38 (m, 2H), 2.22-2.02 (m, 4H), 1.14 (t, J=7.2 Hz, 1H), 1.06 (s, 3H), 0.96 (s, 3H); ESMS clcd for C₂₄H₂₉N₃O₄: 423.22. Found: 424.2 (M+H)⁺.

### Example 19

### Synthesis of 4-(2-Chloro-phenyl)-2-[2-(2-hydroxy-3-phenoxy-propylamino)-ethoxymethyl]-7,7-dimethyl-5-oxo-1,4,5,6,7,8-hexahydro-quinoline-3-carboxylic acid ethyl ester

¹H-NMR (CDCl₃) δ (ppm), 7.62(s, 1H), 7.40-7.23(m, 4H), 7.13(t, J=7.5 Hz, 1H), 7.05-6.97(m, 4H), 5.39(s, 1H), 4.77(d, J=8.1 Hz, 2H), 4.13(bs, 1H), 4.02-4.08(m, 4H), 3.71-3.65(m, 2H), 3.56(s, 3H), 2.96-2.85(m, 4H), 2.41-2.25(m, 2H), 2.22-2.07(m, 2H), 1.16(t, J=7.2 Hz,3H), 1.05(s, 3H), 0.93(s, 3H); ESMS clcd for C₃₂H₃₉ClN₂O₆: 582.25. Found: 583.3 (M+H)⁺.

### Example 20

### Synthesis of 2-(2-amino-ethoxymethyl)-4-(2-chloro-phenyl)-7,7-dimethyl-5-oxo-1,4,5,6,7,8-hexahydro-quinoline-3-carboxylic acid methyl ester

¹H-NMR (CDCl₃) δ (ppm), 8.27(s, 1H), 7.36(d, J=7.2 Hz, 1H), 7.26-7.22(m, 1H), 7.18-7.01(m, 2H), 5.39(s, 1H), 4.81 and 4.75(2d, J=16.8 Hz, each 1H), 3.62-3.56(m, 2H), 3.56(s, 3H), 2.98(t, J=5.1 Hz, 2H), 2.40-2.31(m, 2H), 2.22-2.12(m, 2H), 1.67(bs, 2H), 1.07(s, 3H), 0.95(s, 3H); ESMS clcd for C₂₂H₂₇ClN₂O₄: 418.17. Found: 419.2 (M+H)⁺.

### Example 21

### Synthesis of 2-(2-amino-ethoxymethyl)-4-(2-chloro-phenyl)-7,7-dimethyl-5-oxo-1,4,5,6,7,8-hexahydro-quinoline-3-carboxylic acid methyl ester complex with benzenesulfonic acid

¹H-NMR (CDCl₃) δ (ppm), 7.89-7.85(m, 2H), 7.48-7.36(m, 2H), 7.7.30-7.18(m, 3H), 7.10-7.98(m, 2H), 5.34(s, 1H), 4.72(d, J=15 Hz, 1 H), 4.65(d, J=15 Hz, 1H), 3.70-3.62(m, 2H), 3.55(s, 3H), 3.48(s, 1H), 3.18-3.08(m, 2H), 2.16-1.98(m, 4H), 0.84(s, 3H), 0.72(s, 3H); ESMS clcd for C₂₈H₃₃ClN₂O₇S: 577.09. Found: 419.2.

### Example 22

### Synthesis of 2-(2-Amino-ethoxymethyl)-4-{5-chloro-2-[4-(2-hydroxy-3-phenoxy-propylamino)-butoxy]-phenyl}-7,7-dimethyl-5-oxo-1,4,5,6,7,8-hexahydro-quinoline-3-carboxylic acid ethyl ester

¹H-NMR (CDCl₃) δ (ppm), 8.41-8.35(m, 1H), 7.42-7.37(m, 3H), 7.14-7.00(m, 4H), 6.77(d, J=8.7 Hz, 1H), 5.31(s, 1H), 4.94-4.79(m,2H), 4.23-4.00(m, 6H), 3.75-3.68(m, 2H), 3.10-2.80(m, 8H), 2.47-2.45(m, 2H), 2.29-2.24(m, 3H), 2.00-1.85(m, 4H), 1.29(t, J=7.2 Hz, 3H), 1.17(s, 3H), 1.05(s, 3H); ESMS clcd for C₃₆H₄₈ClN₃O₇: 669.32. Found: 670.5 (M+H)⁺.

### Example 23

### Synthesis of 4-(2-Chloro-phenyl)-2-[2-(2-hydroxy-3-phenoxy-propylamino)-ethoxymethyl]-7,7-dimethyl-5-oxo-1,4,5,6,7,8-hexahydroquinoline-3-carboxylic acid methyl ester

¹H-NMR (CDCl₃) δ (ppm), 7.62(s, 1H), 7.37-7.23(m, 4H), 7.11(t, J=7.5 Hz, 1H), 7.05-6.97(m, 2H), 6.92(d, J=87 Hz, 2H), 5.39(s, 1H), 4.77(d, J=8.1 Hz, 2H), 4.13(bs, 1H), 4.02-4.00(m, 2H), 3.71-3.65(m, 2H), 3.56(s, 3H), 2.96-2.85(m, 4H), 2.41-2.25(m, 2H), 2.22-2.07(m, 2H), 1.05(s, 3H), 0.93(s, 3H); ESMS clcd for C₃₁H₃₇ClN₂O₆: 568.23. Found: 569.4 (M+H)⁺.

### Example 24

### Synthesis of 2-(2-Amino-ethoxymethyl)-4-(2-chloro-phenyl)-6,6-dimethyl-5-oxo-1,4,5,6,7,8-hexahydro-quinoline-3-carboxylic acid ethyl ester

¹H-NMR (CDCl₃) δ (ppm), 8.23(s, 1H), 7.36(d, J=7.8 Hz, 1H), 7.23-7.21(m, 1H), 7.12-7.01(m, 2H), 5.36(s, 1H), 4.76(d, J=6.3 Hz, 2H), 4.01(t, J=7.5 Hz, 2H), 3.59(t, J=4.8 Hz, 2H), 2.97(t, J=5.7 Hz, 2H), 2.52-2.48(m, 2H), 1.79(t, J=6.6 Hz, 2H), 1.16(t, J=7.2 Hz, 3H), 1.08(s, 3H), 0.94(s, 3H); ESMS clcd for C₂₃H₂₉ClN₂O₄: 432.18. Found: 433.3 (M+H)⁺.

### Example 25

### Synthesis of 2-(2-Amino-ethoxymethyl)-4-(2-fluoro-phenyl)-7-methyl-5-oxo-1,4,5,6,7,8-hexahydroquinoline-3-carboxylic acid ethyl ester

Mixture of diastereomers. ¹H NMR (300 MHz, CDCl₃), δ (ppm): 8.51, 8.40 (two br s from two diastereomers, total 1H); 6.83-7.35 (m, 4H); 5.68 (br s, 2H); 5.21, 5.19 (two s, total 1H); 4.81 (dd, J=34.8 Hz, 15.3 Hz, 2H); 4.00 (q, J=7.2 Hz, 2H); 3.76 (br s, 2H); 3.18 (br s, 2H); 1.94-2.72 (m, 5H); 1.50 (t, J=7.2 Hz, 3H); 0.91-1.00 (m, 3H); ESMS clcd. for C₂₂H₂₈FN₂O₄ (M+H)⁺: 403.3. Found: 403.3.

### Example 26

### Synthesis of 2-(2-Amino-ethoxymethyl)-4-(2-fluoro-phenyl)-7-isopropyl-5-oxo-1,4,5,6,7,8-hexahydroquinoline-3-carboxylic acid ethyl ester

Mixture of diastereomers. ¹H NMR (300 MHz, CDCl₃), δ (ppm): 8.41, 8.30 (two br s from two diastereomers, total 1H); 6.84-7.37 (m, 4H); 5.23, 5.19 (two s, total 1H); 4.77 (dd, J=25.5 Hz, 16.5 Hz, 2H); 4.00 (q, J=7.5 Hz, 2H); 3.64 (t, J=4.8 Hz, 2H); 3.11 (br s, 2H); 3.02 (t, J=4.8 Hz, 2H); 1.48-2.60 (m, 6H); 1.16 (t, J=7.5 Hz, 3H); 0.86-0.92 (m, 6H); ESMS clcd. for C₂₄H₃₂FN₂O₄ (M+H)⁺: 431.3. Found: 431.4.

### Example 27

### Synthesis of 2-(2-Amino-ethoxymethyl)-4-(2-cyano-phenyl)-7-methyl-5-oxo-1,4,5,6,7,8-hexahydro-quinoline-3-carboxylic acid ethyl ester

Mixture of diastereomers. ¹H NMR (300 MHz, CDCl₃), δ (ppm): 8.90 (br s, 1H); 7.15-7.56 (m, 4H); 5.28, 5.27 (two s, total 1H); 4.79 (s, 2H); 3.95-4.04 (m, 2H); 3.65 (t, J=4.8 Hz, 2H); 2.99 (t, J=4.8 Hz, 2H); 1.94-2.70 (m, 7H); 1.12-1.17 (m, 3H); 1.00-1.06 (m, 3H); ESMS clcd. for C₂₃H₂₈N₃O₄ (M+H)⁺: 410.2. Found: 410.2.

### Example 28

### Synthesis of 2-(2-Amino-ethoxymethyl)-4-(2-cyano-phenyl)-7-isopropyl-5-oxo-1,4,5,6,7,8-hexahydro-quinoline-3-carboxylic acid ethyl ester

Mixture of diastereomers. ¹H NMR (300 MHz, CDCl₃), δ (ppm): 8.85, 8.88 (two br s from two diastereomers, total 1H); 7.18-7.57 (m, 4H); 5.28, 5.25 (two s, total 1H); 4.79 (dd, J=24.3 Hz, 18.3 Hz, 2H); 3.96-4.03 (m, 2H); 3.63-3.72 (m, 2H); 2.93-3.05 (m, 2H); 1.52-2.54 (m, 8H); 1.14 (t, J=7.2 Hz, 3H); 0.87-0.92 (m, 6H); ESMS clcd. for C₂₅H₃₂N₃O₄ (M+H)⁺: 438.2. Found: 438.2.

### Example 29

### Synthesis of 2-(2-Amino-ethoxymethyl)-4-(2-cyano-phenyl)-5-oxo-1,4,5,6,7,8-hexahydroquinoline-3-carboxylic acid ethyl ester

Mixture of diastereomers. ESMS clcd. for C₂₂H₂₆N₃O₄ (M+H)⁺: 396.2. Found: 396.2.

### Example 30

### Synthesis of 2-(7-Amino-ethoxymethyl-4-(3,5-dichlro-phenyl)-7.7-dimethyl-5-oxo-1,4,5,6,7,8-hexahydro-quinoline-3-carboxylic acid ethyl ester

¹H-NMR (CDCl₃) δ (ppm): 8.42 (s, 1H), 7.18 (d, J=2.1 Hz, 2H), 7.11 (d, J=2.1 Hz, 1H), 5.01 (s, 1H), 4.80 (s, 2H), 4.03-4.08 (m, 2H), 3.59-3.64 (m, 2H), 2.98-3.02 (m, 2H), 2.00-2.39 (m, 4H), 1.21 (t, J=6.9 Hz, 3H), 1.08 (s, 3H), 0.97 (s, 3H) ESMS clcd for C₂₃H₂₈Cl₂N₂O₄: 466.14. Found: 467.2 (M+H)⁺.

### Example 31

### Synthesis of 2-(2-Amino-ethoxymethyl)-4-(3,4-dichloro-phenyl)-7,7-dimethyl-5-oxo-1,4,5,6,7,8-hexahydro-quinoline-3-carboxylic acid ethyl ester

¹H-NMR (CDCl₃) δ (ppm): 8.31 (s, 1H), 7.34 (d, J=1.8 Hz, 1H), 7.26 (d, J=9.6 Hz, 1H), 7.16 (dd, J₁=9.6, J₂=1.8 Hz, 1H), 4.98 (s, 1H), 4.79-4.81 (s, 2H), 3.99-4.06 (m, 2H), 3.71-3.75 (m, 2H), 3.02-3.14 (m, 2H), 2.04-2.38 (m, 4H), 1.23 (t, J=6.9 Hz, 3H), 1.03 (s, 3H), 0.91 (s, 3H); ESMS clcd for C₂₃H₂₈Cl₂N₂O₄: 466.14. Found: 467.2 (M+H)⁺.

### Example 32

### Synthesis of 2-(2-Amino-ethoxymethyl)-4-2,3-dichloro-phenyl)-7-isopropyl-5-oxo-1,4,5,6,7,8-hexahydro-quinoline-3-carboxylic acid ethyl ester

¹H-NMR (CDCl₃) δ (ppm): 8.35 (s, 1H), 7.34 (d, J=2.7 Hz, 1H), 7.16 (d, J=8.4 Hz, 1H), 7.01 (dd, J₁=2.7 Hz, J₂=8.4 Hz, 1H), 5.33 (s, 1H), 4.76-4.78 (m, 2H), 4.02 (q, J=6.9 Hz, 2H), 3.50-3.63 (m, 2H), 2.99-3.02 (m, 2H), 2.04-2.35 (m, 4H), 1.17 (t, J=6.9 Hz, 3H), 1.06 (s, 3H), 0.96 (s, 3H); ESMS clcd for C₂₃H₂₈Cl₂N₂O₄: 466.14. Found: 467.2 (M+H)⁺.

### Example 33

### Synthesis of 2-(2-Amino-ethoxymethyl)-4-(2,3-dichloro-phenyl)-7-methyl-5-oxo-1,4,5,6,7,8-hexahydro-quinoline-3-carboxylic acid ethyl ester

¹H-NMR (CDCl₃) δ (ppm): 8.47 (s, 1H), 7.37 (d, J=2.7 Hz, 1H), 7.18 (d, J=8.4 Hz, 1H), 7.06 (dd, J₁=2.7 Hz, J₂=8.4 Hz, 1H), 5.39 (s, 1H), 4.67-4.83 (m, 2H), 3.94 (q, J=6.9 Hz, 2H), 3.70-3.74 (m, 2H), 3.01-3.11 (m, 2H), 2.03-2.58 (m, 4H), 1.14 (t, J=6.9 Hz, 3H), 0.95 (s, 3H); ESMS clcd for C₂₂H₂₆Cl₂N₂O₄: 452.13. Found: 453.1 (M+H)⁺.

### Example 34

### Synthesis of 2-(2-Amino-ethoxymethyl)-4-(2,6-dichloro-phenyl)-7,7-dimethyl-5-oxo-1,4,5,6,7,8-hexahydro-quinoline-3-carboxylic acid ethyl ester

¹H-NMR (CDCl₃) δ (ppm): 8.34 (s, 1H), 7.56-7.59 (m, 3H), 4.97 (s, 1H), 4.79-4.81 (s, 2H), 4.00-4.05 (m, 2H), 3.53-3.59 (m, 2H), 2.98-3.01 (m, 2H), 2.06-2.45 (m, 4H), 1.16 (t, J=6.9 Hz, 3H), 1.05 (s, 3H), 0.96 (s, 3H); ESMS clcd for C₂₃H₂₈Cl₂N₂O₄: 466.14. Found: 467.2 (M+H)⁺.

### Example 35

### Synthesis of 2-(2-Amino-ethoxymethyl)-4-(2,5-dichloro-phenyl)-7,7-dimethyl-5-oxo-1,4,5,6,7,8-hexahydroquinoline-3-carboxylic acid ethyl ester

¹H-NMR (CDCl₃) δ (ppm): 8.35 (s, 1H), 7.34 (d, J=2.7 Hz, 1H), 7.16 (d, J=8.4 Hz, 1H), 7.01 (dd, J₁=2.7 Hz, J₂=8.4 Hz, 1H), 5.33 (s, 1H), 4.76-4.78 (m, 2H), 4.02 (q, J=6.9 Hz, 2H), 3.50-3.63 (m, 2H), 2.99-3.02 (m, 2H), 2.04-2.35 (m, 4H), 1.17 (t, J=6.9 Hz, 3H), 1.06 (s, 3H), 0.96 (s, 3H); ESMS clcd for C₂₃H₂₈Cl₂N₂O₄: 466.14. Found: 467.2 (M+H)⁺.

### Example 36

### Synthesis of 2-(2-Amino-ethoxymethyl)-4-(2,4-dichloro-phenyl)-7,7-dimethyl-5-oxo-1,4,5,6,7,8-hexahydro-quinoline-3-carboxylic acid ethyl ester

¹H-NMR (CDCl₃) δ (ppm): 8.23 (s, 1H), 7.32 (d, J=8.4 Hz, 1H), 7.26 (dd, J₁=8.4 Hz, J₂=2.1 Hz, 1H), 7.09 (dd, J₁=8.4 Hz, J₂=2.1 Hz, 1H), 5.33 (s, 1H), 4.69-4.84 (m, 2H), 4.00 (q, J=6.9 Hz, 2H), 3.67-3.69 (m, 2H), 3.06-3.09 (m, 2H), 2.00-2.35 (m, 4H), 1.16 (t, J=6.9 Hz, 3H), 1.05 (s, 3H), 0.93 (s, 3H); ESMS clcd for C₂₃H₂₈Cl₂N₂O₄: 466.14. Found: 467.2 (M+H)⁺.

### Example 37

### 2-(2-Acetylamino-ethoxymethyl)-4-(2-chloro-phenyl)-7,7-dimethyl-5-oxo-1,4,5,6,7,8-hexahydro-quinoline-3-carboxylic acid ethyl ester

H-NMR (CDCl₃) δ (ppm) 0.94 (s, 3H), 1.07 (s, 3H), 1.14 (t, J=7.2 Hz, 3H), 1.80 (bs, 1H), 2.00 (s, 3H), 2.15 (m, 2H), 2.38 (m, 2H), 3.6 (m, 4H), 3.99 (m, 2H), 4.70 (q, 2H), 5.38 (s, 1H), 7.03(m, 1H), 7.10 (m, 1H), 7.20 (m, 1H), 7.35 (m, 1H), 7.6 (bs, 1H); ESMS clcd for C₂₅H₃₁ClN₂O₅: 474.2. Found: 475.1 (M+H)⁺.

### Example 38

### 2-(2-Amino-ethoxymethyl)-4-(2-chloro-phenyl)-7,7-dimethyl-5-oxo-1,4,5,6,7,8-hexahydro-quinoline-3-carboxylic acid

¹H-NMR (CD₃OD) δ (ppm) 1.11 (s, 3H), 1.20 (s, 3H), 2.13 (m, 3H), 2.53 (m, 2H), 3.3 (t, J=4.2 Hz, 2H), 3.81 (m, 2H), 4.60 (q, J=6.9 Hz, 2H), 5.62 (s, 1H), 7.21(m, 3H), 7.51 (d, J=6.6 Hz, 1H); ESMS clcd for C₂₁H₂₅ClN₂O₄: 404.2. Found: 405.1 (M+H)⁺.

### Example 39

### 2-(2-Amino-ethoxymethyl)-7,7-dimethyl-5-oxo-4-pyridin-3-yl-1,4,5,6,7,8-hexahydro-quinoline-3-carboxylic acid ethyl ester

¹H-NMR (CDCl₃) δ (ppm) 0.93 (s, 3H), 1.08 (s, 3H), 1.18 (t, J=7.2 Hz, 3H), 1.56 (bs, 2H), 2.28 (m, 4H), 2.99 (t, J=4.5 Hz, 2H), 3.60 (t, J=5.1 Hz, 2H), 4.04 (q, J=7.2 Hz, 2H), 4.89 (m, 2H), 5.05 (s, 1H), 7.14 (m, 1H), 7.64, (m, 1H), 8.33 (m, 1H), 8.45 (bs, 1H), 8.53 (d, J=1.5 Hz, 1H); ESMS clcd for C₂₂H₂₉N₃O₄: 399.22. Found: 400.0 (M+H)⁺.

### Example 40

### 4-(2-Chloro-phenyl)-2-(2-ethylamino-ethoxymethyl)-7,7-dimethyl-5-oxo-1,4,5,6,7,8-hexahydro-quinoline-3-carboxylic acid ethyl ester

¹H-NMR (CDCl₃) δ (ppm) 0.94 (s, 3H), 1.07 (s, 3H), 1.18 (m, 6H), 2.29 (m, 4H), 2.90 (q, J=7.1 Hz, 2H), 3.02 (t, J=4.5 Hz, 2H), 3.75 (t, J=5.1 Hz, 2H), 4.04 (m, 2H), 4.84 (m, 2H), 5.45 (s, 1H), 7.14 (m, 2H), 7.38, (m, 1H), 7.49 (m, 1H), 8.37 (bs, 1H); ESMS clcd for C₂₅H₃₃ClN₂O₄: 460.2. Found: 461.2 (M+H)⁺.

### Example 41

### 2-(2-Amino-ethoxymethyl)-4-(2-chloro-phenyl)-7,7-dimethyl-5-oxo-1,4,5,6,7,8-hexahydro-quinoline-3-carbonitrile

¹H-NMR (CDCl₃) δ (ppm) 1.03 (s, 3H), 1.09 (s, 3H), 1.61 (bs, 2H), 2.20(m, 4H), 2.96(m, 2H), 3.58 (m, 2H), 4.35 (m, 2H), 5.18 (s, 1H), 7.25 (m, 4H), 8.65 (bs, 1H); ESMS clcd for C₂₁H₂₄ClN₃O₂: 385.2. Found: 386.4 (M+H)⁺.

### Example 42

### Benzenesulfonate2-(3-ethoxycarbonyl-7,7-dimethyl-5-oxo-4-pyridin-3-yl-1,4,5,6,7,8-hexahydro-quinolin-2-ylmethoxy)-ethyl-ammonium

¹H-NMR (CDCl₃) δ (ppm) 0.84 (s, 3H), 0.95 (s, 3H), 1.21 (t, J=7.2 Hz, 3H), 2.17 (m, 3H), 2.68 (bs, 1H), 3.16 (m, 1H), 3.24 (m, 1H), 3.77 (m, 2H), 4.04 (q, J=7.2 Hz, 2H), 4.70 (m, 2H), 5.02 (s, 1H), 7.2 (m, 5H), 7.63, (d, J=7.2 Hz, 2H), 7.82 (d, J=6.9 Hz, 1H), 8.18 (m, 1H), 8.75 (m, 4H); ESMS clcd for C₂₈H₃₅N₃O₇S: 557.22. Found: 400.4 (M+H−C₆H₆SO₃)⁺.

### Example 43

### 2-(2-Amino-ethoxymethyl)-7,7-dimethyl-5-oxo-4-quinolin-3-yl-1,4,5,6,7,8-hexahydro-quinoline-3-carboxylic acid ethyl ester

¹H-NMR (CDCl₃) δ (ppm) 0.86 (s, 3H), 1.01 (s, 3H), 1.13 (t, J=6.3 Hz, 3H), 1.59 (bs, 2H), 2.22 (m, 4H), 2.92 (m, 2H), 3.55 (m, 2H), 3.97 (q, J=6.3 Hz, 2H), 4.78 (m, 2H), 5.20 (s, 1H), 7.45 (t, J=7.2 Hz, 1H), 7.57 (t, J=6.9 Hz, 1H), 7.73, (d, J=8.4 Hz, 1H), 7.99 (m, 2H), 8.60 (bs, 1H), 8.89 (d, J=1.8 Hz, 1H); ESMS clcd for C₂₆H₃₁N₃O₄: 449.2. Found: 450.1 (M+H)⁺.

### Example 44

### 4-(2-Chloro-phenyl)-7,7-dimethyl-2-(2-methylamino-ethoxymethyl)-5-oxo-1,4,5,6,7,8-hexahydro-quinoline-3-carboxylic acid ethyl ester

¹H-NMR (CDCl₃) δ 0.95 (s, 3H), 1.05 (s, 3H), 1.15 (t, 3H, J=7), 1.6-1.9 (m, 1H), 2.1-2.2 (q, 2H), 2.2-2.4 (q, 2H), 2.5 (d, 3H), 2.8-2.9 (m, 2H), 3.6 (t, 2H, J=5), 3.9-4.1 (m, 2H), 4.7-4.9 (q, 2H), 5.4 (s, 1H), 7.0-7.4 (m, 4H), 8.5 (s, 1H) ppm; ESMS clcd for C₂₄H₃₁ClN₂O₄: 446.1. Found: 447.1 (M+H)⁺.

### Example 45

### 2-(2-Amino-ethoxymethyl)-7,7-dimethyl-5-oxo-4-pyridin-4-yl-1,4,5,6,7,8-hexahydro-quinoline-3-carboxylic acid ethyl ester

¹H-NMR (CDCl₃) δ 0.95 (s, 3H), 1.1 (s, 3H), 1.2 (t, 3H, J=7), 1.45 (bs, 2H), 2.1-2.4 (m, 4H), 2.9-3.1 (m, 2H), 3.6-3.7 (m, 2H), 4.0-4.1 (m, 2H), 4.8 (s, 2H), 5.1 (s, 1H), 7.2-7.3 (m, 2H), 8.4-8.5 (m, 3H)ppm; ESMS clcd for C₂₂H₂₉N₃O₄: 399.2. Found: 400.2 (M+H)⁺.

### Example 46

### 2-(2-Amino-ethoxymethyl)-5-oxo-4-pyridin-3-yl-1,4,5,6,7,8-hexahydro-quinoline-3-carboxylic acid ethyl ester

¹H-NMR (CDCl₃) δ 1.1 (t, 3H, J=7), 1.5 (bs, 2H), 1.8-2.1 (m, 2H), 2.2-2.4 (m, 2H), 2.4-2.6 (m, 2H), 3.0 (t, 2H, J=5), 3.6 (t, 2H, J=5), 4.0-4.1 (m, 2H), 4.8 (q, 2H), 5.1 (s, 1H), 7.0-7.2 (m, 1H), 7.6-7.7 (m, 1H), 8.4 (m, 1H), 8.5 (s, 1H), 8.7 (s, 1H)ppm; ESMS clcd for C₂₀H₂₅N₃O₄: 371.2. Found: 372.2 (M+H)⁺.

### Example 47

### 2-(2-Amino-ethoxymethyl)-4-(4-chloro-1,2,4,5-tetradeutium-phenyl)-7,7-dimethyl-5-oxo-1,4,5,6,7,8-hexahydroquinoline-3-carboxylic acid ethyl ester

¹H-NMR (CDCl₃) δ 0.95 (s, 3H), 1.1 (s, 3H), 1.2 (t, 3H, J=7), 2.2 (q, 2H), 2.4 (q, 2H), 3.1 (m, 2H), 3.4 (bs, 2H), 3.6-3.7 (m, 2H), 4.0-4.1 (m, 2H), 4.8 (q, 2H), 5.0 (s, 1H), 8.2 (s, 1H)ppm; ESMS clcd for C₂₃H₂₅D₄ClN₂O₄: 436.2. Found: 437.2 (M+H)⁺.

### Example 48

### 2-[2-(1,3-Dioxo-1,3-dihydro-isoindol-2-yl)-ethoxymethyl]-7,7-dimethyl-5-oxo-4-pyridin-4-yl-1,4,5,6,7,8-hexahydroquinoline-3-carboxylic acid ethyl ester

¹H-NMR (CDCl₃) δ 1.0 (s, 3H), 1.1 (s, 3H), 1.2 (t, 3H, J=7), 2.2 (q, 2H), 2.5 (q, 2H), 3.7-3.8 (m, 2H), 4.0-4.2 (m, 4H), 4.8 (s, 2H), 5.1 (s, 1H), 7.2 (d, 2H, J=7), 7.7-8.0 (m, 5H), 8.4 (d, 2H, J=7)ppm; ESMS clcd for C₃₀H₃₁N₃O₆: 529.2. Found: 530.2 (M+H)⁺.

### Example 49

### 2-[2-(1,3-Dioxo-1,3-dihydro-isoindol-2-yl)-ethoxymethyl]-5-oxo-4-pyridin-3-yl-1,4,5,6,7,8-hexahydro-quinoline-3-carboxylic acid ethyl ester

¹H-NMR (CDCl₃) δ 1.2 (t, 3H, J=7), 1.9-2.1 (m, 2H), 2.3-2.4 (m, 2H), 2.5-2.8 (m, 2H), 3.7-3.8 (m, 2H), 3.9-4.2 (m, 4H), 4.7 (q, 2H), 5.1 (s, 1H), 7.1 (q, 1H), 7.2 (d, 1H, J=7), 7.7-8.0 (m, 5H), 8.3 (d, 1H, J=5), 8.5 (s, 1H)ppm; ESMS clcd for C₂₈H₂₇N₃O₆: 501.2. Found: 502.2 (M+H)⁺.

### Example 50

### 2-(2-Azido-ethoxymethyl)-7,7-dimethyl-4-(3-nitro-phenyl)-5-oxo-1,4,5,6,7,8-hexahydro-quinoline-3-carboxylic acid ethyl ester

¹H-NMR (CDCl₃) δ 0.95 (s, 3H), 1.05 (s, 3H), 1.2 (t, 3H, J=7), 2.1-2.4 (m, 4H), 3.4-3.6 (m, 2H), 3.7-3.8 (m, 2H), 4.0-4.1 (m, 2H), 4.8 (q, 2H), 5.2 (s, 1H), 7.2-7.4 (m, 2H), 7.7 (d, 1H, J=7), 8.0 (d, 1H, J=7), 8.1 (s,1H)ppm; ESMS clcd for C₂₃H₂₇N₅O₆: 469.2. Found: 470.2 (M+H)⁺.

### Example 51

### 2-(2-azido-ethoxymethyl)-4-benzo[1,3]dioxol-5-yl-7,7-dimethyl-5-oxo-1,4,5,6,7,8-hexahydro-quinoline-3-carboxylic acid ethyl ester

¹H-NMR (CDCl₃) δ (ppm), 7.19(s, 1H), 6.83-6.79(m, 2H), 6.69-6.67(m, 1H), 5.90(s, 2H), 5.01(s, 1H), 4.88 and 4.83(2d, J=16.8 Hz each 1H), 4.12-4.06(m, 2H), 3.83-3.78(m, 2H), 3.56-3.48(m, 2H), 2.37-2.23(m, 4H), 1.25(t, J=10.8 Hz, 3H), 1.11(s, 3H), 1.00(s, 3H); ESMS clcd for C₂₄H₂₈N₄O₆: 468.20. Found: 469.2 (M+H)⁺.

### Example 52

### 2-(2-azido-ethoxymethyl)-4-(2-chloro-phenyl)-1,7,7-trimethyl-5-oxo-1,4,5,6,7,8-hexahydro-quinoline-3-carboxylic acid ethyl ester

¹H-NMR (CDCl₃) δ (ppm), 7.30-7.250(m, 2H), 7.14-7.05(m, 2H), 5.55(s, 1H), 4.94(d, J=12.9 Hz, 1H), 4.76(d, J=12.9 Hz, 1H), 4.16-4.10(m, 2H), 3.75-3.71(m, 2H), 3.44(s, 3H), 3.41-3.38(m, 2H), 2.60(d, J=16.8 Hz, 1H), 2.38(d, J=16.8 Hz, 1H), 2.20(s, 2H), 1.26(t, J=6.9 Hz, 3H), 1.11(s, 3H), 1.03(s, 3H); ESMS clcd for C₂₄H₂₉ClN₄O₄: 472.19. Found: 473.1 (M+H)⁺.

### Example 53

### 2-(2-amino-ethoxymethyl)-4-(2-chloro-phenyl)-5-oxo-4,5,6,7-tetrahydro-1H-[1]pyrindine-3-carboxylic acid ethyl ester

¹H-NMR (CDCl₃) δ (ppm), 9.19(s, 1H), 7.29-7.24(m, 2H), 7.16-7.05(m, 2H), 5.31(s, 1H), 4.83(s, 2H), 3.99-3.92(m, 2H), 3.65-3.59(m, 2H), 3.01(t, J=5.1 Hz, 2H), 2.61-2.58(m, 2H), 2.36-2.51(m, 2H), 1.07(t, J=7.2 Hz, 3H); ESMS clcd for C₂₀H₂₃ClN₂O₄: 390.13. Found: 391.2 (M+H)⁺.

### Example 54

### 9-(2-chloro-phenyl)-6,6-dimethyl-5,6,7,9-tetrahydro-3H,4H-furo[3,4-b]quinoline-1,8-dione

¹H-NMR (CDCl₃) δ (ppm), 10.13(s, 1H), 7.288-7.11(m, 4H), 5.05(s, 1H), 4.86(s, 2H), 2.50-2.39(m, 2H), 2.18(d, J=16.8 Hz, 1H), 2.00(d, J=16.8 Hz, 1H), 1.02(s, 3H), 0.94(s, 3H); ESMS clcd for C₁₉H₁₈ClNO₃: 343.10. Found: 344.0 (M+H)⁺.

### Example 55

### (+)-(R)-2-(2-amino-ethoxymethyl)-4-(2-chloro-phenyl)-7,7-dimethyl-5-oxo-1,4,5,6,7,8-hexahydro-quinoline-3-carboxylic acid ethyl ester; Compound with Benzenesulfonic Acid

¹H-NMR (DMSO-d₆) δ (ppm), 8.66(s, 1H), 7.84-7.80(m, 3H), 7.60-7.58(m, 2H), 7.32-7.09(m, 6H), 5.22(s, 1H), 4.66(d, J=15 Hz, 1 H), 4.60(d, J=15 Hz, 1H), 4.08-3.93(m, 2H), 3.68-3.60(m, 2H), 3.10-3.05(m, 2H), 2.28-2.23(m, 1H), 2.06-2.00(m, 1H) 1.17 (t, J=6.9 Hz, 3H), 1.09(s, 3H), 0.92(s, 3H); ESMS clcd for C₂₉H₃₅ClN₂O₇S: 590.19. Found: 433.2.

### Example 56

### (+)-(R)-2-(2-amino-ethoxymethyl)-4-(2-chloro-phenyl)-7,7-dimethyl-5-oxo-1,4,5,6,7,8-hexahydro-quinoline-3-carboxylic acid ethyl ester

¹H-NMR (CDCl₃) δ (ppm), 8.24(s, 1H), 7.40(d, J=6 Hz, 1H), 7.26-7.21(m, 1H), 7.14-7.02(m, 2H), 5.39(s, 1H) 4.78 and 4.76(2d, J=16.8 Hz,each 1H), 4.03-3.98(m, 2H), 3.64-3.60(m, 2H), 3.02(t, J=4.5 Hz, 2H), 2.42-2.08(m, 6H), 1.15 (t, J=7.2 Hz, 3H),1.07(s, 3H), 0.94(s, 3H); ESMS clcd for C₂₃H₂₉ClN₂O₄: 432.18. Found: 433.2 (M+H)⁺.

### Example 57

### (−)-(S)-2-(2-amino-ethoxymethyl)-4-(2-chloro-phenyl)-7,7-dimethyl-5-oxo-1,4,5,6,7,8-hexahydro-quinoline-3-carboxylic acid ethyl ester

¹H-NMR (CDCl₃) δ (ppm), 8.21(s, 1H), 7.40(d, J=6 Hz, 1H), 7.26-7.21(m, 1H), 7.14-7.02(m, 2H), 5.38(s, 1H), 4.81 and 4.77(2d, J=16.8 Hz, each 1H), 4.02-3.98(m, 2H), 3.75-3.68(m, 2H), 3.12(t, J=4.5 Hz, 2H), 2.40(s, 2H), 2.22-2.06(m, 4H), 1.15 (t, J=7.2 Hz, 3H), 1.05(s, 3H), 0.92(s, 3H); ESMS clcd for C₂₃H₂₉ClN₂O₄: 432.18. Found: 433.2 (M+H)⁺.

### Example 58

### 2-(2-Amino-ethoxymethyl)-4-(1H-indol-3-yl)-7,7-dimethyl-5-oxo-1,4,5,6,7,8-hexahydro-quinoline-3-carboxylic acid ethyl ester

¹H-NMR (300 MHz, CDCl₃): δ 9.67 (s, 1H), 8.04 (s, 1H), 7.78-7.72 (m, 1H), 7.34-7.28 (m, 1H), 7.20-7.06 (m, 2H), 6.71 (s, 1H), 4.80 (s, 1H), 4.69 (s, 1H), 4.18-4.06 (m, 2H), 3.98 (s, 1H), 3.65-3.50 (m, 2H), 2.90-2.65 (m, 2H), 2.56-2.22 (m, 4H), 1.23 (t, J=6.9 Hz, 3H), 1.13 (s, 3H), 1.11 (s, 3H); ES-MS calculated for C25H31N3O4 (M+H)⁺: 437.23. Found: 438.2.

### Example 59

### 4-(2-Chloro-phenyl)-2-ethoxycarbonylmethoxymethyl-7,7-dimethyl-5-oxo-1,4,5,6,7,8-hexahydro-quinoline-3-carboxylic acid ethyl ester

¹H-NMR (300 MHz, CDCl₃): δ 7.89 (s, 1H), 7.39 (dd, J=1.5, 7.8 Hz, 1H), 7.23 (dd, J=1.5, 7.8 Hz, 1H), 7.12 (td, J=1.5, 7.5 Hz, 1H), 7.02 (td, J=1.5, 7.5 Hz, 1H), 5.40 (s, 1H), 4.85 (d, J=4.5 Hz, 2H), 4.27 (q, J=6.9 Hz, 2H), 4.22 (d, J=4.5 Hz, 2H), 4.15-3.92 (m, 2H), 2.44-2.09 (m, 4H), 1.32 (t, J=7.2 Hz, 3H), 1.16 (t, J=6.9 Hz, 3H), 1.08 (s, 3H), 0.96 (s, 3H); ES-MS Calculated for C25H30ClNO6 (M+1)⁺: 475.18. Found: 476.1.

### Example 60

### 4-(2-Chloro-phenyl)-2-(hydroxyl-ethoxymethyl)-7,7-dimethyl-5-oxo-1,4,5,6,7,8-hexahydro-quinoline-3-carboxylic acid ethyl ester

¹H-NMR (300 MHz, CDCl₃): δ 7.65 (s, 1H), 7.39 (dd, J=1.8, 7.8 Hz, 1H), 7.23 (dd, J=1.4, 7.8 Hz, 1H), 7.12 (td, J=1.8, 7.8 Hz, 1H), 7.03 (td, J=1.8, 7.8 Hz, 1H), 5.40 (s, 1H), 4.83 (d, J=16.8 Hz, 1H), 4.76 (d, J=16.8 Hz, 1H), 4.08-3.96 (m, 2H), 3.88 (t, J=4.5 Hz, 2H), 3.78-3.64 (m, 2H), 2.40-2.09 (m, 4H), 1.16 (t, J=7.2 Hz, 3H), 1.07 (s, 3H), 0.97 (s, 3H); ES-MS Calculated for C₂₃H₂₈ClNO₅ (M+H)⁺: 433.17. Found 434.1.

### Example 61

### 9-(2-Chloro-phenyl)-4-ethyl-6,6-dimethyl-5,6,7,9-tetrahydro-3H,4H-furo[3,4b]quinoline-1,8-dione

¹H-NMR (300 MHz, DMSO-d₆): δ 7.28 (d, J=7.5 Hz, 1H), 7.25-7.10 (m, 3H), 5.08-5.02 (m, 3H), 3.70-3.52 (m, 2H), 2.64 (s, 2H), 2.20 (d, J=15.9 Hz, 1H), 1.98 (d, J=15.9 Hz, 1H), 1.25 (t, J=7.2 Hz, 3H), 1.06 (s, 3H), 0.94 (s, 3H), ES-MS calculated for C21H22ClNO3 (M+H)⁺: 371.13. Found: 372.1.

### Example 62

### 4-(2-Chloro-phenyl)-7,7-dimethyl-5-oxo-2-piperazin-1-ylmethyl-1,4,5,6,7,8-hexahydro-quinoline-3-carboxylic acid ethyl ester

¹H-NMR (300 MHz, CDCl₃): δ 8.10 (s, 1H), 7.36 (dd, J=1.8, 7.5 Hz, 1H), 7.23 (dd, J=1.5, 8.1 Hz, 1H), 7.11 (dt, J=1.5, 7.5 Hz, 1H), 7.03 (td, J=1.8, 7.5 Hz, 1H), 5.39 (s, 1H), 4.08-3.96 (m, 2H), 3.77 (s, 2H), 3.48 (s, 1H), 2.98 (t, J=4.8 Hz, 4H), 2.64-2.50 (m, 4H), 2.38-2.09 (m, 4H), 1.16 (t, J=7.2 Hz, 3H), 1.09 (s, 3H), 0.98 (s, 3H); ES-MS calculated for C25H32ClN3O3 (M+H)⁺: 457.21. Found: 458.3.

### Example 63

### 4-(2-Chloro-phenyl)-2-{[(2-hydroxy-ethyl)-methyl-amino]-methyl}-7,7-dimethyl-5-oxo-1,4,5,6,7,8-hexahydro-quinoline-3-carboxylic acid ethyl ester

¹H-NMR (300 MHz, CDCl₃): δ 8.46(s, 1H), 7.38 (dd, J=1.8, 7.8 Hz, 1H), 7.22 (dd, J=1.5, 7.8 Hz, 1H), 7.11 (td, J=1.5, 7.5 Hz, 1H), 7.02 (td, J=1.8, 7.8 Hz, 1H), 5.40 (s, 1H), 4.13-3.91 (m, 3H), 3.84 (d, J=11.1 Hz, 2H), 3.77-3.73 (m, 2H), 2.61 (t, J=5.1 Hz, 2H), 2.40 (s, 3H), 2.34-2.06 (m, 4H), 1.16 (t, J=7.2 Hz, 3H), 1.05 (s, 3H), 0.93 (s, 3H); ES-MS calculated for C₂₄H₃₁ClN₂O₄ (M+H)⁺: 446.20. Found: 447.20.

### Example 64

### 2-(2-Amino-ethoxymethyl)-4-(2-hydroxy-phenyl)-7,7-dimethyl-5-oxo-1,4,5,6,7,8-hexahydro-quinoline-3-carboxylic acid ethyl ester

¹H-NMR (CDCl₃) δ (ppm) 9.3 (br, 1H), 8.6 (br, 1H), 6.7-7.1 (m, 4H), 5.12 (s, 1H), 4.9 (m, 2H), 3.9 (m, 2H), 3.6 (t, J=6 Hz, 2H), 3.0 (t, J=6 Hz, 2H), 2.3 (m, 4H), 1.02 (s, 3H), 1.0 (t, J=8 Hz, 3H), 0.84 (s, 3H); ESMS clcd for C₂₃H₃₀N₂O₅: 414.2. Found: 415.4 (M+H)⁺.

### Example 65

### 2-(2-Amino-ethoxymethyl)-7,7-dimethyl-5-oxo-4-pyridin-3-yl-1,4,5,6,7,8-hexahydro-quinoline-3-carboxylic acid methyl ester

¹H-NMR (CDCl₃) δ (ppm) 8.6 (m, 1H), 8.5 (br, 1H), 8.4 (m, 1H), 7.7 (m, 1H), 7.2 (m, 1H), 5.06 (s, 1H), 4.8 (m, 2H), 3.60 (s, 3H), 3.6 (m, 2H), 3.0 (t, J=6 Hz, 2H), 2.4 (m, 2H), 2.2 (m,2H), 1.06 (s, 3H), 0.96 (s, 3H); ESMS clcd for C₂₁H₂₇N₃O₄: 385.2. Found: 386.1 (M+H)⁺.

### Example 66

### 2-(2-Amino-ethoxymethyl)-1,7,7-trimethyl-5-oxo-4-pyridin-3-yl-1,4,5,6,7,8-hexahydro-quinoline-3-carboxylic acid ethyl ester

¹H-NMR (CDCl₃) δ (ppm) 8.4 (m, 2H), 7.6 (m, 1H), 7.2 (m, 1H), 5.20 (s, 1H), 4.7-5.1 (m, 2H), 4.1 (m, 2H), 3.5 (m, 2H), 3.39 (s, 3H), 2.8 (m, 2H), 2.3-2.6 (m, 2H), 2.2 (m,2H), 1.1 (t, J=8 Hz, 3H), 1.12 (s, 3H), 1.00 (s, 3H); ESMS clcd for C₂₃H₃₁N₃O₄: 413.2. Found: 414.1 (M+H)⁺.

### Example 67

### 2-(2-Hydroxy-ethoxymethyl)-7,7-dimethyl-5-oxo-4-pyridin-3-yl-1,4,5,6,7,8-hexahydro-quinoline-3-carboxylic acid ethyl ester

¹H-NMR (CDCl₃) δ (ppm) 8.6 (br, 1H), 8.4 (m, 2H), 7.9 (br, 1H), 7.7 (m, 1H), 7.2 (m, 1H), 5.06 (s, 1H), 4.8 (m, 2H), 4.0 (m, 2H), 3.8 (m, 2H), 3.7 (m, 2H), 2.1-2.4 (m, 4H), 1.2 (t, J=8 Hz, 3H), 1.06 (s, 3H), 0.90 (s, 3H); ESMS clcd for C₂₂H₂₈N₂O₅: 400.2. Found: 401.1 (M+H)⁺.

### Example 68

### 2-(2-Dimethylamino-ethoxymethyl)-7,7-dimethyl-5-oxo-4-pyridin-3yl-1,4,5,6,7,8,-hexahydro-quinoline-3-carboxylic acid ethyl ester

¹H-NMR (CD₃OD) δ (ppm) 8.82 (s, 1H), 8.56-7.05 (m, 4H), 5.04 (s, 1H), 4.79 (m, 2H), 4.02(dd, J=10 Hz, 2H),3.75-3.57 (m, 2H), 2.65-2.01(m, 6H), 2.37(s, 6H), 1.19(t, J=10 Hz, 3H), 1.09(s, 3H), 0.93(s, 3H); ESMS clcd for C₂₄H₃₃N₃O₄: 427.25. Found: 428.2 (M+H)⁺.

### Example 69

### 2-(4-Amino-butyl)-7,7-dimethyl-5-oxo-4-pyridin-3yl-1,4,5,6,7,8,-hexahydro-quinoline-3-carboxylic acid ethyl ester

¹H-NMR (CD₃OD) δ (ppm) 8.58-7.16 (m, 4H), 5.03 (s, 1H), 4.02(dd, J=10 Hz, 2H), 2.86-1.43 (m, 12H), 1.18 (t, J=10 Hz, 3H), 1.09 (s, 3H), 0.91(s, 3H); ESMS clcd for C₂₃H₃₁N₃O₃: 397.24. Found: 398.2(M+H)⁺.

### Example 70

### 2-(2-Amino-ethoxymethyl)-4-(2-chloro-phenyl)-7,7-dimethyl-4,6,7,8-tetrahydro-1H-quinolin-5-one

¹H NMR (CDCl₃): δ 7.63 (brs, 1H), 7.28 (dd, J=7.5 and 1.2 Hz, 1H), 7.21-7.13 (m, 2H), 7.04 (td, J=6.9 and 2.1 Hz, 1H), 5.06 and 4.98 (ABq, J_{AB}=4.6 Hz, 2H), 3.96 and 3.84 (ABq, J_{AB}=16.2 Hz, 2H), 3.41 (m, 1H), 3.40 (s, 1H), 2.92 (m, 2H), 2.85 (t, J=4.8 Hz, 2H), 2.38 (m, 2H); 2.24 and 2.13 (ABq, J_{AB}=16.2 Hz, 2H), 1.11 (s, 3H), 1.09 (s, 3H); ESMS clcd for C₂₀H₂₅ClN₂O₂: 360.16. Found: 361.4 (M+1)⁺.

### Example 71

### 4-(2-Chloro-phenyl)-2-(2-dimethylamino-ethoxymethyl)-7,7-dimethyl-5-oxo-1,4,5,6,7,8-hexahydro-quinoline-3-carboxylic acid ethyl ester

¹H-NMR (CDCl₃) δ 0.95 (s, 3H), 1.05 (s, 3H), 1.15 (t, 3H, J=7), 1.9-2.3 (m, 2H), 2.4 (s, 2H), 2.6 (s, 6H), 2.9 (t, 2H, J=5), 3.7 (t, 2H, J=5), 3.9-4.1 (m, 2H), 4.8 (q, 2H, J=16, 28), 5.4 (s, 1H), 7.05 (t, 1H, J=8), 7.15 (t, 1H, J=8), 7.25 (t, 1H, J=8), 7.45 (t, 1H, J=8), 8.45 (s, 1H) ppm; ESMS clcd for C₂₅H₃₃ClN₂O₄: 460.2. Found: 461.2 (M+H)⁺.

### Example 72

### (+)-(R)-2-(2-Amino-ethoxymethyl)-7,7-dimethyl-5-oxo-4-pyridin-3-yl-1,4,5,6,7,8-hexahydro-quinoline-3-carboxylic acid ethyl ester

¹H-NMR (CDCl₃) δ (ppm) 0.93 (s, 3H), 1.08 (s, 3H), 1.18 (t, J=7.2 Hz, 3H), 1.56 (bs, 2H), 2.28 (m, 4H), 2.99 (t, J=4.5 Hz, 2H), 3.60 (t, J=5.1 Hz, 2H), 4.04 (q, J=7.2 Hz, 2H), 4.89 (m, 2H), 5.05 (s, 1H), 7.14 (m, 1H), 7.64, (m, 1H), 8.33 (m, 1H), 8.45 (bs, 1H), 8.53 (d, J=1.5 Hz, 1H); ESMS clcd for C₂₂H₂₉N₃O₄: 399.22. Found: 400.0 (M+H)⁺.

### Example 73

### 4-Cyclopropyl-2-hydroxymethyl-7,7-dimethyl-5-oxo-1,4,5,6,7,8-hexahydro-quinoline-3-carbonitrile

¹H-NMR (CDCl₃) δ (ppm), 7.15(s, 1H), 4.39(d, J=11, 2H), 3.18(d, J=12, 1H), 2.50-2.05-1.80(m, 4H), 1.18(s, 3H), 0.96(s, 3H), 0.88-0.75 (m, 1H), 0.58-0.22(m,4H). ESMS clcd for C₁₆H₂₀N₂O₂: 272.15. Found: 273.2 (M+H)⁺.

### Example 74

### 7-spirocyclopentyl-4-cyclopropyl-2-methyl-5-oxo-1,4,5,6,7,8-hexahydro-quinoline-3-carboxylic acid ethyl ester

¹H-NMR (CDCl₃) δ (ppm), 5.63(s, 1H), 4.29-4.07(m, 2H), 3.81(d, J=11, 1H), 3.18(d, J=12, 1H), 2.48-1.79(m, 7H), 1.78-40(m, 8H), 1.27(t, J=11,3H), 0.84-0.67 (m, 1H), 0.28-0.16(m,4H). ESMS clcd for C₂₀H₂₇NO₃: 329.20. Found: 330.2 (M+H)⁺.

### Example 75

### 2,7,7-trimethyl-4-(1-methyl-cyclopropyl)-5-oxo-1,4,5,6,7,8-hexahydro-quinoline-3-carboxylic acid ethyl ester

¹H-NMR (CDCl₃) δ (ppm), 5.61(s, 1H), 4.24-4.10(m, 2H), 3.59(s, 1H), 2.40-2.15(m, 7H), 1.27(t, J=5, 3H), 1.17(s, 3H), 1.21(s, 3H), 0.84 (s, 3H), 0.82-0.11(m,4H). ESMS clcd for C₁₉H₂₇NO₃: 317.20. Found: 318.2 (M+H)⁺.

### Example 76

### 4-Cyclopropyl-2,7,7-trimethyl-5-oxo-1,4,5,6,7,8-hexahydro-quinoline-3-carbonitrile

¹H-NMR (CDCl₃) δ (ppm), 6.07(s, 1H), 3.21(d, J=12, 1H), 2.39-2.02(m, 5H), 1.61(s, 2H), 1.13(s, 6H),0.92-0.80(m,1H), 0.58-0.37(m, 4H). ESMS clcd for C₁₆H₂₀N2O: 256.16. Found: 257.2 (M+H)⁺.

### Example 77

### 2-(2-Amino-ethoxymethyl)-4-cyclopropyl-7,7-dimethyl-5-oxo-1,4,5,6,7,8-hexahydro-quinoline-3-carboxylic acid ethyl ester

¹H-NMR (CDCl₃) δ (ppm), 7.92(s, 1H), 4.78(s, 2H), 4.28-4.02(m, 2H),3.80(d, J=12, 1H), 3.62-3.50(m, 2H), 2.98-2.90(m, 2H), 2.41-1.22(m, 9H), 1.14(s, 3H), 1.10(s, 3H),0.92-0.79(m,1H), 0.33-0.20(m, 4H). ESMS clcd for C₂₀H₃₀N₂O₄: 362.22. Found: 363.2 (M+H)⁺.

### Example 78

### 4-Cyclopropyl-2,7,7-trimethyl-5-oxo-1,4,5,6,7,8-hexahydro-quinoline-3-carboxylic acid ethyl ester

¹H-NMR (CD₃Cl) δ (ppm), 5.92(s, 1H), 4.31-4.08(m, 2H), 3.78(d, J=12, 1H), 2.40-1.22(m, 10H), 1.11(s, 3H), 1.07(s, 3H),0.92-0.79(m,1H), 0.33-0.17(m, 4H). ESMS clcd for C₁₈H₂₅FNO₃: 303.18. Found: 304.2 (M+H)⁺.

### Example 79

### 4-Cyclohexyl-2,7,7-trimethyl-5-oxo-1,4,5,6,7,8-hexahydro-quinoline-3-carboxylic acid ethyl ester

¹H-NMR (CDCl₃) δ (ppm), 5.58 (s, 1H), 4.22-4.08 (m, 2H), 3.99 (d, J=7.2 Hz, 1H), 2.31-2.18 (m, 7H), 1.62-1.54 (m, 7H), 1.28 (m, 4H), 1.12-0.86 (m, 9H); ESMS clcd for C₂₁H₃₁NO₃: 345.23. Found: 346.2 (M+H)⁺.

### Example 80

### 4-Isopropyl-2,7,7-trimethyl-5-oxo-1,4,5,6,7,8-hexahydro-quinoline-3-carboxylic acid ethyl ester

¹H-NMR (CDCl₃) δ (ppm), 5.98 (s, 1H), 4.16-4.12 (m, 2H), 3.99 (d, J=4.5 Hz, 1H), 2.31-2.24 (m, 7H), 1.69 (m, 1H),1.30-1.25 (m, 3H), 1.11 (s, 3H), 1.09 (s, 3H), 0.74 (d, J=4.5 Hz, 6H); ESMS clcd for C₁₈H₂₇NO₃: 305.20. Found: 306.2 (M+H)⁺.

### Example 81

### 2-(2-Amino-ethoxymethyl)-4-isopropyl-7,7-dimethyl-5-oxo-1,4,5,6,7,8-hexahydro-quinoline-3-carboxylic acid ethyl ester

¹H-NMR (CDCl₃) δ (ppm), 7.92 (s, 2H), 4.76 (s, 2H), 4.17-4.08 (m, 2H), 4.01 (d, J=4.5 Hz, 1H), 3.57-3.53 (t, J=4.8 Hz, 2H), 2.96-2.93 (t, J=4.8 Hz, 2H), 2.34-2.26 (m, 4H), 4.62 (s, 2H), 1.30-1.25 (t, J=7.2 Hz, 3H), 1.25-1.09 (ss, 6H), 0.76-0.74 (ss, 6H) ESMS clcd for C₂₀H₃₂N₂O₄: 364.20. Found: 365.2 (M+H)⁺.

### Example 82

### 4-Cyclopentyl-2,7,7-trimethyl-5-oxo-1,4,5,6,7,8-hexahydro-quinoline-3-carboxylic acid ethyl ester

¹H-NMR (CDCl₃) δ (ppm), 5.90 (s, 1H), 4.23-4.11 (m, 2H), 2.32-2.22 (m, 7H), 1.82 (m, 1H),1.61-1.38 (m, 9H), 1.26 (t, J=7.2 Hz, 3H),1.12-1.10 (m, 7H); ESMS clcd for C₂₀H₂₉NO₃: 331.20. Found: 332.2 (M+H)⁺.

### Example 83

### 2-(2-Amino-ethoxymethyl)-4-cyclopentyl-7,7-dimethyl-5-oxo-1,4,5,6,7,8-hexahydro-quinoline-3-carboxylic acid ethyl ester

¹H-NMR (CDCl₃) δ (ppm), 7.94 (s, 2H), 4.75 (s, 2H), 4.16-4.09 (m, 2H), 3.55-3.53 (m, 2H), 2.96-2.92 (m, 2H), 2.34-2.26 (m, 4H), 1.78 (m, 1H), 1.54-1.50 (m, 8H), 1.25 (t, J=7.2 Hz, 3H),1.11-1.09 (m, 8H); ESMS clcd for C₂₂H₃₄N₂O₄: 390.20. Found: 391.2 (M+H)⁺.

### Example 84

### 4-tert-Butyl-2,7,7-trimethyl-5-oxo-1,4,5,6,7,8-hexahydro-quinoline-3-carboxylic acid ethyl ester

¹H-NMR (CDCl₃) δ (ppm), 6.28 (s, 1H), 4.28-4.05 (m, 2H), 3.99 (s, 1H), 2.35-2.24 (m, 7H), 1.27 (t, J=7.2 Hz, 3H), 1.16 (s, 3H), 1.09 (s, 3H), 0.71 (s, 9H); ESMS clcd for C₁₉H₂₉NO₃: 319.20. Found: 320.2 (M+H)⁺.

### Example 85

### 4-Cyclopropyl-7,7-dimethyl-2-morpholin-4-ylmethyl-5-oxo-1,4,5,6,7,8-hexahydro-quinoline-3-carboxylic acid ethyl ester

¹H-NMR (CDCl₃) δ (ppm), 7.82 (s, 1H), 4.27-4.05 (m, 2H), 3.78-3.71 (m, 6H), 2.53-2.50 (m, 4H), 2.29-2.26 (m, 4H), 1.29 (t, J=7.2 Hz, 3H), 1.12-1.10 (ss, 6H), 0.92 (m, 2H), 0.24-0.21 (m, 4H); ESMS clcd for C₂₂H₃₂N₂O₄: 388.20. Found: 389.2 (M+H)⁺.

### Example 86

### 4-Cyclopropyl-7,7-dimethyl-2-(2-morpholin-4-yl-ethoxymethyl)-5-oxo-1,4,5,6,7,8-hexahydro-quinoline-3-carboxylic acid ethyl ester

¹H-NMR (CDCl₃) δ (ppm), 7.38 (s, 1H), 4.75 (s. 2H), 4.22-4.11 (m, 2H), 3.80-3.64 (m, 6H), 2.62-2.51 (m, 5H), 2.34-2.26 (m, 3H), 1.67 (m, 3H), 1.27 (t, J=7.2 Hz, 3H), 1.14 (ss, 6H), 0.82 (m, 1H), 0.23-0.20 (m, 4H); ESMS clcd for C₂₄H₃₆N₂O₅: 432.20. Found: 433.2 (M+H)⁺.

### Example 87

### 4-Cyclopropyl-7,7-dimethyl-5-oxo-2-trifluoromethyl-1,4,5,6,7,8-hexahydro-quinoline-3-carboxylic acid ethyl ester

¹H-NMR (CDCl₃) δ (ppm), 6.26 (s, 1H), 4.15 (q, J=7.2 Hz, 2H), 3.58 (m, 1H), 2.42-2.12 (m, 3H), 1.38-1.22 (m, 3H), 1.05 (m, 6H), 0.92-0.80 (m, 2H), 0.38-0.22 (m, 4H); ESMS clcd for C₁₈H₂₂F₃NO₃: 357.20. Found: 358.2 (M+H)⁺.

### Example 88

### 2,4-Dicyclopropyl-7,7-dimethyl-5-oxo-1,4,5,6,7,8-hexahydro-quinoline-3-carboxylic acid ethyl ester

¹H-NMR (CDCl₃) δ (ppm), 5.48 (s, 1H), 4.25-4.12 (m, 2H), 3.78 (d, J=7.2 Hz, 1H), 2.77-2.64 (m, 1H), 2.27-2.16 (m, 4H), 1.30 (t, J=7.2 Hz, 3H), 1.10-1.1 (ss, 6H), 0.98-0.94 (m, 2H), 0.85-0.52 (m, 3H), 0.25-0.19 (m, 4H); ESMS clcd for C₂₀H₂₇NO₃: 329.20. Found: 330.2 (M+H)⁺.

### Example 89

### 4-Cyclopropyl-2-ethyl-7,7-dimethyl-5-oxo-1,4,5,6,7,8-hexahydro-quinoline-3-carboxylic acid ethyl ester

¹H-NMR (CDCl₃) δ (ppm), 5.72 (s, 1H), 4.22-4.17 (m, 2H), 3.80 (d, J=7.2 Hz, 1H), 2.74-2.71 (m, 2H), 2.37-2.20 (m, 7H), 1.32 (t, J=7.2 Hz, 3H), 1.20-1.10 (m, 6H), 0.92-0.78 (m 1H), 0.24-0.20 (m 4H); ESMS clcd for C₁₉H₂₇NO₃: 317.20. Found: 318.2 (M+H)⁺.

### Example 90

### 4-Cyclopropylmethyl-2,7,7-trimethyl-5-oxo-1,4,5,6,7,8-hexahydro-quinoline-3-carboxylic acid ethyl ester

¹H-NMR (CDCl₃) δ (ppm), 5.62 (s, 1H), 4.17-4.10 (m, 2H), 2.30-2.20 (m, 6H), 1.58 (s, 3H), 1.31-1.25 (m, 4H), 1.09 (s, 6H), 0.58 (m, 1H), 0.32-0.29 (m, 2H), 0.06-0.08 (m 2H); ESMS clcd for C₁₉H₂₇NO₃: 317.20. Found: 318.2 (M+H)⁺.

### Example 91

### 4-Isopropyl-7,7-dimethyl-2-morpholin-4-ylmethyl-5-oxo-1,4,5,6,7,8-hexahydro-quinoline-3-carboxylic acid ethyl ester

¹H-NMR (CDCl₃) δ (ppm), 7.76 (s, 1H), 4.21-4.02 (m, 3H), 3.77-3.71 (m, 6H), 2.52-2.49 (m, 4H), 2.29-2.26 (m, 4H), 1.66 (m 1H), 1.29 (t, J=7.2 Hz, 3H), 1.14 (ss, 6H), 0.74 (d, J=6.9 Hz, 6H); ESMS clcd for C₂₂H₃₄N₂O₄: 390.20. Found: 391.2 (M+H)⁺.

### Example 92

### 4-Isopropyl-7,7-dimethyl-2-(2-morpholin-4-yl-ethoxymethyl)-5-oxo-1,4,5,6,7,8-hexahydro-quinoline-3-carboxylic acid ethyl ester

¹H-NMR (CDCl₃) δ (ppm), 7.42 (s, 1H), 4.75 (s. 2H), 4.22-4.04 (m, 3H), 3.75-3.58 (m, 6H), 2.62-2.49 (m, 6H), 2.34-2.27 (m, 4H), 1.67 (m, 3H), 1.27 (t, J=7.2 Hz, 3H), 1.14 (ss, 6H), 0.74 (ss, 6H); ESMS clcd for C₂₄H₃₈N₂O₅: 434.20. Found: 435.2 (M+H)⁺.

### Example 93

### 2-(2-Amino-ethoxymethyl)-4-cyclohexyl-7,7-dimethyl-5-oxo-1,4,5,6,7,8-hexahydro-quinoline-3-carboxylic acid ethyl ester

¹H-NMR (CDCl₃) δ (ppm), 7.86 (s, 1H), 4.75 (s, 2H), 4.15-3.98 (m, 3H), 3.54 (m, 2H), 2.96-2.92 (m, 2H), 2.33-2.25 (m, 4H), 1.62-1.55 (m, 8H), 1.30-0.82 (14H); ESMS clcd for C₂₃H₃₆N₂O₄: 404.20. Found: 405.2 (M+H)⁺.

### Example 94

### 2-(2-Amino-ethoxymethyl)-7,7-dimethyl-4-(pyrid-3-yl)-5-oxo-1,4,5,6,7,8-hexahydro-quinoline-3-carboxylic acid ethyl ester

ESMS clcd for C₂₂H₂₈N₂O₄: 384.20. Found: 385.2 (M+H)⁺.

### Example 95

### 2-(2-Amino-ethoxymethyl)-7,7-dimethyl-4-(5-methyl-furan-2-yl)-5-oxo-1,4,5,6,7,8-hexahydro-quinoline-3-carboxylic acid ethyl ester

ESMS clcd for C₂₂H₃₀N₂O₅: 402.22. Found: 401.2 (M−H)⁺.

### Example 96

### 2-Propoxymethyl-7,7-dimethyl-4-(pyrid-3-yl)-5-oxo-1,4,5,6,7,8-hexahydro-quinoline-3-carboxylic acid ethyl ester

ESMS clcd for C₂₃H₃₀N₂O₄: 398.22. Found: 399.2 (M+H)⁺.

### Example 97

### 2-Ethoxymethyl-7,7-dimethyl-4-(5-methyl-furan-2-yl)-5-oxo-1,4,5,6,7,8-hexahydro-quinoline-3-carboxylic acid ethyl ester

ESMS clcd for C₂₂H₂₉NO₅: 387.20. Found: 386.2 (M−H)⁺.

### Example 98

### 2,7,7-Trimethyl-4-(5-methyl-furan-2-yl)-5-oxo-1,4,5,6,7,8-hexahydro-quinoline-3-carboxylic acid ethyl ester

ESMS clcd for C₂₀H₂₅NO₄: 343.18. Found: 344.2 (M+H)⁺.

### Example 99

### 2-Isopropoxymethyl-7,7-dimethyl-4-(pyrid-3-yl)-5-oxo-1,4,5,6,7,8-hexahydro-quinoline-3-carboxylic acid ethyl ester

ESMS clcd for C₂₃H₃₀N₂O₄: 398.22. Found: 399.2 (M+H)⁺.

### Example 100

### 2-(2-Amino-ethoxymethyl)-7,7-dimethyl-4-(4-methylpyrid-3-yl)-5-oxo-1,4,5,6,7,8-hexahydro-quinoline-3-carboxylic acid ethyl ester

ESMS clcd for C₂₃H₃₁N₃O₄: 413.23. Found: 414.2 (M+H)⁺.

### Example 101

### 2-(2-Amino-ethoxymethyl)-7,7-dimethyl-4-(4-methoxypyrid-3-yl)-5-oxo-1,4,5,6,7,8-hexahydro-quinoline-3-carboxylic acid ethyl ester

ESMS clcd for C₂₃H₃₁N₃O₅: 429.23. Found: 430.2 (M+H)⁺.

### Example 102

### 2,7,7-Trimethyl-4-(pyrid-3-yl)-5-oxo-1,4,5,6,7,8-hexahydro-quinoline-3-carbonitrile

ESMS clcd for C₁₈H₁₉N₃O: 293.15. Found: 294.2 (M+H)⁺.

### Example 103

### 2-(2-Amino-ethyoxymethyl)-7,7-dimethyl-4-(2-methoxyphenyl)-5-oxo-1,4,5,6,7,8-hexahydro-quinoline-3-carbonitrile

ESMS clcd for C₂₂H₂₇N₃O₃: 381.21. Found: 382.2 (M+H)⁺.

### Example 104

### 2,7,7-Trimethyl-4-(2-methoxyphenyl)-5-oxo-1,4,5,6,7,8-hexahydro-quinoline-3-carbonitrile

ESMS clcd for C₂₀H₂₂N₂O₂: 322.17. Found: 323.2 (M+H)⁺.

### Example 105

### 2-(2-Amino-ethoxymethyl)-7,7-dimethyl-4-(5-chloro-6-methoxy-pyrid-3-yl)-5-oxo-1,4,5,6,7,8-hexahydro-quinoline-3-carboxylic acid ethyl ester

ESMS clcd for C₂₂H₂₇N₃O₃: 463.19. Found: 464.2 (M+H)⁺.

### Example 106

### 2-(2-Amino-ethoxymethyl)-7,7-dimethyl-4-(6-hydroxypyrid-3-yl)-5-oxo-1,4,5,6,7,8-hexahydro-quinoline-3-carboxylic acid ethyl ester

ESMS clcd for C₂₂H₂₉N₃O₅: 415.21. Found: 416.2 (M+H)⁺.

### Example 107

### 2-(2-Amino-ethoxymethyl)-7,7-dimethyl-4-(3-chlorophenyl)-5-oxo-1,4,5,6,7,8-hexahydro-quinoline-3-carboxylic acid ethyl ester

ESMS clcd for C₂₆H₃₄ClN₂O₆: 505.21. Found: 506.2 (M+H)⁺.

### Example 108

### 2-(2-Amino-ethoxymethyl)-7,7-dimethyl-4-(2,4-dichlorophenyl)-5-oxo-1,4,5,6,7,8-hexahydro-quinoline-3-carboxylic acid ethyl ester

ESMS clcd for C₂₃H₂₈Cl₂N₂O₄: 466.14. Found: 467.1 (M+H)⁺.

### Example 109

### 2-(2-Amino-ethoxymethyl)-7,7-dimethyl-4-(3-cyanophenyl)-5-oxo-1,4,5,6,7,8-hexahydro-quinoline-3-carboxylic acid ethyl ester

ESMS clcd for C₂₄H₃₁N₃O₄: 425.23. Found: 497.1 (M+H)⁺.

### Example 110

### 2-(2-Amino-ethoxymethyl)-7,7-dimethyl-5-oxo-4-pyridin-3-yl-1,4,5,6,7,8-hexahydro-quinoline-3-carboxylic acid ethyl ester

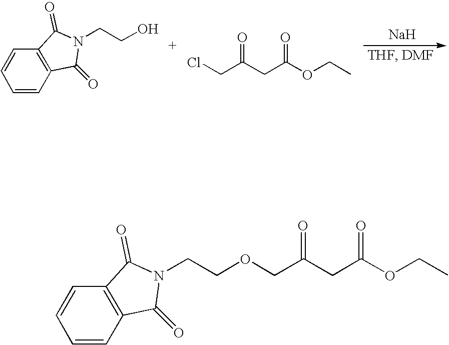

Compound 39 can also be prepared as follows. To a mechanically stirred suspension of NaH (15 g, 0.375 mol, 60% dispersion in mineral oil) in THF (250 mL) and DMF (25 mL) was added N-(2-hydroxyethyl)-phtalimide (43 g, 0.225 mol). The mixture was stirred at room temperature for about 4 h and then cooled to about 0° C. in an ice bath. Ethyl-4-chloroacetate (21.4 mL, 0.15 mol) in THF (4 mL) was then added via an addition funnel over a period of about 0.5 h and the resulting mixture allowed to stir overnight. The mixture was then poured into a 2 L sepratory funnel containing 500 mL ice water and 700 mL EtOAc and separated. The organic layer was washed 2× with 500 mL of water and dried over MgSO₄. The solution was then filtered through a 4-inch plug of silica and then concentrated in vacuo. The resulting yellow oil was then dissolved in 250 mL CH₃CN and washed 2× with 30 mL hexane. The CH₃CN layer was the concentrated in vacuo to yield 19.4 g (40.5%) of 4-[2-(1,3-Dioxo-1,3-dihydro-isoindol-2-yl)-ethoxy]-3-oxo-butyric acid ethyl ester as a yellow oil. This crude compound was used for the next step.

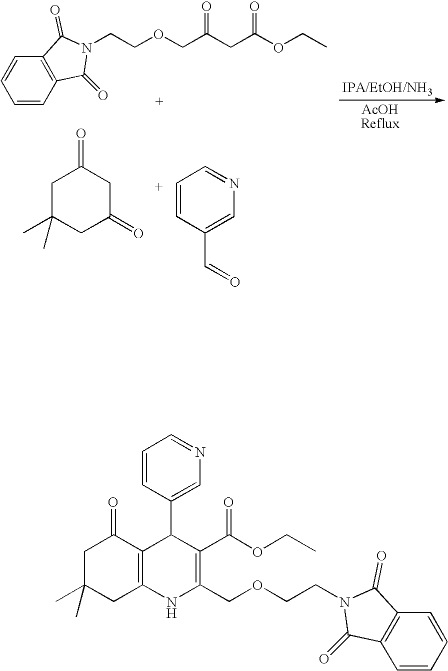

To a suspension of 4-[2-(1,3-Dioxo-1,3-dihydro-isoindol-2-yl)-ethoxy]-3-oxo-butyric acid ethyl ester (19.4 g, 0.061 mol) and 5,5-dimethyl-1,3-cyclohexadiketone (8.5 g, 0.061 mol) in 60 mL IPA and 24 mL AcOH was added 166 mL 2N NH₃/EtOH. 3-Pyridinecarboxaldehyde (5.75 mL, 0.061 mol) was then added and the solution allowed to stir at reflux for about 3 hr and then cooled to room temperature overnight. The resulting solution was partially concentrated in vacuo and then placed in the refrigerator overnight. The precipitate was filtered and washed with Et₂O to yield 14.5 g (45%) of 2-[2-(1,3-Dioxo-1,3-dihydro-isoindol-2-yl)-ethoxymethyl]-7,7-dimethyl-5-oxo-4-pyridin-3-yl-1,4,5,6,7,8-hexahydro-quinoline-3-carboxylic acid ethyl ester as an off-white solid.

¹H-NMR (CDCl₃) δ (ppm) 0.94 (s, 3H), 1.07 (m, 6H), 2.05 (s, 3H), 2.15 (m, 2H), 2.38 (m, 2H), 3.75 (m, 2H), 4.01 (m, 4H), 4.65 (m, 2H), 5.06 (s, 1H), 7.03(m, 1H), 7.7 (m, 6H), 8.37 (d, 1H), 8.51 (s, 1H); ESMS clcd for C₂₅H₃₁ClN₂O₅: 529.2. Found: 530.5 (M+H)⁺.

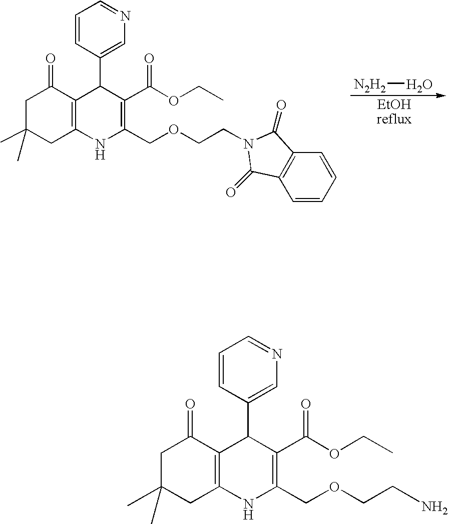

To a solution of Dioxo-1,3-dihydro-isoindol-2-yl)-ethoxymethyl]-7,7-dimethyl-5-oxo-4-pyridin-3-yl-1,4,5,6,7,8-hexahydro-quinoline-3-carboxylic acid ethyl ester (14.5 g, 0.027 mol) in EtOH (250 mL) was added hydrazine monohydrate (6.7 mL, 0.135 mol) and the solution allowed to stir for about 45 min. The solution was then allowed to cool to room temperature and the solid was filtered off and washed with EtOH. The EtOH was then removed in vacuo, the residue re-suspended in CH₂Cl₂, washed with water (3×) and brine (1×), dried over MgSO₄, filtered and concentrated to yield 9 g of 2-(2-Amino-ethoxymethyl)-7,7-dimethyl-5-oxo-4-pyridin-3-yl-1,4,5,6,7,8-hexahydro-quinoline-3-carboxylic acid ethyl ester as a yellow solid. ¹H-NMR (CDCl₃) δ (ppm) 0.93 (s, 3H), 1.08 (s, 3H), 1.18 (t, J=7.2 Hz, 3H), 1.56 (bs, 2H), 2.28 (m, 4H), 2.99 (t, J=4.5 Hz, 2H), 3.60 (t, J=5.1 Hz, 2H), 4.04 (q, J=7.2 Hz, 2H), 4.89 (m, 2H), 5.05 (s, 1H), 7.14 (m, 1H), 7.64, (m, 1H), 8.33 (m, 1H), 8.45 (bs, 1H), 8.53 (d, J=1.5 Hz, 1H); ESMS clcd for C₂₂H₂₉N₃O₄: 399.22. Found: 400.0 (M+H)⁺.

Complete separation of the enantiomers of Compound 39 is accomplished using a Chiralpak AS (4.5 mm×25 cm) column eluting with ethanol/2% triethylamine/CO₂ (15/85) (flow rate: 2 mL/min, 135/100 bar, 35° C.).

Compounds of this invention were evaluated for their effect on plasma glucose, triglyceride and insulin levels in C57BL/6 ob/ob and C57BLKS/J db/db male mice, models of NIDDM.

### Example 111

### Glucose Reduction by Compound 1 in db/db Mouse Model

Animals:

Non-insulin dependent diabetic mellitus (NIDDM) male mice (C57BLKS/J-m+/+Lepr db) weighing 60±5 g (12 weeks of age) provided by the Institute for Animal Reproduction (IAR, Japan) were used. These animals exhibited hyperinsulinemia, hyperglycemia and islet atrophy. The animals were housed in Individually Ventilated Cages Racks (IVC Racks, 36 Mini Isolator systems) throughout the experiment. Each APEC cage was autoclave sterilized and contained 4 mice and then maintained in a hygienic environment under controlled temperature (22-24° C.) and humidity (60-70%) with 12-hour light/dark cycles. The animals were given sterilized lab Chow and sterilized distilled water ad libitum.

Chemicals:

Sterilized distilled water, ELISA Insulin Assay Kit (SPI bio, France), glucose (Merck), Glucose-HA assay kit (Wako, Japan), methylcellulose (Sigma), and troglitazone (BioMOL).

Methods:

Compound 1 was suspended in 0.5% methylcellulose. Compound 1 at 100, 50 and 30 mg/kg as well as vehicle were administered to NIDDM male mice weighing 60±5 g (11-12 weeks of age), orally once daily for 7 consecutive days starting immediately after the first blood sampling (pre-treatment on day 1). Blood sampling was repeated at 90 minutes post-dosing on day 3 and day 7. The dosing volume was 10 ml/kg. Serum glucose level in the pre- and post-treatment periods were determined by enzymatic (Mutaratase-GOD) method. Post-treatment serum glucose value expressed in percentage of respective pre-treatment values were calculated and unpaired Student's t test was then applied for comparison between compound treated and vehicle control groups. Differences were considered significant at P<0.05. In addition, the liver weight and body weight were recorded to determine the ratio of liver to body weight.

Results:

Serum Glucose
% Pre-Treatment Baseline

| Treatment | Dose | Day 3 | Day 7 |
|---|---|---|---|
| Vehicle | 10 ml/kg | 103.5 ± 2.4 | 98.7 ± 2.4 |
| Compound 1 | 100 mg/kg | 85.6 ± 2.4* | 80.9 ± 3.3* |
| | 50 mg/kg | 86.5 ± 3.5* | 82.9 ± 4.4* |
| | 30 mg/kg | 96.7 ± 2.2* | 91.6 ± 3.7 |

*Statistically significant (P < 0.05 vs. corresponding vehicle-treated control)

In this assay for hypoglycemic activity in the lepr db mouse, Compound 1 at 100 and 50 mg/kg caused significant reduction in serum glucose relative to the vehicle treatment group on day 3 and 7. No significant difference in liver weight relative to body weight between vehicle control and any treatment group was observed.

### Example 112

### Glucose Reduction by Compound 9 in db/db Mouse Model

C57BLKS/J-m+/+Lepr db male animals (8 weeks old) were purchased from the Jackson Laboratory (Bar Harbor, Me.) in this study. All animals were acclimated for at least one week, and randomized for equivalent mean blood glucose on day 0. Animals were divided into either vehicle or treatment groups (n=8/group). Compound 9 was formulated in 0.5% methylcellulose (ps400) and administered by oral gavage at 50 mg/kg once daily for 7 consecutive days. Blood glucose was monitored using One Touch Basic glucose monitor (Abbott) 3.5 hours after administration of test compound on day 3 and 7.

A significant reduction in blood glucose was observed post-treatment with Compound 9, as compared to the vehicle group. The percent normalized reductions relative to vehicle control were 13.6% and 21.1% on day 3 and 7, respectively.

### Example 113

### Glucose Reduction by Compound 1 in ob/ob Mouse Model

Genetically mutated obese diabetic mice (C57BL/6 ob/ob) were purchased from the Jackson Laboratory (Bar Harbor, Me.). Eight weeks old male animals were employed in this study. After acclimation, mice were randomized for equivalent mean blood glucose on day 0 with eight mice each group, and received either vehicle or Compound 1 in 0.5% methylcellulose (ps400) by oral gavage once daily for 8 consecutive days. Plasma glucose was monitored 3.5 hours after administration of test compound on day 3 and 8.

In this ob/ob mouse assay, Compound 1 at 50 mg/kg significantly reduced serum glucose as compared to the vehicle treatment group with percent normalized reduction of 30% and 22.3% on day 3 and 8, respectively.

These results establish that the test compound of this invention reduced blood glucose levels by a significant percentage compared to vehicle and that six of eight animals (75%) exhibited normalized glucose (less than or equal to 220 mg/dl) during the study.

### Example 114

### Glucose Reduction by Compound 36 in Glucose-Loaded ob/ob Mouse Model

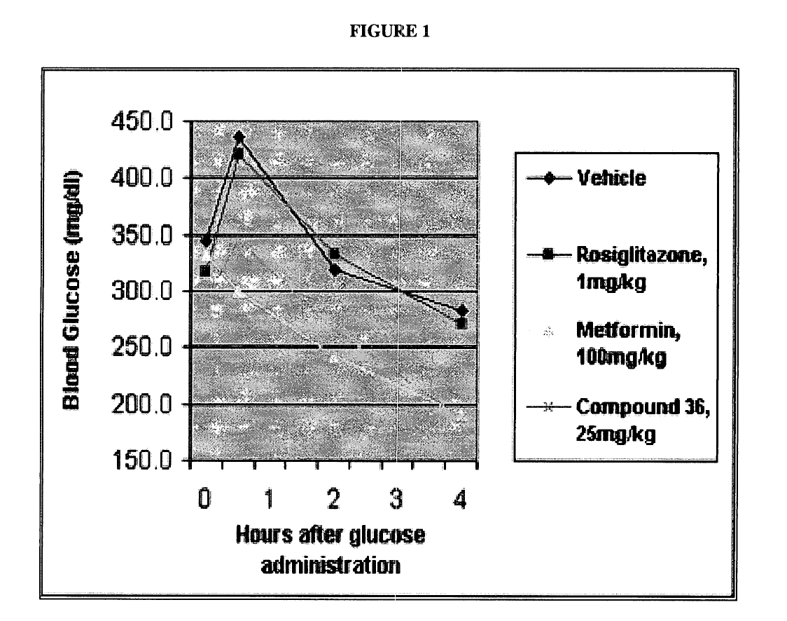

An oral glucose tolerance test was conducted to determine whether Compound 36 sensitized insulin activity by increasing glucose disposal (Nature (1997) 386: 407-410; Diabetes (1990) 39: 121812-1227). Fasted male ob/ob mice were orally administered with 25 mg/kg of Compound 36 followed immediately by an oral glucose administration (3 g/kg with 10 ml/kg). Blood glucose was measured at 0, 30, 120 and 240 min post-glucose loading. Metformin and rosiglitazone were used as references. The result is shown in FIG. 1.

The vehicle and rosiglitazone treated groups experienced elevated glucose level after glucose loading and the glucose level remained in the range of 300 mg/dl at 4 hours post-glucose loading. In contrast, Compound 36 and metformin both reduced blood glucose to around basal levels (approximately 200 mg/dl).

### Example 115

### Glucose, Insulin and Triglyceride Reduction (db/db Mouse Model)

Compound 1 was evaluated for its effect on plasma glucose, triglyceride and insulin in C57BLKS/J db/db male mice.

Compound 1 or vehicle was administered orally once daily for 10 consecutive days. Animals were divided into either vehicle or treatment groups (n=8 per group), and blood glucose, serum triglyceride and insulin were tested at the end of assay by Ani Lytic Inc (Baltimore, Md.). The data are tabulated below:

| | Glucose (mg/dl) | Insulin (ng/ml) | Triglyceride (mg/dl) |
|---|---|---|---|
| Vehicle | 379.63 ± SEM | 13.94 ± SEM | 404.00 ± SEM |
| Compound 1 | 327.50 ± SEM | 11.32 ± SEM | 336.13 ± SEM |
| % Reduction | 14% | 19% | 17% |
| (relative to | | | |
| vehicle) | | | |

As these data demonstrate, Compound 1 effectively reduced elevated glucose, insulin and triglyceride levels associated with this model of Phase II diabetes.

### Example 116

### Glucose Reduction Using Compound 1 in Combination with Rosiglitazone (db/db Mouse Model)

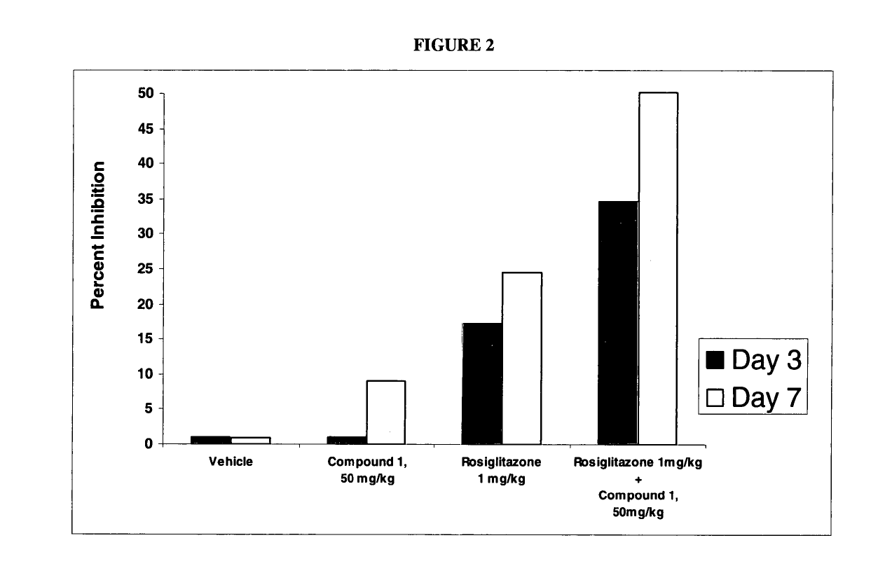

Following the protocols set forth above, 1 mg/kg of rosiglitazone was given to C57BLKS/J db/db male mice (N=5) with 50 mg/kg of Compound 1 to determine the anti-hyperglycemic effect of the combination. Rosiglitazone was orally dosed first, and one hour later Compound 1 was orally administered. Blood glucose was tested at day 3 and day 7 (2.5 hours after consecutive drug dosing). The results are displayed in FIG. 2.

Although Compound 1 and rosiglitazone alone both reduced glucose levels, the combination of Compound 1 with rosiglitazone improved the glucose-lowering efficacy significantly. In the case of the combination therapy, a 74% glucose reduction was observed compared to that in vehicle control group. The glucose-lowering effect appears to reach a plateau by the seventh or eighth day of administration. Furthermore, even 24 hours after 8th dose, the glucose level remained reduced. In summary, the combination demonstrated higher anti-hyperglycemic effect than monotherapy of rosiglitazone or Compound 1, and the benefit lasted at least 24 hours after administration.

### Example 117

### Glucose Reduction Using Compound 36 in Combination with Rosiglitazone (db/db Mouse Model)

C57BLKS/J-m+/+Lepr db were obtained from the Institute for Animal Reproduction (IAR, Japan), and randomized for equivalent mean blood glucose on day 0 (n=8/group). One group served as the vehicle control (0.5% methyl cellulose). Group 2 and 3 received compound 36 (25 and 50 mg/kg, respectively). Group 3 received 0.5 mg/kg rosiglitazone. Group 4 and 5 received 0.5 mg/kg rosiglitazone and one hour later compound 36 (25 and 50 mg/kg, respectively) in combination. All agents were administered once daily for 6 consecutive days starting on day 1. Blood glucose was determined 2.5 hours after receiving vehicle or Compound 36 on day 3 and day 6. Post-treatment serum glucose value was expressed as percent pre-treatment from respective pre-treatment values, and percent reduction relative to vehicle control was calculated by subtracting from the percent pre-treatment of vehicle control.

| | | | Serum Glucose | | | |
|---|---|---|---|---|---|---|
| | | | % Pre-Treatment Baseline | | | |
| | | | (% Normalized Reduction) | | | |
| Treatment | Dose | | Day 3 | | Day 6 | |
| Vehicle | 10 | ml/kg | 105.6 ± 1.8 | (—) | 107.9 ± 3.2 | (—) |
| Compound 36 | 10 | mg/kg | 103.0 ± 2.4 | (2.6) | 100.7 ± 4.6 | (7.2) |
| Compound 36 | 25 | mg/kg | 100.3 ± 2.8 | (5.3) | 99.7 ± 5.4 | (8.2) |
| Rosiglitazone | 0.5 | mg/kg | 90.3 ± 3.1 | (15.3) | 79.1 ± 1.9 | (28.8) |
| Rosiglitazone/Compound 36 | 0.5/10 | mg/kg | 85.1 ± 2.3 | (20.5) | 67.4 ± 2.9 | (40.5) |
| Rosiglitazone/Compound 36 | 0.5/25 | mg/kg | 88.1 ± 1.4 | (17.5) | 65.5 ± 2.5 | (42.4) |

The results of this experiment demonstrate that the tested compound of this invention enhances the efficacy of the known diabetes drug, rosiglitazone. Treatment by 0.5 mg/kg rosiglitazone had only modest effect on serum glucose with 28.8% normalized reduction relative to vehicle control on day 6. Treatment by Compound 36 in combination with rosiglitazone enhanced the anti-hyperglycemic effect of rosiglitazone with 40.5% and 42.4% normalized reduction on day 6 for 10 mg/kg and 25 mg/kg Compound 36, respectively.

### Example 118

### Cardiovascular Effect (Mean Arterial Blood Pressure and Heart Rate)

Male SD rats weighing 350-420 g were anesthetized with 2-3% isoflurane. The femoral artery and vein were catheterized and connected to a mean arterial blood pressure (MAP) and heart rate (HR) monitor and a continuous intravenous infusion pump. Under anesthesia, MAP and HR were recorded 10-20 minutes prior to and during the continuous intravenous infusion of test compounds for 60-70 minutes. The infusion rate, 0.14-0.16 ml/kg/minute, was determined on the basis of vehicle tolerance. The infusion doses, 1.0-1.25 mg/kg/minute, were selected, depending upon the solubility of the compound and effects on MAP and HR of the positive control compounds at the lethal dose. The maximal effective dose or lethal dose of test compounds were determined accordingly.

| | | | | Average Max. % | |
|---|---|---|---|---|---|
| | | | Lethal Dose | Reduction | |
| Groups | N | Infusion Rates | (mg/kg) | BP | HR |
| Vehicle* | 1 | 0.14 ml/kg/min. | — | 0% | 18% |
| Amlodipine | 2 | 1.04 mg/kg/min./ | ˜12.7 | 100% | 100% |
| (base form) | | 0.14 ml/kg/min. | | | |
| Compound 1 | 2 | 1.04 mg/kg/min./ | >70 | 15% | 50% |
| | | 0.14 ml/kg/min. | | | |
| Compound 39 | 3 | 1.04 mg/kg/min./ | >70 | 12.7% | 25.7% |
| | | 0.14 ml/kg/min. | | | |

*10% DMSO + 18% Cr-RH40 + D5W
(DMSO = dimethyl sulfoxide; Cr-RH40 = Cremophor RH-40; D5W = 5% glucose in water))

The data shown above demonstrate that the tested compounds of this invention have significantly reduced cardiovascular effects compared to amlodipine, a conventional dihydropyridine used as a hypertensive agent.

### Example 119

### Blood Glucose Reduction by Compounds 39, 41, and 64 in db/db Mice in 7 day Baseline Glucose Test

Non-insulin dependent diabetic mellitus (NIDDM) male mice (C57BLKS/J-m+/+Lepr db), weighing 50±5 g and 9-10-week old, were used. The animals were housed in Individually Ventilated Cages Racks (IVC Racks) throughout the experiment and all animals were allowed free access to sterilized Lab chow and sterilized distilled water. Compounds 39, 41 and 64 at 50 mg/kg as well as vehicle (0.5% Methylcellulose) were administered orally once daily for 7 consecutive days to test animals after the first blood sampling (pre-treatment). The post-treatment blood samples were withdrawn from the orbital sinus at 3 hours after administration of daily dose on day 3 and on day 7. Serum glucose levels in the pre- and post-treatment were determined by enzymatic (Mutaratase-GOD) methods. Post-treatment serum glucose values were expressed in percentage of respective pre-treatment values. Percentage reductions were expressed in percentage of pre-treatment values of test substance relative to percentage of pre-treatment values of vehicle. All percentages were calculated and unpaired; Student's t test was then applied to establish significant difference between the test compound-treated and vehicle control groups. Differences are considered significant at P<0.05.

As these data demonstrate, Compounds 39, 41 and 64 effectively reduced serum glucose levels relative to vehicle.

% Pre-Treatment Baseline
(% Reduction relative
to vehicle)

| Treatment | Dose | Day 3 | | Day 7 | |
|---|---|---|---|---|---|
| Vehicle | 5 ml/kg | 95.4 | (—) | 100.8 | (—) |
| Compound 39 | 50 mg/kg | 98.5 | (3) | 82.3 | (18) |
| Compound 41 | 50 mg/kg | 93.3 | (2) | 94.1 | (6) |
| Compound 64 | 50 mg/kg | 85.9 | (10) | 88.6 | (12) |

### Example 120

### Blood Glucose Reduction by Compound 39 in an Oral Glucose Tolerance Test

Compound 39 (25, 50 and 100 mg/kg) and vehicle (0.5% Methylcellulose) were administered orally at one hour before glucose loading (2 g/kg, PO) to Lepr db mice fasted for about 4 hours. Blood samples were collected sequentially from the retro-orbital sinus at pre-treatment one day before and about 1 and about 2 hours after glucose loading. Serum glucose values were obtained at about 1 and about 2 hours after glucose loading, percentage reductions were expressed as percentage of corresponding vehicle control values and unpaired Student's t test was then applied for comparison between test substance and vehicle group. Differences are considered significant at P<0.05 level.

As these data demonstrate, Compound 39 effectively reduced serum glucose levels relative to vehicle.

| | | Serum Glucose | | | | |
|---|---|---|---|---|---|---|
| | | (mg/dL) | | | | |
| Treatment | | (% Reduction relative to vehicle) | | | | |
| (OGTT) | Dose | 0 hr | 1 hr | | 2 hr | |
| Vehicle | 5 ml/kg | 374.0 ± 19.3 | 470 ± 15.1 | (—) | 375.3 ± 20.2 | (—) |
| Compound 39 | 100 mg/kg | 340.7 ± 14.2 | 366.2 ± 17.3 | (22.1) | 301.3 ± 28.4 | (19.7) |
| Compound 39 | 50 mg/kg | 371.1 ± 15.2 | 415.6 ± 11.0 | (11.6) | 346 ± 16.8 | (7.8) |
| Compound 39 | 25 mg/kg | 375.9 ± 16.3 | 379.3 ± 18.5 | (19.3) | 331.1 ± 14.5 | (11.8) |

### Example 121

### Combination Studies of Rosiglitazone or Metformin with Compound 39

For combination studies, oral administration of Rosiglitazone (0.5 mg/kg) or Metformin (150 mg/kg) was followed by Compound 39 (50 mg/kg) about one hour later, daily for 7 consecutive days at 24 hours after the first blood sampling (on day 0). The post-treatment blood samples were withdrawn from the orbital sinus at 2.5 hours after 2^{nd} test substance dosing on day 3 and on day 7. Serum glucose levels in the pre-and post-treatment were determined enzymatically (Mutaratase-GOD). Post-treatment serum glucose values were expressed in percentage of respective pre-treatment values. Percentage reductions were expressed in percentage of pre-treatment values of test substance relative to percentage of respective pre-treatment values of vehicle. All percentages were calculated and unpaired; Student's t test was then applied to establish significant difference between the test compound-treated and vehicle control groups. Differences are considered significant at P<0.05.

These data demonstrate that Compound 39 enhances the serum glucose reducing activity of rosiblitazone and metaformin.

| | | % Pre-Treatment Baseline | | | |
|---|---|---|---|---|---|
| | | (% Reduction relative to vehicle) | | | |
| Treatment | Dose | Day 3 | | Day 7 | |
| Vehicle | 5 ml/kg | 93.1 ± 6.4 | (—) | 101.2 ± 5.0 | (—) |
| Rosiglitazone | 0.5 mg/kg | 72.2 ± 6.5 | (22.4) | 61.2 ± 6.6 | (39.5) |
| Rosiglitazone + Compound 39 | 0.5 mg/kg + | 57.6 ± 2.7 | (38.1) | 51.0 ± 6.7 | (49.6) |
| | 50 mg/kg | | | | |
| Metaformin | 150 mg/kg | 68.5 ± 3.7 | (26.4) | 72.2 ± 5.7 | (28.7) |
| Metaformin + Compound 39 | 150 mg/kg + | 48.7 ± 3.9 | (47.7) | 49.8 ± 1.5 | (50.8) |
| | 50 mg/kg | | | | |

### Example 122

### Intraperitoneal and Oral Glucose Tolerance Tests in db/db Mice

To determine the effect of a test compound on blood glucose levels in diabetic mice, intraperitoneal and/or oral glucose tolerance tests (IPGTT and OGTT, respectively) were conducted. Six to eight week old, male db/db mice (BKS.Cg-m+/+Lepr^{db}/J; stock number 000642), homozygous for a mutation in the gene encoding the leptin receptor, were purchased from The Jackson Laboratory (Bar Harbor, Me. USA). Animals were singly housed in conventional caging on a 12 hr/12 hr light/dark cycle, acclimated for at least five days prior to use and fed normal laboratory chow ad libitum between experiments. Studies were conducted with animals between seven and fourteen weeks of age. Animals were used for a maximum of five consecutive studies, with a minimum recovery period of five days between studies.

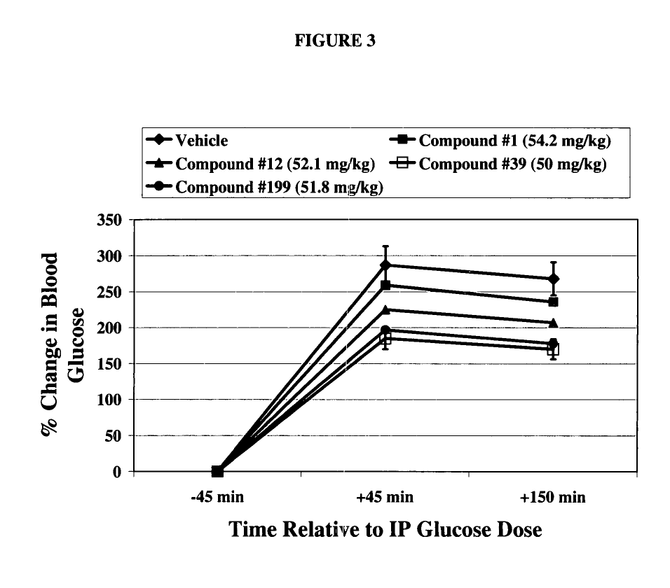

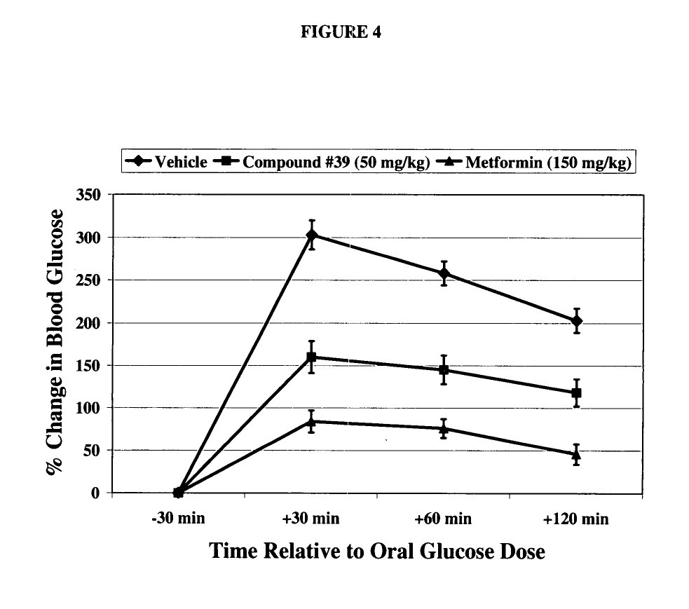

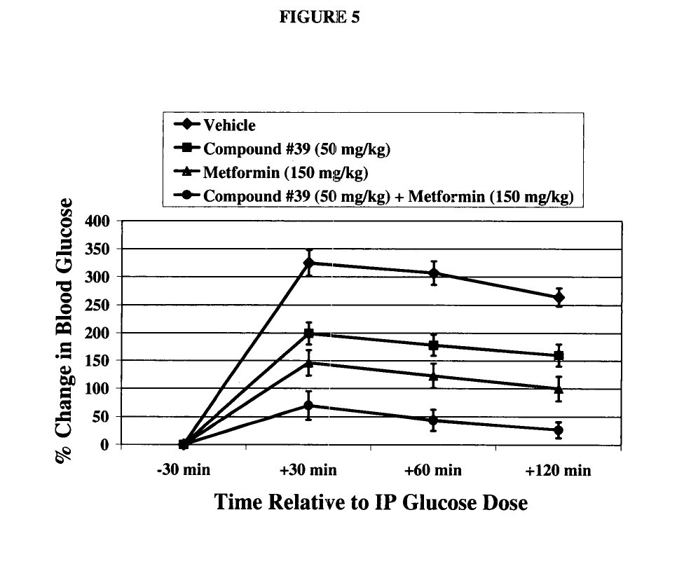

To perform an IPGTT or OGTT, db/db mice were fasted overnight beginning on day 0 at −17.5 to −18.5 hrs relative to the glucose dose on day 1 (all time points below are stated relative to the time of an intraperitoneal or oral glucose dose on day 1). At −4.5 hrs, animals were weighed, blood was collected by tail vein lancing and fasted blood glucose levels determined using a hand-held glucometer (MediSense Precision Xtra or LifeScan OneTouch Ultra). Animals were randomized into dosing groups of 6-7 animals, with each group having a similar average fasted blood glucose level. At −3 hr, animals were dosed with vehicle alone, a test compound or metformin by oral gavage at 5 ml/kg body weight. In studies with combination dosing of a test compound and metformin together in the same group, one drug was dosed at −3 hrs and the other was dosed at −2.5 hrs. In such studies, each group received a dose of vehicle or drug, followed 30 min later by a second dose of vehicle or drug as appropriate in order to keep the total dosage volume constant at 10 ml/kg body weight in all groups. Test compounds and metformin were formulated as suspensions or fully dissolved in 0.5% methylcellulose (400 cps) in water the day before dosing, stored in the dark at 4° C. overnight, and then warmed to room temperature and vortexed vigorously prior to dosing. Glucose in phosphate-buffered saline was dosed at 0 hrs at 2.0 g/kg body weight and 5 ml/kg body weight by intraperitoneal injection (for IPGTT) or by oral gavage (for OGTT). Blood glucose levels were determined as above at −45 min and +45 min, and also +90 min and/or +150 min (see FIG. 3 and the Table in this section). Alternatively, blood glucose levels were determined at −30 min, +30 min, +60 min and +120 min (see FIGS. 4 and 5). Representative compounds were found to decrease glucose intolerance in the IPGTT (FIG. 3) and OGTT (FIG. 4) models in db/db mice. The effect of the oral hypoglycemic drug metformin on glucose tolerance was also enhanced by Compound 39 (FIG. 5).

FIG. 3 displays the results of an IPGTT study to determine the effects of orally dosed test compounds on glucose tolerance in db/db mice. Compound doses were adjusted to be the molar equivalents of 50 mg/kg of Compound 39. The percent change in blood glucose between −45 min and a time point, y, after glucose dosing (e.g., +45 min, +150 min, ect.) was calculated for each animal using formula 1:

$$\begin{matrix}\% change in blood \\ glucose in each \\ mouse between - 45 \\ min . and time point y\end{matrix} = \{ ( * \frac{Blood glucose at time point y}{Blood glucose at - 45 min .} * ) \times 100 \} - 100 \qquad Formula 1$$

As a measurement of glucose excursion, the average of these values for each group of 6 mince was then graphed in FIG. 3. For clarity, error bars are shown for only the vehicle and Compound 39 treated groups. As can be seen in FIG. 3, glucose intolerance was decreased by each compound relative to vehicle-treated animals.

FIG. 4 displays the results of an OGTT study to determine the effect of orally dosed Compound 39 on glucose intolerance in db/db mice. The percent change in blood glucose between −45 min and a time point, y, after glucose dosing was calculated for each animal using formula 1. The average of these values for each group of 7 mice was then graphed in FIG. 4. As can be seen in FIG. 4, glucose intolerance was decreased by Compound 39 relative to vehicle-treated animals.

FIG. 5 displays the results of an IPGTT study to determine the effect of orally dosed Compound 39 either alone or in combination with metformin on glucose intolerance in db/db mice. The percent change in blood glucose between −45 min and a time point, y, after glucose dosing was calculated for each animal using formula 1. The average of thes values for each group of 7 mice was then graphed in FIG. 5. As can be seen in FIG. 5, glucose intolerance was decreased by Compound 39 relative to vehicle-treated animals, and the effect of the oral hypoglycemic drug metformin on glucose intolerance was also enhanced by Compound 39.

The table below shows the average percent change in glucose intolerance in an IPGTT at time point y=+45 minutes after the mice were dosed with glucose for mice treated with a compound of the invention compared to mice treated with vehicle. The percent change in blood glucose between −45 min and +45 min in each animal was calculated using Formula 1 and these values were averaged for each group of 6-7 animal treated with either a test compound or vehicle to give z, the average percent change in blood glucose between −45 min and +45 min for each group. Average percent change in glucose intolerance was calculated using Formula 2, as noted in the table below. In some cases, as noted below, the experiment was repeated one or more time with an additional group of 6-7 mice. In these cases, the results from two or more experiments have been averaged. The data in the table below indicates that the majority of the compounds of the invention decreased glucose intolerance in db/db mice (i.e., fasted mice treated with a compound of the invention typically displayed less increase in blood glucose after a glucose injection than mice treated with vehicle alone).

| | Dose | Ave. % Change |
|---|---|---|
| | (relative | in Glucose |
| | to 50 mg/kg | Intolerance at |
| Compound | Compound 39) | +45 min^{†} |
| Metformin | 150 mg/kg | −48.7 |
| Compound 39 | 1X | −35.4* |
| Compound 39 + | 1X/150 mg/kg | −52.8 |
| Metformin | | |
| Compound 1 | 1X | −9.8 |
| Compound 3 | 1X | −1.3 |
| Compound 6 | 1X | −10.1 |
| Compound 9 | 1X | +1.2 |
| Compound 12 | 1X | −21.6 |
| Compound 16 | 1X | −41.9 |
| Compound 196 | 1X | −3.8 |
| Compound 41 | 1X | −19.0 |
| Compound 68 | 1X | −0.5** |
| Compound 45 | 1X | −1.2 |
| Compound 197 | 1X | −2.6 |
| Compound 195 | 1X | −3.6 |
| Compound 66 | 1X | −45.2 |
| Compound 67 | 1X | −4.0 |
| Compound 69 | 1X | −5.5 |
| Compound 43 | 1X | −16.5** |
| Compound 168 | 1X | −1.3 |
| Compound 96 | 1X | −52.6** |
| Compound 200 | 1X | −22.3 |
| Compound 199 | 1X | −31.4 |
| Compound 201 | 1X | −17.5 |
| Compound 215 | 1X | −35.4*** |
| Compound 202 | 1X | −31.2 |
| Compound 203 | 1X | −27.9 |
| Compound 204 | 1X | −18.1** |
| Compound 194 | 1X | +9.8 |
| Compound 205 | 1X | −7.5 |
| Compound 206 | 1X | −23.7 |
| Compound 207 | 1X | −36.7 |
| Compound 216 | 1X | −50.2** |
| Compound 217 | 1X | −10.4 |
| Compound 218 | 1X | −4.0 |
| Compound 219 | 1X | −24.3** |
| Compound 221 | 1X | −26.0 |
| Compound 222 | 1X | −33.1** |
| Compound 220 | 1X | −36.7** |
| Compound 223 | 1X | −19.9 |
| Compound 224 | 1X | −8.3 |
| Compound 225 | 1X | −16.0 |
| Compound 226 | 1X | −12.8 |
| Compound 227 | 1X | −9.2 |
| Compound 228 | 1X | −19.9 |
| Compound 229 | 1X | −35.6 |
| Compound 230 | 1X | −13.6 |

^{†}The average percent change in glucose tolerance was
calculated using formula 2:

Formula 2

$\begin{matrix}Average \% change \\ in glucose \\ intolerance\end{matrix} = \{ ( * \frac{\begin{matrix}z for a group treated with \\ a test compound\end{matrix}}{\begin{matrix}z for a group treated with \\ vehicle\end{matrix}} * ) \times 100 \} - 100$
*Data shown is the average of thirty experiments
**Data shown is the average of two experiments
***Data shown is the average of three experiments

### Example 123

### Seven Day Baseline Glucose Studies in db/db and KK-A^{y} Mice

To determine the effect of a test compound on blood glucose levels in fed diabetic mice, seven day baseline glucose (7DBG) studies were conducted. Six to eight week old, male db/db mice (C57BLKS/J-m+/+Lepr^{db}/Iar), homozygous for a mutation in the gene encoding the leptin receptor, were purchased from the Institute for Animal Reproduction (Kasumigaura, Japan). Six to eight week old male KK-A^{y} mice (KK-A^{y}/Ta Jcl), a polygenic diabetes model, were purchased from Cler Japan (Tokyo, Japan). Animals were housed four to a cage in micro-isolators on a 12 hr/12 hr light/dark cycle, acclimated for at least one week prior to use and fed normal laboratory chow ad libitum. Studies were conducted with animals between nine and fourteen weeks of age. Animals were used for a maximum of two consecutive studies, with a minimum recovery period of seven days between studies.

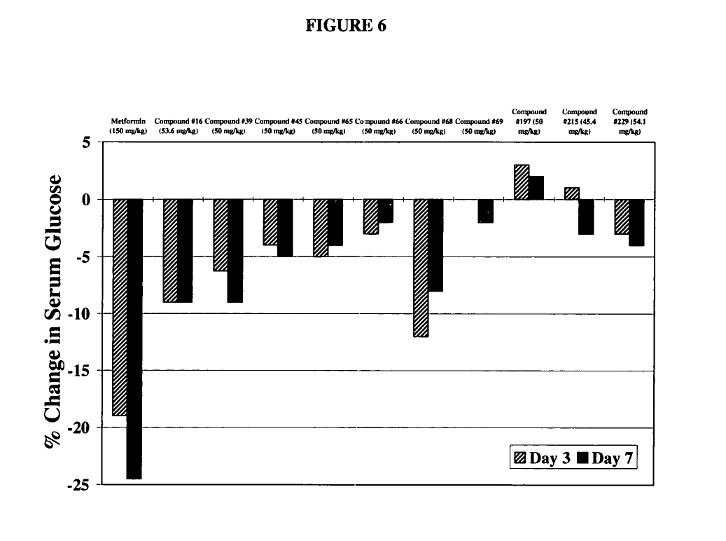

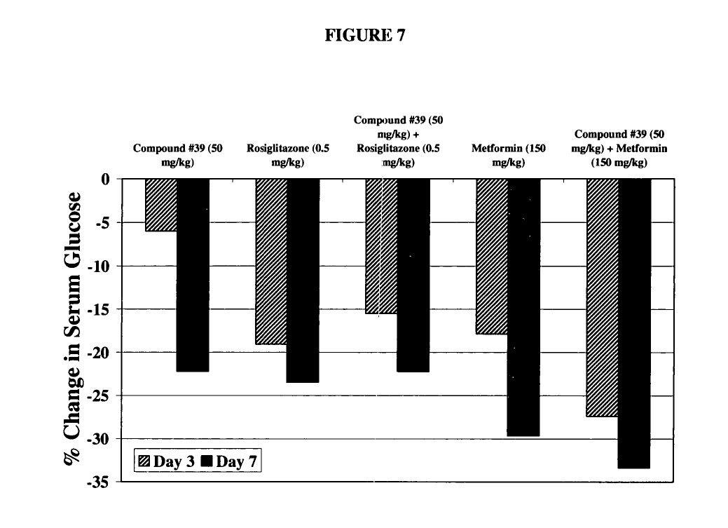

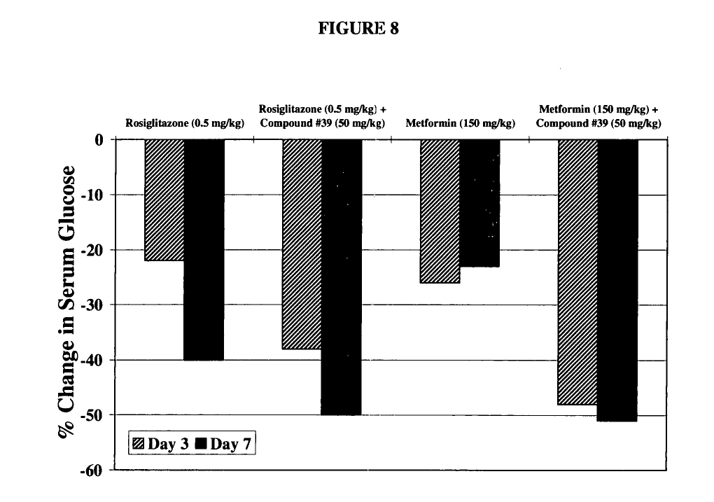

To perform a 7DBG study, the starting glucose levels of db/db or KK-Ay mice in the fed state were determined on day 0, 24 hrs prior to the first drug dose on day 1. Animals were retro-orbitally bled and serum glucose levels measured by the mutarotase-glucose oxidase enzymatic method using a Glucose CII-Test Wako kit (Wako Pure Chemicals Industries, Ltd., Osaka, Japan). Animals were then randomized into dosing groups of 6-8 animals, with each group having a similar starting average glucose level. On days 1 through 7, animals were orally dosed once per day with vehicle, a test compound, rosiglitazone or metformin by oral gavage at 5 ml/kg body weight. In studies with combination dosing of a test compound and rosiglitazone or metformin together in the same group, one drug was dosed at −3 hrs and the other was dosed at −2 hrs. In such studies, each group received a dose of vehicle or drug, followed 60 min later by a second dose of vehicle or drug as appropriate in order to keep the total dosage volume constant at 10 ml/kg body weight in all groups. Test compounds, rosiglitazone and metformin were formulated fresh each day as suspensions or fully dissolved in 0.5% methylcellulose (400 cps) in water. Serum glucose levels were determined 3 hrs after dosing the first compound on days 3 and 7. In almost all cases, compounds of the invention were found to decrease fed serum glucose levels in both db/db (see FIG. 6) and KK-A^{y} (see FIG. 7) mice. The glucose lowering activities of the oral hypoglycemic drugs rosiglitazone and metformin were also enhanced by Compound 39 in both strains of mice (see FIGS. 7 and 8).

FIG. 6 displays the results of a 7DBG study to determine the effects of orally dosing test compounds of the invention daily for seven days on glucose levels in fed db/db mice. The serum glucose levels on days 3 or 7 as a percent of the serum glucose levels on day 0 for individual animals in each group (6-7 mice/group), calculated using Formula 3:

$$\begin{matrix}blood glucose on day 3 or 7 \\ as a \% of blood glucose on \\ day 0 for each mouse\end{matrix} = \{ ( * \frac{Blood glucose on day 3 or 7}{Blood glucose on day 0} * ) \times 100 \} \qquad Formula 3$$

The values from Formula 3 were averaged for each group of 6-7 mice treated with either a test compound (a compound of the invention or metformin), or vehicle. The average values for test compound-treated groups were then normalized relative to the vehicle-treated group using Formula 4:

$$\begin{matrix}\% change in \\ serum glucose\end{matrix} = \{ ( * \frac{\begin{matrix}\begin{matrix}Average \% change in blood glucose on \\ day 3 or 7 as a \% of blood glucose on\end{matrix} \\ day 0 for a group treated with a test compond\end{matrix}}{\begin{matrix}\begin{matrix}Average \% change in blood glucose on day 3 or 7 \\ as a \% of blood glucose on day 0 for a\end{matrix} \\ group treated with vehicle\end{matrix}} * ) 100 \} - 100 \qquad Formula 4$$

Doses of Compounds 16, 215 and 229 were adjusted to be the molar equivalent of a 50 mg/kg dose of Compound 39; other test compounds were dosed at 50 mg/kg. All compounds were tested once, except metformin and Compound 39, which were tested two and four times, respectively. As can be seen from FIG. 6, serum glucose levels were decreased by several compounds relative to vehicle-treated animals.

FIG. 7 displays the results of a 7DBG study to determine the effects of orally dosing Compound 39 daily for seven days with and without rosiglitazone or metformin on glucose levels in fed KK-A^{y} mice. The serum glucose levels on days 3 or 7 as a percent of the serum glucose levels on day 0 for individual animals in each group (8 mice/group), were calculated using Formula 3. The values from Formula 3 were averaged for each group of 8 mice treated with either a test compound (Compound 39, rosiglitazone, or metformin), a combination of test compounds (Compound 39 and rosiglitazone or Compound 39 and metformin), or vehicle. The average values for a test compound-treated groups or a combination group were then normalized relative to the vehicle-treated group using Formula 4. Rosiglitazone and metformin were dosed 60 min prior to dosing Compound 39 and 3 hrs prior to measuring serum glucose levels on days 3 and 7. As can be seen from FIG. 7, treatment with Compound 39 decreased serum glucose and also enhanced the glucose lowering activities of the oral hypoglycemic drugs rosiglitazone and metformin.

FIG. 8 displays the results of a 7DBG study to determine the effects of orally dosing Compound 39 daily for seven days in combination with rosiglitazone or metformin on glucose levels in fed db/db mice. The serum glucose levels on days 3 or 7 as a percent of the serum glucose levels on day 0 for individual animals in each group (6 mice/group), were calculated using Formula 3. The values from formula 3 were averaged for each group of 6 mice treated with either a test compound (rosiglitazone or metformin), a combination of test compounds (Compound 39 and rosiglitazone or Compound 39 and metformin), or vehicle. The average values for a test compound-treated group or a combination group were then normalized relative to the vehicle-treated group using Formula 4. Rosiglitazone and metformin were dosed 60 min prior to dosing Compound 39 and 3 hrs prior to measuring serum glucose levels on days 3 and 7. As can be seen in FIG. 8, treatment with Compound 39 enhanced the glucose lowering activities of the oral hypoglycemic drugs rosiglitazone and metformin.

### Example 124

### Seven Day Baseline Glucose and Oral Glucose Tolerance Test in ZDF Rats

To determine the effect of a test compound on blood glucose levels in fed and fasted diabetic rats, a 7DBG study was conducted, followed immediately by an 8^{th} day of dosing and an OGTT study on the same animals. Male ZDF rats (ZDF/GmiCrl-fa/fa), homozygous for a mutation in the gene encoding the leptin receptor, were purchased from Charles River Laboratories (Wilmington, Mass., USA). Animals were individually housed in clear polycarbonate cages (contact bedding) on a 12 hr/12 hr light/dark cycle, acclimated for at least one week prior to use and fed normal laboratory chow (Purina No. 5008) ad libitum. Studies were conducted with animals starting between eleven and twelve weeks of age. Animals were used for a single study before being replaced.

To perform a 7DBG plus OGTT study, animals were bled by tail vein lancing on day 0, 24 hrs prior to the first drug dose on day 1, and blood glucose levels were measured by a hand-held glucometer. Animals were randomized into dosing groups of 6 animals, with each group having a similar starting average glucose level. On days 1-8, animals were orally dosed once per day with either vehicle or test compound of the invention by oral gavage at 5 ml/kg body weight. After 60 min, each group was orally dosed a second time with either vehicle or metformin by oral gavage at 5 ml/kg body weight (therefore keeping the total volume between the two doses constant at 10 ml/kg body weight in all groups). Test compound and metformin were formulated fresh each day as suspensions or fully dissolved in 0.5% methylcellulose (400 cps) in water. Blood glucose levels were determined daily using a hand-held glucometer 3 hrs after the first dose on days 1 through 7.

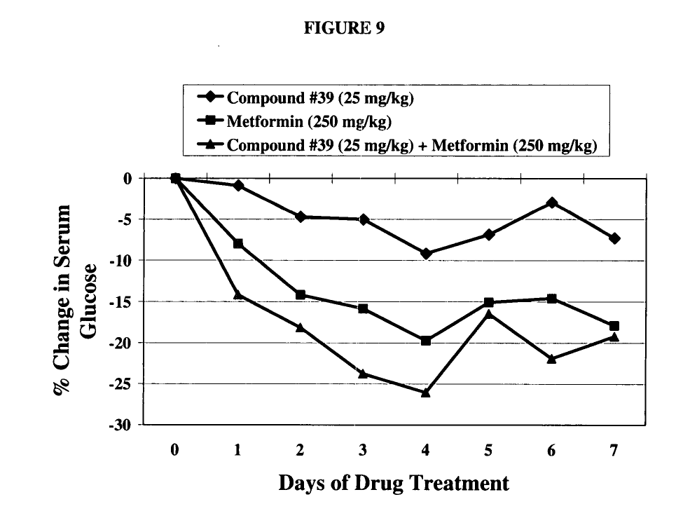

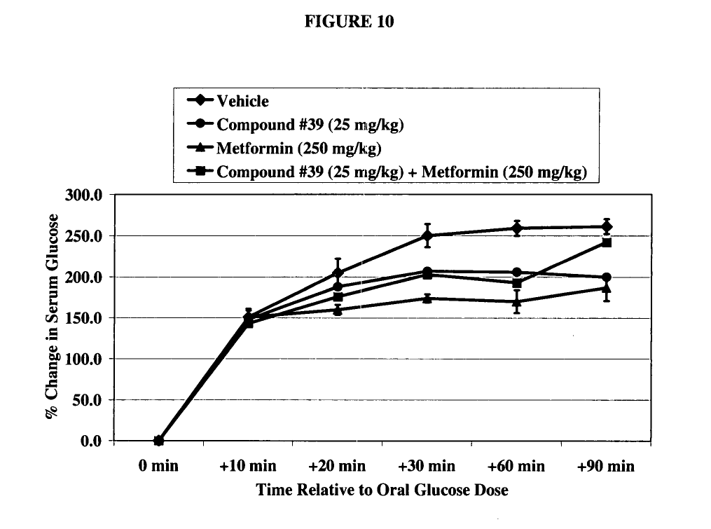

For the OGTT, rats from the 7DBG portion of the study were fasted overnight beginning on day 7 at −12 to −18 hrs relative to the glucose dose on day 8 (all time points below are stated relative to the time of the oral glucose dose on day 8). As noted above, animals were dosed on day 8 on the same schedule as was used for days 1 through 7 of the 7DBG study. Three hours after dosing the test compound, glucose in phosphate-buffered saline was dosed at 2.0 g/kg body weight and 7 ml/kg body weight by oral gavage. Blood was collected from the tail vein or by puncture of the retro-orbital sinus at 0 min, immediately prior to the glucose dose, and also at +10, +20, +30, +60 and +90 min. Serum glucose levels were determined using a laboratory analyzer. After daily dosing for 7 days, Compound 39 was found to lower blood glucose in the fed state and also enhanced the activity of the oral hypoglycemic drug metformin (FIG. 9). After daily dosing for 8 days, Compound 39 was found to decrease glucose intolerance in the OGTT (FIG. 10). However, Compound 39 did not appear to enhance the effect of metformin on glucose tolerance in this assay (FIG. 10). This may be due to the dose of metformin (250 mg/kg) that was used, which may be too high to observe any synergy between the two compounds.

FIG. 9 displays the results of the above described 7DBG study to determine the effects of orally dosing Compound 39 daily for seven days with and without metformin on glucose levels in fed ZDF rats. The serum glucose levels on days 3 or 7 as a percent of the serum glucose levels on day 0 for individual animals in each group (6 rats/group), were calculated using Formula 3. The values from Formula 3 were averaged for each group of 6 rats treated with either Compound 39, metformin, a combination of Compound 39 and metformin, or vehicle. The average values for a test compound-treated group or a combination group were then normalized relative to the vehicle-treated group using Formula 4. Compound 39 was dosed 60 min prior to dosing metformin and 3 hrs prior to measuring serum glucose levels on days 1 through 7. Treatment with Compound 39 decreased serum glucose and also enhanced the glucose lowering activities of the oral hypoglycemic drug metformin.

FIG. 10 displays the results of the OGTT study described above to determine the effects of orally dosing Compound 39 daily for eight days with and without metformin on glucose levels in fasted ZDF rats. The percent change in blood glucose between 0 min. and a time point, y (i.e., +10 min, +20 min., +30 min, +60 min, and +90 min), after glucose dosing was calculated for each animal using Formula 1. The average of these values for each group of 6 rates was then graphed in FIG. 10. The indicated time points are relative to the oral glucose dose. For clarity, error bars are shown for only the vehicle and metformin treated groups. As can be seen from FIG. 10, glucose intolerance was decreased by Compound 39 relative to vehicle-treated animals.

### Example 125

### In Vitro Cytotoxicity Study on Primary Rat Hepatocytes

Male SD rats having body weights of approximately 200 g were used for the study. Hepatocytes were freshly isolated and seeded with 15,000 cells/well in 96 wells coated with 100 μl collagen. The hepatocytes were cultured for 3-4 hours with HCM Bulletkit medium (Clonetics #CC-3198) then treated with 0.4 μM, 2 μM, 10 μM and 50 μM of a test compound for 20 hours. Flepatocytes were then incubated with MTS reagent (Promega #G5421) for 2-3 hours and absorbance in each well at 490 nM was measured to determine the number of living cells in each well (the absorbance at 490 nM is directly proportional to the number of living cells in the well). The EC₅₀ for each test compound was calculated with the Excel Xlfit wizard program based on the optical density values compared with vehicle treated cells.

The results in the table below indicate that the compounds of the invention exhibit no significant hepatocyte toxicity.

| | Average EC₅₀ |
|---|---|
| | (hepatocytes from at |
| Compound Number | least 2 rats) |
| CCl₄ (positive control) | 7 |
| 9 | >50 |
| 12 | >50 |
| 16 | >50 |
| 39 R-enantiomer | >50 |
| 39 S-enantiomer | >50 |
| 41 racemic mixture | >50 |
| 41 R-enantiomer | >50 |
| 41 S-enantiomer | >50 |
| 64 | >50 |
| 66 | >50 |
| 96 | >50 |
| 202 | >50 |
| 206 | >50 |
| 215 | >50 |
| 216 | >50 |
| 219 | >50 |
| 220 | >50 |
| 222 | >50 |

All publications, patent applications, patents, and other documents cited herein are incorporated by reference in the entirety. In case of conflict, the present specification, including definitions, will control. In addition, the materials, methods, and examples are illustrative only and not intended to be limiting.

1. A compound represented by the following structural formula: 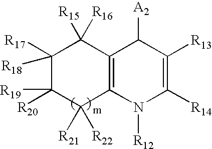
or a pharmaceutically acceptable salt, solvate, clathrate, or prodrug thereof wherein:
m is 0, 1, or 2;
A₂ is an optionally substituted aryl or an optionally substituted heteroaryl;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁, and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that R₁₄ is not lower alkyl, cyclopentyl, phenyl, bromomethyl, trifluoromethyl, —NH₂, nitro, —NHC(O)NH-phenyl, —SH, —SS-heterocycle, —S-(lower alkenyl), or —S-(cycloalkenyl);
provided that when A₂ is o-chlorophenyl, R₁₄ is not a methyl substituted with a heteroaralkoxy;
provided that when A₂ is o-(trifluoromethyl)-phenyl, R₁₄ is not —CH₂—S(O)ᵣ-phenyl, —CH₂—S(O)ᵣ-pyridyl, or —CH₂(CH₃)—S(O)ᵣ-phenyl, wherein r is 0, 1, or 2;
provided that when A₂ is a chlorophenyl, R₁₄ is not —SH, —SCH₃, —SCH₂CH₃, —SCH(CH₂)CH₃, —SCH₂C(O)NH₂, or —SCH₂C(O)NH-(bromophenyl); and
provided that when R₁₄ is —H, A₂ is not thiazolyl.

2. The compound of claim 1, wherein R₁₅ and R₁₆ taken together are ═O and m is 1.

3. The compound of claim 2, wherein A₂ is selected from the group consisting of a substituted or unsubstituted phenyl, a substituted or unsubstituted pyridyl, a substituted or unsubstituted 1-oxo-pyridyl, a substituted or unsubstituted furanyl, a substituted or unsubstituted anthracenyl, a substituted or unsubstituted fluorenyl, a substituted or unsubstituted indenyl, a substituted or unsubstituted azulenyl, a substituted or unsubstituted naphthyl, a substituted or unsubstituted 5,6,7,8-tetrahydronaphthyl, a substituted or unsubstituted benzo[1,3]dioxolyl, a substituted or unsubstituted thienyl, a substituted or unsubstituted pyrrolyl, a substituted or unsubstituted oxazolyl, a substituted or unsubstituted imidazolyl, a substituted or unsubstituted thiazolyl, a substituted or unsubstituted isoxazolyl, a substituted or unsubstituted quinoliny, a substituted or unsubstituted pyrazolyl, a substituted or unsubstituted isothiazolyl, a substituted or unsubstituted pyridazinyl, a substituted or unsubstituted pyrimidinyl, a substituted or unsubstituted pyrazinyl, a substituted or imsubstituted triazinyl, a substituted or unsubstituted triazolyl, a substituted or unsubstituted thiadiazolyl, a substituted or unsubstituted quinolyl, a substituted or unsubstituted isoquniolyl, a substituted or unsubstituted indazolyl, a substituted or unsubstituted benzoxazolyl, a substituted or unsubstituted benzofuryl, a substituted or unsubstituted benzothiazolyl, a substituted or unsubstituted indolizinyl, a substituted or unsubstituted imidazopyridyl, a substituted or unsubstituted isothiazolyl, a substituted or unsubstituted tetrazolyl, a substituted or unsubstituted benzimidazolyl, a substituted or unsubstituted benzoxazolyl, a substituted or unsubstituted benzothiazolyl, a substituted or unsubstituted benzothiadiazolyl, a substituted or unsubstituted benzoxadiazolyl, a substituted or unsubstituted indolyl, a substituted or unsubstituted tetrahydroindolyl, a substituted or unsubstituted azaindolyl, a substituted or unsubstituted imidazopyridyl, a substituted or unsubstituted qunizaolinyl, a substituted or unsubstituted purinyl, a substituted or unsubstituted pyrrolo[2,3]pyrimidyl, a substituted or unsubstituted pyrazolo[3,4]pyrimidyl, a substituted or unsubstituted imidazo[1,2-a]pyridyl, or a substituted or unsubstituted benzo(b)thienyl.

4. The compound of claim 3, wherein A₂ is substituted with one or more substituents selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR₂₈R₂₉, —NR₃₀C(O)R₃₁, a halo, —OR₃₀, cyano, nitro, a haloalkoxy, —C(O)R₃₀, —NR₂₈R₂₉, —SR₃₀, —C(O)OR₃₀, —OC(O)R₃₀, —NR₃₀C(O)NR₂₈R₂₉, —OC(O)NR₂₈R₂₉, —NR₃₀C(O)OR₃₁, —S(O)ᵣR₃₀, and —S(O)ₚNR₂₈R₂₉, wherein:
R₂₈ and R₂₉, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₈ and R₂₉ taken together with the nitrogen to which they are attached is optionally substituted heterocycloalkyl or optionally substituted heteroaryl; and
R₃₀ and R₃₁ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl.

5. The compound of claim 3, wherein A₂ is selected from the group consisting of a substituted or unsubstituted phenyl, a substituted or unsubstituted benzo[1,3]dioxolyl, a substituted or unsubstituted pyridyl, a substituted or unsubstituted indolyl, a substituted or unsubstituted quinolinyl, a substituted or unsubstituted 1-oxo-pyridyl, a substituted or unsubstituted pyridazinyl, a substituted or unsubstituted pyrimidinyl, a substituted or unsubstituted pyrazinyl, a substituted or unsubstituted furanyl, a substituted or unsubstituted thienyl, a substituted or unsubstituted [1,3,5]triazinyl, a substituted or unsubstituted thiazolyl, a substituted or unsubstituted imidazolyl, a substituted or unsubstituted oxazolyl, a substituted or unsubstituted indolizinyl, a substituted or unsubstituted imidazo[1,2-a]pyridyl, a substituted or unsubstituted 2,3-dihydro-benzo[1,4]dioxinyl, and a substituted or unsubstituted naphthyl.

6. The compound of claim 5, wherein A₂ is substituted with one, two or three substituents selected from the group consisting of halo, nitro, —NR₃₂R₃₂, lower alkyl, lower alkoxy, lower alkyl sulfanyl, lower haloalkyl, phenyl, hydroxyl, cyano, and lower alkyl sulfonyl, wherein R₃₂, for each occurrence, is —H or a lower alkyl.

7. The compound of claim 2, wherein R₁₉ and R₂₀ are each, independently, a lower alkyl.

8. The compound of claim 2, wherein R₁₃ is —C(O)O-(lower alkyl), —C(O)OH, cyano, —C(O)NR₃₂R₃₂, —C(O)-(lower alkyl), wherein R₃₂, for each occurrence, is —H or a lower alkyl.

9. The compound of claim 2, wherein R₁₄ is cyclopropyl, ethoxymethyl, 2-amino-ethoxymethyl, 2-azido-ethoxymethyl, 2-(2-hydroxy-3-phenoxy-propylamino)-ethoxymethyl, propoxymethyl, isopropoxymethyl, N-mesyl-2-aminoethoxymethyl, N-acetyl-2-aminoethoxymethyl, N-ethyl-2-aminoethoxymethyl, N-methyl-2-aminoethoxymethyl, 2-(1,3-dioxo-1,3-dihydro-isoindol-2-yl)-ethoxymethyl, morpholin-4-yl-methyl, 2-morpholin-4-yl-ethoxymethyl, N,N-dimethylaminomethyl, carbethoxycarbonylmethoxymethyl, N-(2-hydroxyethyl)-N-methylaminomethyl, piperazin-1-yl-methyl, 2-hydroxyethoxymethyl, N,N-dimethylamino-ethoxymethyl, 4-aminobutyl, imidazol-5-yl-methoxymethyl, imidazol-4-yl-methoxymethyl, 2-imidazol-1-yl-ethoxymethyl, 3-imidazol-1-yl-propyl, 3-pyrazol-1-yl-propyl, propoxymethyl, isopropoxymethyl, methoxyethoxymethyl, pyrrol-3-yl-methoxymethyl, pyrrol-2-yl-methoxymethyl, [1,2,4]triazol-3-yl-methoxymethyl, 2H-pyrazol-3-yl-methoxymethyl, 3H-[1,2,3]triazol-4-yl-methoxymethyl, or 2-pyrrol-1-yl-ethoxymethyl.

10. The compound of claim 1, wherein R₁₄ is —NR₃₉R₄₀ or —OR₄₁, wherein:
R₃₉ and R40 are each, independently, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂; or R₃₉ and R₄₀, taken together with the nitrogen to which they are attached are an optionally substituted heterocycloalkyl or optionally substituted heteroaryl;
R₄₁, is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂;
R₄₂ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₄₃ and R₄₄ are each, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl, or R₄₃ and R₄₄ taken together with the nitrogen to which they are attached are an optionally substituted heterocycloalkyl or optionally substituted heteroaryl.

11. A compound represented by the following structural formula: 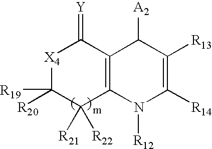
or a pharmaceutically acceptable salt, solvate, clathrate, or prodrug thereof wherein:
m is 0, 1, or 2;
A₂ is an optionally substituted aryl or an optionally substituted heteroaryl;
X₄ is O, S, or —NR₂₃—;
Y is O or S;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that R₁₄ is not a lower alkyl, a halomethyl, phenyl, cyano, or hydroxymethyl;
provided that when R₁₄ is H or —NH₂, A₂ is not a substituted quinolinyl; and
provided that when R₁₄ is 2-(N,N-dimethylamino)-ethyl or methoxymethyl, A₂ is not o-chlorophenyl or o-(trifluoromethyl)-phenyl.

12. The compound of claim 11, wherein Y is ═O, X₄ is —O—, and m is 1.

13. The compound of claim 12, wherein A₂ is selected from the group consisting of a substituted or unsubstituted phenyl, a substituted or unsubstituted pyridyl, a substituted or unsubstituted 1-oxo-pyridyl, a substituted or unsubstituted furanyl, a substituted or unsubstituted anthracenyl, a substituted or unsubstituted fluorenyl, a substituted or unsubstituted indenyl, a substituted or unsubstituted azulenyl, a substituted or unsubstituted naphthyl, a substituted or unsubstituted 5,6,7,8-tetrahydronaphthyl, a substituted or unsubstituted benzo[1,3]dioxolyl, a substituted or unsubstituted benzo[1,4]dioinyl, a substituted or unsubstituted thienyl, a substituted or unsubstituted pyrrolyl, a substituted or unsubstituted oxazolyl, a substituted or unsubstituted imidazolyl, a substituted or unsubstituted thiazolyl, a substituted or unsubstituted isoxazolyl, a substituted or unsubstituted quinolinyl, a substituted or unsubstituted pyrazolyl, a substituted or unsubstituted isothiazolyl, a substituted or unsubstituted pyridazinyl, a substituted or unsubstituted pyrimidinyl, a substituted or unsubstituted pyrazinyl, a substituted or unsubstituted triazinyl, a substituted or unsubstituted triazolyl, a substituted or unsubstituted thiadiazolyl, a substituted or unsubstituted isoquniolinyl, a substituted or unsubstituted indazolyl, a substituted or unsubstituted benzoxazolyl, a substituted or unsubstituted benzofuranyl, a substituted or unsubstituted benzothiazolyl, a substituted or unsubstituted indolizinyl, a substituted or unsubstituted imidazopyridyl, a substituted or unsubstituted isothiazolyl, a substituted or unsubstituted tetrazolyl, a substituted or unsubstituted benzimidazolyl, a substituted or unsubstituted benzoxazolyl, a substituted or unsubstituted benzothiazolyl, a substituted or unsubstituted benzothiadiazolyl, a substituted or unsubstituted benzoxadiazolyl, a substituted or unsubstituted indolyl, a substituted or unsubstituted tetrahydroindolyl, a substituted or unsubstituted azaindolyl, a substituted or unsubstituted imidazopyridyl, a substituted or unsubstituted quinazolinyl, a substituted or unsubstituted purinyl, a substituted or unsubstituted pyrrolo[2,3]pyrimidinyl, a substituted or unsubstituted pyrazolo[3,4]pyrimidinyl, a substituted or unsubstituted imidazo[1,2-a]pyridyl, or a substituted or unsubstituted benzo(b)thienyl.

14. The compound of claim 13, wherein A₂ is substituted with one or more substituents selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR₂₈R₂₉, —NR₃₀C(O)R₃₁, a halo, —OR₃₀, cyano, nitro, a haloalkoxy, —C(O)R₃₀, —NR₂₈R₂₉, —SR₃₀, —C(O)OR₃₀, —OC(O)R₃₀, —NR₃₀C(O)NR₂₈R₂₉, —OC(O)NR₂₈R₂₉, —NR₃₀C(O)OR₃₁, —S(O)ᵣR₃₀, and —S(O)ₚNR₂₈R₂₉, wherein:
R₂₈ and R₂₉, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₈ and R₂₉ taken together with the nitrogen to which they are attached is optionally substituted heterocycloalkyl or optionally substituted heteroaryl; and
R₃₀ and R₃₁ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl.

15. The compound of claim 13, wherein A₂ is selected from the group consisting of a substituted or unsubstituted phenyl, a substituted or unsubstituted benzo[1,3]dioxolyl, a substituted or unsubstituted pyridyl, a substituted or unsubstituted indolyl, a substituted or unsubstituted quinolinyl, a substituted or unsubstituted 1-oxo-pyridyl, a substituted or unsubstituted pyridazinyl, a substituted or unsubstituted pyrimidinyl, a substituted or unsubstituted pyrazinyl, a substituted or unsubstituted furanyl, a substituted or unsubstituted thienyl, a substituted or unsubstituted [1,3,5]triazinyl, a substituted or unsubstituted thiazolyl, a substituted or unsubstituted imidazolyl, a substituted or unsubstituted oxazolyl, a substituted or unsubstituted indolizinyl, a substituted or unsubstituted imidazo[1,2-a]pyridyl, a substituted or unsubstituted 2,3-dihydro-benzo[1,4]dioxinyl, and a substituted or unsubstituted naphthyl.

16. The compound of claim 15, wherein A₂ is substituted with one, two or three substituents selected from the group consisting of halo, nitro, —NR₃₂R₃₂, lower alkyl, lower alkoxy, lower alkyl sulfanyl, lower haloalkyl, phenyl, hydroxyl, cyano, and lower alkyl sulfonyl, wherein R₃₂, for each occurrence, is —H or a lower alkyl.

17. The compound of claim 12, wherein R₁₉ and R₂₀ are each, independently, a lower alkyl.

18. The method of claim 12, wherein R₁₃ is —C(O)O-(lower alkyl), —C(O)OH, cyano, —C(O)NR₃₂R₃₂, —C(O)-(lower alkyl), wherein R₃₂, for each occurrence, is —H or a lower alkyl.

19. The compound of claim 12, wherein R₁₄ is cyclopropyl, ethoxymethyl, 2-amino-ethoxymethyl, 2-azido-ethoxymethyl, 2-(2-hydroxy-3-phenoxy-propylamino)-ethoxymethyl, propoxymethyl, isopropoxymethyl, N-mesyl-2-aminoethoxymethyl, N-acetyl-2-aminoethoxymethyl, N-ethyl-2-aminoethoxymethyl, N-methyl-2-aminoethoxymethyl, 2-(1,3-dioxo-1,3-dihydro-isoindol-2-yl)-ethoxymethyl, morpholin-4-yl-methyl, 2-morpholin-4-yl-ethoxymethyl, N,N-dimethylaminomethyl, carbethoxycarbonylmethoxymethyl, N-(2-hydroxyethyl)-N-methylaminomethyl, piperazin-1-yl-methyl, 2-hydroxyethoxymethyl, N,N-dimethylamino-ethoxymethyl, 4-aminobutyl, imidazol-5-yl-methoxymethyl, imidazol-4-yl-methoxymethyl, 2-imidazol-1-yl-ethoxymethyl, 3-imidazol-1-yl-propyl, 3-pyrazol-1-yl-propyl, propoxymethyl, isopropoxymethyl, methoxyethoxymethyl, pyrrol-3-yl-methoxymethyl, pyrrol-2-yl-methoxymethyl, [1,2,4]triazol-3-yl-methoxymethyl, 2H-pyrazol-3-yl-methoxymethyl, 3H-[1,2,3]triazol-4-yl-methoxymethyl, or 2-pyrrol-1-yl-ethoxymethyl.

20. The compound of claim 12, wherein R₁₄ is —NR₃₉R₄₀ or —OR₄₁, wherein:
R₃₉ and R₄₀ are each, independently, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂; or R₃₉ and R₄₀, taken together with the nitrogen to which they are attached are an optionally substituted heterocycloalkyl or optionally substituted heteroaryl;
R₄₁ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)R₄₂, or —S(O)R₄₂;
R₄₂ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₄₃ and R₄₄ are each, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl, or R₄₃ and R₄₄ taken together with the nitrogen to which they are attached are an optionally substituted heterocycloalkyl or optionally substituted heteroaryl.

21. A compound represented by the following structural formula: 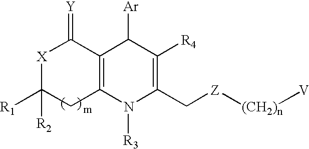
or a pharmaceutically acceptable salt, solvate, clathrate, hydrate or polymorph thereof wherein:
Ar is a mono- or poly-substituted or unsubstituted aromatic or heteroaromatic ring, wherein if the ring is substituted, the substituents are independently selected from the group consisting of substituted or unsubstituted lower alkyl, -halo, —CN, —N(R₅)(R₅), —OR₆, —C(O)R₅, —C(O)₂R₅, —OC(O)R₅, —NO₂, and —C(O)N(R₅)(R₅), or two adjacent carbon atoms on the ring are linked by the group —O—(CH₂)_{q}—O— to form a bicyclic ring system, wherein q is an integer selected from 1, 2, 3 or 4;
V is H, -halo, N₃, —NO₂, —CN, —OH, —N(R₅)(R₇), —OR₅, —C(O)R₅, —OC(O)R₅, —C(O)NHC(O)R₅, substituted or unsubstituted 3-7 membered monocyclic heterocycle or substituted or unsubstituted 8-12 membered bicyclic heterocycle;
X is selected from the group consisting of O, S, —NR5, and —C(R₅)(R₅);
Y is O or S;
Z is —O—, —S—, —N(R₅)—, —C(O)—, —OC(O)—, —C(O)N(R₅)C(O)—, substituted or unsubstituted —(C₁-C₁₀)alkyl-, substituted or unsubstituted —(C₂-C₁₀)alkenyl-, substituted or unsubstituted —(C₂-C₁₀)alkynyl-, substituted or unsubstituted —(C₃-C₁₀)cycloalkyl-, substituted or unsubstituted —(C₈-C₁₄)bicycloalkyl-, substituted or unsubstituted —(C₅-C₁₀)cycloalkenyl-, substituted or unsubstituted —(C₃-C₁₀)heterocycle-, substituted or unsubstituted phenyl, substituted or unsubstituted naphthyl, substituted or unsubstituted benzyl, —C(O)O—, —C(O)OC(R₅)(R₅)—, —N(R₅)C(O)—, —N(R₅)C(O)NR₅—, —C(O)NR₅—, —OC(O)O—, —S(O)N(R₅)—, —S(O)—, or —S(O)₂—;
R₁ and R₂ are at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;
R₃ is at each occurrence independently —H, —C(O)R₅ or substituted or unsubstituted —(C₁-C₁₀)alkyl;
R₄ is at each occurrence independently —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)OR₅, —S(O)N(R₅)(R₅), —SR₅, —S(O)R₅, —S(O)₂R₅ or a substituted or unsubstituted bioisosteric replacement of an ester;
each R₅ is at each occurrence independently H or substituted or unsubstituted —(C₁-C₁₀)alkyl;
each R₆ is at each occurrence independently H, substituted or unsubstituted —(C₁-C₁₀)alkyl or —(CH₂)ₚ—N(R₅)—(C₁-C₆)alkyl optionally substituted with one or more —OR₅ or —O-aryl groups;
R₇ is selected from the group consisting of H and substituted or unsubstituted —(C₁-C₁₀)alkyl optionally substituted with one or more —OR₅ or —O-aryl groups;
n is an integer selected from 1-10;
m is an integer selected from 0-2; and
p is an integer selected from 1-6; and
wherein if X is —(CH₂)— or A is a bicyclic ring, R₁ and R₂ are not both H.

22. A compound according to claim 21, wherein:
V is NH₂ or N₃;
X is O or CH₂;
R₁ and R₂ are independently H or lower alkyl;
R₄ is CO₂-lower alkyl;
R₃, R₅, R₆ and R₇ are each independently H or lower alkyl;
n is an integer selected from 1-5; and
p is an integer selected from 1-4.

23. A compound represented by the following structural formula: 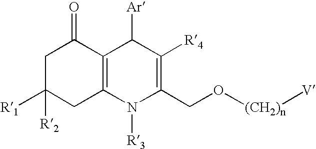
or a pharmaceutically acceptable salt, solvate, clathrate, hydrate or polymorph thereof, wherein:
Ar′ is phenyl or pyridyl, which may be unsubstituted or independently substituted with one or more substituted or unsubstituted lower alkyl, -halo, —CN, —N(R′₅)(R′₅), —OR′₅, —C(O)R′₅, —C(O)₂R′₅, —OC(O)R′₅, —NO₂, or —C(O)N(R′₅)(R′₅) groups, or two adjacent carbon atoms on the phenyl or pyridyl are linked by the group —O—(CH₂)_{q}—O— to form a bicyclic ring system, wherein q is an integer selected from 1, 2, 3 or 4;
V′ is H, N(R′₁₁)(R′₁₁), N₃, substituted or unsubstituted 3-7 membered monocyclic heterocycle or substituted or unsubstituted 8-12 membered bicyclic heterocycle;
each R′₁ and R′₂ may be independently selected from H and substituted or unsubstituted lower alkyl;
R′₃ is —C(O)R₅, —H, or substituted or unsubstituted lower alkyl;
R′₄ is —CN, —CO₂-lower alkyl, —C(O)NHR₅ or a bioisosteric replacement of an ester;
each R′₅ is at each occurrence independently H or substituted or unsubstituted —(C₁-C₁₀) alkyl;
R₁₁′ is at each occurrence independently selected from —H, —OH, —N(R₅)(R₅), —OR₅, —C(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —S(O)N(R₅)(R₅), —SR₅, —S(O)R₅, and —S(O)₂R₅; and
n is an integer selected from the group consisting of 1, 2, 3 and 4; and
wherein R₁ and R₂ are not both H.

24. The compound according to claim 23, wherein
Ar′ is an ortho-substituted or unsubstituted phenyl or pyridyl, wherein if the phenyl or pyridyl is substituted, the substituents are independently selected from the group consisting of substituted or unsubstituted lower alkyl, -halo, —CN, —N(R′₅)(R′₅), —OR′₅, —C(O)R′₅, —C(O)₂R′₅, —OC(O)R′₅, —NO₂, and —C(O)N(R′₅)(R′₅);
V′ is NH₂ or N₃;
R′₁ and R′₂ may be independently selected from H and substituted or unsubstituted lower alkyl;
R′₃ is H, —C(O)R₅ or substituted or unsubstituted lower alkyl;
R′₄ is —CO₂-lower alkyl;
each R′₅ is independently H or substituted or unsubstituted —(C₁-C₁₀) alkyl; and
n is 2 or 3.

25. A compound represented by the following structural formula: 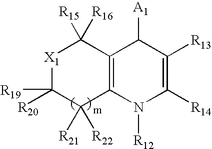
or a pharmaceutically acceptable salt, solvate, clathrate, or prodrug thereof wherein:
m is 0, 1, or 2;
A₁ is an optionally substituted alkyl, an optionally substituted alkenyl, an optionally substituted alkynyl, an optionally substituted cycloalkyl, an optionally substituted cycloalkenyl, or an optionally substituted heterocycloalkyl;
X₁ is O, S, —NR₂₃—, or >CR₁₇R₁₈;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is methyl, A₁ is not a lower alkyl or a lower alkenyl group, wherein the lower alkyl or the lower alkenyl group is substituted with phenyl, nitrophenyl, pyridyl, cyclohexyl, phenylmethoxy, or —S(O)ᵣCH₃, wherein r is 0, 1, or 2;
provided that when R₁₄ is isopropyl or cyclopentyl, R₁₃ is not p-(trifluoromethyl)benzoyl;
provided that when R₁₃ is cyano, R₁₄ is not —SR₂₃;
provided that when R₁₃ is cyano, —C(O)OCH₂CH₃, benzoyl, or acetyl, all of A₁, R₁₄, R₁₉, and R₂₀ are not methyl; and
provided that the compound is not 2,7,7-trimethyl-4-(hex-1-yl)-5-oxo-1,4,5,6,7,8-hexahydroquinoline-3-carboxylic acid ethyl ester, 2-amino-4-ethyl-5-oxo-7,7-dimethyl-1,4,5,6,7,8-hexahydroquinoline-3-carbonitrile, 2-amino-4-methyl-5-oxo-1,4,5,6,7,8-hexahydroquinoline-3-carboxylic acid ethyl ester, 2-phenyl-4-(2-phenylethyn-1-yl)-5-oxo-1,4,5,6,7,8-hexahydroquinoline-3-carboxylic acid ethyl ester, 2-methyl-4-tetrahydrothienyl-5-oxo-1,4,5,6,7,8-hexahydro-quinoline-3-carboxylic acid ethyl ester, 2,7,7-trimethyl-4-cyclohexyl-5-oxo-1,4,5,6,7,8-hexahydroquinoline-3-carbonitrile, 2,7,7-trimethyl-4-isopropyl-5-oxo-1,4,5,6,7,8-hexahydroquinoline-3-carboxylic acid ethyl ester, 2,7,7-trimethyl-4-cyclopentyl-5-oxo-1,4,5,6,7,8-hexahydroquinoline-3-carboxylic acid ethyl ester, 2,4-dimethyl-5-oxo-1,4,5,6,7,8-hexahydroquinoline-3-carboxylic acid ethyl ester, or 2,4-dimethyl-5-oxo-4,7-dihydro-1H-furo[3,4-b]pyridine-3-carboxylic acid methyl ester.

26. The compound of claim 25, wherein R₁₅ and R₁₆ together are ═O and m is 1.

27. The compound of claim 26, wherein A₁ is a substituted or unsubstituted alkyl or a substituted or tinsubstituted cycloalkyl.

28. The compound of claim 27, wherein A₁ is substituted with one or more substituents selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR₂₈R₂₉, —NR₃₀C(O)R₃₁, a halo, —OR₃₀, cyano, nitro, a haloalkoxy, —C(O)R₃₀, —NR₂₈R₂₉, —SR₃₀, —C(O)OR₃₀, —OC(O)R₃₀, —NR₃₀C(O)NR₂₈R₂₉, —OC(O)NR₂₈R₂₉, —NR₃₀C(O)OR₃₁, —S(O)ᵣR₃₀, —S(O)ᵣNR₂₈R₂₉, ═O, ═S, and ═N—R₃₀, wherein:
R₂₈ and R₂₉, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₈ and R₂₉ taken together with the nitrogen to which they are attached is optionally substituted heterocycloalkyl or optionally substituted heteroaryl; and
R₃₀ and R₃₁ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl.

29. The compound of claim 27, wherein A₁ is selected from the group consisting of methyl, ethyl, n-propyl, n-butyl, n-pentyl, n-hexyl, n-heptyl, n-octyl, n-nonyl, n-decyl, isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, cyclopropyl, 1-methylcyclopropyl, cyclopropylmethyl, cyclobutyl, cyclopentyl, cyclohexyl, cycloheptyl, cyclooctyl, cyclononyl, and cyclodecyl.

30. The compound of claim 29, wherein A₁ is methyl, isopropyl, cyclopropyl, cyclopentyl, cyclohexyl, 1-methylcyclopropyl, or cyclopropylmethyl.

31. The compound of claim 26, wherein R₁₉ and R₂₀ are each, independently, a lower alkyl.

32. The compound of claim 26, wherein R₁₃ is —C(O)O-(lower alkyl), —C(O)OH, cyano, —C(O)NR₃₂R₃₂, —C(O)-(lower alkyl), wherein R₃₂, for each occurrence, is —H or a lower alkyl.

33. The compound of claim 26, wherein R₁₄ is cyclopropyl, ethoxymethyl, 2-amino-ethoxymethyl, 2-azido-ethoxymethyl, 2-(2-hydroxy-3-phenoxy-propylamino)-ethoxymethyl, propoxymethyl, isopropoxymethyl, N-mesyl-2-aminoethoxymethyl, N-acetyl-2-aminoethoxymethyl, N-ethyl-2-aminoethoxymethyl, N-methyl-2-aminoethoxymethyl, 2-(1,3-dioxo-1,3-dihydro-isoindol-2-yl)-ethoxymethyl, morpholin-4-yl-methyl, 2-morpholin-4-yl-ethoxymethyl, N,N-dimethylaminomethyl, carbethoxycarbonylmethoxymethyl, N-(2-hydroxyethyl)-N-methylaminomethyl, piperazin-1-yl-methyl, 2-hydroxyethoxymethyl, N,N-dimethylamino-ethoxymethyl, 4-aminobutyl, imidazol-5-yl-methoxymethyl, imidazol-4-yl-methoxymethyl, 2-imidazol-1-yl-ethoxymethyl, 3-imidazol-1-yl-propyl, 3-pyrazol-1-yl-propyl, propoxymethyl, isopropoxymethyl, methoxyethoxymethyl, pyrrol-3-yl-methoxymethyl, pyrrol-2-yl-methoxymethyl, [1,2,4]triazol-3-yl-methoxymethyl, 2H-pyrazol-3-yl-methoxymethyl, 3H-[1,2,3]triazol-4-yl-methoxymethyl, or 2-pyrrol-1-yl-ethoxymethyl.

34. The compound of claim 26, wherein R₁₄ is —NR₃₉R₄₀ or —OR₄₁, wherein:
R₃₉ and R₄₀ are each, independently, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂; or R₃₉ and R₄₀, taken together with the nitrogen to which they are attached are an optionally substituted heterocycloalkyl or optionally substituted heteroaryl;
R₄₁ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃NR₄₄, —S(O)₂R₄₂, or —S(O)R₄₂;
R₄₂ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₄₃ and R₄₄ are each, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl, or R₄₃ and R₄₄ taken together with the nitrogen to which they are attached are an optionally substituted heterocycloalkyl or optionally substituted heteroaryl.

35. The compound of claim 26, wherein X₁ is —O—.

36. The compound of claim 26, wherein X₁ is >CR₁₇R₁₈.

37. A compound represented by the following structural formula: 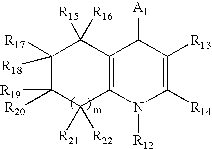
or a pharmaceutically acceptable salt, solvate, clathrate, or prodrug thereof wherein:
m is 0, 1, or 2;
A₁ is an optionally substituted alkyl, an optionally substituted alkenyl, an optionally substituted alkynyl, an optionally substituted cycloalkyl, an optionally substituted cycloalkenyl, or an optionally substituted heterocycloalkyl;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is methyl, A₁ is not a lower alkyl or a lower alkenyl group, wherein the lower alkyl or the lower alkenyl group is substituted with phenyl, nitrophenyl, pyridyl, cyclohexyl, phenylmethoxy, or —S(O)ᵣCH₃, wherein r is 0, 1, or 2;
provided that when R₁₄ is isopropyl or cyclopentyl, R₁₃ is not p-(trifluoromethyl)benzoyl;
provided that when R₁₃ is cyano, R₁₄ is not —SR₂₃;
provided that when R₁₃ is cyano, —C(O)OCH₂CH₃, benzoyl, or acetyl, all of A₁, R₁₄, R₁₉, and R₂₀ are not methyl; and
provided that the compound is not 2,7,7-trimethyl-4-(hex-1-yl)-5-oxo-1,4,5,6,7,8-hexahydroquinoline-3-carboxylic acid ethyl ester, 2-amino-4-ethyl-5-oxo-7,7-dimethyl-1,4,5,6,7,8-hexahydroquinoline-3-carbonitrile, 2-amino-4-methyl-5-oxo-1,4,5,6,7,8-hexahydroquinoline-3-carboxylic acid ethyl ester, 2-phenyl-4-(2-phenylethyn-1-yl)-5-oxo-1,4,5,6,7,8-hexahydroquinoline-3-carboxylic acid ethyl ester, 2-methyl-4-tetrahydrothienyl-5-oxo-1,4,5,6,7,8-hexahydro-quinoline-3-carboxylic acid ethyl ester, 2,7,7-trimethyl-4-cyclohexyl-5-oxo-1,4,5,6,7,8-hexahydroquinoline-3-carbonitrile, 2,7,7-trimethyl-4-isopropyl-5-oxo-1,4,5,6,7,8-hexahydroquinoline-3-carboxylic acid ethyl ester, 2,7,7-trimethyl-4-cyclopentyl-5-oxo-1,4,5,6,7,8-hexahydroquinoline-3-carboxylic acid ethyl ester, or 2,4-dimethyl-5-oxo-1,4,5,6,7,8-hexahydroquinoline-3-carboxylic acid ethyl ester.

38. The compound of claim 37, wherein R₁₅ and R₁₆ together are ═O and m is 1.

39. The compound of claim 38, wherein A₁ is a substituted or unsubstituted alkyl or a substituted or unsubstituted cycloalkyl.

40. The compound of claim 39, wherein A₁ is substituted with one or more substituents selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR₂₈R₂₉, —NR₃₀C(O)R₃₁, a halo, —OR₃₀, cyano, nitro, a haloalkoxy, —C(O)R₃₀, —NR₂₈R₂₉, —SR₃₀, —C(O)OR₃₀, —OC(O)R₃₀, —NR₃₀C(O)NR₂₈R₂₉, —OC(O)NR₂₈R₂₉, —NR₃₀C(O)OR₃₁, —S(O)ᵣR₃₀, —S(O)ᵣNR₂₈R₂₉, ═O, ═S and ═N—R₃₀, wherein:
R₂₈ and R₂₉, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₈ and R₂₉ taken together with the nitrogen to which they are attached is optionally substituted heterocycloalkyl or optionally substituted heteroaryl; and
R₃₀ and R₃₁ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl.

41. The compound of claim 39, wherein A₁ is selected from the group consisting of methyl, ethyl, n-propyl, n-butyl, n-pentyl, n-hexyl, n-heptyl, n-octyl, n-nonyl, n-decyl, isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, cyclopropyl, 1-methylcyclopropyl, cyclopropylmethyl, cyclobutyl, cyclopentyl, cyclohexyl, cycloheptyl, cyclooctyl, cyclononyl, and cyclodecyl.

42. The compound of claim 41, wherein A₁ is methyl, isopropyl, cyclopropyl, cyclopentyl, cyclohexyl, 1-methylcyclopropyl, or cyclopropylmethyl.

43. The compound of claim 38, wherein R₁₉ and R₂₀ are each, independently, a lower alkyl.

44. The compound of claim 38, wherein R₁₃ is —C(O)O-(lower alkyl), —C(O)OH, cyano, —C(O)NR₃₂R₃₂, —C(O)-(lower alkyl), wherein R₃₂, for each occurrence, is —H or a lower alkyl.

45. The compound of claim 38, wherein R₁₄ is cyclopropyl, ethoxymethyl, 2-amino-ethoxymethyl, 2-azido-ethoxymethyl, 2-(2-hydroxy-3-phenoxy-propylamino)-ethoxymethyl, propoxymethyl, isopropoxymethyl, N-mesyl-2-aminoethoxymethyl, N-acetyl-2-aminoethoxymethyl, N-ethyl-2-aminoethoxymethyl, N-methyl-2-aminoethoxymethyl, 2-(1,3-dioxo-1,3-dihydro-isoindol-2-yl)-ethoxymethyl, morpholin-4-yl-methyl, 2-morpholin-4-yl-ethoxymethyl, N,N-dimethylaminomethyl, carbethoxycarbonylmethoxymethyl, N-(2-hydroxyethyl)-N-methylarninomethyl, piperazin-1-yl-methyl, 2-hydroxyethoxymethyl, N,N-dimethylamino-ethoxymethyl, 4-aminobutyl, imidazol-5-yl-methoxymethyl, imidazol-4-yl-methoxymethyl, 2-imidazol-1-yl-ethoxymethyl, 3-imidazol-1-yl-propyl, 3-pyrazol-1-yl-propyl, propoxymethyl, isopropoxymethyl, methoxyethoxymethyl, pyrrol-3-yl-methoxymethyl, pyrrol-2-yl-methoxymethyl, [1,2,4]triazol-3-yl-methoxymethyl, 2H-pyrazol-3-yl-methoxymethyl, 3H-[1,2,3]triazol-4-yl-methoxymethyl, or 2-pyrrol-1-yl-ethoxymethyl.

46. The compound of claim 38, wherein R₁₄ is —NR₃₉R₄₀ or —OR₄₁, wherein:
R₃₉ and R₄₀ are each, independently, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂; or R₃₉ and R₄₀, taken together with the nitrogen to which they are attached are an optionally substituted heterocycloalkyl or optionally substituted heteroaryl;
R₄₁, is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂;
R₄₂ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₄₃ and R₄₄ are each, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl, or R₄₃ and R₄₄ taken together with the nitrogen to which they are attached are an optionally substituted heterocycloalkyl or optionally substituted heteroaryl.

47. A compound represented by the following structural formula: 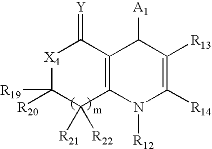
or a pharmaceutically acceptable salt, solvate, clathrate, or prodrug thereof wherein:
m is 0, 1, or 2;
A₁ is an optionally substituted alkyl, an optionally substituted alkenyl, an optionally substituted alkynyl, an optionally substituted cycloalkyl, an optionally substituted cycloalkenyl, or an optionally substituted heterocycloalkyl;
X₄ is O, S, or —NR₂₃—;
Y is O or S;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₃R₂₅)R₂₃; or —CH(SR₂₃)R₂₃;
R₁₄ is H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl,
provided that the compound is not 2,4-dimethyl-5-oxo-4,7-dihydro-1H-furo[3,4-b]pyridine-3-carboxylic acid methyl ester.

48. The compound of claim 47, wherein Y is ═O, X₄ is —O—, and m is 1.

49. The compound of claim 48, wherein A₁ is a substituted or unsubstituted alkyl or a substituted or unsubstituted cycloalkyl.

50. The compound of claim 49, wherein A₁ is substituted with one or more substituents selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR₂₈R₂₉, —NR₃₀C(O)R₃₁, a halo, —OR₃₀, cyano, nitro, a haloalkoxy, —C(O)R₃₀, —NR₂₈R₂₉, —SR₃₀, —C(O)OR₃₀, —OC(O)R₃₀, —NR₃₀C(O)NR₂₈R₂₉, —OC(O)NR₂₈R₂₉, —NR₃₀C(O)OR₃₁, —S(O)ᵣR₃₀, —S(O)ᵣNR₂₈R₂₉, ═O, ═S ═N—R₃₀, wherein:
R₂₈ and R₂₉, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₈ and R₂₉ taken together with the nitrogen to which they are attached is optionally substituted heterocycloalkyl or optionally substituted heteroaryl; and
R₃₀ and R₃₁ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl.

51. The compound of claim 49, wherein A₁ is selected from the group consisting of methyl, ethyl, n-propyl, n-butyl, n-pentyl, n-hexyl, n-heptyl, n-octyl, n-nonyl, n-decyl, isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, cyclopropyl, 1-methylcyclopropyl, cyclopropylmethyl, cyclobutyl, cyclopentyl, cyclohexyl, cycloheptyl, cyclooctyl, cyclononyl, and cyclodecyl.

52. The compound of claim 51, wherein A₁ is methyl, isopropyl, cyclopropyl, cyclopentyl, cyclohexyl, 1-methylcyclopropyl, or cyclopropylmethyl.

53. The compound of claim 48, wherein R₁₉ and R₂₀ are each, independently, a lower alkyl.

54. The compound of claim 48, wherein R₁₃ is —C(O)O-(lower alkyl), —C(O)OH, cyano, —C(O)NR₃₂R₃₂, —C(O)-(lower alkyl), wherein R₃₂, for each occurrence, is —H or a lower alkyl.

55. The compound of claim 48, wherein R₁₄ is cyclopropyl, ethoxymethyl, 2-amino-ethoxymethyl, 2-azido-ethoxymethyl, 2-(2-hydroxy-3-phenoxy-propylamino)-ethoxymethyl, propoxymethyl, isopropoxymethyl, N-mesyl-2-aminoethoxymethyl, N-acetyl-2-aminoethoxymethyl, N-ethyl-2-aminoethoxymethyl, N-methyl-2-aminoethoxymethyl, 2-(1,3-dioxo-1,3-dihydro-isoindol-2-yl)-ethoxymethyl, morpholin-4-yl-methyl, 2-morpholin-4-yl-ethoxymethyl, N,N-dimethylaminomethyl, carbethoxycarbonylmethoxymethyl, N-(2-hydroxyethyl)—N-methylaminomethyl, piperazin-1-yl-methyl, 2-hydroxyethoxymethyl, N,N-dimethylamino-ethoxymethyl, 4-aminobutyl, imidazol-5-yl-methoxymethyl, imidazol-4-yl-methoxymethyl, 2-imidazol-1-yl-ethoxymethyl, 3-imidazol-1-yl-propyl, 3-pyrazol-1-yl-propyl, propoxymethyl, isopropoxymethyl, methoxyethoxymethyl, pyrrol-3-yl-methoxymethyl, pyrrol-2-yl-methoxymethyl, [1,2,4]triazol-3-yl-methoxymethyl, 2H-pyrazol-3-yl-methoxymethyl, 3H-[1,2,3]triazol-4-yl-methoxymethyl, or 2-pyrrol-1-yl-ethoxymethyl.

56. The compound of claim 58, wherein R₁₄ is —NR₃₉R₄₀ or —OR₄₁, wherein:
R₃₉ and R₄₀ are each, independently, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂; or R₃₉ and R₄₀, taken together with the nitrogen to which they are attached are an optionally substituted heterocycloalkyl or optionally substituted heteroaryl;
R₄₁ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂;
R₄₂ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₄₃ and R₄₄ are each, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl, or R₄₃ and R₄₄ taken together with the nitrogen to which they are attached are an optionally substituted heterocycloalkyl or optionally substituted heteroaryl.

57. A compound represented by the following structural formula: 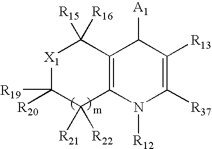
or a pharmaceutically acceptable salt, solvate, clathrate, or prodrug thereof wherein:
m is 0, 1, or 2;
A₁ is an optionally substituted alkyl, an optionally substituted alkenyl, an optionally substituted alkynyl, an optionally substituted cycloalkyl, an optionally substituted cycloalkenyl, or an optionally substituted heterocycloalkyl;
X₁ is O, S, —NR₂₃—, or >CR₁₇R₁₈;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₃₇ is -halo, —NO₂, —CN, —OH, —N(R₃₃)(R₃₃), —OR₃₃, —C(O)R₃₄, —OC(O)R₃₄, —C(O)NHC(O)R₃₃, substituted or unsubstituted —(C₂-C₁₀)alkenyl, substituted or unsubstituted —(C₂-C₁₀)alkynyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted naphthyl, substituted or unsubstituted benzyl, a substituted or unsubstituted heteroalkyl, —(C₁-C₆)alkyl-Z₁-(C₁-C₁₀)alkyl-R₃₅, —(C₁-C₁₀)alkyl-R₃₆, —(C₁-C₁₀)alkyl-N(R₃₄)(R₃₄), —CO ₂R₃₄, —NHC(O)R₃₄, —NHC(O)NHR₃₄, —C(O)NHR₃₄, —OC(O)R₃₄, —OC(O)OR₃₄, or —S(O)N(R₃₄)(R₃₄);
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₇ is H, alkyl, aryl or acetyl;
Z₁, for each occurrence, is independently, —O—, —S—, —N(R₃₄)—, —C(O)—, —OC(O)—, —C(O)N(R₃₄)C(O)—, substituted or unsubstituted —(C₃-C₁₀)cycloalkyl-, substituted or unsubstituted —(C₈-C₁₄)bicycloalkyl-, substituted or unsubstituted —(C₅-C₁₀)cycloalkenyl-, substituted or unsubstituted —(C₃-C₁₀)heterocycle-, substituted or unsubstituted phenyl, substituted or unsubstituted naphthyl, substituted or unsubstituted benzyl, —C(O)O—, —C(O)OC(R₃₄)(R₃₄)—, —N(R₃₄)C(O)—, —N(R₃₄)C(O)NR₃₄—, —C(O)NR₃₄—, —OC(O)O—, —S(O)N(R₃₄)—, —S(O)—, or —S(O)₂—;
R₃₃, for each occurrence, is, independently, a substituted or unsubstituted alkyl;
R₃₄, for each occurrence, is, independently, —H or a substituted or unsubstituted alkyl;
R₃₅, for each occurrence, is, independently, selected from —H, halo, —CN, —N₃, —NO₂, —CN, —OH, —N(R₃₄)(R₃₄), —OR₃₄, —C(O)R₃₄, —OC(O)R₃₄, —C(O)NHC(O)R₃₄, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₃₄, —C(O)OCH(R₃₄)(R₃₄), —NHC(O)R₃₄, —NHC(O)NHR₃₄, —C(O)NHR₃₄, —OC(O)R₃₄, —OC(O)OR₃₄, —NR₃₄S(O)₂R₃₃, —S(O)N(R₃₄)(R₃₄), —SR₃₄, —S(O)R₃₄, and —S(O)₂R₃₄; and
R₃₆, for each occurrence, is, independently, selected from halo, —CN, —N₃, —NO₂, —CN, —OH, —N(R₃₄)(R₃₄), —OR₃₄, —C(O)R₃₄, —OC(O)R₃₄, —C(O)NHC(O)R₃₄, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₃₄, —C(O)OCH(R₃₄)(R₃₄), —NHC(O)R₃₄, —NHC(O)NHR₃₄, —C(O)NHR₃₄, —OC(O)R₃₄, —OC(O)OR₃₄, —S(O)N(R₃₄)(R₃₄), —SR₃₄, —S(O)R₃₄, and —S(O)₂R₃₄.

58. The compound of claim 57, wherein R₁₅ and R₁₆ taken together are ═O, X₁ is >CR₁₇R₁₈, and m is 1.

59. The compound of claim 58, wherein A₁ is a substituted or unsubstituted alkyl or a substituted or unsubstituted cycloalkyl.

60. The compound of claim 59, wherein A₁ is substituted with one or more substituents selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR₂₈R₂₉, —NR₃₀C(O)R₃₁, a halo, —OR₃₀, cyano, nitro, a haloalkoxy, —C(O)R₃₀, —NR₂₈R₂₉, —SR₃₀, —C(O)OR₃₀, —OC(O)R₃₀, —NR₃₀C(O)NR₂₈R₂₉, —OC(O)NR₂₈R₂₉, —NR₃₀C(O)OR₃₁, —S(O)ᵣR₃₀, —S(O)ᵣNR₂₈R₂₉, ═O, ═S ═N—R₃₀, wherein:
R₂₈ and R₂₉, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₈ and R₂₉ taken together with the nitrogen to which they are attached is optionally substituted heterocycloalkyl or optionally substituted heteroaryl; and
R₃₀ and R₃₁ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl.

61. The compound of claim 59, wherein A₁ is selected from the group consisting of methyl, ethyl, n-propyl, n-butyl, n-pentyl, n-hexyl, n-heptyl, n-octyl, n-nonyl, n-decyl, isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl, cyclopropyl, 1-methylcyclopropyl, cyclopropylmethyl, cyclobutyl, cyclopentyl, cyclohexyl, cycloheptyl, cyclooctyl, cyclononyl, and cyclodecyl.

62. The compound of claim 61, wherein A₁ is methyl, isopropyl, cyclopropyl, cyclopentyl, cyclohexyl, 1-methylcyclopropyl, or cyclopropylmethyl.

63. The compound of claim 58, wherein R₁₉ and R₂₀ are each, independently, a lower alkyl.

64. The compound of claim 58, wherein R₁₃ is —C(O)O-(lower alkyl), —C(O)OH, cyano, —C(O)NR₃₂R₃₂, —C(O)-(lower alkyl), wherein R₃₂, for each occurrence, is —H or a lower alkyl.

65. The compound of claim 58, wherein R₃₇ is ethoxymethyl, 2-amino-ethoxymethyl, 2-azido-ethoxymethyl, propoxymethyl, isopropoxymethyl, N-mesyl-2-aminoethoxymethyl, N-acetyl-2-aminoethoxymethyl, N-ethyl-2-aminoethoxymethyl, N-methyl-2-aminoethoxymethyl, 2-(1,3-dioxo-1,3-dihydro-isoindol-2-yl)-ethoxymethyl, morpholin-4-yl-methyl, 2-morpholin-4-yl-ethoxymethyl, N,N-dimethylaminomethyl, carbethoxycarbonylmethoxymethyl, N-(2-hydroxyethyl)—N-methylaminomethyl, piperazin-1-yl-methyl, 2-hydroxyethoxymethyl, N,N-dimethylamino-ethoxymethyl, 4-aminobutyl, imidazol-5-yl-methoxymethyl, imidazol-4-yl-methoxymethyl, 2-imidazol-1-yl-ethoxymethyl, 3-imidazol-1-yl-propyl, 3-pyrazol-1-yl-propyl, isopropoxymethyl, methoxyethoxymethyl, pyrrol-3-yl-methoxymethyl, pyrrol-2-yl-methoxymethyl, [1,2,4]triazol-3-yl-methoxymethyl, 2H-pyrazol-3-yl-methoxymethyl, 3H-[1,2,3]triazol-4-yl-methoxymethyl, or 2-pyrrol-1-yl-ethoxymethyl.

66. The compound of claim 58, wherein R₃₇ is —NR₃₉R₄₀ or —OR₄₁, wherein:
R₃₉ and R₄₀ are each, independently, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂; or R₃₉ and R₄₀, taken together with the nitrogen to which they are attached are an optionally substituted heterocycloalkyl or optionally substituted heteroaryl;
R₄₁ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂;
R₄₂ is —H, an optionally substituted alkyl, an optionally substituted alkenyl, an optionally substituted alkynyl, anoptionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;and
R₄₃ and R₄₄ are each, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl, or R₄₃ and R₄₄ taken together with the nitrogen to which they are attached are an optionally substituted heterocycloalkyl or optionally substituted heteroaryl.

67. A compound selected from the group consisting of:
2-(2-amino-ethoxymethyl)-4-(2-chloro-phenyl)-7,7-dimethyl-5-oxo-1,4,5,6,7,8- hexahydro-quinoline-3-carboxylic acid ethyl ester;
2-(2-azido-ethoxymethyl)-4-(2-chloro-phenyl)-7,7-dimethyl-5-oxo-1,4,5,6,7,8-hexahydro-quinoline-3-carboxylic acid ethyl ester;
2-(2-amino-ethoxymethyl)-4-benzo[1,3]dioxol-5-yl-7,7-dimethyl-5-oxo-1,4,5,6,7,8-hexahydro-quinoline-3-carboxylic acid ethyl ester;
2-(2-amino-ethoxymethyl)-4-(2-chloro-phenyl)-1,7,7-trimethyl-5-oxo-1,4,5,6,7,8-hexahydro-quinoline-3-carboxylic acid ethyl ester;
2-(2-amino-ethoxymethyl)-4-(2-chloro-phenyl)-7,7-dimethyl-5-oxo-1,4,5,6,7,8-hexahydro-quinoline-3-carboxylic acid ethyl ester (complex with benzenesulfonic acid);
2-(2-amino-ethoxymethyl)-4-(2,3-dichloro-phenyl)-7,7-dimethyl-5-oxo-1,4,5,6,7,8-hexahydro-quinoline-3-carboxylic acid ethyl ester:
2-(2-amino-ethoxymethyl)-7,7-dimethyl-4-(3- nitro-phenyl)-5-oxo-1,4,5,6,7,8-hexahydro-quinoline-3-carboxylic acid ethyl ester;
2-(2-amino-ethoxymethyl)-4-(3-amino-phenyl)-7,7-dimethyl-5-oxo-1,4,5,6,7,8-hexahydro-quinoline-3-carboxylic acid ethyl ester;
2-(2-amino-ethoxymethyl)-7,7-dimethyl-5-oxo-4-pyridin-2-yl-1,4,5,6,7,8-hexahydro-quinoline-3-carboxylic acid ethyl ester;
2-(2-amino-ethoxymethyl)-4-(2-chloro-phenyl)-5-oxo-1,4,5,6,7,8-hexahydro-quinoline-3-carboxylic acid ethyl ester;
2-(2-amino-ethoxymethyl)-7,7-dimethyl-4-(2-nitro-phenyl)-5-oxo-1,4,5,6,7,8-hexahydro-quinoline-3-carboxylic acid ethyl ester;
2-(2-amino-ethoxymethyl)-4-(2-fluoro-phenyl)-7,7-dimethyl-5-oxo-1,4,5,6,7,8-hexahydro-quinoline-3-carboxylic acid ethyl ester;
2-(2-amino-ethoxymethyl)-4-(2-chloro-phenyl)-7-isopropyl-5-oxo-1,4,5,6,7,8-hexa-hydroquinoline-3-carboxylic acid ethyl ester;
2-(2-amino-ethoxymethyl)-4-(2-chloro-phenyl)-7- methyl-5-oxo-1,4,5,6,7,8-hexa-hydroquinoline-3-carboxylic acid ethyl ester;
2-(2-Amino-ethoxymethyl)-4-(4-chloro-phenyl)-7,7-dimethyl-5-oxo-1,4,5,6,7,8-hexahydro-quinoline-3-carboxylic acid ethyl ester;
2-(2-Amino-ethoxymethyl)-4-(2-methoxy-phenyl)-7,7-dimethyl-5-oxo-1,4,5,6,7,8-hexahydro-quinoline-3-carboxylic acid ethyl ester;
2-(2-Amino-ethoxymethyl)-4-(2-cyano-phenyl)-7,7-dimethyl-5-oxo-1,4,5,6,7,8-hexahydro-quinoline-3-carboxylic acid ethyl ester;
4-(2-chloro-phenyl)-2-[2-(2-hydroxy-3-phenoxy-propylamino)-ethoxymethyl]-7,7-dimethyl-5-oxo-1,4,5,6,7,8-hexahydro-quinoline-3-carboxylic acid ethyl ester;
2-(2-amino-ethoxymethyl)-4-(2-chloro-phenyl)-7,7-dimethyl-5-oxo-1,4,5,6,7,8-hexahydro-quinoline-3-carboxylic acid methyl ester;
2-(2-amino-ethoxymethyl)-4-(2-chloro-phenyl)-7,7-dimethyl-5-oxo-1,4,5,6,7,8-hexahydroquinoline-3-carboxylic acid methyl ester (complex with benzenesulfonic acid);
2-(2-Amino-ethoxymethyl)-4-{5-chloro-2-[4-(2-hydroxy-3-phenoxy-propylamino)-butoxy]-phenyl }-7,7-dimethyl-5-oxo-1,4,5,6,7,8-hexahydroquinoline-3-carboxylic acid ethyl ester;
4-(2-chloro-phenyl)-2-[2-(2-hydroxy-3-phenoxy-propylamino)-ethoxymethyl1-7,7-dimethyl-5-oxo-1,4,5,6,7,8-hexahydro-quinoline-3-carboxylic acid methyl ester;
2-(2-Amino-ethoxymethyl)-4-(2-chloro-phenyl)-6,6-dimethyl-5-oxo-1,4,5,6,7,8-hexahydro-quinoline-3-carboxylic acid ethyl ester;
2-(2-Amino-ethoxymethyl)-4-(2-fluoro-phenyl)-7-methyl-5-oxo-1,4,5,6,7,8-hexahydroquinoline-3-carboxylic acid ethyl ester;
2-(2-Amino-ethoxymethyl)-4-(2-fluoro-phenyl)-7-isopropyl-5-oxo-1,4,5,6,7,8-hexahydro-quinoline-3-carboxylic acid ethyl ester;
2-(2-Amino-ethoxymethyl)-4-(2-cyano-phenyl)-7-methyl-5-oxo-1,4,5,6,7,8-hexahydro-quinoline-3-carboxylic acid ethyl ester;
2-(2-Amino-ethoxymethyl)-4-(2-cyano-phenyl)-7-isopropyl-5-oxo-1,4,5,6,7,8-hexahydro-quinolone-3-carboxylic acid ethyl ester;
2-(2-Amino-ethoxymethyl)-4-(2-cyano-phenyl)-5-oxo-1,4,5,6,7,8-hexahydro-quinoline-3-carboxylic acid ethyl ester;
2-(2-Amino-ethoxymethyl)-4-(3,5-dichloro-phenyl)-7,7-dimethyl-5-oxo-1,4,5,6,7,8-hexahydro-quinoline-3-carboxylic acid ethyl ester;
2-(2-Amino-ethoxymethyl)-4-(3,4-dichloro-phenyl)-7,7-dimethyl-5-oxo-1,4,5,6,7,8-hexahydro-quinoline-3-carboxylic acid ethyl ester;
2-(2-Amino-ethoxymethyl)-4-(2,3-dichloro-phenyl)-7-isopropyl-5-oxo-1,4,5,6,7,8-hexahydro-quinoline-3-carboxylic acid ethyl ester;
2-(2-Amino-ethoxymethyl)-4-(2,3-dichloro-phenyl)-7-methyl-5-oxo-1,4,5,6,7,8-hexahydro-quinoline-3-carboxylic acid ethyl ester;
2-(2-Amino-ethoxymethyl)-4-(2,6-dichloro-phenyl)-7,7-dimethyl-5-oxo-1,4,5,6,7,8-hexahydroquinoline-3-carboxylic acid ethyl ester;
2-(2-Amino-ethoxymethyl)-4-(2,5-dichloro-phenyl)-7-methyl-5-oxo-1,4,5,6,7,8-hexahydro-quinoline-3-carboxylic acid ethyl ester;
2-(2-Amino-ethoxymethyl)-4-(2,4-dichloro-phenyl)-7-methyl-5-oxo-1,4,5,6,7,8-hexahydro-quinoline-3-carboxylic acid ethyl ester;
S-(−)-2-(2-amino-ethoxymethyl)-4-(2-chloro-phenyl)-7,7-dimethyl-5-oxo-1,4,5,6,7,8-hexahydro-quinoline-3-carboxylic acid ethyl ester (complex with benzenesulfonic acid);
2-(2-Acetylamino-ethoxymethyl)-4-(2-chloro-phenyl)-7,7-dimethyl-5-oxo-1,4,5,6,7,8-hexahydro-quinoline-3-carboxylic acid ethyl ester;
2-(2-Amino-ethoxymethyl)-4-(2-chloro-phenyl)-7,7-dimethyl-5-oxo-1,4,5,6,7,8-hexahydro-quinoline-3-carboxylic acid;
2-(2-Amino-ethoxymethyl)-7,7-dimethyl-5-oxo-4-pyridin-3-yl-1,4,5,6,7,8-hexahydro-quinoline-3-carboxylic acid ethyl ester;
4-(2-chloro-phenyl)-2-(2-ethylamino-ethoxymethyl)-7,7-dimethyl-5-oxo-1,4,5,6,7,8-hexahydro-quinoline-3-carboxylic acid ethyl ester;
2-(2-Amino-ethoxymethyl)-4-(2-chloro-phenyl)-7,7-dimethyl-5-oxo-1,4,5,6,7,8-hexahydro-quinoline-3-carbonitrile;
Benzenesulfonate2-(3-ethoxycarbonyl-7,7-dimethyl-5-oxo-4-pyridin-3-yl-1,4,5,6,7,8-hexahydro-quinolin-2-ylmethoxy)-ethyl-ammonium;
2-(2-Amino-ethoxymethyl)-7,7-dimethyl-5-oxo-4-quinolin-3-yl-1,4,5,6,7,8-hexahydro-quinoline-3-carboxylic acid ethyl ester;
4-(2-chloro-phenyl)-7,7-dimethyl-2-(2-methylamino-ethoxymethyl)-5-oxo-1,4,5,6,7,8-hexahydro-quinoline-3-carboxylic acid ethyl ester;
2-(2-Amino-ethoxymethyl)-7,7-dimethyl-5-oxo-4-pyridin-4-yl-1,4,5,6,7,8-hexahydro-quinoline-3-carboxylic acid ethyl ester;
2-(2-Amino-ethoxymethyl)-5-oxo-4-pyridin-3-yl-1,4,5,6,7,8-hexahydro-quinoline-3-carboxylic acid ethyl ester;
2-(2-Amino-ethoxymethyl)-4-(4-chloro-1,2,4,5-tetradeutium-phenyl)-7,7-dimethyl-5-oxo-1,4,5,6,7,8-hexahydro-quinoline-3-carboxylic acid ethyl ester;
2-[2-(1,3-Dioxo-1,3-dihydro-isoindol-2-yl)-ethoxymethyll-7,7-dimethyl-5-oxo-4-pyridin-4-yl-1,4,5,6,7,8-hexahydro-quinoline-3-carboxylic acid ethyl ester;
2-[2-(1,3-Dioxo-1,3-dihydro-isoindol-2-yl)-ethoxymethyl]-5-oxo-4-pyridin-3-yl-1,4,5,6,7,8-hexahydro-quinoline-3-carboxylic acid ethyl ester;
2-(2-Azido-ethoxymethyl)-7,7-dimethyl-4-(3-nitro-phenyl)-5-oxo-1,4,5,6,7,8-hexahydro-quinoline-3-carboxylic acid ethyl ester;
2-(2-azido-ethoxymethyl)-4-benzo[1,3]dioxol-5-yl-7,7-dimethyl-5-oxo-1,4,5,6,7,8-hexahydro-quinoline-3-carboxylic acid ethyl ester;
2-(2-azido-ethoxymethyl)-4-(2-chloro-phenyl)-1,7,7-trimethyl-5-oxo-1,4,5,6,7,8-hexahydro-quinoline-3-carboxylic acid ethyl ester;
2-(2-amino-ethoxymethyl)-4-(2-chloro-phenyl)-5-oxo-4,5,6,7-tetrahydro-1H-[1]pyrindine-3-carboxylic acid ethyl ester;
9-(2-chloro-phenyl)-6,6-dimethyl-5,6,7,9-tetrahydro-3H,4H-furo[3,4-b]quinoline-1,8-dione;
(+)-(R)-2-(2-amino-ethoxymethyl)-4-(2-chloro-phenyl)-7,7-dimethyl-5-oxo-1,4,5,6,7,8-hexah,dro-quinoline-3-carboxylic acid ethyl ester: compound with benzenesulfonic acid;
(+)-(R)-2-(2-amino-ethoxymethyl)-4-(2-chloro-phenyl)-7,7-dimethyl-5-oxo-1,4,5,6,7,8-hexahydro-quinoline-3-carboxylic acid ethyl ester;
(−)-(S)-2-(2-amino-ethoxymethyl)-4-(2-chloro-phenyl)-7,7-dimethyl-5-oxo-1,4,5,6,7,8-hexahydro-quinoline-3-carboxylic acid ethyl ester;
2-(2-Amino-ethoxymethyl)-4-(1H-indol-3-yl)-7,7-dimethyl-5-oxo-1,4,5,6,7,8-hexahydro-quinoline-3-carboxylic acid ethyl ester;
4-(2-chloro-phenyl)-2-ethoxycarbonylmethoxymethyl-7,7-dimethyl-5-oxo-1,4,5,6,7,8-hexahydro-quinoline-3-carboxylic acid ethyl ester;
4-(2-chloro-phenyl)-2-(hydroxyl-ethoxymethyl)-7,7-dimethyl-5-oxo-1,4,5,6,7,8-hexahydro-quinoline-3-carboxylic acid ethyl ester;
9-(2-chloro-phenyl)-4-ethyl-6,6-dimethyl-5,6,7,9-tetrahydro-3H,4H-furo[3,4b]quinoline-1,8-dione;
4-(2-chloro-phenyl)-7,7-dimethyl-5-oxo-2-piperazin-1-ylmethyl-1,4,5,6,7,8-hexahydro-quinoline-3-carboxylic acid ethyl ester;
4-(2-chloro-phenyl)-2-{[(2-hydroxy-ethyl)-methyl-amino]-methyl}-7,7-dimethyl-5-oxo-1,4,5,6,7.8-hexahydro-quinoline-3-carboxylic acid ethyl ester;
2-(2-Amino-ethoxymethyl)-4-(2-hydroxy-phenyl)-7,7-dimethyl-5-oxo-1,4,5,6,7,8-hexahydro-quinoline-3-carboxylic acid ethyl ester;
2-(2-Amino-ethoxymethyl)-7,7-dimethyl-5-oxo-4-pyridin-3-yl-1,4,5,6,7,8-hexahydro-quinoline-3-carboxylic acid methyl ester;
2-(2-Amino-ethoxymethyl)-1,7,7-trimethyl-5-oxo-4-pyridin-3-yl-1,4,5,6,7,8-hexahydro-quinoline-3-carboxylic acid ethyl ester;
2-(2-hydroxy-ethoxymethyl)-7,7-dimethyl-5-oxo-4-pyridin-3-yl-1,4,5,6,7,8-hexahydro-quinoline-3-carboxylic acid ethyl ester;
2-(2-Dimethylamino-ethoxymethyl)-7,7-dimethyl-5-oxo-4-pyridin-3yl-1,4,5,6,7,8,-hexahydro-quinoline-3-carboxylic acid ethyl ester;
2-(4-Amino-butyl)-7,7-dimethyl-5-oxo-4-pyridin-3yl-1,4,5,6,7,8,-hexahydro-quinoline-3-carboxylic acid ethyl ester;
2-(2-Amino-ethoxymethyl)-4-(2-chloro-phenyl)-7,7-dimethyl-4,6,7,8-tetrahydro-1H-quinolin-5-one;
4-(2-chloro-phenyl)-2-(2-dimethylamino-ethoxymethyl)-7,7-dimethyl-5-oxo-1,4,5,6,7,8-hexahydro-quinoline-3-carboxylic acid ethyl ester;
(+)-(R)-2-(2-Amino-ethoxymethyl)-7,7-dimethyl-5-oxo-4-pyridin-3-yl-1,4,5,6,7,8-hexahydro-quinoline-3-carboxylic acid ethyl ester;
(−)-(S)-2-(2-Amino-ethoxymethyl)-7,7-dimethyl-5-oxo-4-pyridin-3-yl-1,4,5,6,7,8-hexahydro-quinoline-3-carboxylic acid ethyl ester;
2-(2-Amino-ethoxymethyl)-7,7-dimethyl-5-oxo-4-pyridin-3-yl-1,4,5,6,7,8-hexahydro-quinoline-3-carbonitrile;
2-(2-Amino-ethoxymethyl)-7,7-dimethyl-5-oxo-4-pyridin-4-yl-1,4,5,6,7,8-hexahydro-quinoline-3-carbonitrile;
2-(2-Amino-ethoxymethyl)-7,7-dimethyl-5-oxo-4-(6-trifluoromethyl-pyridin-3-yl)-1,4,5,6,7,8-hexahydro-quinoline-3-carboxylic acid ethyl ester;
2-(2-Amino-ethoxymethyl)-4-(4-chloro-pyridin-3-yl)-7,7-dimethyl-5-oxo-1,4,5,6,7,8-hexahydro-quinoline-3-carboxylic acid ethyl ester;
2-(2-Amino-ethoxymethyl)-7,7-dimethyl-5-oxo-4-(6-trifluoromethyl-pyridin-3-yl)-1,4,5,6,7,8-hexahydro-quinoline-3-carbonitrile;
2-(2-Amino-ethoxymethyl)-4-(4-chloro-pyridin-3-yl)-7,7-dimethyl-5-oxo-1,4,5,6,7,8-hexahydro-quinoline-3-carbonitrile;
2-(2-Amino-ethoxymethyl)-4-(3-chloro-pyridin-4-yl)-7,7-dimethyl-5-oxo-1,4,5,6,7,8-hexahydro-quinoline-3-carboxylic acid ethyl ester;
2-(2-Amino-ethoxymethyl)-4-(3-chloro-pyridin-4-yl)-7,7-dimethyl-5-oxo-1,4,5,6,7,8-hexahydro-quinoline-3-carbonitrile
2-(2-Amino-ethoxymethyl)-7,7-dimethyl-5-oxo-4-(1-oxy-pyridin-4-yl)-1,4,5,6,7,8-hexahydro-quinoline-3-carboxylic acid ethyl ester;
2-(2-Amino-ethoxymethyl)-7,7-dimethyl-5-oxo-4-(1-oxy-pyridin-4-yl)-1,4,5,6,7,8-hexahydro-quinoline-3-carbonitrile;
2-(2-Amino-ethoxymethyl)-7,7-dimethyl-5-oxo-4-(1-oxy-pyridin-3-yl)-1,4,5,6,7,8-hexahydro-quinoline-3-carboxylic acid ethyl ester;
2-(2-Amino-ethoxymethyl)-7,7-dimethyl-5-oxo-4-(1-oxy-pyridin-3-yl)-1,4,5,6,7,8-hexahydro-quinoline-3-carbonitrile;
2-(2-Amino-ethoxymethyl)-7,7-dimethyl-5-oxo-4-pyridazin-4-yl-1,4,5,6,7,8-hexahydro-quinoline-3-carboxylic acid ethyl ester;
2-(2-Amino-ethoxymethyl)-7,7-dimethyl-5-oxo-4-pyridazin-4-yl-1,4,5,6,7,8-hexahydro-quinoline-3-carbonitrile;
2-(2-Amino-ethoxymethyl)-7,7-dimethyl-5-oxo-4-pyridazin-3-yl-1,4,5,6,7,8-hexahydro-quinoline-3-carboxylic acid ethyl ester;
2-(2-Amino-ethoxymethyl)-7,7-dimethyl-5-oxo-4-pyridazin-3-yl-1,4,5,6,7,8-hexahydro-quinoline-3-carbonitrile;
2-(2-Amino-ethoxymethyl)-7,7-dimethyl-5-oxo-4-pyrimidin-5-yl-1,4,5,6,7,8-hexahydro-quinoline-3-carboxylic acid ethyl ester;
2-(2-Amino-ethoxymethyl)-7,7-dimethyl-5-oxo-4-pyrimidin-5-yl-1,4,5,6,7,8-hexahydro-quinoline-3-carbonitrile;
2-(2-Amino-ethoxymethyl)-7,7-dimethyl-5-oxo-4-pyrazin-2-yl-1,4,5,6,7,8-hexahydro-quinoline-3-carboxylic acid ethyl ester;
2-(2-Amino-ethoxymethyl)-7,7-dimethyl-5-oxo-4-pyrazin-2-yl-1,4,5,6,7,8-hexahydro-quinoline-3-carbonitrile;
2-(2-Amino-ethoxymethyl)-7,7-dimethyl-5-oxo-4-pyrimidin-4-yl-1,4,5,6,7,8-hexahydro-quinoline-3-carboxylic acid ethyl ester;
2-(2-Amino-ethoxymethyl)-7,7-dimethyl-5-oxo-4-pyrimidin-4-yl-1,4,5,6,7,8-hexahydro-quinoline-3-carbonitrile;
2-(2-Amino-ethoxymethyl)-4-furan-2-yl-7,7-dimethyl-5-oxo-1,4,5,6,7,8-hexahydro-quinoline-3-carboxylic acid ethyl ester;
2-(2-Amino-ethoxymethyl)-4-furan-2-yl-7,7-dimethyl-5-oxo-1,4,5,6,7,8-hexahydro-quinoline-3-carbonitrile;
2-(2-Amino-ethoxymethyl)-7,7-dimethyl-5-oxo-4-thiophen-2-yl-1,4,5,6,7,8-hexahydro-quinoline-3-carboxylic acid ethyl ester;
2-(2-Amino-ethoxymethyl)-7,7-dimethyl-5-oxo-4-thiophen-2-yl-1,4,5,6,7,8-hexahydro-quinoline-3-carbonitrile;
2-(2-Amino-ethoxymethyl)-7,7-dimethyl-5-oxo-4-[1,3,5]triazin-2-yl-1,4,5,6,7,8-hexahydro-quinoline-3-carboxylic acid ethyl ester;
2-(2-Amino-ethoxymethyl)-7,7-dimethyl-5-oxo-4-[1,3,5]triazin-2-yl-1,4,5,6,7,8-hexahydro-quinoline-3-carbonitrile;
2-(2-Amino-ethoxymethyl)-7,7-dimethyl-5-oxo-4-thiazol-5-yl-1,4,5,6,7,8-hexahydro-quinoline-3-carboxylic acid ethyl ester;
2-(2-Amino-ethoxymethyl)-7,7-dimethyl-5-oxo-4-thiazol-5-yl-1,4,5,6,7,8-hexahydro-quinoline-3-carbonitrile;
2-(2-Amino-ethoxymethyl)-7,7-dimethyl-5-oxo-4-thiazol-2-yl-1,4,5,6,7,8-hexahydro-quinoline-3-carboxylic acid ethyl ester
2-(2-Amino-ethoxymethyl)-7,7-dimethyl-5-oxo-4-thiazol-2-yl-1,4,5,6,78-hexahydro-quinoline-3-carbonitrile;
2-(2-Amino-ethoxymethyl)-4-(1H-imidazol-2-yl)-7,7-dimethyl-5-oxo-1,4,5,6,7,8-hexahydro-quinoline-3-carboxylic acid ethyl ester;
2-(2-Amino-ethoxymethyl)-4-(1H-imidazol-2-yl)-7,7-dimethyl-5-oxo-1,4,5,6,7,8-hexahydro-quinoline-3-carbonitrile;
2-(2-Amino-ethoxymethyl)-4-(3H-imidazol-4-yl)-7,7-dimethyl-5-oxo-1,4,5,6,7,8-hexahydro-quinoline-3-carboxylic acid ethyl ester;
2-(2-Amino-ethoxymethyl)-4-(3H-imidazol-4-yl)-7,7-dimethyl-5-oxo-1,4,5,6,7,8-hexahydro-quinoline-3-carbonitrile;
2-(2-Amino-ethoxymethyl)-7,7-dimethyl-4-oxazol-5-yl-5-oxo-1,4,5,6,7,8-hexahydro-quinoline-3-carboxylic acid ethyl ester;
2-(2-Amino-ethoxymethyl)-7,7-dimethyl-4-oxazol-5-yl-5-oxo-1,4,5,6,7,8-hexahydro-quinoline-3-carbonitrile;
2-(2-Amino-ethoxymethyl)-7,7-dimethyl-4-oxazol-2-yl-5-oxo-1,4,5,6,7,8-hexahydro-quinoline-3-carboxylic acid ethyl ester;
2-(2-Amino-ethoxymethyl)-7,7-dimethyl-4-oxazol-2-yl-5-oxo-1,4,5,6,7,8-hexahydro-quinoline-3-carbonitrile;
2-(2-Amino-ethoxymethyl)-4-indolizin-6-yl-7,7-dimethyl-5-oxo-1,4,5,6,7,8-hexahydro-quinoline-3-carboxylic acid ethyl ester;
2-(2-Amino-ethoxymethyl)-4-indolizin-6-yl-7,7-dimethyl-5-oxo-1,4,5,6,7,8-hexahydro-quinoline-3-carbonitrile;
2-(2-Amino-ethoxymethyl)-4-indolizin-7-yl-7,7-dimethyl-5-oxo-1,4,5,6,7,8-hexahydro-quinoline-3-carboxylic acid ethyl ester;
2-(2-Amino-ethoxymethyl)-4-indolizin-7-yl-7,7-dimethyl-5-oxo-1,4,5,6,7,8-hexahydro-quinoline-3-carbonitrile;
2-(2-Amino-ethoxymethyl)-4-imidazo[1,2-a]pyridin-7-yl-7,7-dimethyl-5-oxo-1,4,5,6,7,8-hexahydro-quinoline-3-carboxylic acid ethyl ester;
2-(2-Amino-ethoxymethyl)-4-imidazo[1,2-a]pyridin-7-yl-7,7-dimethyl-5-oxo-1,4,5,6,7,8-hexahydro-quinoline-3-carbonitrile;
2-(2-Amino-ethoxymethyl)-4-imidazo[1,5-a]pyridin-7-yl-7,7-dimethyl-5-oxo-1,4,5,6,7,8-hexahydro-quinoline-3-carboxylic acid ethyl ester;
2-(2-Amino-ethoxymethyl)-4-imidazo[1,5-a]pyridin-7-yl-7,7-dimethyl-5-oxo-1,4,5,6,7,8-hexahydro-quinoline-3-carbonitrile;
2-(2-Amino-ethoxymethyl)-4-imidazo[1,5-a]pyridin-6-yl-7,7-dimethyl-5-oxo-1,4,5,6,7,8-hexahydro-quinoline-3-carboxylic acid ethyl ester;
2-(2-Amino-ethoxymethyl)-4-imidazo[1,5-a]pyridin-6-yl-7,7-dimethyl-5-oxo-1,4,5,6,7,8-hexahydro-quinoline-3-carbonitrile;
2-(2-Amino-ethoxymethyl)-4-imidazo[1,2-a]pyridin-6-yl-7,7-dimethyl-5-oxo-1,4,5,6,7,8-hexahydro-quinoline-3-carboxylic acid ethyl ester;
2-(2-Amino-ethoxymethyl)-4-imidazo[1,2-a]pyridin-6-yl-7,7-dimethyl-5-oxo-1,4,5,6,7,8-hexahydro-quinoline-3-carbonitrile;
2-(2-Amino-ethoxymethyl)-7,7-dimethyl-5-oxo-4-thiophen-3-yl-1,4,5,6,7,8-hexahydro-quinoline-3-carboxylic acid ethyl ester;
2-(2-Amino-ethoxymethyl)-7,7-dimethyl-5-oxo-4-thiophen-3-yl-1,4,5,6,7,8-hexahydro-quinoline-3-carbonitrile;
2-(2-Amino-ethoxymethyl)-4-furan-3-yl-7,7-dimethyl-5-oxo-1,4,5,6,7,8-hexahydro-quinoline-3-carboxylic acid ethyl ester;
2-(2-Amino-ethoxymethyl)-4-furan-3-yl-7,7-dimethyl-5-oxo-1,4,5,6,7,8-hexahydro-quinoline-3-carbonitrile;
2-(2-Amino-ethoxymethyl)-7,7-dimethyl-4-oxazol-4-yl-5-oxo-1,4,5,6,7,8-hexahydro-quinoline-3-carboxylic acid ethyl ester;
2-(2-Amino-ethoxymethyl)-7,7-dimethyl-4-oxazol-4-yl-5-oxo-1,4,5,6,7,8-hexahydro-quinoline-3-carbonitrile;
2-(2-Amino-ethoxymethyl)-7,7-dimethyl-5-oxo-4-thiazol-4-yl-1,4,5,6,7,8-hexahydro-quinoline-3-carboxylic acid ethyl ester;
2-(2-Amino-ethoxymethyl)-7,7-dimethyl-5-oxo-4-thiazol-4-yl-1,4,5,6,7,8-hexahydro-quinoline-3-carbonitrile
2-(2-Amino-ethoxymethyl)-4-imidazo[1,5-a]pyridin-1-yl-7,7-dimethyl-5-oxo-1,4,5,6,7,8-hexahydro-quinoline-3-carboxylic acid ethyl ester;
2-(2-Amino-ethoxymethyl)-4-imidazo 1,5-a]pyridin-1-yl-7,7-dimethyl-5-oxo-1,4,5,6,7,8-hexahydro-quinoline-3-carbonitrile;
2-(2-Amino-ethoxymethyl)-4-imidazo[1,5-a]pyridin-3-yl-7,7-dimethyl-5-oxo-1,4,5,6,7,8-hexahydro-quinoline-3-carboxylic acid ethyl ester;
2-(2-Amino-ethoxymethyl)-4-imidazo[1,5-a]pyridin-3-yl-7,7-5-oxo-1,4,5,6,7,8-hexahydro-quinoline-3-carbonitrile;
2-(2-Amino-ethoxymethyl)-4-imidazo[1,2-a]pyridin-3-yl-7,7-dimethyl-5-oxo-1,4,5,6,7,8-hexahydro-quinoline-3-carboxylic acid ethyl ester;
2-(2-Amino-ethoxymethyl)-4-imidazo[1,2-a]pyridin-3-yl-7,7-dimethyl-5-oxo-1,4,5,6,7,8-hexahydro-quinoline-3-carbonitrile;
2-(2-Amino-ethoxymethyl)-7,7-dimethyl-5-oxo-4-pyridin-3-yl-1,4,5,6,7,8-hexahydro-quinoline-3-carboxylic acid propyl ester;
2-(2-Amino-ethoxymethyl)-7,7-dimethyl-5-oxo-4-pyridin-3-yl-1,4,5,6,7,8-hexahydro-quinoline-3-carboxylic acid amide;
2-(2-Amino-ethoxymethyl)-7,7-dimethyl-5-oxo-4-pyridin-3-yl-1,4,5,6,7,8-hexahydro-quinoline-3-carboxylic acid methylamide;
2-(2-Amino-ethoxymethyl)-7,7-dimethyl-5-oxo-4-pyridin-3-yl-1,4,5,6,7,8-hexahydro-quinoline-3-carboxylic acid ethylamide;
3-Acetyl-2-(2-amino-ethoxymethyl)-7,7-dimethyl-4-pyridin-3-yl-4,6,7,8-tetrahydro-1H-quinolin-5-one;
2-(2-Amino-ethoxymethyl)-7,7-dimethyl-3-propionyl-4-pyridin-3-yl-4,6,7,8-tetrahydro-1H-quinolin-5-one;
2-(2-Amino-ethoxymethyl)-3-butyryl-7,7-dimethyl-4-pyridin-3-yl-4,6,7,8-tetrahydro-1 H-quinolin-5-one;
2-(2-Amino-ethoxymethyl)-7,7-dimethyl-3-(5-methyl-oxazol-2-yl)-4-pyridin-3-yl-4,6,7,8-tetrahydro-1 H-quinolin-5-one;
2-(2-Amino-ethoxymethyl)-7,7-dimethyl-3-(3-methyl-[1,2,4]oxadiazol-5-yl)-4-pyridin-3-yl-4,6,7,8-tetrahydro-1H-quinolin-5-one;
2-(2-Amino-ethoxymethyl)-7,7-dimethyl-3-(5-methyl-[1,2,4]oxadiazol-3-yl)-4-pyridin-3-yl-4,6,7,8-tetrahydro-1H-quinolin-5-one;
2-(2-Amino-ethoxymethyl)-7,7-dimethyl-3-oxazol-2-yl-4-pyridin-3-yl-4,6,7,8-tetrahydro-1H-quinolin-5-one;
2-(3H-Imidazol-4-ylmethoxymethyl)-7,7-dimethyl-5-oxo-4-pyridin-3-yl-1,4,5,6,7,8-hexahydro-quinoline-3-carboxylic acid ethyl ester;
2-(1H-Imidazol-2-ylmethoxymethyl)-7,7-dimethyl-5-oxo-4-pyridin-3-yl-1,4,5,6,7,8-hexahydro-quinoline-3-carboxylic acid ethyl ester;
2-(1H-Imidazol701-4-ylmethoxymethyl)-7,7-dimethyl-5-oxo-4-pyridin-3-yl-1,4,5,6,7,8-hexahydro-quinoline-3-carboxylic acid ethyl ester;
7,7-Dimethyl-5-oxo-4-pyridin-3-yl-2-(1H-pyrrol-3-ylmethoxymethyl)-1,4,5,6,7,8-hexahydro-quinoline-3-carboxylic acid ethyl ester;
7,7-Dimethyl-5-oxo-4-pyridin-3-yl-2-(1H-pyrrol-2-ylmethoxymethyl)-1,4,5,6,7,8-hexahydro-quinoline-3-carboxylic acid ethyl ester;
7,7-Dimethyl-5-oxo-4-pyridin-3-yl-2-(2H-[1,2,4]triazol-3-ylmethoxymethyl)-1,4,5,6,7,8-hexahydro-quinoline-3-carboxylic acid ethyl ester;
7,7-Dimethyl-5-oxo-2-(2H-pyrazol-3-ylmethoxymethyl)-4-pyridin-3-yl-1,4,5,6,7,8-hexahydro-quinoline-3-carboxylic acid ethyl ester;
7,7-Dimethyl-5-oxo-4-pyridin-3-yl-2-(3H-[1,2,3]triazol-4-ylmethoxymethyl)-1,4,5,6,7,8-hexahydro-quinoline-3-carboxylic acid ethyl ester;
7,7-Dimethyl-5-oxo-4-pyridin-3-yl-2-(2-pyrrol-1-yl-ethoxymethyl)-1,4,5,6,7,8-hexahydro-quinoline-3-carboxylic acid ethyl ester;
2-(2-Imidazol-1-yl-ethoxymethyl)-7,7-dimethyl-5-oxo-4-pyridin-3-yl-1,4,5,6,7,8-hexahydro-quinoline-3-carboxylic acid ethyl ester;
2-(3-Imidazol-1-yl-propyl)-7,7-dimethyl-5-oxo-4-pyridin-3-yl-1,4,5,6,7,8-hexahydro-quinoline-3-carboxylic acid ethyl ester;
7,7-Dimethyl-5-oxo-2-(3-pyrazol-1-yl-propyl)-4-pyridin-3-yl-1,4,5,6,7,8-hexahydro-quinoline-3-carboxylic acid ethyl ester;
7,7-Dimethyl-2-(2-morpholin-4-yl-ethoxymethyl)-5-oxo-4-pyridin-3-yl-1,4,5,6,7,8-hexahydro-quinoline-3-carboxylic acid ethyl ester;
2-(2-Amino-ethoxymethyl)-7,7-dimethyl-5-oxo-4-phenyl-1,4,5,6,7,8-hexahydro-quinoline-3-carboxylic acid ethyl ester;
2-(2-Amino-ethoxymethyl)-7,7-dimethyl-5-oxo-4-(2-trifluoromethyl-phenyl)-1,4,5,6,7,8-hexahydro-quinoline-3-carboxylic acid ethyl ester;
2-(2-Amino-ethoxymethyl)-7,7-dimethyl-5-oxo-4-(3-trifluoromethyl-phenyl)-1,4,5,6,7,8-hexahydro-quinoline-3-carboxylic acid ethyl ester;
2-(2-Amino-ethoxymethyl)-7,7-dimethyl-4-(4-nitro-phenyl)-5-oxo-1,4,5,6,7,8-hexahydro-quinoline-3-carboxylic acid ethyl ester;
2-(2-Amino-ethoxymethyl)-4-(3-hydroxy-phenyl)-7,7-dimethyl-5-oxo-1,4,5,6,7,8-hexahydro-quinoline-3-carboxylic acid ethyl ester;
2-(2-Amino-ethoxymethyl)-4-(3-methoxy-phenyl)-7,7-dimethyl-5-oxo-1,4,5,6,7,8-hexahydro-quinoline-3-carboxylic acid ethyl ester;
2-(2-Amino-ethoxymethyl)-4-(4-hydroxy-phenyl)-7,7-dimethyl-5-oxo-1,4,5,6,7,8-hexahydro-quinoline-3-carboxylic acid ethyl ester;
2-(2-Amino-ethoxymethyl)-4-(4-methoxy-phenyl)-7,7-dimethyl-5-oxo-1,4,5,6,7,8-hexahydro-quinoline-3-carboxylic acid ethyl ester;
2-(2-Amino-ethoxymethyl)-4-(3,5-dihydroxy-phenyl)-7,7-dimethyl-5-oxo-1,4,5,6,7,8-hexahydro-quinoline-3-carboxylic acid ethyl ester;
2-(2-Amino-ethoxymethyl)-4-(3,5-dimethoxy-phenyl)-7,7-dimethyl-5-oxo-1,4,5,6,7,8-hexahydro-quinoline-3-carboxylic acid ethyl ester;
2-(2-Amino-ethoxymethyl)-7,7-dimethyl-5-oxo-4-(3,4,5-trimethoxy-phenyl)-1,4,5,6,7,8-hexahydro-quinoline-3-carboxylic acid ethyl ester;
2-(2-Amino-ethoxymethyl)-7,7-dimethyl-5-oxo-4-(3,4,5-trihydroxy-phenyl)-1,4,5,6,7,8-hexahydro-quinoline-3-carboxylic acid ethyl ester;
2-(2-Amino-ethoxymethyl)-7,7-dimethyl-5-oxo-4-o-tolyl-1,4,5,6,7,8-hexahydro-quinoline-3-carboxylic acid ethyl ester;
2-(2-Amino-ethoxymethyl)-7,7-dimethyl-5-oxo-4-m-tolyl-1,4,5,6,7,8-hexahydro-quinoline-3-carboxylic acid ethyl ester;
2-(2-Amino-ethoxymethyl)-7,7-dimethyl-5-oxo-4-p-tolyl-1,4,5,6,7,8-hexahydro-quinoline-3-carboxylic acid ethyl ester;
2-(2-Amino-ethoxymethyl)-7,7-dimethyl-4-naphthalen-2-yl-5-oxo-1,4,5,6,7,8-hexahydro-quinoline-3-carboxylic acid ethyl ester;
2-(2-Amino-ethoxymethyl)-4-biphenyl-4-yl-7,7-dimethyl-5-oxo-1,4,5,6,7,8-hexahydro-quinoline-3-carboxylic acid ethyl ester;
2-(2-Amino-ethoxymethyl)-7,7-dimethyl-5-oxo-4-(2,4,6-trimethoxy-phenyl)-1,4,5,6,7,8-hexahydro-quinoline-3-carboxylic acid ethyl ester;
2-(2-Amino-ethoxymethyl)-4-(4-methoxy-3-methyl-phenyl)-7,7-dimethyl-5-oxo-1,4,5,6,7,8-hexahydro-quinoline-3-carboxylic acid ethyl ester;
2-(2-Amino-ethoxymethyl)-4-(2,3-dihydro-benzo [1,4]dioxin-6-yl)-7,7-dimethyl-5-oxo-1,4,5,6,7,8-hexahydro-quinoline-3-carboxylic acid ethyl ester;
2-(2-Amino-ethoxymethyl)-4-(4-methanesulfonyl-phenyl)-7,7-dimethyl-5-oxo-1,4,5,6,7,8-hexahydro-quinoline-3-carboxylic acid ethyl ester;
2-(2-Amino-ethoxymethyl)-4-(4-cyano-phenyl)-7,7-dimethyl-5-oxo-1,4,5,6,7,8-hexahydro-quinoline-3-carboxylic acid ethyl ester;
2-(2-Amino-ethoxymethyl)-4-(4-methoxy-naphthalen-1-yl)-7,7-dimethyl-5-oxo-1,4,5,6,7,8-hexahydro-quinoline-3-carboxylic acid ethyl ester;
2-(2-Amino-ethoxymethyl)-4-(2-methoxy-naphthalen-1-yl)-7,7-dimethyl-5-oxo-1,4,5,6,7,8-hexahydro-quinoline-3-carboxylic acid ethyl ester
2-(2-Amino-ethoxymethyl)-7,7-dimethyl-4-(4-methylsulfanyl-phenyl)-5-oxo-1,4,5,6,7,8-hexahydro-quinoline-3-carboxylic acid ethyl ester;
2-(2-Amino-ethoxymethyl)-4-(2-chloro-4-fluoro-phenyl)-7,7-dimethyl-5-oxo-1,4,5,6,7,8-hexahydro-quinoline-3-carboxylic acid ethyl ester;
2-(2-Amino-ethoxymethyl)-4-(2,4-dimethoxy-phenyl)-7,7-dimethyl-5-oxo-1,4,5,6,7,8-hexahydro-quinoline-3-carboxylic acid ethyl ester;
2-(2-Amino-ethoxymethyl)-4-(2,5-dimethoxy-phenyl)-7,7-dimethyl-5-oxo-1,4,5,6,7,8-hexahydro-quinoline-3-carboxylic acid ethyl ester;
2-(2-Amino-ethoxymethyl)-4-(2-fluoro-5-methoxy-phenyl)-7,7-dimethyl-5-oxo-1,4,5,6,7,8-hexahydro-quinoline-3-carboxylic acid ethyl ester; and
2-(2-Amino-ethoxymethyl)-7,7-dimethyl-3-nitro-4-pyridin-3-yl-4,6,7,8-tetrahydro-1H-quinolin-5-one;
2-(2-Amino-ethoxymethyl)-7,7-dimethyl-4-(2-methoxypyrid-3-yl)-5-oxo-1,4,5,6,7,8-hexahydro-quinoline-3-carboxylic acid ethyl ester;
2-(2-Amino-ethoxymethyl)-7,7-dimethyl-4-(3-chlorophenyl)-5-oxo-1,4,5,6,7,8-hexahydro-quinoline-3-carboxylic acid ethyl ester;
2-(2-Amino-ethoxymethyl)-7,7-dimethyl-4-(2,4-dichlorophenyl)-5-oxo-1,4,5,6,7,8-hexahydro-quinoline-3-carboxylic acid ethyl ester;
2-(2-Amino-ethoxymethyl)-7,7-dimethyl-4-(3-cyanophenyl)-5-oxo-1,4,5,6,7,8-hexahydro-quinoline-3-carboxylic acid ethyl ester;
2-(2-Amino-ethoxymethyl)-7,7-dimethyl-4-(2-chlorophenyl)-5-oxo-1,4,5,6,7,8-hexahydro-quinoline-3-carboxylic acid ethyl ester;
2-(2-Amino-ethoxymethyl)-7,7-dimethyl-4-(4-methylpyrid-3-yl)-5-oxo-1,4,5,6,7,8-hexahydro-quinoline-3-carboxylic acid ethyl ester;
2,7,7-Trimethyl-4-(2-methoxyphenyl)-5-oxo-1,4,5,6,7,8-hexahydro-quinoline-3-carboxylic acid ethyl ester;
2-(2-Amino-ethoxymethyl)-7,7-dimethyl-4-(4-methoxypyrid-3-yl)-5-oxo-1,4,5,6,7,8-hexahydro-quinoline-3-carboxylic acid ethyl ester;
2,7,7-Trimethyl-4-(pyrid-3-yl)-5-oxo-1,4,5,6,7,8-hexahydro-quinoline-3-carboxylic acid ethyl ester;
2,7,7-Trimethyl-4-(pyrid-3-yl)-5-oxo-1,4,5,6,7,8-hexahydro-quinoline-3-carbonitrile;
2-(2-Amino-ethyoxymethyl)-7,7-dimethyl-4-(2-methoxyphenyl)-5-oxo-1,4,5,6,7,8-hexahydro-quinoline-3-carbonitrile;
2-(2-Amino-ethoxymethyl)-7,7-dimethyl-4-(pyrid-3-yl)-5-oxo-1,4,5,6,7,8-hexahydro-quinoline-3-carboxylic acid ethyl ester;
2-(2-Amino-ethoxymethyl)-7,7-dimethyl-4-(5-methyl-furan-2-yl)-5-oxo-1,4,5,6,7,8-hexahydro-quinoline-3-carboxylic acid ethyl ester;
2-Propoxymethyl-7,7-dimethyl-4-(pyrid-3-yl)-5-oxo-1,4,5,6,7,8-hexahydro-quinoline-3-carboxylic acid ethyl ester;
2-Ethoxymethyl-7,7-dimethyl-4-(5-methyl-furan-2-yl)-5-oxo-1,4,5,6,7,8-hexahydro-quinoline-3-carboxylic acid ethyl ester;
2-(2-Amino-ethoxymethyl)-7,7-dimethyl-4-(5-chloro-6-methoxypyrid-3-yl)-5-oxo-1,4,5,6,7,8-hexahydro-quinoline-3-carboxylic acid ethyl ester;
2,7,7-Trimethyl-4-(5-methyl-furan-2-yl)-5-oxo-1,4,5,6,7,8-hexahydro-quinoline-3-carboxylic acid ethyl ester;
2-Isopropoxymethyl-7,7-dimethyl-4-(pyrid-3-yl)-5-oxo-1,4,5,6,7,8-hexahydro-quinoline-3-carboxylic acid ethyl ester;
2-(2-Amino-ethoxymethyl)-7,7-dimethyl-4-(6-hydroxypyrid-3-yl)-5-oxo-1,4,5,6,7,8-hexahydro-quinoline-3-carboxylic acid ethyl ester;
2,7,7-Trimethyl-4-(2-methoxyphenyl)-5-oxo-1,4,5,6,7,8-hexahydro-quinoline-3-carbonitrile;
2,7,7-Trimethyl-4-(2-chlorophenyl)-5-oxo-1,4,5,6,7,8-hexahydro-quinoline-3-carboxylic acid ethyl ester;
2-Methoxymethyl-4-cyclopropyl-5-oxo-7,7-dimethyl-1,4,5,6,7,8-hexahydro-quinoline-3-carboxylic acid ethyl ester;
2,7,7-Trimethyl-4-cyclopropyl-5-oxo-1,4,5,6,7,8-hexahydro-quinoline-3-carboxylic acid methyl ester;
2,4,7,7-Tetramethyl-5-oxo-1,4,5,6,7,8-hexahydro-quinoline-3-carboxylic acid ethyl ester;
2,7,7-Trimethyl-4-cyclohexyl-5-oxo-1,4,5,6,7,8-hexahydro-quinoline-3-carboxylic acid ethyl ester;
2,7,7-Trimethyl-4-propyl-5-oxo-1,4,5,6,7,8-hexahydro-quinoline-3-carboxylic acid ethyl ester;
2-(2-Amino-ethoxymethyl)-4-propyl-5-oxo-7,7-dimethyl-1,4,5,6,7,8-hexahydro-quinoline-3-carboxylic acid ethyl ester;
2,7,7-Trimethyl-4-cyclopentyl-5-oxo-1,4,5,6,7,8-hexahydro-quinoline-3-carboxylic acid ethyl ester
2,7,7-Trimethyl-4-cyclopropyl-5-oxo-1,4,5,6,7,8-hexahydro-quinoline-3-carbonitrile;
2-(Amino-ethoxymethyl)-4-cyclopentyl-5-oxo-7,7-dimethyl-1,4,5,6,7,8-hexahydro-quinoline-3-carboxylic acid ethyl ester;
2,7,7-Trimethyl-4-(t-butyl)-5-oxo-1,4,5,6,7,8-hexahydro-quinoline-3-carboxylic acid ethyl ester;
2,7,7-Trimethyl-4-(1-methyl-cyclopropyl)-5-oxo-1,4,5,6,7,8-hexahydro-quinoline-3-carboxylic acid ethyl ester;
2-Methyl-4-cyclopropyl-5-oxo-spiro[1,4,5,6,7,8-hexahydro-quinoline-7,1′-cyclopentane]-3-carboxylic acid ethyl ester;
2-(Morpholin-4-yl-methyl)-4-cyclopropyl-5-oxo-7,7-dimethyl-1,4,5,6,7,8-hexahydro-quinoline-3-carboxylic acid ethyl ester;
2-(Hydroxymethyl)-4-cyclopropyl-5-oxo-7,7-dimethyl-1,4,5,6,7,8-hexahydro-quinoline-3-carbonitrile;
2-[2-(Morpholin-4-yl)-ethoxymethyl]-4-cyclopropyl-5-oxo-7,7-dimethyl-1,4,5,6,7,8-hexahydro-quinoline-3-carboxylic acid ethyl ester;
2-(2-Amino-ethoxymethyl)-4-trifluoromethyl-5-oxo-7,7-dimethyl-1,4,5,6,7,8-hexahydro-quinoline-3-carboxylic acid ethyl ester;
2-Trifluoromethyl-4-cyclopropyl-5-oxo-7,7-dimethyl-1,4,5,6,7,8-hexahydro-quinoline-3-carboxylic acid ethyl ester;
2,4-Dicyclopropyl-5-oxo-7,7-dimethyl-1,4,5,6,7,8-hexahydro-quinoline-3-carboxylic acid ethyl ester;
2-Ethyl-4-cyclopropyl-5-oxo-7,7-dimethyl-1,4,5,6,7,8-hexahydro-quinoline-3-carboxylic acid ethyl ester;
2,7,7-Trimethyl-4-(cyclopropyl-methyl)-5-oxo-1,4,5,6,7,8-hexahydro-quinoline-3-carboxylic acid ethyl ester;
2-(Morpholin-4-yl-methyl)-4-(isopropyl)-5-oxo-7,7-dimethyl-1,4,5,6,7,8-hexahydro-quinoline-3-carboxylic acid ethyl ester;
2-[2-(Morpholin-4-yl)-ethoxymethyl]-4-(isopropyl)-5-oxo-7,7-dimethyl-1,4,5,6,7,8-hexahydro-quinoline-3-carboxylic acid ethyl ester; and
2-(2-Amino-ethoxymethyl)-4-cyclohexyl-5-oxo-7,7-dimethyl-1,4,5,6,7,8-hexahydro-quinoline-3-carboxylic acid ethyl ester;
and pharmaceutically acceptable salts, solvates, clathrates or polymorphs thereof.

68. A pharmaceutical composition comprising a compound according to claim 1, 2, 11, 12, 21, 23, 25, 26, 37, 38, 47, 48, 57, 58, or 67 and a pharmaceutically acceptable carrier or vehicle.

69. The pharmaceutical composition according to claim 68, further comprising a one or more additional therapeutic agents.

70. The pharmaceutical composition according to claim 69, wherein the additional therapeutic agent is selected from anti-diabetic agents, anti-obesity agents, lipid lowering agents or a suitable mixture thereof.

71. A method for treating or preventing a metabolic disorder in a subject, comprising administering to the subject an effective amount of a compound represented by the following structural formula: 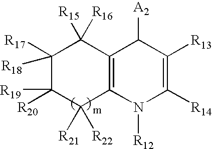
or a pharmaceutically acceptable salt, solvate, clathrate, or prodrug thereof wherein:
m is 0, 1, or2;
A₂ is an optionally substituted aryl or an optionally substituted heteroaryl;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉ and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is methyl, A₂ is not chlorophenyl, dichlorophenyl, p-nitrophenyl, or 5-chloro-benzo[1,3]dioxolyl;
provided that when R₁₄ is isopropyl or cyclopentyl, either R₁₃ is not p-(trifluoromethyl)benzoyl or A₂ is not p-fluorophenyl; and
provided that R₁₄ is not —NH₂.

72. A method for treating or preventing diabetes in a subject, comprising administering to the subject an effective amount of a compound represented by the following structural formula: 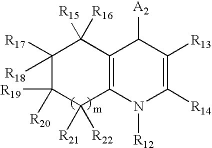
or a pharmaceutically acceptable salt, solvate, clathrate, or prodrug thereof wherein:
m is 0, 1, or 2;
A₂ is an optionally substituted aryl or an optionally substituted heteroaryl;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is methyl, A₂ is not chlorophenyl or 5-chloro-benzo[1,3]dioxolyl; and
provided that when R₁₄ is cyclopentyl, R₁₃ is not p-(trifluoromethyl)-benzoyl.

73. A method for reducing blood glucose levels in a subject, comprising administering to a subject an effective amount of a compound represented by the following structural formula: 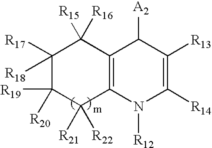
or a pharmaceutically acceptable salt, solvate, clathrate, or prodrug thereof wherein:
m is 0, 1,or 2;
A₂ is an optionally substituted aryl or an optionally substituted heteroaryl;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is methyl, A₂ is not chlorophenyl or 5-chloro-benzo[1,3]dioxolyl; and
provided that when R₁₄ is cyclopentyl, R₁₃ is not p-(trifluoromethyl)-benzoyl.

74. A method for increasing insulin sensitivity in a subject, comprising administering to the subject an effective amount of a compound represented by the following structural formula: 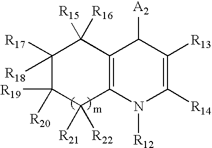
or a pharmaceutically acceptable salt, solvate, clathrate or prodrug thereof wherein:
m is 0, 1, or 2;
A₂ is an optionally substituted aryl or an optionally substituted heteroaryl;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each , independently, —H, —OR₂₃, or -NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is methyl, A₂ is not chlorophenyl or 5-chloro-benzo[1,3]dioxolyl; and
provided that when R₁₄ is cyclopentyl, R₁₃ is not p-(trifluoromethyl)-benzoyl.

75. A method for lowering triglyceride levels in a subject, comprising administering to the subject an effective amount of a compound represented by the following structural formula: 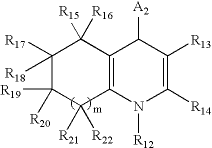
or a pharmaceutically acceptable salt, solvate, clathrate, or prodrug thereof wherein:
m is 0, 1, or 2;
A₂ is an optionally substituted aryl or an optionally substituted heteroaryl;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is isopropyl or cyclopentyl, either R₁₃ is not p-(trifluoromethyl)benzoyl or A₂ is not p-fluorophenyl;
provided that when R₁₄ is methyl, A₂ is not chlorophenyl, dichlorophenyl, p-nitrophenyl, or 5-chloro-benzo[1,3]dioxolyl; and
provided that R₁₄ is not —NH₂.

76. The method of claim 71, 72, 73, 74, or 75, wherein R₁₅ and R₁₆ together are ═O and m is 1.

77. The method of claim 76, wherein A₂ is selected from the group consisting of a substituted or unsubstituted phenyl, a substituted or unsubstituted benzo[1,3]dioxolyl, a substituted or unsubstituted pyridyl, a substituted or unsubstituted indolyl, a substituted or unsubstituted quinolinyl, a substituted or unsubstituted 1-oxo-pyridyl, a substituted or unsubstituted pyridazinyl, a substituted or unsubstituted pyrimidinyl, a substituted or unsubstituted pyrazinyl, a substituted or unsubstituted furanyl, a substituted or unsubstituted thienyl, a substituted or unsubstituted [1,3,5]triazinyl, a substituted or unsubstituted thiazolyl, a substituted or unsubstituted imidazolyl, a substituted or unsubstituted oxazolyl, a substituted or unsubstituted indolizinyl, a substituted or unsubstituted imidazo[1,2-a]pyridyl, a substituted or unsubstituted 2,3-dihydro-benzo[1,4]dioxinyl, and a substituted or unsubstituted naphthyl.

78. The method of claim 77, wherein A₂ is substituted with one, two or three substituents selected from the group consisting of halo, nitro, —NR₃₂R₃₂, lower alkyl, lower alkoxy, lower alkyl sulfanyl, lower haloalkyl, phenyl, hydroxyl, cyano, and lower alkyl sulfonyl, wherein R₃₂, for each occurrence, is —H or a lower alkyl.

79. The method of claim 76, wherein R₁₉ and R₂₀ are each, independently, a lower alkyl.

80. The method of claim 76, wherein R₁₃ is —C(O)O-(lower alkyl), —C(O)OH, cyano, —C(O)NR₃₂R₃₂, —C(O)-(lower alkyl), wherein R₃₂, for each occurrence, is —H or a lower alkyl.

81. The method of claim 76, wherein R₁₄ is cyclopropyl, ethoxymethyl, 2-amino-ethoxymethyl, 2-azido-ethoxymethyl, 2-(2-hydroxy-3-phenoxy-propylamino)-ethoxymethyl, propoxymethyl, isopropoxymethyl, N-mesyl-2-aminoethoxymethyl, N-acetyl-2-aminoethoxymethyl, N-ethyl-2-aminoethoxymethyl, N-methyl-2-aminoethoxymethyl, 2-(1,3-dioxo-1,3-dihydro-isoindol-2-yl)-ethoxymethyl, morpholin-4-yl-methyl, 2-morpholin-4-yl-ethoxymethyl, N,N-dimethylaminomethyl, carbethoxycarbonylmethoxymethyl, N-(2-hydroxyethyl)-N-methylaminomethyl, piperazin-1-yl-methyl, 2-hydroxyethoxymethyl, N,N-dimethylamino-ethoxymethyl, 4-aminobutyl, imidazol-5-yl-methoxymethyl, imidazol-4-yl-methoxymethyl, 2-imidazol-1-yl-ethoxymethyl, 3-imidazol-1-yl-propyl, 3-pyrazol-1-yl-propyl, propoxymethyl, isopropoxymethyl, methoxyethoxymethyl, pyrrol-3-yl-methoxymethyl, pyrrol-2-yl-methoxymethyl, [1,2,4]triazol-3-yl-methoxymethyl, 2H-pyrazol-3-yl-methoxymethyl, 3H-[1,2,3]triazol-4-yl-methoxymethyl, or 2-pyrrol-1-yl-ethoxymethyl.

82. The method of claim 76, wherein R₁₄ is a lower alkyl, a lower haloalkyl, a cycloalkyl, a —(C₁-C₆)alkyl-NHR₃₈, a —(C₁-C₆)alkyl-O—(C₁-C₆)alkyl-NHR₃₈, wherein R₃₈, for each occurrence is —S(O)—(C₁-C₆)alkyl, —S(O)₂—(C₁-C₆)alkyl, and —C(O)—(C₁-C₆)alkyl.

83. The method of claim 76, wherein R₁₄ is —NR₃₉R₄₀ or —OR₄₁, wherein:
R₃₉ and R₄₀ are each, independently, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂; or R₃₉ and R₄₀, taken together with the nitrogen to which they are attached are an optionally substituted heterocycloalkyl or optionally substituted heteroaryl;
R₄₁ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂;
R₄₂ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;and
R₄₃ and R₄₄ are each, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl, or R₄₃ and R₄₄ taken together with the nitrogen to which they are attached are an optionally substituted heterocycloalkyl or optionally substituted heteroaryl.

84. A method for treating or preventing a metabolic disorder in a subject, comprising administering to the subject an effective amount of a compound represented by the following structural formula: 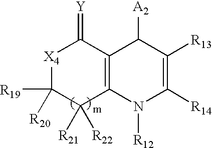
or a pharmaceutically acceptable salt, solvate, clathrate, or prodrug thereof wherein:
m is 0, 1, or 2;
A₂ is an optionally substituted aryl or an optionally substituted heteroaryl;
X₄ is O, S, or —NR₂₃—;
Y is O or S;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is methyl, A₂ is not chlorothienyl, methylthienyl, 1-oxo-pyridin-3-yl, 1-oxo-2-chloropyridin-3-yl, 1-oxo-2-methylpyridin-3-yl, 2-phenyl-4-oxo-thiochromenyl, a substituted 4-oxo-benzopyranyl, or a substituted phenyl; and
provided that when R₁₄ is methoxymethyl or (CH₃)₂NCH₂CH₂—, A₂ is not o-chlorophenyl.

85. A method for treating or preventing diabetes in a subject, comprising administering to the subject an effective amount of a compound represented by the following structural formula: 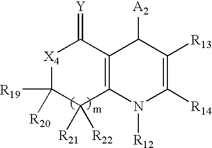
or a pharmaceutically acceptable salt, solvate, clathrate, or prodrug thereof wherein:
m is 0, 1, or 2;
A₂ is an optionally substituted aryl or an optionally substituted heteroaryl;
X₄ is O, S, or —NR₂₃—;
Y is O or S;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉ and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is methyl, A₂ is not chlorothienyl, methylthienyl, 2-phenyl-4-oxo-thiochromenyl, a substituted 4-oxo-benzopyranyl, or a substituted phenyl; and
provided that when R₁₄ is methoxymethyl or (CH₃)₂NCH₂CH₂—, A₂ is not o-chlorophenyl.

86. A method for reducing blood glucose levels in a subject, comprising administering to a subject an effective amount of a compound represented by the following structural formula: 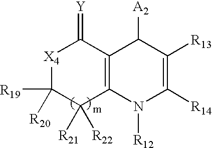
or a pharmaceutically acceptable salt, solvate, clathrate, or prodrug thereof wherein:
m is 0, 1, or 2;
A₂ is an optionally substituted aryl or an optionally substituted heteroaryl;
X₄ is O, S, or —NR₂₃—;
Y is O or S;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is methyl, A₂ is not chlorothienyl, methylthienyl, 2-phenyl-4-oxo-thiochromenyl, a substituted 4-oxo-benzopyranyl, or a substituted phenyl; and
provided that when R₁₄ is methoxymethyl or (CH₃)₂NCH₂CH₂—, A₂ is not o-chlorophenyl.

87. A method for increasing insulin sensitivity in a subject, comprising administering to the subject an effective amount of a compound represented by the following structural formula: 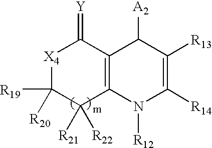
or a pharmaceutically acceptable salt, solvate, clathrate, or prodrug thereof wherein:
m is 0, 1,or 2;
A₂ is an optionally substituted aryl or an optionally substituted heteroaryl;
X₄ is O, S, or —NR₂₃—;
Y is O or S;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is methyl, A₂ is not chlorothienyl, methylthienyl, 2-phenyl-4-oxo-thiochromenyl, a substituted 4-oxo-benzopyranyl, or a substituted phenyl; and
provided that when R₁₄ is methoxymethyl or (CH₃)₂NCH₂CH₂—, A₂ is not o-chlorophenyl.

88. A method for lowering triglyceride levels in a subject, comprising administering to the subject an effective amount of a compound represented by the following structural formula: 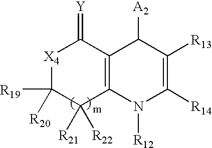
or a pharmaceutically acceptable salt, solvate, clathrate, or prodrug thereof wherein:
m is 0, 1, or 2;
A₂ is an optionally substituted aryl or an optionally substituted heteroaryl;
X₄ is O, S, or —NR₂₃—;
Y is O or S;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is methyl, A₂ is not 1-oxo-pyridin-3-yl, 1-oxo-2-chloropyridin-3-yl, or 1-oxo-2-methylpyridin-3-yl.

89. The method of claim 84, 85, 86, 87, or 88, wherein Y is ═O, X₄ is —O— and m is 1.

90. The method of claim 89, wherein A₂ is selected from the group consisting of a substituted or unsubstituted phenyl, a substituted or unsubstituted benzo[1,3]dioxolyl, a substituted or unsubstituted pyridyl, a substituted or unsubstituted indolyl, a substituted or unsubstituted quinolinyl, a substituted or unsubstituted 1-oxo-pyridyl, a substituted or unsubstituted pyridazinyl, a substituted or unsubstituted pyrimidinyl, a substituted or unsubstituted pyrazinyl, a substituted or unsubstituted furanyl, a substituted or unsubstituted thienyl, a substituted or unsubstituted [1,3,5]triazinyl, a substituted or unsubstituted thiazolyl, a substituted or unsubstituted imidazolyl, a substituted or unsubstituted oxazolyl, a substituted or unsubstituted indolizinyl, a substituted or unsubstituted imidazo[1,2-a]pyridyl, a substituted or unsubstituted 2,3-dihydro-benzo[1,4]dioxinyl, and a substituted or unsubstituted naphthyl.

91. The method of claim 90, wherein A₂ is substituted with one, two or three substituents selected from the group consisting of halo, nitro, —NR₃₂R₃₂, lower alkyl, lower alkoxy, lower alkyl sulfanyl, lower haloalkyl, phenyl, hydroxyl, cyano, and lower alkyl sulfonyl, wherein R₃₂, for each occurrence, is —H or a lower alkyl.

92. The method of claim 89, wherein R₁₉ and R₂₀ are each, independently, a lower alkyl.

93. The method of claim 89, wherein R₁₃ is —C(O)O-(lower alkyl), —C(O)OH, cyano, —C(O)NR₃₂R₃₂, —C(O)-(lower alkyl), wherein R₃₂, for each occurrence, is —H or a lower alkyl.

94. The method of claim 89, wherein R₁₄ is cyclopropyl, ethoxymethyl, 2-amino-ethoxymethyl, 2-azido-ethoxymethyl, 2-(2-hydroxy-3-phenoxy-propylamino)-ethoxymethyl, propoxymethyl, isopropoxymethyl, N-mesyl-2-aminoethoxymethyl, N-acetyl-2-aminoethoxymethyl, N-ethyl-2-aminoethoxymethyl, N-methyl-2-aminoethoxymethyl, 2-(1,3-dioxo-1,3-dihydro-isoindol-2-yl)-ethoxymethyl, morpholin-4-yl-methyl, 2-morpholin-4-yl-ethoxymethyl, N,N-dimethylaminomethyl, carbethoxycarbonylmethoxymethyl, N-(2-hydroxyethyl)—N-methylaminomethyl, piperazin-1-yl-methyl, 2-hydroxyethoxymethyl, N,N-dimethylamino-ethoxymethyl, 4-aminobutyl, imidazol-5-yl-methoxymethyl, imidazol-4-yl-methoxymethyl, 2-imidazol-1-yl-ethoxymethyl, 3-imidazol-1-yl-propyl, 3-pyrazol-1-yl-propyl, propoxymethyl, isopropoxymethyl, methoxyethoxymethyl, pyrrol-3-yl-methoxymethyl, pyrrol-2-yl-methoxymethyl, [1,2,4]triazol-3-yl-methoxymethyl, 2H-pyrazol-3-yl-methoxymethyl, 3H-[1,2,3]triazol-4-yl-methoxymethyl, or 2-pyrrol-1-yl-ethoxymethyl.

95. The method of claim 79, wherein R₁₄ is a lower alkyl, a lower haloalkyl, a cycloalkyl, a —(C₁-C₆)alkyl-NHR₃₈, a —(C₁-C₆)alkyl-O—(C₁-C₆)alkyl-NHR₃₈, wherein R₃₈, for each occurrence, is —S(O)—(C₁-C₆)alkyl, —S(O)₂—(C₁-C₆)alkyl, and —C(O)—(C₁-C₆)alkyl.

96. The method of claim 89, wherein R₁₄ is —NR₃₉R₄₀ or —OR₄₁, wherein:
R₃₉ and R₄₀ are each, independently, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂; or R₃₉ and R₄₀, taken together with the nitrogen to which they are attached are an optionally substituted heterocycloalkyl or optionally substituted heteroaryl;
R₄₁ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂;
R₄₂ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;and
R₄₃ and R₄₄ are each, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl, or R₄₃ and R₄₄ taken together with the nitrogen to which they are attached are an optionally substituted heterocycloalkyl or optionally substituted heteroaryl.

97. A method for treating or preventing a metabolic disorder, comprising administering to a subject in need thereof an effective amount of a compound represented by the following structural formula: 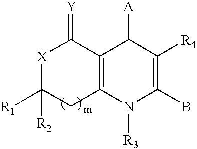
or a pharmaceutically acceptable salt, solvate, clathrate, hydrate, polymorph or prodrug thereof wherein:
A and B are independently selected from —H, -halo, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloatkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —(C₁-C₆)alkyl-Z-(C₁-C₁₀)alkyl-R₁₁, —(C₁-C₁₀)alkyl-R₁₁, —(C₁-C₁₀)alkyl-N(R₅)(R₅), —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, —S(O)₂R₅ and a substituted or unsubstituted aromatic or heteroaromatic ring, wherein if the ring is substituted, the substituents are independently selected from the group consisting of substituted or unsubstituted lower alkyl, -halo, —CN, —N(R₅)(R₅), —OR₆, —C(O)R₅, —C(O)₂R₅, —OC(O)R₅, —NO₂, and —C(O)N(R₅)(R₅), or two adjacent carbon atoms on the ring are linked by the group —O—(CH₂)_{q}—O— to form a bicyclic ring system, wherein q is an integer selected from 1, 2, 3 or 4;
X is selected from the group consisting of O, S, —NR₅, and —C(R₅)(R₅);
Y is O or S;
Z is at each occurrence independently —O—, —S—, —N(R₅)—, —C(O)—, —OC(O)—, —C(O)N(R₅)C(O)—, substituted or unsubstituted —(C₁-C₁₀)alkyl-, substituted or unsubstituted —(C₂-C₁₀)alkenyl-, substituted or unsubstituted —(C₂-C₁₀)alkynyl-, substituted or unsubstituted —(C₃-C₁₀)cycloalkyl-, substituted or unsubstituted —(C₈-C₁₄)bicycloalkyl-, substituted or unsubstituted —(C₅-C₁₀)cycloalkenyl-, substituted or unsubstituted —(C₃-C₁₀)heterocycle-, substituted or unsubstituted phenyl, substituted or unsubstituted naphthyl, substituted or unsubstituted benzyl, —C(O)O—, —C(O)OC(R₅)(R₅)—, —N(R₅)C(O)—, —N(R₅)C(O)NR₅—, —C(O)NR₅—, —OC(O)O—, —S(O)N(R₅)—, —S(O)— or —S(O)₂—;
R₁ and R₂ are at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;
R₃ is at each occurrence independently —H, —C(O)R₅ or substituted or unsubstituted —(C₁-C₁₀)alkyl;
R₄ is at each occurrence independently —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phneiyi, substituted o unsubstitited naphthyl, substituted or unsubstituted benzyl, —CO₂R₅, —C(O)OCH(R₅)(R₅), —NHC(O)R₅, —NHC(O)NHR₅, —C(O)NHR₅, —OC(O)OR₅, —S(O)N(R₅)(R₅), —SR₅, —S(O)R₅, —S(O)₂R₅ or a substituted or unsubstituted bioisosteric replacement of an ester;
each R₅ is at each occurrence independently H or substituted or unsubstituted —(C₁-C₁₀)alkyl;
each R₆ is at each occurrence independently H, substituted or unsubstituted —(C₁-C₁₀)alkyl or —(CH₂)ₚ—N(R₅)—(C₁-C₆)alkyl optionally substituted with one or more —OR₅ or —O-aryl groups;
R₁₁ is at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;
m is an integer selected from 0-2;
p is an integer selected from 1-6;
wherein if X is O m is not 0; and
if R₃ is other than H, R_{1 and R}₂ are not both H.

98. The method according to claim 97, wherein the disorder is diabetes mellitus, a condition associated with diabetes mellitus or complication of diabetes mellitus.

99. A method for reducing blood glucose levels in a subject in need thereof, comprising administering to the subject an effective amount of a compound represented by the following structural formula: 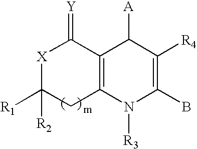
or a pharmaceutically acceptable salt, solvate, clathrate, hydrate, polymorph or prodrug thereof wherein:
A and B are independently selected from —H, -halo, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —(C₁-C₆)alkyl-Z-(C₁-C₁₀)alkyl-R₁₁, —(C₁-C₁₀)alkyl-R₁₁, —(C₁-C₁₀)alkyl-N(R₅)(R₅), —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, —S(O)₂R₅ and a substituted or unsubstituted aromatic or heteroaromatic ring, wherein if the ring is substituted, the substituents are independently selected from the group consisting of substituted or unsubstituted lower alkyl, -halo, —CN, —N(R₅)(R₅), —OR₆, —C(O)R₅, —C(O)₂R₅, —OC(O)R₅, —NO₂, and —C(O)N(R₅)(R₅), or two adjacent carbon atoms on the ring are linked by the group —O—(CH2)_{q}—O— to form a bicyclic ring system, wherein q is an integer selected from 1, 2, 3 or 4;
X is selected from the group consisting of O, S, —NR₅, and —C(R₅)(R₅);
Y is O or S;
Z is at each occurrence independently —O—, —S—, —N(R₅)—, —C(O)—, —OC(O)—, —C(O)N(R₅)C(O)—, substituted or unsubstituted —(C₁-C₁₀)alkyl-, substituted or unsubstituted —(C₂-C₁₀)alkenyl-, substituted or unsubstituted —(C₂-C₁₀)alkynyl-, substituted or unsubstituted —(C₃-C₁₀)cycloalkyl-, substituted or unsubstituted —(C₈-C₁₄)bicycloalkyl-, substituted or unsubstituted —(C₅-C₁₀)cycloalkenyl-, substituted or unsubstituted —(C₃-C₁₀)heterocycle-, substituted or unsubstituted phenyl, substituted or unsubstituted naphthyl, substituted or unsubstituted benzyl, —C(O)O—, —C(O)OC(R₅)(R₅)—, —N(R₅)C(O)—, —N(R₅)C(O)NR₅—, —C(O)NR₅—, —OC(O)O—, —S(O)N(R₅)—, —S(O)— or —S(O)₂—;
R_{1 and R}₂ are at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;
R₃ is at each occurrence independently —H, —C(O)R₅ or substituted or unsubstituted —(C₁-C₁₀)alkyl;
R₄ is at each occurrence independently —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)OR₅, —S(O)N(R₅)(R₅), —SR₅, —S(O)R₅, —S(O)₂R₅ or a substituted or unsubstituted bioisosteric replacement of an ester;
each R₅ is at each occurrence independently H or substituted or unsubstituted —(C₁-C₁₀)alkyl;
each R₆ is at each occurrence independently H, substituted or unsubstituted —(C₁-C₁₀)alkyl or —(CH₂)ₚ—N(R₅)—(C₁-C₆)alkyl optionally substituted with one or more —OR₅ or —O-aryl groups;
R₁₁ is at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;
m is an integer selected from 0-2; and
p is an integer selected from 1-6;
wherein if X is O, m is not 0; and
if R₃ is other than H, R₁ and R₂ are not both H.

100. A method for increasing insulin sensitivity in a subject in need thereof, comprising administering to the subject an effective amount of a compound represented by the following structural formula: 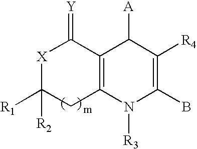
or a pharmaceutically acceptable salt, solvate, clathrate, hydrate, polymorph or prodrug thereof wherein:
A and B are independently selected from —H, -halo, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —(C₁-C₆)alkyl-Z-(C₁-C₁₀)alkyl-R₁₁, —(C₁-C₁₀)alkyl-R₁₁, —(C₁-C₁₀)alkyl-N(R₅)(R₅), —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, —S(O)₂R₅ and a substituted or unsubstituted aromatic or heteroaromatic ring, wherein if the ring is substituted, the substituents are independently selected from the group consisting of substituted or unsubstituted lower alkyl, -halo, —CN, —N(R₅)(R₅), —OR₆, —C(O)R₅, —C(O)₂R₅, —OC(O)R₅, —NO₂, and —C(O)N(R₅)(R₅), or two adjacent carbon atoms on the ring are linked by the group —O—(CH₂)_{q}—O— to form a bicyclic ring system, wherein q is an integer selected from 1, 2, 3 or 4;
X is selected from the group consisting of O, S, —NR₅, and —C(R₅)(R₅);
Y is O or S;
Z is at each occurrence independently —O—, —S—, —N(R₅)—, —C(O)—, —OC(O)—, —C(O)N(R₅)C(O)—, substituted or unsubstituted —(C₁-C₁₀)alkyl-, substituted or unsubstituted —(C₂-C₁₀)alkenyl-, substituted or unsubstituted —(C₂-C₁₀)alkynyl-, substituted or unsubstituted —(C₃-C₁₀)cycloalkyl-, substituted or unsubstituted —(C₈-C₁₄)bicycloalkyl-, substituted or unsubstituted —(C₅-C₁₀)cycloalkenyl-, substituted or unsubstituted —(C₃-C₁₀)heterocycle-, substituted or unsubstituted phenyl, substituted or unsubstituted naphthyl, substituted or unsubstituted benzyl, —C(O)O—, —C(O)OC(R₅)(R₅)—, —N(R₅)C(O)—, —N(R₅)C(O)NR₅—, —C(O)NR₅—, —OC(O)O—, —S(O)N(R₅)—, —S(O)— or —S(O)₂—;
R_{1 and R}₂ are at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naplithyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;
R₃ is at each occurrence independently —H, —C(O)R₅ or substituted or unsubstituted —(C₁-C₁₀)alkyl;
R₄ is at each occurrence independently —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C_{C}₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)OR₅, —S(O)N(R₅)(R₅), —SR₅, —S(O)R₅, —S(O)₂R₅ or a substituted or unsubstituted bioisosteric replacement of an ester;
each R₅ is at each occurrence independently H or substituted or unsubstituted —(C₁-C₁₀)alkyl;
each R₆ is at each occurrence independently H, substituted or unsubstituted —(C₁-C₁₀)alkyl or —(CH₂)ₚ—N(R₅)—(C₁-C₆)alkyl optionally substituted with one or more —OR₅ or —O-aryl groups;
R₁₁ is at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;
m is an integer selected from 0-2; and
p is an integer selected from 1-6.

101. A method for lowering elevated triglyceride levels in a subject in need thereof, comprising administering to the subject an effective amount of a compound represented by the following structural formula: 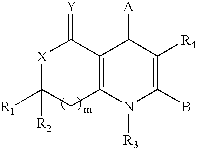
or a pharmaceutically acceptable salt, solvate, clathrate, hydrate, polymorph or prodrug thereof wherein:
A and B are independently selected from —H, -halo, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —(C₁-C₆)alkyl-Z-(C₁-C₁₀)alkyl-R₁₁, —(C₁-C₁₀)alkyl-R₁₁, —(C₁-₁₀)alkyl-N(R₅)(R₅), —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, —S(O)₂R₅ and a substituted or unsubstituted aromatic or heteroaromatic ring, wherein if the ring is substituted, the substituents are independently selected from the group consisting of substituted or unsubstituted lower alkyl, -halo, —CN, —N(R₅)(R₅), —OR₆, —C(O)R₅, —C(O)₂R₅, —OC(O)R₅, —NO₂, and —C(O)N(R₅)(R₅), or two adjacent carbon atoms on the ring are linked by the group —O—(CH₂)_{q}—O— to form a bicyclic ring system, wherein q is an integer selected from 1, 2, 3 or 4;
X is selected from the group consisting of O, S, —NR₅, and —C(R₅)(R₅);
Y is O or S;
Z is at each occurrence independently—O—, —S—, —N(R₅)—, —C(O)—, —OC(O)—, —C(O)N(R₅)C(O)—, substituted or unsubstituted —(C₁-C₁₀)alkyl-, substituted or unsubstituted —(C₂-C₁₀)alkenyl-, substituted or unsubstituted —(C₂-C₁₀)alkynyl-, substituted or unsubstituted —(C₃-C₁₀)cycloalkyl-, substituted or unsubstituted —(C₈-C₁₄)bicycloalkyl-, substituted or unsubstituted —(C₅-C₁₀)cycloalkenyl-, substituted or unsubstitited —(C₃-C₁₀)heterocycle-, substituted or unsubstituted phenyl, substituted or unsubstituted naphthyl, substituted or unsubstituted benzyl, —C(O)O—, —C(O)OC(R₅)(R₅)—, —N(R₅)C(O)—, —N(R₅)C(O)NR₅—, —C(O)NR₅—, —OC(O)O—, —S(O)N(R₅)—, —S(O)— or —S(O)₂—;
R₁ and R₂ are at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;
R₃ is at each occurrence independently —H, —C(O)R₅ or substituted or unsubstituted —(C₁-C₁₀)alkyl;
R₄ is at each occurrence independently —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)OR₅, —S(O)N(R₅)(R₅), —SR₅, —S(O)R₅, —S(O)₂R₅ or a substituted or unsubstituted bioisosteric replacement of an ester;
each R₅ is at each occurrence independently H or substituted or unsubstituted —(C₁-C₁₀)alkyl;
each R₆ is at each occurrence independently H, substituted or unsubstituted —(C₁-C₁₀)alkyl or —(CH₂)ₚ—N(R₅)—(C₁-C₆)alkyl optionally substituted with one or more —OR₅ or —O-aryl groups;
R₁₁ is at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;
m is an integer selected from 0-2; and
p is an integer selected from 1-6.

102. The method of claim 97, 99, 100, or 101, wherein the compound has the structure: 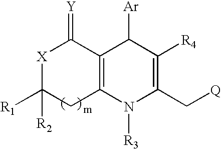
or a pharmaceutically acceptable salt, solvate, clathrate, hydrate, polymorph or prodrug thereof, wherein:
Ar is a substituted or unsubstituted aromatic or heteroaromatic ring, wherein if the ring is substituted, the substituents are independently selected from the group consisting of substituted or unsubstituted lower alkyl, -halo, —CN, —N(R₅)(R₅), —OR₆, —C(O)R₅, —C(O)₂R₅, —OC(O)R₅, —NO₂, and —C(O)N(R₅)(R₅), or two adjacent carbon atoms on the ring are linked by the group —O—(CH₂)_{q}—O— to form a bicyclic ring system, wherein q is an integer selected from 1, 2, 3 or 4;
Q is H, -halo, —NO₂, —CN, —OH, —N(R₅)(R₅), —OR₅, —C(O)R₅, —OC(O)R₅, —C(O)NHC(O)R₅, substituted or unsubstituted —(C₂-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —(C₀-C₆)alkyl-Z-(C₁-C₁₀)alkyl-R₁₁, —(C₁-C₁₀)alkyl-R₁₁, —CO₂R₅, —C(O)OCH(R₅)(R₅), —NHC(O)R_{—NHC(O)NHR}₅, —C(O)NHR₅, —OC(O)R₅, —OC(O)OR₅, —S(O)N(R₅)(R₅), —SR₅, —S(O)R₅, —S(O)₂R₅ or a substituted or unsubstituted aromatic or heteroaromatic ring, wherein if the ring is substituted, the substituents are independently selected from the group consisting of substituted or unsubstituted lower alkyl, -halo, —CN, —N(R₅)(R₅), —OR₆, —C(O)R₅, —C(O)₂R₅, —OC(O)R₅, —NO₂, and —C(O)N(R₅)(R₅);
X is selected from the group consisting of O, S, —NR₅, and —C(R₅)(R₅);
Y is O or S;
Z is at each occurrence independently —O—, —S—, —N(R₅)—, —C(O)—, —OC(O)—, —C(O)N(R₅)C(O)—, substituted or unsubstituted —(C₁-C₁₀)alkyl-, substituted or unsubstituted —(C₂-C₁₀)alkenyl-, substituted or unsubstituted —(C₂-C₁₀)alkynyl-, substituted or unsubstituted —(C₃-C₁₀)cycloalkyl-, substituted or unsubstituted —(C₈-C₁₄)bicycloalkyl-, substituted or unsubstituted —(C₅-C₁₀)cycloalkenyl-, substituted or unsubstituted —(C₃-C₁₀)heterocycle-, substituted or unsubstituted phenyl, substituted or unsubstituted naphthyl, substituted or unsubstituted benzyl, —C(O)O—, —C(O)OC(R₅)(R₅)—, —N(R₅)C(O)—, —N(R₅)C(O)NR₅—, —C(O)NR₅—, —OC(O)O—, —S(O)N(R₅)—, —S(O)—, or —S(O)₂—;
R₁ and R₂ are at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)atkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;
R₃ is at each occurrence independently —H, —C(O)R₅ or substituted or unsubstituted —(C₁-C₁₀)alkyl;
R₄ is at each occurrence independently —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)atkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)OR₅, —S(O)N(R₅)(R₅), —SR₅, —S(O)R₅, —S(O)₂R₅ or a substituted or unsubstituted bioisosteric replacement of an ester;
each R₅ is at each occurrence independently H or substituted or unsubstituted —(C₁-C₁₀)alkyl;
each R₆ is at each occurrence H, substituted or unsubstituted —(C₁-C₁₀)alkyl or —(CH₂)ₚ—N(R₅)—(C₁-C₆)alkyl optionally substituted with one or more —OR₅ or —O-aryl groups;
R₁₁ is at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;
m is an integer selected from 0-2; and
p is an integer selected from 1-6.

103. The method of claim 97, 99, 100, or 101 wherein the compound has the structure: 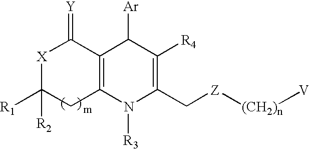
or a pharmaceutically acceptable salt, solvate, clathrate, hydrate, polymorph or prodrug thereof wherein:
Ar is a mono- or poly-substituted or unsubstituted aromatic or heteroaromatic ring, wherein if the ring is substituted, the substituents are independently selected from the group consisting of substituted or unsubstituted lower alkyl, -halo, —CN, —N(R₅)(R₅), —OR₆, —C(O)R₅, —C(O)₂R₅, —OC(O)R₅, —NO₂, and —C(O)N(R₅)(R₅), or two adjacent carbon atoms on the ring are linked by the group —O—(CH₂)_{q}—O— to form a bicyclic ring system, wherein q is an integer selected from 1, 2, 3 or 4;
V is H, -halo, N₃, —NO₂, —CN, —OH, —N(R₅)(R₇), —OR₅, —C(O)R₅, —OC(O)R₅, —C(O)NHC(O)R₅, substituted or unsubstituted 3-7 membered monocyclic heterocycle or substituted or unsubstituted 8-12 membered bicyclic heterocycle;
X is selected from the group consisting of O, S, —NR₅, and —C(R₅)(R₅);
Y is O or S;
Z is —O—, —S—, —N(R₅)—, —C(O)—, —OC(O)—, —C(O)N(R₅)C(O)—, substituted or unsubstituted —(C₁-C₁₀)alkyl-, substituted or unsubstituted —(C₂-C₁₀)alkenyl-, substituted or unsubstituted —(C₂-C₁₀)alkynyl-, substituted or unsubstituted —(C₃-C₁₀)cycloalkyl-, substituted or unsubstituted —(C₈-C₁₄)bicycloalkyl-, substituted or unsubstituted —(C₅-C₁₀)cycloalkenyl-, substituted or unsubstituted —(C₃-C₁₀)heterocycle-, substituted or unsubstituted phenyl, substituted or unsubstituted naphthyl, substituted or unsubstituted benzyl, —C(O)O—, —C(O)OC(R₅)(R₅)—, —N(R₅)C(O)—, —N(R₅)C(O)NR₅—, —C(O)NR₅—, —OC(O)O—, —S(O)N(R₅)—, —S(O)—, or —S(O)2—;
R₁ and R₂ are at each occurrence independently selected from —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)R₅, —OC(O)OR₅, —S(O)N(R₅)(R₅), —SR₅, —S(O)R₅, and —S(O)₂R₅;
R₃ is at each occurrence independently —H, —C(O)R₅ or substituted or unsubstituted —(C₁-C₁₀)alkyl;
R₄ is at each occurrence independently —H, -halo, —CN, —N₃, —NO₂, —CN, —OH, —N(R₅)(R₅), —OR₅, —C(O)R₅, —OC(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl, substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —OC(O)OR₅, —S(O)N(R₅)(R₅), —SR₅, —S(O)R₅, —S(O)₂R₅ or a substituted or unsubstituted bioisosteric replacement of an ester;
each R₅ is at each occurrence independently H or substituted or unsubstituted —(C₁-C₁₀)alkyl;
each R₆ is at each occurrence independently H, substituted or unsubstituted —(C₁-C₁₀)alkyl or —(CH₂)ₚ—N(R₅)—(C₁-C₆)alkyl optionally substituted with one or more —OR₅ or —O-aryl groups;
R₇ is selected from the group consisting of H and substituted or unsubstituted —(C₁-C₁₀)alkyl optionally substituted with one or more —OR₅ or —O-aryl groups;
n is an integer selected from 1-10;
m is an integer selected from 0-2; and
p is an integer selected from 1-6.

104. The method of claim 97, 99, 100, or 101, wherein the compound has the structure: 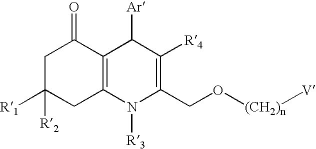
or a pharmaceutically acceptable salt, solvate, clathrate, hydrate, polymorph or prodrug thereof wherein:
AR′ is phenyl or pyridyl, which may be unsubstituted or independently substituted with one or more substituted or unsubstituted lower alkyl, -halo, —CN, —N(R′₅)(R′₅), —OR′₅, —C(O)R′₅, —C(O)₂R′₅, —OC(O)R′₅, —NO₂, or —C(O)N(R′₅)(R′₅) groups, or two adjacent carbon atoms on the phenyl or pyridyl are linked by the group —O—(CH₂)_{q}—O— to form a bicyclic ring system, wherein q is an integer selected from 1, 2, 3 or 4;
V′ is H, N(R′₁₁)(R′₁₁), N₃, substituted or unsubstituted 3-7 membered monocyclic heterocycle or substituted or unsubstituted 8-12 membered bicyclic heterocycle;
each R′₁, and R′₂ may be independently selected from H and substituted or unsubstituted lower alkyl;
R′₃ is —C(O)R₅, —H, or substituted or unsubstituted lower alkyl;
R′₄ is —CN, —CO₂-lower alkyl, —C(O)NHR₅ or a bioisosteric replacement of an ester;
each R′₅ is at each occurrence independently H or substituted or unsubstituted —(C₁-C₁₀) alkyl;
R₁₁′ is at each occurrence independently selected from —H, —OH, —N(R₅)(R₅), —OR₅, —C(O)R₅, —C(O)NHC(O)R₅, substituted or unsubstituted —(C₁-C₁₀)alkyl, substituted or unsubstituted —(C₂-C₁₀)alkenyl, substituted or unsubstituted —(C₂-C₁₀)alkynyl, substituted or unsubstituted —(C₃-C₁₀)cycloalkyl, substituted or unsubstituted —(C₈-C₁₄)bicycloalkyl, substituted or unsubstituted —(C₅-C₁₀)cycloalkenyl. substituted or unsubstituted 3-7 membered monocyclic heterocycle, substituted or unsubstituted 8-12 membered bicyclic heterocycle, substituted or unsubstituted phenyl, substituted or unsubstituted naphthyl, substituted or unsubstituted benzyl, —CO₂R₅, —C(O)OCH(R₅)(R₅), —NHC(O)R₅, —NHC(O)NHR₅, —C(O)NHR₅, —S(O)N(R₅)(R₅), —SR₅, —S(O)R₅, and —S(O)₂R₅; and
n is an integer selected from the group consisting of 1, 2, 3 and 4.

105. A method for treating or preventing a metabolic disorder in a subject, comprising administering to the subject an effective amount of a compound represented by the following structural formula: 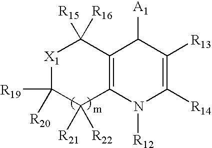
or a pharmaceutically acceptable salt, solvate, clathrate, or prodrug thereof wherein:
m is 0, 1, or 2;
A₁ is an optionally substituted alkyl, an optionally substituted alkenyl, an optionally substituted alkynyl, an optionally substituted cycloalkyl, an optionally substituted cycloalkenyl, or an optionally substituted heterocycloalkyl;
X₁ is O, S, —NR₂₃—, or >CR₁₇R₁₈;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independenitly, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is isopropyl or cyclopentyl, R₁₃ is not p-(trifluoromethyl)benzoyl.

106. A method for treating or preventing diabetes in a subject, comprising administering to the subject an effective amount of a compound represented by the following structural formula: 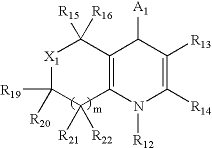
or a pharmaceutically acceptable salt, solvate, clathrate, or prodrug thereof wherein:
m is 0, 1, or 2;
A₁ is an optionally substituted alkyl, an optionally substituted alkenyl, an optionally substituted alkynyl, an optionally substituted cycloalkyl, an optionally substituted cycloalkenyl, or an optionally substituted heterocycloalkyl;
X₁ is O, S, —NR₂₃—, or >CR₁₇R₁₈;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkeny, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is isopropyl or cyclopentyl, R₁₃ is not p-(trifluoromethyl)benzoyl.

107. A method for reducing blood glucose levels in a subject, comprising administering to a subject an effective amount of a compound represented by the following structural formula: 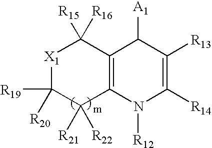
or a pharmaceutically acceptable salt, solvate, clathrate, or prodrug thereof wherein:
m is 0, 1,or 2;
A₁ is an optionally substituted alkyl, an optionally substituted alkenyl, an optionally substituted alkynyl, an optionally substituted cycloalkyl, an optionally substituted cycloalkenyl, or an optionally substituted heterocycloalkyl;
X₁, is O, S, —NR₂₃—, or >CR₁₇R₁₈;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁, and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is isopropyl or cyclopentyl, R₁₃ is not p-(trifluoromethyl)benzoyl.

108. A method for increasing insulin sensitivity in a subject, comprising administering to the subject an effective amount of a compound represented by the following structural formula: 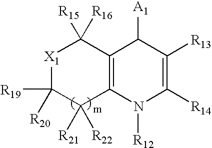
or a pharmaceutically acceptable salt, solvate, clathrate, or prodrug thereof wherein:
m is 0, 1, or 2;
A₁ is an optionally substituted alkyl, an optionally substituted alkenyl, an optionally substituted alkynyl, an optionally substituted cycloalkyl, an optionally substituted cycloalkenyl, or an optionally substituted heterocycloalkyl;
X₁ is O, S, —NR₂₃—, or >CR₁₇R₁8;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is isopropyl or cyclopentyl, R₁₃ is not p-(trifluoromethyl)benzoyl.

109. A method for lowering triglyceride levels in a subject, comprising administering to the subject an effective amount of a compound represented by the following structural formula: 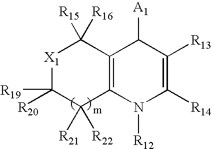
or a pharmaceutically acceptable salt, solvate, clathrate, or prodrug thereof wherein:
m is 0, 1,or 2;
A₁ is an optionally substituted alkyl, an optionally substituted alkenyl, an optionally substituted alkynyl, an optionally substituted cycloalkyl, an optionally substituted cycloalkenyl, or an optionally substituted heterocycloalkyl;
X₁ is O, S, —NR₂₃—, or >CR₁₇R₁₈;
R₁₂ is —H or an alkyl;
R₁₃ is —C(O)OR₂₃, —C(O)R₂₃, —C(O)NR₂₄R₂₅, —CN, —CH(OR₂₃)R₂₃, —C(═NR₂₃)R₂₃, —C(S)R₂₃, —C(S)OR₂₃, —C(S)NR₂₄R₂₅, —CH(NR₂₄R₂₅)R₂₃; or —CH(SR₂₃)R₂₃;
R₁₄ is H or a substituent;
R₁₅ and R₁₆ are each, independently, —H, —OR₂₃, or —NR₂₄R₂₅; or R₁₅ and R₁₆ taken together are ═O, ═S or ═NR₂₆, provided that at least one of R₁₅ or R₁₆ is not —H;
R₁₇ and R₁₈ are each, independently, —H or a substituent;
R₁₉, and R₂₀ are each, independently, —H or a substituent; or R₁₉ and R₂₀, together with the carbon to which they are attached, form an optionally substituted cycloalkyl;
R₂₁ and R₂₂, for each occurrence, are, independently, —H or a substituent;
R₂₃, for each occurrence, is, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;
R₂₄ and R₂₅, for each occurrence, are, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₂₄ and R₂₅, taken together with the nitrogen to which they are attached are an optionally substituted heterocycloalkyl or optionally substituted heteroaryl;
R₂₆ is —H, halo, —OR₂₇, —NR₂₇R₂₇, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; and
R₂₇ is H, alkyl, aryl or acetyl;
provided that when R₁₄ is isopropyl or cyclopentyl, R₁₃ is not p-(trifluoromethyl)benzoyl.

110. The method of claim 105, 106, 107, 108, or 109, wherein R₁₅ and R₁₆ together are ═O, X₁ is >CR₁₇R₁₈, and m is 1.

111. The method of claim 110, wherein A₁ is methyl, isopropyl, cyclopropyl, cyclopentyl, cyclohexyl, 1-methylcyclopropyl, or cyclopropylmethyl.

112. The method of claim 110, wherein R₁₉ and R₂₀ are each, independently, a lower alkyl.

113. The method of claim 110, wherein R₁₃ is —C(O)O-(lower alkyl), —C(O)OH, cyano, —C(O)NR₃₂R₃₂, —C(O)-(lower alkyl), wherein R₃₂, for each occurrence, is —H or a lower alkyl.

114. The method of claim 110, wherein R₁₄ is cyclopropyl, ethoxymethyl, 2-amino-ethoxymethyl, 2-azido-ethoxymethyl, 2-(2-hydroxy-3-phenoxy-propylamino)-ethoxymethyl, propoxymethyl, isopropoxymethyl, N-mesyl-2-aminoethoxymethyl, N-acetyl-2-aminoethoxymethyl, N-ethyl-2-aminoethoxymethyl, N-methyl-2-aminoethoxymethyl, 2-(1,3-dioxo-1,3-dihydro-isoindol-2-yl)-ethoxymethyl, morpholin-4-yl-methyl, 2-morpholin-4-yl-ethoxymethyl, N,N-dimethylaminomethyl, carbethoxycarbonylmethoxymethyl, N-(2-hydroxyethyl)—N-methylaminomethyl, piperazin-1-yl-methyl, 2-hydroxyethoxymethyl, N,N-dimethylamino-ethoxymethyl, 4-aminobutyl, imidazol-5-yl-methoxymethyl, imidazol-4-yl-methoxymethyl, 2-imidazol-1-yl-ethoxymethyl, 3-imidazol-1-yl-propyl, 3-pyrazol-1-yl-propyl, propoxymethyl, isopropoxymethyl, methoxyethoxymethyl, pyrrol-3-yl-methoxymethyl, pyrrol-2-yl-methoxymethyl, [1,2,4]triazol-3-yl-methoxymethyl, 2H-pyrazol-3-yl-methoxymethyl, 3H-[1,2,3]triazol-4-yl-methoxymethyl, or 2-pyrrol-1-yl-ethoxymethyl.

115. The method of claim 110, wherein R₁₄ is a lower alkyl, a lower haloalkyl, a cycloalkyl, a —(C₁-C₆)alkyl-NHR₃₈, a —(C₁-C₆)alkyl-O—(C₁-C₆)alkyl-NHR₃₈, wherein R₃₈, for each occurrence, is —S(O)—(C₁-C₆)alkyl, —S(O)₂—(C₁-C₆)alkyl, and —C(O)—(C₁-C₆)alkyl.

116. The method of claim 110, wherein R₁₄ is —NR₃₉R₄₀ or —OR₄₁, wherein:
R₃₉ and R₄₀ are each, independently, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂; or R₃₉ and R₄₀, taken together with the nitrogen to which they are attached are an optionally substituted heterocycloalkyl or optionally substituted heteroaryl;
R₄₁ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, an optionally substituted heteraralkyl, —C(O)R₄₂, —C(O)OR₄₂, —C(O)NR₄₃R₄₄, —S(O)₂R₄₂, or —S(O)R₄₂;
R₄₂ is —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl;and
R₄₃ and R₄₄ are each, independently, —H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl, or R₄₃ and R₄₄ taken together with the nitrogen to which they are attached are an optionally substituted heterocycloalkyl or optionally substituted heteroaryl.

117. A kit comprising a compound according to claim 1, 2, 11, 12, 21, 23, 25, 26, 37, 38, 47, 48, 57, 58, or 67 and a device for administering the compound.

118. The kit of claim 117, comprising a label or printed instructions for use of the kit.

119. A 3-substituted-1,4-dihydropyridine compound characterized by an ability to reduce elevated blood glucose levels without a significant cardiovascular effect, wherein the core scaffold of the compound is a 1,4-dihydropyridine.

120. A 4-substituted-1,4,5,6,7,8-hexahydroquinoline compound characterized by an ability to reduce elevated blood glucose levels without a significant cardiovascular effect, wherein the core scaffold of the compound is a 1,4,5,6,7,8-hexahydro-quinoline.

121. The compound of claim 120, further comprising a 5-oxo substituent.

122. The compound of claim 120, wherein the molecular weight of the compound is about 300 g/mol to about 500 g/mol.

123. A pharmaceutical composition comprising a compound of claim 120, or a pharmaceutically acceptable salt, solvate, or hydrate thereof, and a pharmaceutically acceptable carrier, diluent or excipient.

124. A method for reducing blood glucose levels in a subject in need thereof, comprising administering to the subject an effective amount of a compound of claim 120.

125. A method for increasing insulin sensitivity in a subject in need thereof, comprising administering to the subject an effective amount of a compound of claim 120.

126. A method for lowering elevated triglyceride levels in a subject in need thereof, comprising administering to the subject an effective amount of a compound of claim 120.

127. The method of claim 71, 72, 73, 74, 75, 84, 85, 86, 87, 88, 97, 98, 99, 100, 103, 105, 106, 107, 108, 109, 119, or 120, further comprising administering one or more additional therapeutic agent selected from the group consisting of anti-diabetic agents, anti-obesity agents, lipid lowering agents, and combinations thereof.
